(19) United States
(12) Patent Application Publication
(10) Pub. No.: US 2024/0285681 A1
(43) Pub. Date: Aug. 29, 2024

(54) CELLS FOR TREATING INFECTIONS

(71) Applicant: LIfT BioSciences Ltd, Brighton (GB)

(72) Inventor: Alex Blyth, Brighton (GB)

(21) Appl. No.: 17/785,008

(22) PCT Filed: Dec. 11, 2020
(86) PCT No.: PCT/GB2020/053197

§ 371 Date: Jun. 13, 2022

(30) Foreign Application Priority Data
Dec. 12, 2019 (GB) 1918341.7

(51) Int. Cl. A61K 35/15; A61P 31/04; C12N 5/0787

(52) U.S. Cl. CPC A61K 35/15; A61P 31/04; C12N 5/0642; C12N 2502/70; C12N 2506/45

(57) ABSTRACT

The invention relates to a granulocyte or stem cell (preferably granulocyte) for use in treating an infection. The invention also relates to said methods for identifying said granulocytes and stem cells capable of differentiating into said granulocytes, compositions and kits comprising the same, as well as uses of the same for treating an infection.

The present invention relates to a cell-based therapy suitable for treating infections.

Pathogens such as bacteria, fungi and viruses cause a multitude of diseases, many of which are contagious and life threatening. The majority of infections are currently treated with antimicrobial drugs (e.g. chemical substances), which generally act by fatally disrupting a cellular/molecular process of the pathogen.

For example, antibiotics have been used in the treatment of bacterial infections for over 100 years, many of which disrupt DNA or protein synthesis of target bacteria. These antibiotics typically act on multiplying bacteria and have little efficacy against non-multiplying bacteria or persister cells, and therefore fail to clear the whole bacterial population. As a result, longer durations of antibiotic therapy are required, exacerbating the emergence of antibiotic resistance. This increasing emergence of antibiotic resistance has resulted in infectious disease becoming the second leading cause of mortality in the world, with drug-resistant bacteria killing approximately 25,000 people per year in Europe alone.

This rise in antibiotic resistance has not been offset by an increase in the availability of new antibiotics, as the discovery and synthesis of new antibiotics has proven notoriously difficult. The same is true of chemical drugs which target other types of pathogen (e.g. anti-fungal and anti-viral agents). Furthermore, these drugs are generally very specific in nature, requiring the appropriate drug treatment and dosage to be identified for each type of infection, such that very few therapies are suitable for treating a multitude of infectious diseases.

There therefore exists a need for alternative therapies for the treatment of infectious diseases, which overcome the issues of drug (e.g. antibiotic) resistance and new infectious strains for which there are no therapies or vaccinations currently available (e.g. new viruses) and which can be employed in the treatment of various infectious diseases. There also exists a need for methods of identifying and producing such therapies.

The present invention addresses one or more of the above mentioned problems.

The present invention is predicated on the surprising finding that granulocytes can be isolated that have particular efficacy in the treatment of infections and/or overcome/reduce the need for conventional therapeutics, such as chemical antibiotics, against which pathogens are becoming increasingly resistant.

Thus, in one aspect the invention provides a granulocyte of the invention for use in treating an infection. In a related aspect, the invention also provides use of a granulocyte of the invention in the manufacture of a medicament for treating an infection as well as a method for treating an infection, the method comprising administering to a subject in need thereof the granulocyte according to the invention.

Granulocytes, such as neutrophils, generally act by ingesting and killing microorganisms or dying cells, or by secreting toxic proteins. They can recognise and kill both bacteria and fungi (e.g. following recognition through the presence of Pathogen Recognition Receptors (PRRs)), as well as viruses and larger (e.g. macroparasitic) pathogens such as helminths (“worms”). Thus, the generic modes of action of granulocyte cells make them particularly suitable for combating a variety of pathogen types (and thus a variety of infections/infectious diseases).

The present inventors have demonstrated the utility of granulocytes for killing different types of bacteria (both Gram negative and Gram positive). Advantageously, the granulocytes of the invention show improved efficacy against antibiotic-resistant (e.g. multiple antibiotic-resistant) bacteria. Moreover, the inventors have surprisingly shown that such granulocytes kill bacteria more rapidly than conventional chemical antibiotics.

Antibiotic resistance represents one of the largest public health concerns globally. An example of an antibiotic resistant bacterium is methicillin-resistant Staphylococcus aureus (MRSA), which has acquired resistance to a number of antibiotics (e.g. multiple drug resistance to beta-lactam antibiotics). Surprisingly, the present inventors have succeeded in providing granulocytes (and stem cells that differentiate into granulocytes) that have particular efficacy against MRSA.

In one aspect, there is provided a method for producing a granulocyte for treating an infection, the method comprising:
a. providing a stem cell obtainable from a sample from a donor, wherein a granulocyte of the donor kills a greater % of infective agents or cells infected by an infective agent when compared to a control;
b. differentiating the stem cell into a granulocyte; and
c. optionally isolating the granulocyte.

In another aspect, there is provided a method for producing a stem cell for treating an infection, the method comprising:
a. providing a stem cell obtainable from a sample from a donor, wherein a granulocyte of the donor kills a greater % of infective agents or cells infected by an infective agent when compared to a control;
b. differentiating the stem cell into a different stem cell (preferably a precursor cell); and
c. optionally isolating the different stem cell (preferably the precursor cell).

In embodiments where the stem cell is a precursor cell, the different stem cell may be a different precursor cell.

A control sample for use in a method of the invention may be a sample comprising granulocytes from a donor that has granulocytes that are unsuitable for treating an infection (e.g. granulocytes that do not kill greater than 41.23% of the infective agent or cells infected by an infective agent in a method/assay described herein). In other embodiments, the control sample referred to may be a sample comprising granulocytes from a donor that has granulocytes that are suitable for treating an infection (e.g. granulocytes that do kill greater than 41.23% of the infective agent or cells infected by an infective agent in a method/assay described herein), in which case the method may be used to detect donors having granulocytes with optimal therapeutic activity. Preferably, a control sample for use in a method of the invention is a sample comprising granulocytes from a donor that has granulocytes that are unsuitable for treating an infection.

Preferably, the control sample comprises an infective agent or cells infected by an infective agent of the same type and a granulocyte obtainable from a different donor or wherein the granulocyte kills greater than 41.23% of the infective agent or cells infected by an infective agent in an admixture.

Another aspect provides a method for selecting a granulocyte for treating an infection, said method comprising:
a. contacting an infective agent or a cell infected by an infective agent with a granulocyte obtainable from a donor to form a test sample, and incubating said test sample; and
b. obtaining a granulocyte from a sample from said donor when the % of infective agent or cells infected by an infective agent killed in the test sample is greater than the % of infective agent or cells infected by an infective agent killed in a control sample, wherein the control sample comprises an infective agent or cells infected by an infective agent of the same type and a granulocyte obtainable from a different donor.

In another aspect, there is provided an in vitro method for obtaining a granulocyte for treating an infection, said method comprising obtaining a granulocyte from a sample obtainable from a donor wherein said donor produces granulocytes for treating an infection.

In one embodiment a granulocyte is obtained from a sample from said donor when the % of infective agent or cells infected by an infective agent killed in the test sample is at least 5% greater than the % of infective agent or cells infected by an infective agent killed in the control sample. In some embodiments the % of infective agent or cells infected by an infective agent killed in the test sample is at least 10%, 15%, 20%, 25%, 30%, 40%, 50%, 60%, 70% or 80% greater than the % of infective agent or cells infected by an infective agent killed in the control sample. The skilled person would appreciate that reference to “kill”, “killing an infective agent”, “an infective agent killed” or the like encompasses “inactivate”, “inactivating an infective agent” or “an infective agent inactivated” or the like.

The methods of the invention may comprise the use of an infective agent or cells infected by an infective agent. In a preferred embodiment, methods of the invention comprise the use of an infective agent.

In one embodiment a granulocyte is produced when the % of infective agent or cells infected by an infective agent killed in the sample from a donor is at least 5% greater than the % of infective agent or cells infected by an infective agent killed in the control sample. In some embodiments the % of infective agent or cells infected by an infective agent killed in the test sample is at least 10%, 15%, 20%, 25%, 30%, 40%, 50%, 60%, 70% or 80% greater than the % of infective agent or cells infected by an infective agent killed in the control sample. The control sample preferably comprises an infective agent or cells infected by an infective agent of the same type and a granulocyte obtainable from a different donor.

Reference to “treating an infection” embraces alleviating the symptoms of an infection.

In a further finding, the present inventors have surprisingly found that it is possible to select for stem cells (e.g. haematopoietic stem cells) that are capable of differentiating into granulocytes having the ability to kill pathogens (e.g. bacteria). Once such a stem cell (e.g. haematopoietic stem cell) has been selected from a donor, said cell can be either stored for subsequent therapeutic purposes, or used directly as a medicament, for example in the treatment of an infection. Advantageously stem cells (e.g. haematopoietic stem cells) obtainable by such a method of the invention can be immortalised thus providing a stable cell line that can be stored and/or propagated indefinitely. The present invention thus reduces the need for multiple rare donors, and/or for direct transfer of granulocytes collected from a donor to an infected patient. Thus the invention provides a viable, scalable, safe and/or reliable therapy.

For the first time, the present inventors have shown that the infective agent (e.g. bacteria) killing efficacy of granulocytes (e.g. neutrophils) is genetically-defined, rather than epigenetically-defined. In particular, it has been shown that granulocytes derived from stem cells (e.g. haematopoietic stem cells) isolated from a donor (who produces granulocytes suitable for treating an infection) have greater infective agent killing efficacy than mature granulocytes isolated directly from the same donor.

Advantageously, donors found to have granulocytes with a high infective agent killing activity can be used as a source of stem cells (e.g. haematopoietic stem cells) which can be differentiated into granulocytes with similarly high infective agent killing activity.

Such stem cells can advantageously be stored, and used for the production of high volumes of granulocytes for use in treating infections, thus overcoming problems of isolating sufficient quantities of fresh granulocytes from a donor.

Thus, a further aspect of the invention provides a method for obtaining a stem cell for treating an infection, said method comprising:
a. contacting an infective agent or cell infected by an infective agent with a granulocyte obtainable from a donor to form a test sample, and incubating said test sample; and
b. obtaining a stem cell from a sample from said donor when the % of infective agent or cells infected by an infective agent killed in the test sample is greater than the % of pathogens or pathogen-infected cells killed in a control sample, wherein the control sample comprises an infective agent or cells infected by an infective agent of the same type and a granulocyte obtainable from a different donor.

In another aspect, there is provided an in vitro method for obtaining a stem cell for treating an infection, said method comprising obtaining a stem cell from a sample obtainable from a donor wherein said donor produces granulocytes for treating an infection.

In one embodiment a stem cell is obtained when the % of infective agent or cells infected by an infective agent killed in the sample from a donor is at least 5% greater than the % of infective agent or cells infected by an infective agent killed in the control sample. In some embodiments the % of infective agent or cells infected by an infective agent killed in the test sample is at least 10%, 15%, 20%, 25%, 30%, 40%, 50%, 60%, 70% or 80% greater than the % of infective agent or cells infected by an infective agent killed in the control sample.

The skilled person understands that where the methods of the invention comprise a comparison step between two samples (e.g. between a “test sample” and a “control sample”) that conditions (e.g. assay conditions during the method) should be kept consistent. For example, the concentration ratio of granulocytes to infective agent or cells infected by an infective agent should be the same, as should the time conditions, etc. Where a comparison is made between two samples herein, suitably the samples are equivalent. For example, the samples being compared may be the same sample types (e.g. blood) and subjected to the same processing steps. In some embodiments the only difference between samples is the donor from which said samples are obtained. For example, in embodiments where the proportion of cells having a particular property is being determined, the total number of cells in each sample may be the same so that a proper comparison can be made.

The % of infective agent or cells infected by an infective agent killed in a control may be determined either prior to carrying out the present method or at the same time as carrying out the present method (preferably at the same time).

In some embodiments the method may comprise the use of a plurality of different test samples comprising granulocytes from further donors (e.g. second, third, fourth donors, etc.).

According to one aspect, there is provided a method for obtaining a stem cell for treating an infection, said method comprising:
a. admixing a granulocyte obtainable from a donor with an infective agent or cells infected by an infective agent to form an admixture;
b. incubating said admixture;
c. measuring the % of infective agent or cells infected by an infective agent killed in said admixture; and
d. obtaining a stem cell from a sample from said donor when said granulocyte kills greater than 41.23% of the infective agent or cells infected by an infective agent in the admixture (suitably when the granulocyte from said subject kills at least 60%, preferably at least 80% or 90%, of the infective agent or cells infected by an infective agent in the test sample).

In a related aspect there is provided a method for selecting a granulocyte for treating an infection, said method comprising:
a. admixing a granulocyte with an infective agent or cells infected by an infective agent;
b. incubating said admixture;
c. measuring the % of infective agent or cells infected by an infective agent killed in said admixture; and
d. selecting a granulocyte that kills greater than 41.23% of the infective agent or cells infected by an infective agent in the admixture (suitably when the granulocyte from said subject kills at least 50% or 60%, preferably at least 80% or 90%, of the infective agent or cells infected by an infective agent in the test sample).

In one embodiment an infection is an infection with an infective agent (used synonymously herein with the term “infectious agent”). An infective agent may refer to a bacterium, a fungus, a virus, a macroparasite (e.g. a helminth), or a combination thereof. Preferably, an infective agent is a bacterium or a virus. For example, in one embodiment, an infective agent is a bacterium. In an alternative embodiment, an infective agent is a virus. In a preferred embodiment an infective agent is a pathogen.

Thus, in one embodiment the infection is one or more selected from bacterial, fungal, viral, macroparasitic, or a combination thereof (preferably bacterial).

As used herein the term “pathogen” refers to a microorganism that can cause disease and may also encompass opportunistic pathogens. The pathogen may be one or more selected from a pathogenic bacterium, a pathogenic fungus, a pathogenic virus, a pathogenic macroparasite (e.g. a pathogenic helminth), or a combination thereof. Preferably, the pathogen is a pathogenic bacterium or a pathogenic virus. For example, in one embodiment the pathogen is a pathogenic bacterium. In an alternative embodiment, the pathogen is a pathogenic virus.

As used herein, a “cell infected by an infective agent” refers to a cell that is infected by an intracellular infective agent. Said intracellular infective agent is preferably a pathogen and the cell is therefore a “cell infected by a pathogen”. In one embodiment a cell may be infected by an intracellular bacterium or a virus, preferably a virus.

In one embodiment an infective agent is a Gram-negative bacterium or a Gram-positive bacterium. Preferably, an infective agent is a Gram-positive bacterium, such as a bacterium from the genus Staphylococcus.

A bacterium may be selected from one or more of Staphylococcus spp., multidrug resistant gram-negative bacteria (MRDGN bacteria), vancomycin-resistant Enterococcus (VRE), Mycobacterium spp., carbapenem-resistant Enterobacteriaceae (CRE) gut bacteria, Acinetobacter spp., Actinomyces spp., Propionibacterium spp., Anaplasma spp., Bacillus spp., Arcanobacterium spp., Bacteroides spp., Bartonella spp., Brucella spp., Yersinia spp., Burkholderia spp., Campylobacter spp., Streptococcus spp., Haemophilus spp., Clostridium spp., Corynebacterium spp., Echinococcus spp., Ehrlichia spp., Enterococcus spp., Rickettsia spp., Fusobacterium spp., Neisseria spp., Klebsiella spp., Helicobacter spp., Escherichia spp., Kingella spp., Legionella spp., Listeria spp., Borrelia spp., Mycoplasma spp., Chlamydia spp., Nocardia spp., Pasteurella spp., Bordetella spp., Prevotella spp., Chlamydophila spp., Coxiella spp., Salmonella spp., Group A Streptococcus spp., Shigella spp., Staphylococcus spp., Treponema spp., Vibrio spp., Francisella spp., Pseudomonas spp. and Ureaplasma spp.

In one embodiment the bacterium is selected from one or more of methicillin resistant Staphylococcus aureus (MRSA), multi-drug resistant Mycobacterium tuberculosis (MDR-TB), Pseudomonas aeruginosa, Pseudomonas oryzihabitans, Pseudomonas plecoglossicida, Acinetobacter baumannii, Actinomyces israelii, Actinomyces gerencseriae, Propionibacterium propionicus, Bacillus anthracis, Arcanobacterium haemolyticum, Bacillus cereus, Yersinia pestis, Mycobacterium ulcerans, Campylobacter jejuni, Bartonella bacilliformis, Bartonella henselae, Haemophilus ducreyi, Clostridium difficile, Corynebacterium diphtheria, Burkholderia mallei, Neisseria gonorrhoeae, Klebsiella granulomatis, Streptococcus pyogenes, Streptococcus agalactiae, Haemophilus influenzae, Helicobacter pylori, Escherichia coli (e.g. O157:H7, O111 and O104:H4), Kingella kingae, Legionella pneumophila, Listeria monocytogenes, Burkholderia pseudomallei, Neisseria meningitidis, Mycoplasma pneumoniae, Mycoplasma genitalium, Chlamydia trachomatis, Bordetella pertussis, Streptococcus pneumoniae, Chlamydophila psittaci, Coxiella burnetii, Treponema pallidum, Clostridium tetani, Chlamydophila pneumoniae, Vibrio cholera, Mycobacterium tuberculosis, Salmonella enterica subsp. enterica, serovartyphi, Ureaplasma urealyticum, and Francisella tularensis. Preferably Mycobacterium tuberculosis.

Preferably, in one embodiment the bacterium is selected from one or more of methicillin resistant Staphylococcus aureus (MRSA), multidrug resistant gram-negative bacteria (MRDGN bacteria), vancomycin-resistant Enterococcus (VRE), multi-drug resistant Mycobacterium tuberculosis (MDR-TB), and carbapenem-resistant Enterobacteriaceae (CRE) gut bacteria.

A virus may be selected from one or more family selected from Adenoviridae, Picornaviridae, Herpesviridae, Coronaviridae, Hepadnaviridae, Flaviviridae, Retroviridae, Orthomyxoviridae, Paramyxoviridae, Papovaviridae, Polyomavirus, Rhabdoviridae, Togaviridae and Bunyaviridae.

In one embodiment the virus may be selected from one or more of HIV-1 (Human immunodeficiency virus), HIV-2, Junin virus, BK virus, Machupo virus, Sabia virus, Varicella zoster virus (VZV), Alphavirus, Colorado tick fever virus (CTFV), Rhinoviruses, Crimean-Congo hemorrhagic fever virus, Cytomegalovirus, Dengue virus, Ebolavirus (EBOV), Parvovirus B19, Human herpesvirus 6 (HHV-6), Human herpesvirus 7 (HHV-7), Enteroviruses (e.g. EV71), Coxsackie A virus, Sin Nombre virus, Heartland virus, Hanta virus, Hendra virus, Hepatitis A virus, Hepatitis B virus, Hepatitis C virus, Hepatitis D Virus, Hepatitis E virus, Herpes simplex virus 1 and 2 (HSV-1 and HSV-2), Human bocavirus (HBoV), Human metapneumovirus (hMPV), Human papillomaviruses, Human parainfluenza viruses (HPIV), Epstein-Barr virus (EBV), Lassa virus, Lymphocytic choriomeningitis virus (LCMV), Marburg virus, Measles virus, Middle East respiratory syndrome coronavirus, Molluscum contagiosum virus (MCV), Monkeypox virus, Mumps virus, Nipah virus, Norovirus, Poliovirus, JC virus, Respiratory syncytial virus (RSV), Rhinovirus, Rift Valley fever virus, Rotavirus, Rubella virus, SARS coronavirus, SARS-CoV-2, Variola major, Variola minor, Venezuelan equine encephalitis virus, Guanarito virus, West Nile virus, Yellow fever virus, and Zika virus.

A fungus may be selected from one or more of Aspergillus spp., Piedraia spp., Blastomyces spp., Candida spp., Fonsecaea spp., Coccidioides spp., Cryptococcus spp., Cryptosporidium spp., Geotrichum spp., Histoplasma spp., Microsporidia phylum, Paracoccidioides spp., Pneumocystis spp., Sporothrix spp., Trichophyton spp., Epidermophyton spp., Hortaea spp., Malassezia spp., Trichosporon spp., and Mucorales order.

In one embodiment the pathogen is a fungus selected from one or more of Aspergillus fumigatus, Aspergillus flavus, Piedraia hortae, Blastomyces dermatitidis, Candida albicans, Fonsecaea pedrosoi, Coccidioides immitis, Coccidioides posadasii, Cryptococcus neoformans, Geotrichum candidum, Histoplasma capsulatum, Paracoccidioides brasiliensis, Pneumocystis jirovecii, Sporothrix schenckii, Trichophyton tonsurans, Epidermophyton floccosum, Hortaea werneckii, and Trichosporon beigelii.

A macroparasite may be one or more selected from Angiostrongylus spp., Entamoeba Anisakis spp., Ascaris spp., Babesia spp., Balantidium spp., Baylisascaris spp., Blastocystis spp., Capillaria spp., Trypanosoma spp., Clonorchis spp., Ancylostoma spp., Cyclospora spp., Taenia spp., Desmodesmus spp., Dientamoeba spp., Dracunculus spp., Enterobius spp., Fasciola spp., Filarioidea superfamily, Giardia spp., Gnathostoma spp., Necator spp., Hymenolepis spp., Isospora spp., Leptospira spp., Wuchereria spp., Rhinosporidium spp., Brugia spp., Plasmodium spp., Onchocerca spp., Opisthorchis spp., Paragonimus spp., Naegleria spp., Schistosoma spp., Strongyloides spp., Toxocara spp., Toxoplasma spp., Trichinella spp., Trichomonas spp., and Trichuris spp.

In one embodiment the macroparasite is selected from one or more of Entamoeba histolytica, Ascaris lumbricoides, Balantidium coli, Trypanosoma brucei, Trypanosoma cruzi, Clonorchis sinensis, Cyclospora cayetanensis, Taenia solium, Desmodesmus armatus, Dientamoeba fragilis, Dracunculus medinensis, Enterobius vermicularis, Fasciolopsis buski, Giardia lamblia, Necator americanus, Hymenolepis nana, Hymenolepis diminuta, Isospora belli, Wuchereria bancrofti, Rhinosporidium seeberi, Brugia malayi, Plasmodium vivax, Plasmodium falciparum, Plasmodium malariae, Plasmodium ovale, Plasmodium knowlesi Onchocerca volvulus, Opisthorchis viverrini, Opisthorchis felineus, Naegleria fowleri, Strongyloides stercoralis, Toxoplasma gondii, Trichinella spiralis, Trichuris trichiura, and Trichomonas vaginalis.

In one embodiment, the infective agent is an antibiotic-resistant bacterium (e.g. MRSA), preferably a multi-antibiotic resistant bacterium. An antibiotic resistant bacterium may be resistant to beta-lactams, such as methicillin.

Antibiotic resistance may be assessed using any technique known in the art, such as the Kirby-Baure method, Stokes method, Etest, and/or agar and broth dilution methods for minimum inhibitory concentration (MIC) determination.

In one embodiment a bacterium is resistant to one or more of a penicillin, a penicillinase-resistant penicillin, a cephalosporin, a beta-lactamase inhibitor, a tetracycline and combinations thereof, or pharmaceutically acceptable salts thereof.

In one embodiment a bacterium is resistant to one or more of: vancomycin, nafcillin, oxacillin, teicoplanin, penicillin, methicillin, flucloxacillin, dicloxacillin, cefazolin, cephalothin, cephalexin, cefuroxime, clindamycin, cefazolin, amoxicillin/clavulanate, ampicillin/sulbactam, lincomycin, erythromycin, trimethoprim, sulfamethoxazole, daptomycin, linezolid, rifampin, ciprofloxacin, gentamycin, tetracycline, doxycycline, minocylcine, tigecycline and combinations thereof or pharmaceutically acceptable salts thereof. In one embodiment a bacterium may be resistant to vancomycin and/or teicoplanin, or pharmaceutically acceptable salts thereof.

A multi-antibiotic resistant bacterium is resistant to at least 2, 3, 4, 5, 6, 7, 8, 9 or 10 antibiotics (e.g. chemical antibiotics).

In one embodiment, a granulocyte or stem cell of the invention kills an infective agent by phagocytosing a cell infected by the infective agent. For example, in one embodiment, a granulocyte or stem cell of the invention kills a virus by phagocytosing a cell infected by the virus. In one embodiment, a granulocyte or stem cell of the invention kills a bacterium by phagocytosing a cell infected by the bacterium. In one embodiment, a granulocyte or stem cell of the invention kills an infective agent by releasing one or more factors which kill the infective agent. For example, in one embodiment, a granulocyte or stem cell of the invention kills a virus by releasing one or more factors which kill the virus. In one embodiment, a granulocyte or stem cell of the invention kills a bacterium by releasing one or more factors which kill the bacterium. In some embodiments, a granulocyte or stem cell of the invention kills an infective agent by a combination of the above.

In one embodiment the invention provides a method for selecting a granulocyte suitable for treating multi-antibiotic resistant Pseudomonas aeruginosa, said method comprising:
a. admixing a granulocyte with multi-antibiotic resistant Pseudomonas aeruginosa cells to form an admixture;
b. incubating said admixture;
c. measuring the % of multi-antibiotic resistant Pseudomonas aeruginosa cells killed in said admixture; and
d. selecting a granulocyte that kills greater than 41.23% of the multi-antibiotic resistant Pseudomonas aeruginosa cells in the admixture.

In one embodiment a method comprises selecting a granulocyte that kills at least 50%, 60%, 70% or 80% of the multi-antibiotic resistant Pseudomonas aeruginosa cells in the admixture.

Preferably, the invention provides a method for selecting a granulocyte suitable for treating MRSA, said method comprising:
a. admixing a granulocyte with MRSA cells to form an admixture;
b. incubating said admixture;
c. measuring the % of MRSA cells killed in said admixture; and
d. selecting a granulocyte that kills greater than 41.23% of the MRSA cells in the admixture.

In one embodiment a method comprises selecting a granulocyte that kills at least 50%, 60% or 70% of the MRSA cells in the admixture.

The incubation step or contacting between a granulocyte and an infective agent/infective agent-infected cell may be carried out for between 1 hour and 100 hours. Preferably, the incubation step or contacting between a granulocyte and an infective agent/infective agent-infected cell may be carried out for between 5 hours and 75 hours, for example between 10 hours and 20 hours. The incubation step or contacting between a granulocyte and an infective agent/infective agent-infected cell may be carried out for between 6 hours to 6 days. Suitably, the incubation step or contacting between a granulocyte and an infective agent/infective agent-infected cell may be carried out for between 6 hours and 2 days, for example for between 12 hours to 36 hours, such as between 16 to 24 hours. In one embodiment the incubation step is carried out for 24 hours. In another embodiment the incubation step or contacting between a granulocyte and an infective agent/infective agent-infected cell is carried out for 48 hours. The incubation step or contacting between a granulocyte and an infective agent/infective agent-infected cell may be carried out at any temperature suitable for cell growth and viability, for example at a temperature between 35° C. to 42° C., suitably at 37 or 39° C. Preferably the incubation step or contacting between a granulocyte and an infective agent/infective agent-infected cell step is carried out at 37 or 39° C. for 24 hours. Preferably the incubation step or contacting between a granulocyte and an infective agent/infective agent-infected cell is carried out for 16-24 hours at 30-40° C. (e.g. 37° C.).

The above-mentioned conditions may be particularly suitable when incubating/contacting a granulocyte with a cell infected by an infective agent.

The incubation step or contacting between a granulocyte and an infective agent/infective agent-infected cell may be carried out for between 30 minutes and 24 hours (e.g. prior to assessing % killing). Preferably, the incubation step or contacting between a granulocyte and an infective agent/infective agent-infected cell may be carried out for between 1-3 hours, for example for 2 hours. In other words, the assessment of % killing may be determined following contacting/incubating for 2 hours. The incubation step or contacting between a granulocyte and an infective agent/infective agent-infected cell may be carried out at any temperature suitable for cell growth and viability, for example at a temperature between 35° C. to 42° C., suitably at 37° C.

The above-mentioned conditions may be particularly suitable when incubating/contacting a granulocyte with an infective agent, such as a bacterium.

In one embodiment a contacting or incubation step is carried out in solution. In other words, the infective agent or cells infected with an infective agent may be growing in solution (i.e. not adhered to/growing on a surface, such as a surface of a plate).

Preferably, where the infective agent is a bacterium a contacting or incubation step is carried out in solution. In contrast, where the method employs cells infected with an infective agent it is preferred that said cells are growing on or adhered to a surface, such as a surface of a plate.

In one embodiment said contacting or incubation step is carried out under agitation, e.g. at 100-250 rpm, such as 120 rpm.

In one embodiment, where the method employs cells infected with an infective agent, the methods of the invention may comprise the use of at least a 1:1, 5:1 or 10:1 ratio of granulocytes to cells. Preferably the methods comprise the use of a 5:1 ratio of granulocytes to cells. More preferably the methods comprise the use of a 10:1 ratio of granulocytes to cells.

The % of cells killed can be measured by reference to the total number of starting cells. The number of cells killed can be measured using any suitable means, for example by viability staining (e.g. trypan blue staining), and microscopy, or using other automated means, for example by cell electronic sensing equipment, such as the RT-CES™ system available from ACEA Biosciences, Inc. (11585 Sorrento Valley Rd., Suite 103, San Diego, CA 92121, USA). In some embodiments the % of cells killed may be determined within 24 hours (e.g. of incubating a cell and a granulocyte). The % of cells killed is preferably the maximum number of cells killed when carrying out a method of the invention.

The number of cells killed can be also be measured using the ACEA Biosciences xCELLigence RTCA DP Analyzer system®. The xCELLigence System is a real-time cell analyser, allowing for label-free and dynamic monitoring of cellular phenotypic changes continuously by measuring electrical impedance. Such measurements may be carried out as detailed in the Examples. Said System is commercially available from ACEA Biosciences 6779 Mesa Ridge Road #100, San Diego, CA 92121 USA.

In one embodiment, where an infective agent is a bacterium, a ratio of at least 1:10, 1:5, 1:3 or 1:2 granulocytes to colony forming units may be used. Preferably a 1:2 ratio of granulocytes to colony forming units is used. More preferably a 1:1 ratio of granulocytes to colony forming units is used.

In one embodiment a method of the invention further comprises obtaining a stem cell from a sample from a donor from whom a selected granulocyte is obtainable.

Furthermore, the present inventors have surprisingly identified a number of genes (and expression levels thereof) that are associated with a granulocyte's suitability for treating an infection. Advantageously, the expression levels of such genes can be determined using transcriptomic or proteomic techniques to sensitively and specifically identify and/or rapidly identify granulocytes with therapeutic efficacy and/or donors producing such granulocytes.

Moreover, by determining the expression of the one or more genes described herein, the present invention allows for the preparation of substantially homogenous populations of granulocytes suitable for treating an infection (e.g. where at least 90% of the granulocytes present are granulocytes suitable for treating an infection).

In one aspect the present invention provides a method for determining the suitability of a granulocyte for treating an infection, the method comprising:
a. comparing a measured expression level of one or more genes by the granulocyte, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. determining the suitability of the granulocyte for treating an infection based on the comparison.

Representative sequences for the genes for use in the invention are described in the Sequence Listing herein, together with the appropriate Ensembl Accession numbers. A gene for use in the invention may be one or more shown as SEQ ID NOs: 1-24 or 83-87 or a variant thereof. A gene for use in a method of the invention may comprise (or consist of) a nucleotide sequence having at least 70%, 80%, 90% or 95% sequence identity to any one of SEQ ID NOs: 1-24 or 83-87. Preferably, a gene for use in a method of the invention comprises (more preferably consists of) any one of SEQ ID NOs: 1-24 or 83-87.

In one aspect the present invention provides a method for determining the suitability of a granulocyte for treating an infection, the method comprising:
a. measuring an expression level of one or more genes by the granulocyte, wherein the one or more genes are associated with suitability for treating an infection and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. determining the suitability of the granulocyte for treating an infection based on the comparison.

In another aspect the invention provides a method for identifying whether or not a donor produces granulocytes suitable for treating an infection, the method comprising:
a. comparing a measured expression level of one or more genes by a granulocyte comprised in a sample obtainable from the donor, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. identifying whether or not the donor produces granulocytes suitable for treating an infection based on the comparison.

In a related aspect the invention provides a method for identifying whether or not a donor produces granulocytes for treating an infection, the method comprising:
a. measuring an expression level of one or more genes by a granulocyte comprised in a sample obtainable from the donor, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2;
b. comparing the measured expression level with the expression level of the same one or more genes in a reference standard; and
c. identifying whether or not the donor produces granulocytes for treating an infection based on the comparison.

In a preferred embodiment, the methods referred to herein comprise measuring and/or comparing a measured expression level of GM2A, PLEC, CYBB, DOCK8, and/or PPP3CB and optionally one or more further genes. Most preferably, the methods referred to herein comprise measuring and/or comparing a measured expression level of GM2A and optionally one or more further genes. Advantageously, the expression of said genes is highly statistically-significantly different between granulocytes that are suitable for treating an infection and granulocytes that are unsuitable for treating an infection. Thus, measuring, and/or comparing a measured expression level of at least one of those genes has particularly high predictive value. Thus, a gene used in any method described herein may be GM2A, PLEC, CYBB, DOCK8, and/or PPP3CB and optionally one or more further genes. Thus, a protein used in any method described herein may be GM2A, PLEC, CYBB, DOCK8, and/or PPP3CB and optionally one or more further proteins. Most preferably, GM2A or GM2A and optionally one or more further genes/proteins.

In one embodiment, the methods referred to herein are in vitro methods, such as ex vivo methods.

The term “donor” as used herein refers to a subject (suitably a human subject) from whom a sample is obtainable (e.g. obtained). Any suitable sample from which a stem cell or granulocyte cell is obtainable may be obtainable from the donor. The donor may be selected based on one or more of the following characteristics: sex, age, medical history, and/or blood group type. In one embodiment, a donor may be selected if said donor is a healthy donor. In one embodiment, a donor may be selected if said donor does not have an infection. In one embodiment a donor may be selected if said donor is a female. In another embodiment a donor may be selected if said donor is above the age of 40. Suitably, a donor may be selected if said donor is a female above the age of 40. Without wishing to be bound by theory, it is believed that females over 40 have a higher likelihood of producing granulocytes (e.g. neutrophils) that are suitable for treating an infection.

In one embodiment, a donor may be selected if said donor has been exposed to (e.g. vaccinated against) an infection of interest. For example, when obtaining a granulocyte or stem cell suitable for treating a viral infection (e.g. a Coronaviridae infection), a donor may be selected if said donor has been exposed to (or vaccinated against) the viral infection (e.g. the Coronaviridae infection) of interest. In one embodiment, a donor may be selected if said donor has developed immunity against the infection of interest. For example, when obtaining a granulocyte or stem cell suitable for treating a viral infection (e.g. a Coronaviridae infection), a donor may be selected if said donor has developed immunity against the viral infection (e.g. the Coronaviridae infection) of interest.

The term “measuring” as used in reference to expression of one or more genes of the invention encompasses measuring both negative (e.g. no expression) and positive expression (e.g. expression). In one embodiment the expression is positive expression.

Measuring expression may be carried out by any means known to the person skilled in the art. In some embodiments expression may be measured using high-throughput techniques. For example, measuring expression may be at the level of transcription (e.g. transcriptomic techniques) or translation (e.g. proteomic techniques). Alternatively or additionally, the invention may employ the use of genomics, e.g. to detect the presence or absence of SNPs, promoter sequences, gene copy number (e.g. duplications), and/or enhancers or other relevant genetic features, preferably those that determine the expression level of one or more genes of the invention. High-throughput techniques can be used to analyse whole genomes, proteomes and transcriptomes rapidly, providing data, including the expression levels, of all of the genes, polypeptides and transcripts in a cell. Proteomics is a technique for analysing the proteome of a cell (e.g. at a particular point in time). The proteome is different in different cell types. Typically, proteomics is carried out by mass-spectrometry, including tandem mass-spectrometry, and gel based techniques, including differential in-gel electrophoresis. Proteomics can be used to detect polypeptides expressed in a particular cell type and generate a proteomic profile to allow for the identification of specific cell types.

In one embodiment, mRNA of a target gene can be detected and quantified by e.g. Northern blotting or by quantitative reverse transcription PCR (RT-PCR). Single cell gene expression analysis may also be performed using commercially available systems (e.g. Fluidigm Dynamic Array). Alternatively, or in addition, gene expression levels can be determined by analysing polypeptide levels e.g. by using Western blotting techniques such as ELISA-based assays.

Thus, in one embodiment, gene expression levels are determined by measuring the mRNA/cDNA levels of the genes of the present invention, such as RNA sequencing (RNA-Seq).

In a preferred embodiment, gene expression levels are determined by measuring the polypeptide levels produced by the genes of the present invention, such as by way of mass spectrometry, e.g. liquid chromatography and mass spectrometry (LC-MS/MS).

In one embodiment a granulocyte (or stem cell) for treating an infection may be detected using an enzyme-linked immunosorbent assay (ELISA) or a Luminex assay (commercially available from R&D Systems, USA).

Thus, in one embodiment a method of the invention comprises measuring an expression level of one or more polypeptides by a granulocyte, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

Representative sequences for the polypeptides for use in the invention are described in the Sequence Listing herein, together with the appropriate UniProt Accession numbers. A polypeptide for use in the invention may be one or more shown as SEQ ID NOs: 25-82 or a variant thereof, such as a transcript isoform therefore. A polypeptide for use in a method of the invention may comprise (or consist of) a polypeptide sequence having at least 20%, 30%, 40%, 50%, or 60% sequence identity to any one of SEQ ID NOs: 25-82. In one embodiment a polypeptide for use in a method of the invention may comprise (or consist of) a polypeptide sequence having at least 70%, 80%, 90% or 95% sequence identity to any one of SEQ ID NOs: 25-82. Preferably, a polypeptide for use in a method of the invention comprises (more preferably consists of) any one of SEQ ID NOs: 25-82.

In one embodiment a method of the invention comprises measuring and/or comparing an amount of one or more polypeptides produced by a granulocyte, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In one embodiment a method of the invention comprises measuring and/or comparing an expression level of one or more polypeptides by a stem cell, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In one embodiment a method of the invention comprises measuring and/or comparing an amount of one or more polypeptides produced by a stem cell, wherein the one or more proteins are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In one embodiment a method of the invention employs a genome wide association study, which is compared to a reference standard (e.g. a reference standard from a reference population, such as a reference standard from: a suitable or unsuitable donor, or a suitable or unsuitable granulocyte, or a subject that is suitable or unsuitable for treatment with a granulocyte or stem cell of the invention.

Methods suitable for establishing a baseline or reference value for comparing expression levels are conventional techniques known to those skilled in the art.

The term “increased” as used herein in reference to expression of the one or more genes of the invention may refer to an expression level that is statistically-significantly increased when compared to a reference standard. Such a gene may be considered to be upregulated.

In one embodiment increased expression means greater than 1-fold, 1.25-fold to about 10-fold or more expression relative to a reference standard. In some embodiments, increased expression means greater than at least about 1.1-fold, 1.2-fold, 1.25-fold, 1.5-fold, 1.75-fold, 2-fold, 4-fold, 5-fold, 10-fold, 15-fold, 20-fold, 25-fold, 30-fold, 35-fold, 40-fold, 50-fold, 75-fold, 100-fold, 150-fold, 200-fold, or at least about 300-fold expression when compared to a reference standard.

The term “decreased” as used herein in reference to expression of the one or more genes of the invention may refer to an expression level that is statistically-significantly decreased when compared to a reference standard. Such a gene may be considered to be downregulated.

In one embodiment decreased expression means less than −1-fold, −1.25-fold to about −10-fold or more expression relative to a reference standard. In some embodiments, decreased expression means less than at least about −1.1-fold, −1.2-fold, −1.25-fold, −1.5-fold, −1.75-fold, −2-fold, −4-fold, −5-fold, −10-fold, −15-fold, −20-fold, 25-fold, −30-fold, −35-fold, −40-fold, −50-fold, −75-fold, −100-fold, −150-fold, −200-fold, or at least about −300-fold expression when compared to a reference standard.

The fold change difference can be in absolute terms (e.g. CPM: counts per million) or Log 2CPM (a standard measure in the field) of the expression level in a sample. Preferably the fold change is Log 2 fold change. In one embodiment a Log 2 change is an increase of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6 or 2.7. In one embodiment a Log 2 change is a decrease of 0.1 or more, 0.2 or more, 0.3 or more, 0.4 or more, 0.5 or more, 0.6 or more, 0.7 or more, 0.8 or more, 0.9 or more, 1.0 or more, 1.1 or more, 1.2 or more or 1.3 or more. A decrease may be indicated by the presence of a “−” symbol prior to the value.

In one embodiment said fold-change is measured/is determined by RNA sequencing (RNA-Seq), e.g. in toto.

The term “unchanged” or “the same” as used herein in reference to expression of the one or more genes of the invention may refer to an expression level that is not statistically-significantly different to a reference standard. Preferably, an expression level that is the same as a reference standard.

The expression level may be an average such as a mean expression level. In one embodiment statistical significance is determined using two-way ANOVA, e.g. where n is at least 3 and data are presented as mean+/− standard error of mean.

In one embodiment the methods of the invention comprise measuring expression of combinations of the genes described herein.

The term “one or more” when used in the context of a gene described herein may mean at least 2, 3, 4, 5, 6, 7, 8, 9, 10, 11, 12, 13, 14, 15, 16, 17, 18, 19, 20, 21 or 22 of the genes. Preferably, the term “one of more” means all of the genes. Likewise, the term “one or more” when used in the context of a polypeptide described herein may mean at least 2, 3, 4, 5, 6, 7, 8, 9, 10, 11, 12, 13, 14, 15, 16, 17, 18, 19, 20, 21 or 22 of the polypeptides. Preferably, the term “one of more” means all of the polypeptides.

The expression of one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2 correlates with a granulocyte's suitability for treating an infection. Said genes are therefore referred to herein as genes associated with suitability for treating an infection. Thus, the term “one or more genes associated with suitability for treating an infection” (and the like) may in be synonymous with (and thus replaced with) the term “one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2”.

Without wishing to be bound by theory, the inventors believe that, based on the data obtained in the Examples and the inventors' theorized mechanism of action, the one or more genes may have the following functions making them suitable for treating an infection:
a. locating and/or binding to an infective agent or a cell infected by an infective agent: ANXA1, ATG7, ITGB1, SYK, DOCK8, CYBB, RAC1, CAP37, COMP, and PLEC;
b. killing an infective agent or a cell infected by an infective agent: CYBB, SLC2A1, GZMK, CTSG, ATM, PERM, ACSL1, GM2A, and CAP37; and/or
c. recruitment of immune mediators: BCAP31, TAPBP, IKBKB, PPP3CB and PSMB2.

In a preferred embodiment a method of the invention comprises measuring the expression of ANXA1. Advantageously, the inventors have shown that low levels of ANXA1 expression are associated with high infective agent or infective agent-infected cell killing activity and therefore suitability for treating an infection. Without wishing to be bound by theory, it is believed that ANXA1 modulates chemotaxis and/or motility of granulocytes and, in particular, that low expression of ANXA1 promotes granulocyte motility and thus location/binding to an infective agent or infective agent-infected cells.

In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased in a granulocyte that is suitable for treating an infection when compared to a granulocyte that is unsuitable for treating an infection. Alternatively or additionally, in one embodiment expression of ANXA1 and/or PPP3CB is decreased in a granulocyte that is suitable for treating an infection when compared to a granulocyte that is unsuitable for treating an infection.

In one embodiment a method of the invention may further comprise measuring expression of one or more genes selected from: S100A9 and S100A8. In one embodiment expression of S100A9 and/or S100A8 may be increased in a granulocyte of the invention when compared to a reference standard, when the reference standard is from a granulocyte that is unsuitable for treating an infection.

In one embodiment a method of the invention comprises measuring and/or comparing expression of CTSG and at least one further gene selected from: CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2. Similarly, a granulocyte of the invention may comprise increased expression of CTSG and:
at least one further gene selected from: CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 when compared to a reference standard, wherein the reference standard is from a granulocyte unsuitable for treating an infection; or
decreased expression of ANXA1 and/or PPP3CB when compared to a reference standard, wherein the reference standard is from a granulocyte unsuitable for treating an infection.

In a particularly preferred embodiment, a method of the invention comprises measuring and/or comparing expression of GM2A and at least one further gene selected from: CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, CTSG, and PSMB2. Similarly, a granulocyte of the invention may comprise increased expression of GM2A and:
at least one further gene selected from: CAP37, ITGB1, CYBB, SYK, DOCK8, COMP, ATG7, SLC2A1, GZMK, ATM, IKBKB, BCAP31, TAPBP, PERM, PLEC, ACSL1, RAC1, CTSG, and PSMB2 when compared to a reference standard, wherein the reference standard is from a granulocyte unsuitable for treating an infection; or
decreased expression of ANXA1 and/or PPP3CB when compared to a reference standard, wherein the reference standard is from a granulocyte unsuitable for treating an infection.

In one embodiment a method of the invention comprises measuring and/or comparing expression of CAP37 and at least one further gene selected from: CTSG, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2. Similarly, a granulocyte of the invention may comprise increased expression of CAP37 and:
at least one further gene selected from: CTSG, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 when compared to a reference standard, wherein the reference standard is from a granulocyte (or stem cell) unsuitable for treating an infection; or
decreased expression of ANXA1 and/or PPP3CB when compared to a reference standard, wherein the reference standard is from a granulocyte unsuitable for treating an infection.

In another embodiment a method of the invention comprises measuring and/or comparing expression of ANXA1 and at least one further gene selected from: CTSG, CAP37, ITGB1, CYBB, SYK, DOCK8, COMP, ATG7, SLC2A1, GZMK, ATM, IKBKB, BCAP31, TAPBP, PPP3CB, PERM, PLEC, ACSL1, RAC1, GM2A, and PSMB2. Similarly, a granulocyte of the invention may comprise decreased expression of ANXA1 and:
at least one further gene selected from: CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 when compared to a reference standard, wherein the reference standard is from a granulocyte unsuitable for treating an infection; or
decreased expression of PPP3CB when compared to a reference standard, wherein the reference standard is from a granulocyte unsuitable for treating an infection.

The term “for treating an infection” as used herein means “suitable for treating an infection”. In one embodiment a granulocyte that is “suitable for treating an infection” as used herein means that a granulocyte is capable of killing greater than 41.23% of MRSA strain USA300 cells in the “MRSA assay” described herein. In one embodiment a granulocyte is capable of killing at least 50% (e.g. at least 60%) of MRSA strain USA300 cells in the “MRSA assay” described herein. Preferably a granulocyte is capable of killing at least 70% (e.g. at least 90% or 95%) of MRSA strain USA300 cells in the “MRSA assay” described herein. In one embodiment reference to a stem cell “for treating an infection” or that is “suitable for treating an infection” means that said stem cell is capable of differentiating into a granulocyte that is suitable for treating an infection.

In contrast, in one embodiment, a granulocyte that is “not suitable for treating an infection” or is “unsuitable for treating an infection” as used herein is a granulocyte that is not capable of killing greater than 41.23% of MRSA strain USA300 cells in the “MRSA assay” described herein, i.e. a granulocyte that kills less than or equal to 41.23% of MRSA strain USA300 cells in the “MRSA assay” described herein. In one embodiment a granulocyte that is “not suitable for treating an infection” or is “unsuitable for treating an infection” is a granulocyte that is not capable of killing at least 50% (e.g. at least 60%) of MRSA strain USA300 cells in the “MRSA assay” described herein, i.e. a granulocyte that kills less than 50% (e.g. less than 60%) of MRSA strain USA300 cells in the “MRSA assay” described herein. Likewise, in one embodiment, reference to a stem cell “not suitable for treating an infection” or that is “unsuitable for treating an infection” means that said stem cell is not capable of differentiating into a granulocyte that is suitable for treating an infection and/or that differentiates into a granulocyte that is unsuitable for treating an infection.

The “MRSA assay” is carried out as follows:
a. admixing 100 μl of a 1×10⁷ CFU/ml solution of MRSA strain USA300 in RPMI 1640 with 100 μl of a solution containing 1×10⁷ granulocytes/ml;
b. incubating the admixture at 37° C. under shaking at 120 rpm;
c. taking a sample at 2 hours (diluting in sterile RPMI as needed) and plating on Tryptic Soy Agar;
d. incubating the plated sample at 37° C. for 24 hours;
e. counting the bacterial colonies; and
f. quantifying the total CFU content; and
g. calculating the % of MRSA cells killed based on the CFU content in steps a. and f using the formula ((CFU content_{no effector}−CFU content_{effector})/CFU content_{no effector})×100.

In a particularly preferred embodiment the term “suitable for treating an infection” as used herein further means that a granulocyte kills less than 15% of healthy (non-infected) cells in the “healthy (non-infected) cell assay” described herein. Preferably a granulocyte kills less than 10% (e.g. less than 5% or less than 1%) of healthy (non-infected) cells in the “healthy (non-infected) cell assay” described herein.

The “healthy (non-infected) cell assay” is carried out using an ACEA Biosciences xCELLigence RTCA DP Analyzer system® according to the manufacturer's instructions and as follows:
a. 6000 healthy (non-infected) cells are placed in the bottom of a 16 well plate;
b. cells are grown to confluence as determined by plateauing of Cell Index (CI) values (i.e. the ‘normalisation point’);
c. 60,000 granulocytes are added (i.e. giving a ratio of 10 granulocytes to 1 non-pathogen-infected cells) and incubated at 37° C.; and
d. the % of healthy (non-infected) cells killed is the maximum % of non-pathogen-infected cells killed by 48 hours after the addition of the granulocytes as determined using the following formula: ((Cell Index_{no effector}−Cell Index_{effector})/Cell Index_{no effector})×100.

Preferably the healthy (non-infected) cells are MCF-12F, which are commercially available from the American Type Culture Collection, 10801 University Boulevard. Manassas, VA 20110 USA and have catalogue number ATCC® CRL-10783™. In another embodiment the healthy (non-infected) cells are liver cells (e.g. primary non-transplantable liver tissue cells).

The expression level of one or more genes of the invention may be compared to a reference standard. The comparison may be carried out by any suitable technique known to the person skilled in the art, e.g. a bioinformatics technique. The detected gene expression in the reference standard may have been obtained (e.g. quantified) previously to a method of the invention. The expression level of the genes described herein is suitably known in said reference standard. A reference standard is preferably from the same sample type as that referred to in a method of the invention. For example, both the sample and reference standard may be blood samples.

In one embodiment the term “sample” as used herein (e.g. in reference to a sample from a donor) may be any sample comprising a granulocyte. The sample may be any suitable biofluid sample from which a granulocyte is obtainable. A sample may be a blood sample, such as a peripheral blood sample. The term “blood” as used herein encompasses whole blood, blood serum, and blood plasma. Blood may be subjected to centrifugation in order to separate red blood cells, white blood cells, and plasma. Following centrifugation, the mononuclear cell layer may be removed for use in the present invention.

The reference standard may be a proteomic profile (indicating an amount of polypeptide expressed by a granulocyte), a transcriptomic profile (indicating an amount of gene expression by a granulocyte, e.g. measured by way of RNA produced by said granulocyte) or a genomic profile. A genomic profile may be used to detect the presence or absence of SNPs, promoter sequences, gene copy number (e.g. duplications), and/or enhance or other relevant genetic features, preferably those that determine the expression level of one or more genes of the invention. The skilled person will appreciate that both the proteomic and transcriptomic profiles are measures of gene expression and will employ the appropriate reference standard depending on the technique used to measure gene expression in accordance with the invention. For example, where proteomics is used in practising the present invention the skilled person will employ a reference standard that is a proteomic profile, where transcriptomics is used in practising the present invention the skilled person will employ a reference standard that is a transcriptomic profile, and where genomics is used in practising the present invention the skilled person will employ a reference standard that is a genomic profile. A reference standard may refer to a database (e.g. a genomic database), e.g. which may include data from one or more sources, such as one or more subjects and/or cells.

A reference standard is preferably a reference standard for a granulocyte that is unsuitable for treating an infection (e.g. a transcriptomic or proteomic profile of a granulocyte that is unsuitable for treating an infection).

In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating an infection. In one embodiment expression of ANXA1 and/or PPP3CB is decreased when compared to a reference standard, when the reference standard is from a granulocyte unsuitable for treating an infection. In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating an infection and expression of ANXA1 and/or PPP3CB is decreased when compared to a reference standard, when the reference standard is from a granulocyte unsuitable for treating an infection.

A reference standard may be a reference standard for a granulocyte that is suitable for treating an infection (e.g. a transcriptomic or proteomic profile of a granulocyte that is suitable for treating infection). In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte suitable for treating an infection. In one embodiment expression of ANXA1 and/or PPP3CB is decreased or the same when compared to a reference standard, when the reference standard is from a granulocyte suitable for treating an infection.

In some embodiments the present invention may comprise the use of a reference standard for a granulocyte that is unsuitable for treating infection and a reference standard for a granulocyte that is suitable for treating an infection.

A method of the invention may comprise determining the suitability of a granulocyte for treating an infection based on a comparison between a measured expression level of one or more genes of the invention and a reference standard.

In one embodiment a granulocyte is determined as being suitable for treating an infection when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to the reference standard when the reference standard is from a granulocyte unsuitable for treating an infection; and/or
ii. a measured expression level of ANXA1 and/or PPP3CB is decreased when compared to the reference standard, when the reference standard is from a granulocyte unsuitable for treating an infection; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to the reference standard, when the reference standard is from a granulocyte suitable for treating an infection; and/or
iv. a measured expression level of ANXA1 and/or PPP3CB is decreased or the same when compared to the reference standard, when the reference standard is from a granulocyte suitable for treating an infection.

In one embodiment a granulocyte is determined as being unsuitable for treating an infection when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to the reference standard, when the reference standard is from a granulocyte unsuitable for treating an infection; and/or
ii. a measured expression level of ANXA1 and/or PPP3CB is increased or the same when compared to the reference standard, when the reference standard is from a granulocyte unsuitable for treating an infection; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to reference standard, when the reference standard is from a granulocyte suitable for treating an infection; and/or
iv. a measured expression level of ANXA1 and/or PPP3CB is increased when compared to the reference standard, when the reference standard is from a granulocyte suitable for treating an infection.

The methods of the invention may further comprise selecting (or deselecting/discarding) a granulocyte based on the outcome of the method. In one embodiment, where a granulocyte has been determined to be suitable for treating an infection, a granulocyte may be obtained from a sample from which the tested granulocyte was originally obtained. Alternatively, or additionally a stem cell may be obtained from said sample.

Accordingly, in one aspect, there is provided an in vitro method for obtaining a granulocyte for treating an infection, said method comprising obtaining a granulocyte from a sample obtainable from a donor wherein said donor produces granulocytes comprising:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

In a related aspect, there is provided an in vitro method for obtaining a stem cell for treating an infection, said method comprising obtaining a stem cell from a sample obtainable from a donor wherein said donor produces granulocytes comprising:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

A method of the invention may comprise identifying whether or not a donor produces granulocytes suitable for treating an infection based on a comparison between a measured expression level of one or more genes of the invention and a reference standard.

In one embodiment a donor is identified as being a donor that produces granulocytes suitable for treating an infection when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to the reference standard when the reference standard is from a granulocyte unsuitable for treating an infection; and/or
ii. a measured expression level of ANXA1 and/or PPP3CB is decreased when compared to the reference standard, when the reference standard is from a granulocyte unsuitable for treating an infection; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to the reference standard, when the reference standard is from a granulocyte suitable for treating an infection; and/or
iv. a measured expression level of ANXA1 and/or PPP3CB is decreased or the same when compared to the reference standard, when the reference standard is from a granulocyte suitable for treating infection.

In one embodiment a donor is not identified as being a donor that produces granulocytes suitable for treating an infection (or is identified as a donor that produces granulocytes that are unsuitable for treating an infection) when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to the reference standard, when the reference standard is from a granulocyte unsuitable for treating an infection; and/or
ii. a measured expression level of ANXA1 and/or PPP3CB is increased or the same when compared to the reference standard, when the reference standard is from a granulocyte unsuitable for treating an infection; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to reference standard, when the reference standard is from a granulocyte suitable for treating an infection; and/or
iv. a measured expression level of ANXA1 and/or PPP3CB is increased when compared to the reference standard, when the reference standard is from a granulocyte suitable for treating an infection.

The methods of the invention may further comprise selecting (or deselecting) a donor based on the outcome of the method. In one embodiment, where a donor has been identified as being a donor that produces granulocytes suitable for treating an infection, a granulocyte may be obtained from a sample obtainable from said donor.

In one aspect the invention provides a granulocyte obtainable by a method of the invention.

Alternatively, or additionally a stem cell may be obtained from a sample obtainable from said donor. Thus, in one aspect the invention provides a method comprising:
a. identifying a donor that produces granulocytes suitable for treating an infection according to a method of the invention; and
b. obtaining a stem cell from a sample obtainable from the donor.

Thus, in one aspect, the invention provides a stem cell obtainable by a method of the invention. The stem cell is capable of differentiating into a granulocyte for treating an infection, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

The term “obtainable” as used herein also encompasses the term “obtained”. In one embodiment the term “obtainable” means obtained.

In a related aspect, there is provided a stem cell which is capable of differentiating into a granulocyte for treating an infection, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

The term “stem cell” as used herein encompasses any cell that is capable of differentiating into a granulocyte (preferably a neutrophil). For example, the term “stem cell” may encompass totipotent, pluripotent, multipotent, or unipotent cells. In one embodiment the term “stem cell” encompasses a haematopoietic stem cell, as well as a precursor cell (e.g. differentiated from a haematopoietic stem cell), wherein said precursor cell is capable of differentiating into a granulocyte (preferably a neutrophil). Preferably the term “stem cell” as used herein does not encompass a human embryonic stem cell.

A stem cell may be part of a stem cell culture.

The “stem cell” may be a natural stem cell or an artificial stem cell. In one embodiment a natural stem cell may be a cell of the haematopoiesis pathway or a cell equivalent thereto. In one embodiment an artificial stem cell may be an induced pluripotent stem cell (iPSC) or a cell equivalent thereto.

In one embodiment, an iPSC is obtainable from a somatic cell, such as a somatic cell of a donor. Generation of iPSCs is a well-known technique in the art, see Yu et al (2007), Science, 318:1917-1920 the teaching of which is incorporated herein by reference.

In another embodiment, an iPSC is obtainable from a stem cell (e.g. obtainable from a donor), such as from a stem cell of the hematopoietic pathway. Preferably an iPSC is obtainable from a hematopoietic stem cell or a precursor cell described herein.

In one embodiment, a stem cell is a nuclear transfer embryonic stem cell (NT-ESC) or equivalent thereto. In one embodiment, an NT-ESC is obtainable by injecting the nucleus of a cell from the donor into an egg cell from which the original nucleus has been removed. Generation of NT-ESCs is a well-known technique in the art, see Tachibana M, Amato P, Sparman M, et al (2013), Cell, 154(2): 465-466 the teaching of which is incorporated herein by reference.

In one embodiment where a stem cell is obtained from a sample from a donor, said stem cell may be isolated from said sample. In another embodiment where a stem cell is obtained from a sample from a donor, said sample is a sample comprising stem cells or somatic cells and the stem cell is obtained by inducing pluripotency of and/or reprograming a cell (e.g. a somatic cell) in said sample to obtain a stem cell (e.g. an iPSC).

In one embodiment the cell is reprogrammed into an induced pluripotent stem. The cell which is reprogrammed may be a hematopoietic progenitor cell, a mononuclear myeloid cell or a peripheral blood mononuclear cell using methods based on the disclosure in Ohmine et al, Stem Cell Res Ther 2011 November; 2(6):46 and/or Rim et al, J Vis Exp 2016; (118) which are incorporated herein by reference.

In another embodiment the cell is reprogrammed into a multipotent stem cell, for example a hematopoietic stem cell, or a progenitor cell, for example a multilineage blood progenitor. The cell which is reprogrammed may be a fibroblast or a blood cell using methods based on the disclosure in Riddell et al, Cell 2014; 157(3) 549-64 and/or Szabo et al, Nature 2010; 468(7323) 521-526 which are incorporated herein by reference.

In another embodiment where a stem cell is obtained from a sample from a donor, said sample is a sample comprising stem cells or somatic cells and the stem cell is obtained by injecting the nucleus of a cell (e.g. a somatic cell) in said sample into an egg cell (e.g. from which the original nucleus has been removed) to obtain an NT-ESC.

In a preferred embodiment a stem cell is a haematopoietic stem cell. A haematopoietic stem cell may, in one embodiment, be selected on the basis of cell surface polypeptide markers, for example selected from CD34 (e.g. UniProt accession number P28906), CD59 (e.g. UniProt accession number P13987), Thy1 (e.g. UniProt accession number P04216), CD38 (e.g. UniProt accession number P28907), C-kit (e.g. UniProt accession number P10721), and lin. In one embodiment a haematopoietic stem cell comprises the cell surface polypeptide markers CD34⁺, CD59⁺, Thy1⁺, CD38^{low/-}, C-kit^{low/-}, and lin⁻. Preferably a haematopoietic cell expresses CD34. Antibodies to detect the presence or absence of said markers are commercially available and may be obtained from BD Biosciences Europe, ebioscience, Beckman Coulter and Pharmingen, for example.

Most preferably, a stem cell is a precursor cell (which may be referred to herein as a “granulocyte precursor cell”). In one embodiment a precursor cell is a granulocyte-committed progenitor, preferably a neutrophil-committed progenitor. A precursor cell may be one or more selected from a common myeloid progenitor cell, a myeloblast, a promyelocyte (e.g. a N. promyelocyte), a myelocyte (e.g. a N. myelocyte), a metamyelocyte (e.g. a N. metamyelocyte), a band (e.g. an N. band), or combinations thereof. Preferably, a precursor cell is a N. promyelocyte.

A stem cell of the present invention is preferably an isolated stem cell, e.g. a stem cell that has been isolated from its physiological surroundings, such as an ex vivo stem cell.

A stem cell may be differentiated into a granulocyte. A stem cell (e.g. a haematopoietic stem cell, an iPSC, or a NT-ESC) may be differentiated into another type of stem cell (e.g. a precursor cell). Differentiation may be carried out using any suitable method, such as a method based on the disclosure in Lengerke et al, Ann N Y Acad Sci, 2009 September; 1176:219-217, Pawlowski et al, Stem Cell Reports 2017 Apr. 11; 8(4):803-812, Doulatov et al, Cell Stem Cell, 2013, Oct. 3; 13(4)459-470, Lieber et al, Blood, 2004 Feb. 1; 103(3):852-9, and/or Choi et al, Nat. Protoc., 2011 March; 6(3):296-313, and/or Timmins et. al. Biotechnology and bioengineering. 2009; 104(4):832-40, which are incorporated herein by reference.

In one aspect, the invention provides a method for preparing a stem cell for treating an infection, the method comprising:
a. identifying a donor that produces granulocytes for treating an infection according to a method of the invention; and
b. obtaining the stem cell from a sample obtainable from said donor.

In another aspect, the invention provides a method for producing a stem cell for treating an infection, the method comprising:
a. providing a stem cell obtainable from a sample from a donor wherein said donor produces granulocytes comprising:
   i. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
   ii. decreased expression of ANXA1 and/or PPP3CB when compared to a reference standard, wherein the reference standard is from a granulocyte unsuitable for treating an infection; and
b. differentiating the stem cell into a different stem cell (preferably a precursor cell); and
c. optionally isolating the different stem cell (preferably the precursor cell).

In one embodiment, the sample comprises a somatic cell and obtaining the stem cell from the sample comprises reprograming the somatic cell into a stem cell.

In one aspect the invention provides a method for producing a granulocyte for treating an infection, the method comprising:
a. providing a cell; and
b. converting the cell into a granulocyte having an expression profile described herein, for example wherein:
   i. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating an infection; or
   ii. the measured expression level of ANXA1 and/or PPP3CB is decreased in the granulocyte when compared to the reference standard, when the reference standard is from a granulocyte unsuitable for treating an infection; or
   iii. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating an infection; or
   iv. the measured expression level of ANXA1 and/or PPP3CB is decreased or the same when compared to the reference standard, when the reference standard is from a granulocyte suitable for treating an infection; and
c. optionally isolating the granulocyte.

In one embodiment a method for producing a granulocyte for treating an infection comprises:
a. providing a cell; and
b. converting the cell into a granulocyte having an expression profile described herein, for example wherein:
   i. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating an infection; and/or (preferably and)
   ii. the measured expression level of ANXA1 and/or PPP3CB is decreased in the granulocyte when compared to the reference standard, when the reference standard is from a granulocyte unsuitable for treating an infection; and/or (preferably and)
   iii. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating an infection; and/or (preferably and)
   iv. the measured expression level of ANXA1 and/or PPP3CB is decreased or the same when compared to the reference standard, when the reference standard is from a granulocyte suitable for treating an infection; and
c. optionally isolating the granulocyte.

The cell may be a stem cell according to the invention or a somatic/differentiated cell optionally from a donor who produces granulocytes suitable for treating an infection as determined by a method of the invention. In one embodiment, converting the cell into a granulocyte comprises transdifferentiating a somatic/differentiated cell into a granulocyte based on standard techniques known in the art, for example those based on Szabo et al, Nature 2010; 468(7323) 521-526

In one embodiment converting the cell into a granulocyte comprises differentiating a stem cell into a granulocyte based on standard techniques known in the art, for example those referenced herein. For example, in one embodiment a method of differentiating a stem cell comprises admixing said stem cell with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, a method of differentiating a stem cell comprises admixing said stem cell with IFN-gamma and GM-CSF. In preferable embodiments, a method of differentiating a stem cell comprises admixing said stem cell with TNF-alpha.

In one embodiment the invention provides a method of differentiating a stem cell comprising admixing said stem cell with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS).

In one embodiment the invention provides a method of differentiating a stem cell comprising admixing said stem cell with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta.

The term “admix” as used herein means mixing one or more components together in any order, whether sequentially or simultaneously. The result of said admixing is an admixture. In one embodiment “admix” means contacting a first component with a second component (e.g. a stem cell and GM-CSF).

In one embodiment differentiation of a stem cell comprises culturing said stem cell with one or more feeder cell(s). Suitably, a feeder cell may be an OP9 cell. OP9 cells (ATCC® CRL-2749™) are commercially available from the American Type Culture Collection United Kingdom (U.K.), Guernsey, Ireland, Jersey and Liechtenstein, LGC Standards, Queens Road, Teddington, Middlesex, TW11 0LY, UK. In one embodiment a stem cell may be cultured with one or more feeder cell(s) and Flt-3 ligand, thrombopoietin, fetal bovine serum (FBS), or combinations thereof.

Thus in one embodiment, a pharmaceutical composition or cell culture of the invention may further comprise a feeder cell, such as an OP9 cell.

A stem cell may be immortalised. The person skilled in the art is familiar with immortalisation techniques, which include inter alia introduction of a viral gene that deregulates the cell cycle (e.g. the adenovirus type 5 E1 gene), and artificial expression of telomerase. Immortalisation advantageously allows for the preparation of a cell line which can be stably cultured in vitro. Thus, in one aspect the invention provides an immortalised cell line obtainable (e.g. obtained) from a selected stem cell, as well as a stable stem cell culture. Suitably an immortalised cell line or stable stem cell culture is obtainable (e.g. obtained) by a method of the present invention.

The term “stable” as used in reference to a stem cell culture or cell line means that the cell culture or cell line has been modified such that it is more amenable to in vitro cell culture than an unmodified cell (i.e. a cell obtained from a donor and subjected directly to in vitro cell culture). Said “stable” cell culture or cell line is therefore capable of undergoing more rounds of replication (preferably for prolonged periods of time) when compared to an unmodified cell.

In one aspect the invention provides a method for selecting whether or not a subject is suitable for treatment with a granulocyte or a stem cell for treating an infection, the method comprising:
a. comparing a measured expression level of one or more genes by a granulocyte comprised in a sample obtainable from the subject, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2 with the expression level of the same one or more genes in a reference standard; and
b. identifying whether or not the subject is suitable for treatment with a granulocyte or a stem cell for treating an infection based on the comparison.

In one aspect the invention provides a method for selecting whether or not a subject is suitable for treatment with a granulocyte or a stem cell for treating an infection, the method comprising:
a. measuring an expression level of one or more genes by a granulocyte comprised in a sample obtainable from the subject, wherein the one or more genes are associated with suitability for treating an infection and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. identifying whether or not the subject is suitable for treatment with a granulocyte or a stem cell for treating an infection based on the comparison.

The foregoing method allows for the identification of subjects who have granulocytes that are unsuitable for treating an infection and who are appropriate candidates for treatment with a granulocyte or stem cell of the invention. Advantageously, patients who are most likely to respond positively to treatment can be selected, thereby allowing for more cost-effective and/or economical prescribing of the granulocyte and/or stem cell of the invention and/or avoiding selection of an incorrect patient cohort for clinical trials.

In one embodiment a subject is identified as being suitable for treatment with a granulocyte or stem cell of the invention when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to a reference standard, when the reference standard is from a granulocyte unsuitable for treating an infection; or
ii. a measured expression level of ANXA1 and/or PPP3CB is increased or the same when compared to a reference standard, when the reference standard is from a granulocyte unsuitable for treating an infection; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to a reference standard, when the reference standard is from a granulocyte suitable for treating an infection; or
iv. a measured expression level of ANXA1 and/or PPP3CB is increased when compared to a reference standard, when the reference standard is from a granulocyte suitable for treating an infection.

In one embodiment a subject is identified as being unsuitable for treatment with a granulocyte or stem cell of the invention when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating an infection; or
ii. a measured expression level of ANXA1 and/or PPP3CB is decreased when compared to a reference standard, when the reference standard is from a granulocyte unsuitable for treating infection; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte suitable for treating an infection; or
iv. a measured expression level of ANXA1 and/or PPP3CB is decreased or the same when compared to a reference standard, when the reference standard is from a granulocyte suitable for treating an infection.

The terms “subject” and “patient” are used synonymously herein. The “subject” may be a mammal, and preferably the subject is a human subject.

The term “granulocyte” encompasses the following cell types: neutrophils, basophils, and eosinophils. Preferably the granulocyte is a neutrophil. A granulocyte may express the cell surface polypeptide markers CD11b (e.g. UniProt accession number P11215) and CD15. A granulocyte may also produce reactive oxygen species (O₂⁻). Preferably the granulocyte is CD11b high. Alternatively or additionally, the granulocyte may have a higher density than granulocytes unsuitable for treating an infection, and/or a positive cell surface charge (e.g. a net cell charge).

A granulocyte of the present invention is preferably an isolated granulocyte, e.g. a granulocyte that has been isolated from its physiological surroundings, such as an ex vivo granulocyte.

In some embodiments the granulocyte is obtainable from a sample obtainable from a donor. In another embodiment a granulocyte may be an engineered granulocyte. Such a granulocyte may be produced by a method comprising:
a. providing a granulocyte; and
b. engineering the granulocyte to:
   i. increase expression of one or more genes selected from: ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2; and/or
   ii. decrease expression of ANXA1 and/or PPP3CB;
thereby producing the engineered granulocyte, wherein the engineered granulocyte is suitable for treating an infection.

The granulocyte provided for use in the method is preferably a granulocyte that is unsuitable for treating an infection. Thus, in some embodiments a method of the invention converts a cell that is unsuitable for treating an infection into a granulocyte that is suitable for treating an infection. Said granulocyte may be identified by a method described herein, obtained from a donor identified by a method described herein (e.g. from a subject suitable for treatment with a granulocyte or stem cell of the invention). In some embodiments the engineered granulocyte may be used as a reference standard in a method of the invention (i.e. as a reference standard from a granulocyte suitable for treating an infection).

The granulocyte provided for use in the method is preferably a granulocyte that is unsuitable for treating an infection. Thus, in some embodiments a method of the invention converts a cell that is unsuitable for treating an infection into a granulocyte that is suitable for treating an infection. Said granulocyte may be identified by a method described herein and/or obtained from a donor identified by a method described herein (e.g. from a subject suitable for treatment with a granulocyte or stem cell of the invention).

In a related aspect the invention provides a method for producing an engineered stem cell, the method comprising:
a. providing a stem cell; and
b. engineering the stem cell to:
   i. increase expression of one or more genes selected from: ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2; and/or
   ii. decrease expression of ANXA1 and/or PPP3CB;

thereby producing the engineered stem cell, wherein the engineered stem cell is suitable for treating an infection. In some embodiments the engineered stem cell may be used as a reference standard in a method of the invention (i.e. as a reference standard from a stem cell suitable for treating an infection).

In a related aspect, the invention provides a method for producing an engineered stem cell, the method comprising:
a. providing a stem cell; and
b. engineering the stem cell such that it is capable of differentiating into a granulocyte having:
   i. increased expression of one or more genes selected from: ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2; and/or
   ii. decreased expression of ANXA1 and/or PPP3CB;
thereby producing the engineered stem cell, wherein the engineered stem cell is suitable for treating an infection. In some embodiments the engineered stem cell may be used as a reference standard in a method of the invention (i.e. as a reference standard from a stem cell suitable for treating an infection).

The stem cell provided for use in the method is preferably a stem cell that is unsuitable for treating an infection. Thus, in some embodiments a method of the invention converts a stem cell that is unsuitable for treating an infection into a stem cell that is suitable for treating an infection. Said stem cell may be identified by a method described herein, obtained from a donor identified by a method described herein (e.g. from a subject suitable for treatment with a granulocyte or stem cell of the invention).

Engineering a stem cell or granulocyte may be carried out in vivo, for example in one aspect the invention comprises engineering a stem cell or granulocyte in a subject, preferably engineering a stem cell in a subject. In one embodiment the invention may comprise engineering a stem cell or granulocyte in situ in a subject (e.g. in the bone marrow of a subject). Suitably, said subject may be a subject that produces stem cells or granulocytes that are unsuitable for treating an infection, a subject that is suitable for treatment with a granulocyte or stem cell of the invention, or combinations thereof.

The engineering may be carried out using any means known to the person skilled in the art. In one embodiment expression may be increased or decreased using genome editing. In one embodiment expression may be increased or decreased using CRISPR (e.g. the DNA-snipping CRISPR-associated endonuclease Cas9 genome-editing system), TALENS, adenoviruses (AV), retroviruses, vectors (e.g. inducible and/or over-expressible vectors), transgene insertion, cisgene over- or under-expression, silencing, or epigenetic modulation of promoter regions through histone deacetylase (HDAC) inhibitors, or combinations thereof. The expression of genes associated with suitability for treating an infection can be modulated in stem cells (e.g. myeloblasts) using methods of culturing (adapted from Gupta D, Shah H P, Malu K, Berliner N, Gaines P. Differentiation and characterization of myeloid cells. Curr Protoc Immunol. 2014; 104: Unit 22F 25), and in any suitable cells by using CRISPR methods (adapted from N. E. Sanjana, O. Shalem, F. Zhang Improved vectors and genome-wide libraries for CRISPR screening Nat. Methods, 11 (2014), pp. 783-784), TALEN systems (adapted from A. A. Nemudryi, K. R. Valetdinova, S. P. Medvedev, and S. M. Zakian TALEN and CRISPR/Cas genome editing systems: tools of discovery. Acta Naturae. 2014; 6(3); 19-40), and Zinc finger proteins (adapted from M. C. Keightley et al. The Pu.1 target gene Zbtb11 regulates neutrophil development through its integrase-like HHCC zinc finger. Nat Commun. 2017; 8; 14911) to generate cells with key genes knocked-in or knocked-out. Where the cell is a stem cell, said cell can be differentiated to produce granulocytes. Briefly, by using lentiviral transduction of single guide CRISPR-Cas9 vectors, pre-validated CRISPR (guide) gRNA sequences to genes associated with suitability for treating an infection in the lentiviral vector lentiCRISPRv2 can be ordered from GenScript or AddGene. CRISPR knockout experiments may use targeting sequences within exons, whereas CRISPR activation or repression experiments may use targets within promoters. ANXA1 for instance, can be knocked-out of a cell to improve bacteria killing activity using a pre-validated gRNA targeting its exon. Lentiviral vectors may be prepared and suitable cells transduced (according to previously published protocols (Satchwell T J, Hawley B R, Bell A J, Ribeiro M L, Toye A M. The cytoskeletal binding domain of band 3 is required for multiprotein complex formation and retention during erythropoiesis. Haematologica 2015; 100(1):133-142). Verification of CRISPR on- and off-target effects can be confirmed via whole genome sequencing by comparing the genomic differences between the unedited control and the modified samples. Modified myeloblasts may then be differentiated and identified using the methods of Gupta and colleagues (2014). In one embodiment said approaches may be applied to granulocytes or stem cells.

In some embodiments the engineering may be modulation of one or more cytokines driving lineage in stem cells and/or genes associated with suitability for treating an infection.

In one aspect of the invention, there is provided a granulocyte (or engineered granulocyte) for treating an infection, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

In one embodiment a granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and
b. decreased expression of ANXA1 and/or PPP3CB when compared to a reference standard, wherein the reference standard is from a granulocyte unsuitable for treating an infection.

In a particularly preferred embodiment a granulocyte of the invention has a positively charged cell surface (or a more positively charged cell surface when compared to a granulocyte that is unsuitable for treating an infection).

The skilled person would appreciate that the granulocytes or stem cells of the invention do not need to be activated ex vivo to be suitable for treating an infection. To the extent that the granulocytes or stem cells of the invention are activated ex vivo, the skilled person would appreciate that any activation would further increase the infection killing activity of the granulocytes or stem cells. Accordingly, in some embodiments, the granulocyte or stem cell for treating an infection has not been activated ex vivo. For example, in some embodiments, the granulocyte or stem cell for treating an infection has not been activated ex vivo with flagellin. In some embodiments, the granulocyte or stem cell for treating an infection has not been activated ex vivo with a chemokine, cytokine or glucocorticoid. For example, in some embodiments, the granulocyte or stem cell for treating an infection has not been activated ex vivo with G-CSF. In some embodiments, the granulocyte or stem cell for treating an infection has not been activated ex vivo with prednisone. In some embodiments, the granulocyte or stem cell for treating an infection has not been activated ex vivo with the infective agent to be treated. For example, the granulocyte or stem cell for treating an infection may not have been activated ex vivo with the bacterium, virus, fungi, or pathogen of interest. In some embodiments, the granulocyte or stem cell for treating an infection has not been activated ex vivo with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, the granulocyte or stem cell for treating an infection has not been activated ex vivo with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. Thus, in some embodiments, the granulocyte or stem cell for treating an infection is an unactivated granulocyte or stem cell.

Accordingly, in some embodiments of a method of the invention, the method does not comprise activating a granulocyte or stem cell for treating an infection ex vivo. For example, in some embodiments, the method does not comprise activating the granulocyte or stem cell for treating an infection ex vivo with flagellin. In some embodiments, the method does not comprise activating the granulocyte or stem cell for treating an infection ex vivo with a chemokine, cytokine or glucocorticoid. For example, in some embodiments, the method does not comprise activating the granulocyte or stem cell for treating an infection ex vivo with G-CSF. In some embodiments, the method does not comprise activating the granulocyte or stem cell for treating an infection ex vivo with prednisone. In some embodiments, the method does not comprise activating the granulocyte or stem cell for treating an infection ex vivo with a chemokine or cytokine. In some embodiments, the method does not comprise activating the granulocyte or stem cell for treating an infection ex vivo with the infective agent to be treated. For example, the method may not comprise activating the granulocyte or stem cell for treating an infection ex vivo with the bacterium, virus, fungi, or pathogen of interest. In some embodiments, the method does not comprise activating the granulocyte or stem cell for treating an infection with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, the method does not comprise activating the granulocyte or stem cell for treating an infection with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. In some embodiments, the method does not comprise activating the granulocyte or stem cell for treating an infection ex vivo with IFN-gamma and GM-CSF.

In alternative embodiments, the granulocyte or stem cell for treating an infection has been activated ex vivo. For example, in some embodiments, the granulocyte or stem cell for treating an infection has been activated ex vivo with flagellin. In some embodiments, the granulocyte or stem cell for treating an infection has been activated ex vivo with a chemokine, cytokine or glucocorticoid. For example, in some embodiments, the granulocyte or stem cell for treating an infection has been activated ex vivo with G-CSF. In some embodiments, the granulocyte or stem cell for treating an infection has been activated ex vivo with prednisone. In some embodiments, the granulocyte or stem cell for treating an infection has been activated ex vivo with a chemokine or cytokine. In preferred embodiments, the granulocyte or stem cell for treating an infection has been activated ex vivo with the infective agent to be treated. For example, the granulocyte or stem cell for treating an infection may have been activated ex vivo with the bacterium, virus, fungi, or pathogen of interest. In some embodiments, the granulocyte or stem cell for treating an infection has been activated with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, the granulocyte or stem cell for treating an infection has been activated with IFN-gamma and GM-CSF. In preferred embodiments, the granulocyte or stem cell for treating an infection has been activated with TNF-alpha. Thus, in some embodiments, the granulocyte or stem cell for treating an infection is an activated granulocyte or stem cell.

Accordingly, in some embodiments of a method of the invention, the method comprises activating a granulocyte or stem cell for treating an infection ex vivo. For example, in some embodiments, the method comprises activating the granulocyte or stem cell ex vivo with flagellin. In some embodiments, the method comprises activating the granulocyte or stem cell for treating an infection ex vivo with a chemokine, cytokine or glucocorticoid. For example, in some embodiments, the method comprises activating the granulocyte or stem cell for treating an infection ex vivo with G-CSF. In some embodiments, the method comprises activating the granulocyte or stem cell for treating an infection ex vivo with prednisone. In some embodiments, the method comprises activating the granulocyte or stem cell for treating an infection ex vivo with a chemokine or cytokine. In preferred embodiments, the method comprises activating the granulocyte or stem cell for treating an infection ex vivo with the infective agent of interest. For example, the method may comprise activating the granulocyte or stem cell for treating an infection ex vivo with the bacterium, virus, fungi, or pathogen of interest. In some embodiments, the method comprises activating the granulocyte or stem cell for treating an infection with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, the method comprises activating the granulocyte or stem cell for treating an infection with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. In some embodiments, the method comprises activating the granulocyte or stem cell for treating an infection ex vivo with IFN-gamma and GM-CSF.

The present invention may further comprise the validation of a granulocyte or stem cell's suitability for treating an infection by a different means, e.g. by way of cell surface charge and/or by way of a functional assay.

In one embodiment granulocyte cell surface charge correlates with suitability for treating an infection, with granulocytes (e.g. neutrophils) that are more positively charged (or less negatively charged) being suitable for treating an infection and/or more efficacious in treating an infection. The level of cell surface charge may be determined when compared to a reference standard, preferably wherein the reference standard is from a granulocyte that is unsuitable for treating an infection. In one embodiment a stem cell may be considered as suitable for treating an infection if it is capable of differentiating into a granulocyte having a more positively charged (or less negatively charged) cell surface. A cell surface charge can be determined using any suitable technique known in the art. In one embodiment the cell surface charge is determined using electrophoresis. An electrophoretic mobility assay may be one described in “Cell Electrophoresis” edited by Johann Bauer (ISBN 0-8493-8918-6 published by CRC Press, Inc.) the teaching of which is incorporated herein in its entirety. In another embodiment cell surface charge can be determined using negatively and/or positively charged means. In one embodiment, a granulocyte has a positive cell surface charge when it can be bound by a negatively charged means, and not a positively charged means. In one embodiment, a granulocyte has a negative cell surface charge when it can be bound by a positively charged means, and not a negatively charged means. Such negatively and/or positively charged means may also be used to measure the concentration of a granulocyte cell in a sample. A positively charged means may be a positively charged particle, nanoprobe or nanoparticle, or a cation exchange media. Suitable nanoparticles may be prepared by conjugating superparamagnetic Iron(II, III) oxide (Fe₃O₄) nanoparticles (NPs) with (3-Aminopropyl)triethoxysilane (APTES) to form a thin layer of Silicon dioxide (SiO₂) shell on the NPs' surface upon reaction with Tetraethyl orthosilicate (TEOS) and ammonium hydroxide (NH₄OH). Fluorescein isothiocyanates (FITCs) may be embedded in the SiO₂ shell, thus exposing the Si-linked hydroxyl groups (SiO₂—OH) and creating the negative surface charge. Branched poly(ethylene imine) (PEI) molecules may be used to not only to cover the SiO₂—OH groups in a non-covalently manner but also to expose the additional amine groups that carry the positive charges. Thus, in one embodiment a negatively charged nanoparticle is prepared by conjugating Fe₃O₄ nanoparticles with APTES to form a thin layer of SiO₂ shell on the nanoparticle surface upon reaction with Tetraethyl orthosilicate (TEOS) and ammonium hydroxide (NH₄OH), and embedding a FITC in the SiO₂ shell, thus exposing the SiO₂—OH groups (creating the negative surface charge). In another embodiment, a positively charged nanoparticle is prepared by contacting a negatively charged nanoparticle (as described herein) with a PEI molecule (e.g. to expose additional amine groups that carry a positive charge). In one embodiment, the negatively charged means (e.g. nanoparticle) may have a negative surface charge of at least −5 mV, −10 mV, −20 mV, −30 mV, or −40 mV. Preferably, the negatively charged means (e.g. nanoparticle) has may have a negative surface charge of at least −35 mV. In one embodiment, the positively charged means (e.g. nanoparticle) may have a positive surface charge of at least +5 mV, +10 mV, +20 mV, +30 mV, or +40 mV. Preferably, the positively charged means (e.g. nanoparticle) has may have a positive surface charge of at least +35 mV. The surface charge of said positively or negatively charged means (e.g. nanoparticle) may refer to the surface zeta potential of the positively or negatively charged means (e.g. nanoparticle). The surface zeta potential may be measured with a Dynamic light scattering particle size analyser (e.g. the Zetasizer Nano-ZS90, Malvern, UK). In one aspect the present invention involves isolating granulocytes comprising a (more) positive cell surface charge by way of said charge. For example, said cells may be isolated using a negatively charged means, such as a negatively charged particle, nanoprobe or nanoparticle, or an anion exchange media. Such techniques may be used to measure the cell surface charge of granulocytes or the concentration of granulocytes having a positive cell surface charge in the foregoing embodiments. The cells may be isolated from negatively charged, neutrally charged, or less positively charged granulocytes. In one embodiment, a positively or negatively charged means (e.g. nanoparticle) may be detectable by fluorescence. In another embodiment, a positively or negatively charged means (e.g. nanoparticle) may be capable of being captured by way of magnetism, thus allowing isolation of a cell that interacts with said means.

A functional assay for validating the suitability of a granulocyte or stem cell for treating an infection may comprise:
a. contacting an infective agent or cells infected with an infective agent with a granulocyte to form a test sample;
b. incubating the test sample; and
c. measuring the % of infective agent or cells infected with an infective agent killed in the test sample.

To validate a stem cell's suitability, said stem cell may be differentiated into a granulocyte which is employed in the above-mentioned assay.

In one embodiment a granulocyte or stem cell is validated according to the assay when the granulocyte kills greater than 41.23% (e.g. at least 50%) of infective agent or cells infected with an infective agent in the test sample. In one embodiment, a granulocyte or stem cell is validated according to the assay when the granulocyte kills at least 60% or 70% of infective agent or cells infected with an infective agent in the test sample. In one embodiment, a granulocyte or stem cell is validated according to the assay when the granulocyte kills at least 80% or 90% of infective agent or cells infected with an infective agent in the test sample.

The incubation step may be carried out for between 1 hour and 100 hours. Suitably, the incubation step may be carried out for between 5 hours and 75 hours, for example between 10 hours and 20 hours. The incubation step may be carried out for between 6 hours to 6 days. Suitably, the incubation step may be carried out for between 6 hours and 2 days, for example for between 12 hours to 36 hours, such as between 16 to 24 hours. In one embodiment the incubation step is carried out for 24 hours. In another embodiment the incubation step is carried out for 48 hours. The incubation step may be carried out at any temperature suitable for cell growth and viability, for example at a temperature between 35° C. to 42° C., suitably at 37 or 39° C. Preferably the incubation step is carried out at 37 or 39° C. for 24 hours. Preferably the incubation step is carried out for 16-24 hours at 30-40° C. (e.g. 37° C.).

The % of infective agent or cells infected by an infective agent killed as described herein can be measured by reference to the total number of starting infective agent or cells infected by an infective agent. The amount of infective agent or number of cells infected by an infective agent killed can be measured using any suitable means, for example by counting colony forming units in culture, viability staining (e.g. trypan blue staining), and microscopy. In some embodiments the % of infective agent or cells infected by an infective agent killed may be determined within 24 hours (e.g. of incubating an infective agent or cells infected by an infective agent, and a granulocyte). The % of infective agent or cells infected by an infective agent killed is preferably the maximum amount of infective agent (e.g. number of infective agent cells killed) or cells infected by an infective agent killed when carrying out a method of the invention.

In one embodiment, where an infective agent is a bacterium, a ratio of at least 1:10, 1:5, 1:3 or 1:2 granulocytes to colony forming units may be used. Preferably a 1:2 ratio of granulocytes to colony forming units is used. More preferably a 1:1 ratio of granulocytes to colony forming units is used.

A granulocyte or stem cell described herein may be part of a cell culture (e.g. an in vitro cell culture). Accordingly, in one aspect, there is provided an in vitro culture of granulocytes of the invention. In a related aspect, there is provided an in vitro culture of stem cells of the invention.

A granulocyte or stem cell of the invention may be subjected to one or more further processing steps, such as cryogenic freezing. The further processing step may include admixing said granulocyte or stem cell with a preservation medium, for example a cryogenic preservation medium.

In one aspect the invention provides a composition for treating an infection, the composition comprising granulocytes: wherein at least 90% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

In one aspect the invention provides a composition for treating an infection, the composition comprising granulocytes: wherein at least 95% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

In one aspect the invention provides a composition for treating an infection, the composition comprising granulocytes: wherein at least 99% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

In one aspect the invention provides a composition for treating an infection, the composition comprising granulocytes: wherein at least 100% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and/or
b. decreased expression of ANXA1 and/or PPP3CB when compared to a reference standard, wherein the reference standard is from a granulocyte unsuitable for treating an infection.

In one embodiment at least 90%, 95%, 99% or 100% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating an infection; and
b. decreased expression of ANXA1 and/or PPP3CB when compared to a reference standard, wherein the reference standard is from a granulocyte unsuitable for treating an infection.

Advantageously, the compositions of the invention contain a substantially homogeneous population of granulocytes that are suitable for treating an infection.

The invention also provides a method for isolating granulocytes suitable for treating an infection based on the expression of one or more genes of the invention. Such methods may provide a substantially homogeneous population of granulocytes that are suitable for treating an infection. In one embodiment, granulocytes for treating an infection are isolated using the expression of one or more cell-surface expressed polypeptides selected from: ATG7, CYBB, DOCK8, CTSG, S100A9, COMP, S100A8, CTSG, SYK, ITGB1, SLC2A1, GZMK, ANXA1, RAC1, and CAP37, preferably one or more selected from: ATG7, S100A9, COMP, S100A8, CTSG, SYK, ITGB1, SLC2A1, GZMK, ANXA1, RAC1, and CAP37. The isolation may be performed using any suitable technique. In one embodiment a method for isolating granulocytes comprises the use of a binding means that binds to a polypeptide of the invention. Preferably the binding means is an antibody. Antibodies to detect the presence or absence of polypeptides of the invention are commercially available: anti-CYBB antibody (Cat #M03328, BosterBio), anti-DOCK8 antibody (Ab227529, AbCam), anti-ATG7 antibody (Cat #HPA007639, Atlas Antibodies), anti-S100A9 antibody (HPA004193, Atlas Antibodies), anti-ACSL1 antibody (Cat #HPA011964, Atlas Antibodies), anti-ATM antibody (Cat #HPA067142, Atlas Antibodies), anti-COMP antibody (Cat #AF3134, R&D Systems), anti-TAPBP antibody (Cat #HPA007066, Atlas Antibodies), anti-S100A8 antibody (Cat #AF4570, R&D Systems), anti-PLEC antibody (Cat #HPA029906, Atlas Antibodies), anti-BCAP31 antibody (Cat #HPA003906, Atlas Antibodies), anti-CTSG antibody (Cat #C35667, Sab Biotech), anti-SYK antibody (Cat #Ab40781, AbCam), anti-ITGB1 antibody (Cat #P260111, Sino Biological), anti-PSMB2 antibody (Cat #HPA026322, Atlas Antibodies), anti-GM2A antibody (Cat #HPA008063, Atlas Antibodies), anti-SLC2A1 antibody (Cat #HPA031345, Atlas Antibodies), anti-GZMK antibody (Cat #HPA063181, Atlas Antibodies), anti-IKBKB antibody (Cat #HPA001249, Atlas Antibodies), anti-PPP3CB antibody (Cat #HPA008233, Atlas Antibodies), anti-ANXA1 antibody (Cat #HPA011271, Atlas Antibodies), anti-PERM antibody (Cat #HPA021147, Atlas Antibodies), anti-RAC1 antibody (Cat #HPA047820, Atlas Antibodies), and anti-CAP37 antibody (Cat #HPA055851, Atlas Antibodies). The method may comprise the use of flow cytometric techniques, preferably fluorescence activated cell sorting (FACS), e.g. together with appropriate ‘gating’. Flow cytometric techniques may be particularly suitable when the method employs the use of a binding means coupled to a detectable label, such as a fluorophore.

In one aspect there is provided a method for isolating a granulocyte for treating an infection, the method comprising:
a) contacting a sample of granulocytes with a binding means, wherein the binding means binds to one or more polypeptides selected from CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2; and
b) isolating the granulocyte based on the presence or absence of binding between the binding means and the one or more polypeptides.

Binding may be determined to be present when the amount of binding is statistically significant (e.g. when compared to a ‘background’ control). Binding may be determined to be absent when the amount of binding is statistically insignificant (e.g. when compared to a ‘background’ control). Preferably, binding is determined to be absent when there is no binding whatsoever.

In one embodiment the invention comprises detecting the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2. When said one or more polypeptides are detected (i.e. where there is binding between the binding means and the polypeptide) the granulocyte may be isolated. Suitably, said isolated granulocyte may be a granulocyte for treating an infection.

In another embodiment the invention may comprise detecting the absence of ANXA1 and/or PPP3CB (e.g. not detecting ANXA1 and/or PPP3CB). When said one or more polypeptides are not detected (i.e. where there is an absence of binding between the binding means and the polypeptide) the granulocyte may be isolated. Suitably, said isolated granulocyte may be a granulocyte for treating an infection.

Preferably, the invention comprises detecting:
the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
the invention absence of ANXA1 and/or PPP3CB. In one embodiment, when said polypeptides are detected or not detected (as indicated), the granulocyte is isolated.

In one embodiment a method of isolating a granulocyte comprises the use of an immobilised binding means (e.g. a binding means conjugated to a bead, such as a magnetic bead, or chromatographic resin) to isolate a granulocyte of the invention. Such methods may be immuno-affinity methods.

The method may comprise quantifying the amount of binding between the binding means and the one or more polypeptides or between the binding means and the granulocyte. The method may comprise isolating a granulocyte for treating an infection based on the quantified amount of binding.

In one embodiment a granulocyte for treating an infection is isolated when there is a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2.

In one embodiment a granulocyte for treating an infection is isolated when there is a low level of binding between a binding means and ANXA1 and/or PPP3CB.

Preferably, a granulocyte for treating an infection is isolated when there is:
a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
a low level of binding between a binding means and ANXA1 and/or PPP3CB.

A high/low level of binding is preferably relative to a level of binding between the same binding means and polypeptide under the same conditions for a granulocyte that is unsuitable for treating an infection.

The term “isolating” may mean providing a population of granulocytes in which at least 50%, 60%, 70%, 80% or 90% (preferably at least 95%, 99% or 100%) are granulocytes suitable for treating an infection. In other words, the term “isolating” may mean removing at least 50%, 60%, 70%, 80% or 90% (preferably at least 95%, 99% or 100%) of granulocytes that are unsuitable for treating an infection from a population of granulocytes.

Thus, the methods for isolating suitably allow for the separation of a granulocyte for treating an infection from granulocyte that is unsuitable for treating an infection.

In some embodiments a method described herein comprises discarding granulocytes that are unsuitable for treating an infection.

In one aspect the invention provides a pharmaceutical composition comprising:
a. a granulocyte, stem cell, or composition of the invention; and
b. a pharmaceutically acceptable carrier, excipient, adjuvant, and/or salt.

The term “pharmaceutically acceptable carrier, excipient, adjuvant, and/or salt” as used herein means a carrier that can be administered to a subject (e.g. a patient) intravenously, intra-arterially, intraperitoneally, intrathecally or combinations thereof (preferably intravenously) without causing harm to said subject. In one embodiment a pharmaceutically acceptable carrier is an injectable carrier, such as a sterile physiological saline solution. In one embodiment a pharmaceutically acceptable carrier, excipient, adjuvant, and/or salt may be Plasma-Lyte A (e.g. commercially available from Baxter, USA), dextrose, sodium chloride, human serum albumin, dextran (e.g. dextran 40 (LMD)), dextrose, DMS or combinations thereof. Plasma-Lyte A may be present at a concentration of 10-50% v/v (preferably 31.25% v/v). 5% dextrose/0.45% sodium chloride may be present at a concentration of 10-50% v/v (preferably 31.25% v/v). 25% HAS may be present at 10-30% v/v (preferably 20% v/v). 10% Dextran 40 (LMD)/5% dextrose may be present at a concentration of 1-30% v/v (preferably 10% v/v). DMS may be present at 1-15% v/v (preferably 7.5% v/v).

The pharmaceutical composition may comprise a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. Suitably, the pharmaceutical composition comprises IFN-gamma and GM-CSF. Preferably, the pharmaceutical composition comprises TNF-alpha. Particularly preferably, the pharmaceutical composition comprises a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). Preferably, the pharmaceutical composition comprises a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta.

In a related aspect the invention provides a kit comprising a granulocyte, stem cell, composition or pharmaceutical composition of the invention; and instructions for use of the same in medicine (e.g. in treating an infection). Suitably, the instructions may be for the use of the same in treating an infection described in any one of the foregoing embodiments. In some embodiments the instructions also detail an appropriate dosage regimen (e.g. as described in a foregoing embodiment). In one embodiment the instructions are for use of said kit in treating an infection, preferably MRSA.

The invention may further comprise depositing a granulocyte, stem cell, composition or pharmaceutical composition of the invention in a cell bank, and thus in a related aspect provides a granulocyte, stem cell, composition or pharmaceutical composition. The term “cell bank” as used herein refers to a storage facility which maintains a cell under suitable conditions for cell viability. For example, the cell may be stored in a metabolically dormant state (e.g. cryogenically frozen). Suitably, a cell comprised within a cell bank is catalogued for appropriate retrieval (e.g. based on blood group, and/or human leukocyte antigen (HLA) type). In one embodiment a cell may be catalogued based on the type of infection it (or a cell differentiated therefrom) kills. Where the cell bank is a granulocyte cell bank, said cell bank may be replenished using a stem cell of the invention. In some embodiments a stem cell or granulocyte obtained from a donor may be stored and later administered to said donor (e.g. if said donor is diagnosed with an infection), thus constituting a personalised medicine.

A granulocyte or stem cell of the invention may be formulated in any suitable manner, based on its downstream application (e.g. storage in a cell bank, or use in therapy).

Thus, one aspect of the invention provides a cell bank comprising the stem cell, granulocyte, composition, or pharmaceutical composition of the present invention.

The present invention provides granulocytes, stem cells, pharmaceutical compositions, and kits for use in medicine, particularly in the treatment of an infection.

Thus in one aspect the invention provides a granulocyte of the invention for use in treating an infection. In another aspect the invention provides a stem cell of the invention for use in treating an infection. In another aspect the invention provides a composition of the invention for use in treating an infection. In another aspect the invention provides a pharmaceutical composition of the invention for use in treating an infection. In another aspect the invention provides a kit of the invention for use in treating an infection. Similarly, the invention provides in one aspect use of a granulocyte, stem cell, composition, pharmaceutical composition, or kit of the invention in the manufacture of a medicament for treating an infection. In a related aspect there is provided a method for treating an infection comprising: administering to a subject in need thereof a granulocyte, stem cell, composition, pharmaceutical composition, or kit of the invention.

In one embodiment the infection is an infection by any infective agent described herein. Preferably any pathogen described herein. In one embodiment an infection is a nosocomial infection.

Preferably an infection treated by the present invention is tuberculosis.

In some embodiments a stem cell may be differentiated into a granulocyte prior to administration. In preferred embodiments a stem cell may be differentiated into a different stem cell (preferably a precursor cell) prior to administration. In other embodiments a stem cell may be administered to a subject. A stem cell may be administered by any suitable technique known in the art. In one embodiment a subject may be given a stem cell transplant, such as a bone marrow transplant.

Prior to administration there may be a matching step between a medicament of the invention (e.g. a granulocyte, stem cell, composition, pharmaceutical composition or kit of the invention) and the subject to be treated. Matching may be based on data derived from the donor from which the stem cell, or granulocyte is derived, and similar data obtained from the subject to be treated. Matching may be achieved on the basis of blood group type, human leukocyte antigen (HLA) type similarity, or combinations thereof.

A granulocyte, stem cell, composition, pharmaceutical composition or kit of the invention may be administered to a subject in a therapeutically effective amount or a prophylactically effective amount.

The term “treat” or “treating” as used herein encompasses prophylactic treatment (e.g. to prevent onset of a disease) as well as corrective treatment (treatment of a subject already suffering from a disease). Preferably “treat” or “treating” as used herein means corrective treatment.

The term “treat” or “treating” as used herein refers to the disorder and/or a symptom thereof.

A “therapeutically effective amount” is any amount of the granulocyte, stem cell, composition, pharmaceutical composition or kit of the invention, which when administered alone or in combination to a subject for treating an infection (or a symptom thereof) is sufficient to effect such treatment of the disorder, or symptom thereof.

A “prophylactically effective amount” is any amount of the granulocyte, stem cell, composition, pharmaceutical composition or kit of the invention that, when administered alone or in combination to a subject inhibits or delays the onset or reoccurrence of an infection (or a symptom thereof). In some embodiments, the prophylactically effective amount prevents the onset or reoccurrence of an infection entirely. “Inhibiting” the onset means either lessening the likelihood of onset of an infection (or symptom thereof), or preventing the onset entirely.

In one embodiment a granulocyte is administered to a subject. Preferably, the granulocyte is a neutrophil.

In one embodiment a stem cell is administered to a subject. Preferably, the stem cell is a precursor cell, e.g. selected from a common myeloid progenitor cell, a myeloblast, a promyelocyte (e.g. a N. promyelocyte), a myelocyte (e.g. a N. myelocyte), a metamyelocyte (e.g. a N. metamyelocyte), a band (e.g. an N. band), or combinations thereof.

In some embodiments a stem cell and a granulocyte are administered to a subject. Preferably, a precursor cell and a neutrophil are administered to a subject.

An appropriate dosage range is one that produces the desired therapeutic effect (e.g. wherein the granulocyte, stem cell, composition, pharmaceutical composition or kit of the invention is dosed in a therapeutically or prophylactically effective amount).

A typical treatment regimen may include administering from 10⁶, 10⁷, 10⁸ or 10⁹ cells (e.g. granulocyte cells or stem cells) to a subject, or up to 10¹², 10¹³ or 10¹⁴ cells to a subject. In one embodiment a treatment regimen includes administering a dose of at least 1×10⁹ cells to a subject. Suitably, a treatment regimen may include administering a dose of at least 2×10⁹ cells or at least 5×10⁹ cells to a subject. In one embodiment a treatment regimen may include administering a dose of at least 1×10¹⁰ cells or at least 5×10¹⁰ cells to a subject. At least 1×10¹¹ or at least 2×10¹¹ cells may be administered to a subject. In some embodiments between 1×10⁹ to 3×10¹¹ or 1×10¹⁰ to 3×10¹¹ cells are administered to a subject. Suitably, between 5×10¹⁰ to 2.5×10¹¹ cells are administered to a subject. In one embodiment when the cell is a stem cell, e.g. a precursor cell as defined herein, a treatment regimen includes administering a dose between 1/100^{th} and 1/700^{th}, preferably a dose between 1/200^{th} and 1/400^{th}, such as 1/300^{th}, of the dose when compared to the dose of granulocytes administered.

A subject for treatment may be dosed once, twice, three times, four times, five times, or six times per week. Alternatively a subject may be dosed daily (e.g. once or twice daily). In other embodiments a subject may be dosed once weekly or bi-weekly. Preferably the dose is weekly. The skilled person will appreciate that the dose can be tailored based on the needs of the subject, and efficacy of the medicament. For example, where the medicament is highly efficacious, the dose may be lowered.

In one embodiment a subject for treatment is dosed weekly (e.g. once weekly) with at least 2×10⁹ cells or at least 2×10¹⁰ cells. Suitably, a subject for treatment may be dosed weekly with at least 1×10¹¹ or at least 2×10¹¹ cells.

The treatment term can be varied based on the response of the subject to the treatment, and/or the type and/or severity of the infection. For example, the subject for treatment may be dosed for at least 1 or 2 weeks. Suitably the subject for treatment may be dosed for at least 3 or 4 weeks. In one embodiment the subject for treatment is dosed for at least 5 or 6 weeks, suitably at least 7 or 8 weeks.

In one embodiment a subject for treatment is dosed for 4-8 weeks with at least 2×10⁹ cells, wherein said cells are administered once weekly. Suitably a subject for treatment is dosed for 8 weeks with at least 2×10⁹ cells (preferably at least 2×10¹⁰ or 2×10¹¹ cells), wherein said cells are administered once weekly.

Administration may be by any suitable technique or route, including but not limited to intravenous injection, intra-arterial injection, intraperitoneal injection, intrathecal injection, or combinations thereof. Suitably the medicament may be administered intravenously.

In one embodiment a medicament may be administered to an infected wound (e.g. as part of wound care). The medicament may comprise a stem cell or granulocyte of the invention. Preferably the medicament comprises a granulocyte of the invention.

In one embodiment, the medicament comprising a granulocyte of the invention is administered (e.g. sequentially or simultaneously) with infrared light treatment. In another embodiment, the medicament comprising a stem cell of the invention is administered (e.g. sequentially or simultaneously) with infrared light treatment. In another embodiment a donor or subject may be subjected to infrared light treatment. Said treatment may increase granulocyte function and proliferation.

The infrared light may have a wavelength of between 500-1500 nm, such as 750-1200 nm. In one embodiment, the subject is subjected to short bursts of high power (for example between 230-1500 W, preferably 300-1000 W, e.g. 300, 500, or 1000 W) near-infrared light. In one embodiment the subject is subjected to said near-infrared light for at least 30 seconds, e.g. at least 1, 10, 15, 20, 30, 40, or 60 minutes. The subject may be subjected for up to 5 times a day (e.g. 1, 2, 3 or 4 times per day) for up to 6 weeks (e.g. up to 1, 2, 3, 4 or 5 weeks).

In one embodiment, the subject is subjected to longer periods of low power (for example 50-220W, e.g. 100-200 W) near-infrared light. In one embodiment the subject is subjected to said near-infrared light for 1-10 hours, e.g. 2-6 hours, for up to 6 weeks (e.g. up to 1, 2, 3, 4 or 5 weeks).

In one embodiment, the subject is subjected to a combination of high power and low power near-infrared light.

A white blood cell growth factor may be administered with a medicament of the invention. The administration may be sequential or simultaneous (suitably simultaneous). Suitable white blood cell growth factors may include a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta, or combinations thereof. Suitably, the white blood cell growth factors comprises IFN-gamma and GM-CSF. Preferably, the white blood cell growth factors comprises TNF-alpha. Suitably the white blood cell growth factors may comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). Suitably the white blood cell growth factors may comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. Particular examples of the foregoing include but are not limited to LEUKINE® brand sargramostim, NEUPOGEN® brand filgrastim, and NEULAST A® brand 5 PEG-filgrastim.

In one embodiment a stem cell may be administered (e.g. sequentially or simultaneously, preferably simultaneously) with a granulocyte-colony stimulating factor; and a growth hormone; and a serotonin; and an interleukin. In one embodiment a granulocyte precursor cell (e.g. a granulocyte precursor cell culture) is administered (e.g. sequentially or simultaneously, preferably simultaneously) with a granulocyte-colony stimulating factor; and a growth hormone; and a serotonin; and an interleukin.

In one aspect the present invention provides a method for determining the suitability of a stem cell for treating an infection, the method comprising:
a. comparing a measured expression level of one or more genes by the stem cell, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. determining the suitability of the stem cell for treating an infection based on the comparison.

In one aspect the present invention provides a method for determining the suitability of a stem cell for treating an infection, the method comprising:
a. measuring an expression level of one or more genes by the stem cell, wherein the one or more genes are associated with suitability for treating an infection and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. determining the suitability of the stem cell for treating an infection based on the comparison.

In another aspect the invention provides a method for identifying whether or not a donor produces stem cells suitable for treating an infection, the method comprising:
a. comparing a measured expression level of one or more genes by a stem cell comprised in a sample obtainable from the donor, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. identifying whether or not the donor produces stem cells suitable for treating an infection based on the comparison.

In a related aspect the invention provides a method for identifying whether or not a donor produces stem cells for treating an infection, the method comprising:
a. measuring an expression level of one or more genes by a stem cell comprised in a sample obtainable from the donor, wherein the one or more genes are associated with suitability for treating an infection and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. identifying whether or not the donor produces stem cells for treating an infection based on the comparison.

In one embodiment a stem cell is determined to be suitable for treating an infection or a donor is identified as producing stem cells suitable for treating an infection when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to the reference standard when the reference standard is from a stem cell unsuitable for treating an infection; or
ii. a measured expression level of ANXA1 and/or PPP3CB is decreased when compared to the reference standard, when the reference standard is from a stem cell unsuitable for treating an infection; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to the reference standard, when the reference standard is from a stem cell suitable for treating an infection; or
iv. a measured expression level of ANXA1 and/or PPP3CB is decreased or the same when compared to the reference standard, when the reference standard is from a stem cell suitable for treating an infection.

Alternatively, in one embodiment a stem cell is determined to be unsuitable for treating an infection or a donor is identified as producing stem cells unsuitable for treating an infection when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to the reference standard, when the reference standard is from a stem cell unsuitable for treating an infection; or
ii. a measured expression level of ANXA1 and/or PPP3CB is increased or the same when compared to the reference standard, when the reference standard is from a stem cell unsuitable for treating an infection; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to reference standard, when the reference standard is from a stem cell suitable for treating an infection; or
iv. a measured expression level of ANXA1 and/or PPP3CB is increased when compared to the reference standard, when the reference standard is from a stem cell suitable for treating an infection.

In one aspect the invention provides a stem cell, wherein the stem cell comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a stem cell unsuitable for treating an infection; and/or
b. decreased expression of ANXA1 and/or PPP3CB when compared to a reference standard, wherein the reference standard is from a stem cell unsuitable for treating an infection.

In one aspect the invention provides a method for selecting whether or not a subject is suitable for treatment with a stem cell or granulocyte for treating an infection, the method comprising:
a. comparing a measured expression level of one or more genes by a stem cell comprised in a sample obtainable from the subject, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2 with the expression level of the same one or more genes in a reference standard; and
b. identifying whether or not the subject is suitable for treatment with a stem cell or granulocyte for treating an infection based on the comparison.

In one aspect the invention provides a method for selecting whether or not a subject is suitable for treatment with a stem cell or granulocyte for treating an infection, the method comprising:
a. measuring an expression level of one or more genes by a stem cell comprised in a sample obtainable from the subject, wherein the one or more genes are associated with suitability for treating an infection and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2;
b. comparing the measured expression level with the expression level of the same one or more genes in a reference standard; and
c. identifying whether or not the subject is suitable for treatment with a stem cell or granulocyte for treating an infection based on the comparison.

In one embodiment, step c. of any one of the foregoing aspects comprises:
identifying the subject as suitable for treatment with a granulocyte or a stem cell for treating an infection when:
i. the measured expression level of one or more of CTSG, CAP37, ITGB1, CYBB, SYK, DOCK8, COMP, ATG7, SLC2A1, GZMKATM, IKBKB, BCAP31, TAPBP, PERM, PLEC, ACSL1, RAC1, GM2A, and PSMB2 is decreased or the same when compared to the reference standard, when the reference standard is from a stem cell unsuitable for treating an infection; or
ii. the measured expression level of ANXA1 and/or PPP3CB is increased or the same when compared to the reference standard, when the reference standard is from a stem cell unsuitable for treating an infection; or
iii. the measured expression level of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 is decreased when compared to the reference standard, when the reference standard is from a stem cell suitable for treating an infection; or
iv. the measured expression level of ANXA1 and/or PPP3CB is increased when compared to the reference standard, when the reference standard is from a stem cell suitable for treating an infection; or
identifying the subject as unsuitable for treatment with a granulocyte or a stem cell for treating an infection when:
v. the measured expression level of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 is increased when compared to the reference standard when the reference standard is from a stem cell unsuitable for treating an infection; or
vi. the measured expression level of ANXA1 and/or PPP3CB is decreased when compared to the reference standard, when the reference standard is from a stem cell unsuitable for treating an infection; or
vii. the measured expression level of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 is increased or the same when compared to the reference standard, when the reference standard is from a stem cell suitable for treating an infection; or
viii. the measured expression level of ANXA1 and/or PPP3CB is decreased or the same when compared to the reference standard, when the reference standard is from a stem cell suitable for treating an infection.

In some embodiments of any of the foregoing aspects, the method does not comprise measuring the expression level of CD10 and/or CD101 by a granulocyte or stem cell comprised in a sample from a donor. In some embodiments of any of the foregoing aspects, the method does not comprise comparing the measured expression level of CD10 and/or CD101 with the expression level with the same genes in a reference standard.

In alternative embodiments of any of the foregoing aspects, the method comprises measuring the expression level of CD10 and/or CD101 by a granulocyte or stem cell comprised in a sample from a donor. In some embodiments of any of the foregoing aspects, the method comprises comparing the measured expression level of CD10 and/or CD101 with the expression level with the same genes in a reference standard.

In one aspect the invention provides a composition for treating an infection, the composition comprising stem cells: wherein at least 90% (preferably at least 95%, 99% or 100%) of the stem cells comprised in the composition have:
a. increased expression of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB21TGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a stem cell unsuitable for treating an infection; and/or
b. decreased expression of ANXA1 and/or PPP3CB when compared to a reference standard, wherein the reference standard is from a stem cell unsuitable for treating an infection.

The invention also provides a method for isolating stem cells suitable for treating an infection based on the expression of one or more genes of the invention. Such methods may provide a substantially homogeneous population of stem cells that are suitable for treating an infection. In one embodiment, stem cells for treating an infection are isolated using the expression of one or more cell-surface expressed polypeptides selected from: ATG7, CYBB, DOCK8, CTSG, S100A9, COMP, S100A8, CTSG, SYK, ITGB1, SLC2A1, GZMK, ANXA1, RAC1, and CAP37, preferably one or more selected from: ATG7, S100A9, COMP, S100A8, CTSG, SYK, ITGB1, SLC2A1, GZMK, ANXA1, RAC1, and CAP37. The isolation may be performed using any suitable technique. In one embodiment a method for isolating granulocytes comprises the use of a binding means that binds to a protein of the invention. Preferably the binding means is an antibody. Antibodies to detect the presence or absence of polypeptides of the invention are commercially available and may be one or more of the antibodies described herein. The method may comprise the use of flow cytometric techniques, preferably fluorescence activated cell sorting (FACS), e.g. together with appropriate ‘gating’. Flow cytometric techniques may be particularly suitable when the method employs the use of a binding means coupled to a detectable label, such as a fluorophore.

In one aspect there is provided a method for isolating a stem cell for treating an infection, the method comprising:
a) contacting a sample of stem cells with a binding means, wherein the binding means binds to one or more polypeptides selected from CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2; and
b) isolating the stem cell based on the presence or absence of binding between the binding means and the one or more polypeptides.

Binding may be determined to be present when the amount of binding is statistically significant (e.g. when compared to a ‘background’ control). Binding may be determined to be absent when the amount of binding is statistically insignificant (e.g. when compared to a ‘background’ control). Preferably, binding is determined to be absent when there is no binding whatsoever.

In one embodiment the invention comprises detecting the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2. When said one or more polypeptides are detected (i.e. where there is binding between the binding means and the polypeptide) the stem cell may be isolated. Suitably, said isolated stem cell may be a stem cell for treating an infection.

In another embodiment the invention may comprise detecting the absence of ANXA1 and/or PPP3CB (e.g. not detecting ANXA1 and/or PPP3CB). When said one or more polypeptides are not detected (i.e. where there is an absence of binding between the binding means and the polypeptide) the stem cell may be isolated. Suitably, said isolated stem cell may be a stem cell for treating an infection.

Preferably, the invention comprises detecting:
the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
the absence of ANXA1 and/or PPP3CB. In one embodiment, when said polypeptides are detected or not detected (as indicated), the stem cell is isolated.

In one embodiment a method of isolating a stem cell comprises the use of an immobilised binding means (e.g. a binding means conjugated to a bead, such as a magnetic bead, or chromatographic resin) to isolate a stem cell of the invention. Such methods may be immuno-affinity methods.

The method may comprise quantifying the amount of binding between the binding means and the one or more polypeptides or between the binding means and the stem cell. The method may comprise isolating a stem cell for treating an infection based on the quantified amount of binding.

In one embodiment a stem cell for treating an infection is isolated when there is a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2.

In one embodiment a stem cell for treating an infection is isolated when there is a low level of binding between a binding means and ANXA1 and/or PPP3CB.

Preferably, a stem cell for treating an infection is isolated when there is:
a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
a low level of binding between a binding means and ANXA1 and/or PPP3CB.

A high/low level of binding is preferably relative to a level of binding between the same binding means and polypeptide under the same conditions for a stem cell that is unsuitable for treating an infection.

The term “isolating” may mean providing a population of stem cells in which at least 50%, 60%, 70%, 80% or 90% (preferably at least 95%, 99% or 100%) are stem cells suitable for treating an infection. In other words, the term “isolating” may mean removing at least 50%, 60%, 70%, 80% or 90% (preferably at least 95%, 99% or 100%) of stem cells that are unsuitable for treating an infection from a population of stem cells.

Thus, the methods for isolating suitably allow for the separation of a stem cell for treating an infection from stem cell that is unsuitable for treating an infection.

In some embodiments a method described herein comprises discarding stem cells that are unsuitable for treating an infection.

In one aspect the invention provides a stem cell for treating an infection obtainable by a method of the invention (e.g. a stem cell capable of differentiating into granulocytes that are suitable to treat an infection). In one aspect the invention provides a stem cell for use in treating an infection (together with associated methods of treatment employing the same).

In one aspect there is provided a method for producing a stem cell for treating an infection, the method comprising:
a. providing a cell; and
b. converting the cell into a stem cell having an expression profile described herein, for example a stem cell that is capable of differentiating into a granulocyte having an expression profile described herein, e.g. wherein:
   i. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating an infection; or
   ii. the measured expression level of ANXA1 and/or PPP3CB is decreased in the granulocyte when compared to the reference standard, when the reference standard is from a granulocyte unsuitable for treating an infection; or
   iii. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating an infection; or
   iv. the measured expression level of ANXA1 and/or PPP3CB is decreased or the same when compared to the reference standard, when the reference standard is from a granulocyte suitable for treating an infection; and
c. optionally isolating the stem cell.

In one embodiment the cell is a somatic/differentiated cell, optionally from a donor who produces granulocytes suitable for treating an infection, for example as determined according to a method of the invention.

In one embodiment the methods of the invention comprise:
not obtaining a stem cell from a sample from a donor when a granulocyte kills less than or equal to 41.23% of infective agent or cells infected with an infective agent;
not obtaining a stem cell from a sample from a donor when the % of infective agent or cells infected with an infective agent is the same or less than the % killed in a control sample;
not selecting a granulocyte when a granulocyte kills less than or equal to 41.23% of infective agent or cells infected with an infective agent; and/or
not obtaining a granulocyte from a sample from a donor when the % of infective agent or cells infected with an infective agent is the same or less than the % killed in a control sample.

In some embodiments of any of the foregoing aspects, a granulocyte unsuitable for treating an infection is a viable granulocyte.

Embodiments related to the various methods of the invention are intended to be applied equally to other methods, the granulocytes, stem cells, compositions, pharmaceutical compositions or uses, and vice versa.

### Sequence Identity

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ length of the longer sequence plus the number of gaps introduced int o the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
lysine
histidine
Acidic: glutamic acid
aspartic acid
Polar: glutamine
asparagine
Hydrophobic: leucine
isoleucine
valine
Aromatic: phenylalanine
tryptophan
tyrosine
Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a granulocyte” includes a plurality of such candidate agents and reference to “the granulocyte” includes reference to one or more haematopoietic cells and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Figures and Examples

### BRIEF DESCRIPTION OF THE DRAWINGS

Embodiments of the invention will now be described, by way of example only, with reference to the following Figures, in which:

FIG. 1 demonstrates validation of an in vitro assay of neutrophil mediated bacterial killing by neutrophils in suspension at concentrations of 1×10⁵, 5×10⁵ or 1×10⁶/100 μl which were incubated with 1×10⁶ cfu P. aeruginosa RP73 for 2 hours at 37° C. under 120 rpm of shaking and quantified on TSA plates to calculate percentage of bacterial killing. Data are expressed as Mean±SEM of bacterial killing (%), n=2, *=P<0.05, **=P<0.01, ***=P<0.001.

FIG. 2 shows comparison of neutrophil mediated bacterial killing against Gram-negative and Gram-positive bacteria. 1×10⁶/100 μl neutrophils were incubated with either 1×10⁶ cfu P. aeruginosa RP73 (A) or MRSA USA300 (B) for 2 hours at 37° C. under 120 rpm of shaking and quantified on TSA plates to calculate percentage of bacterial killing. Data are expressed as Mean±SEM of bacterial killing (%), n=20.

FIG. 3 shows effects of tobramycin or vancomycin on bacterial killing at 2 hours. 1×10⁶ cfu P. aeruginosa RP73 (A) or MRSA USA300 (B) were treated with either 1, 10 and 100 μg/ml tobramycin (RP73) or vancomycin (MRSA) for 2 hours at 37° C. under 120 rpm of shaking and quantified on TSA plates to calculate percentage of bacterial killing. Data are expressed as Mean±SEM of bacterial killing (%), n=2, *=P<0.05, **=P<0.01, ***=P<0.001.

FIG. 4 shows comparison of neutrophil BKA against antibiotic therapy in vitro. 1×10⁶ cfu P. aeruginosa RP73 (A) or MRSA USA300 (B) were treated with either 1 μg/ml tobramycin (RP73), 1 μg/ml vancomycin (MRSA) or 1×10⁶ neutrophils for 2 hours at 37° C. under 120 rpm of shaking and quantified on TSA plates to calculate percentage of bacterial killing. Data are expressed as Mean±SEM of bacterial killing (%), n=2-3, *=P<0.05, **=P<0.01, ***=P<0.001.

FIG. 5 shows BKA assay results for Donor Derived Neutrophils and Stem Cell Derived Neutrophils for donors A, B, and C against MRSA.

FIG. 6 shows BKA assay results for Donor Derived Neutrophils and Stem Cell Derived Neutrophils for donors A, B, and C against P. aeruginosa RP73.

FIG. 7 shows significantly (***P<0.001, **P<0.01, *P<0.05) upregulated expression of ITGB1, SYK, DOCK8 and CYBB in high BKA neutrophils (>41.23% MRSA BKA) compared with low BKA control neutrophils. Data are mean (n=3 donor samples in duplicate)±standard error of mean compared by two-way ANOVA.

FIG. 8 shows significantly (**P<0.01, *P<0.05) upregulated expression of PLEC and COMP in high BKA neutrophils (>41.23% MRSA BKA) compared with low BKA control neutrophils. Data are mean (n=3 donor samples in duplicate)±standard error of mean compared by two-way ANOVA.

FIG. 9 shows significantly (**P<0.01, *P<0.05) upregulated expression of ATG7, SLC2A1, S100A9, ACSL1, CTSG, PSMB2, ATM and S100A8 in high BKA neutrophils (>41.23% MRSA BKA) compared with low BKA control neutrophils. Data are mean (n=3 donor samples in duplicate)±standard error of mean compared by two-way ANOVA.

FIG. 10 shows significantly (**P<0.01, *P<0.05) downregulated expression of ANXA1 in high BKA neutrophils (>41.23% MRSA BKA) compared with low BKA control neutrophils. Data are mean (n=3 donor samples in duplicate)±standard error of mean compared by two-way ANOVA.

FIG. 11 shows significantly (**P<0.01, *P<0.05) upregulated expression of BCAP31 and TAPBP in high BKA neutrophils (>41.23% MRSA BKA) compared with low BKA control neutrophils. Data are mean (n=3 donor samples in duplicate)±standard error of mean compared by two-way ANOVA.

### SEQUENCE LISTING

Gene Sequences

| SEQ ID NO. | GENE | Accession No. | Ensembl Release No. |
|---|---|---|---|
| 1 | CTSG | ENSG00000100448 | 97 |
| 2 | CAP37 | ENSG00000172232 | 97 |
| 3 | ITGB1 | ENSG00000150093 | 97 |
| 4 | CYBB | ENSG00000165168 | 97 |
| 5 | SYK | ENSG00000165025 | 97 |
| 6 | DOCK8 | ENSG00000107099 | 97 |
| 7 | COMP | ENSG00000105664 | 97 |
| 8 | ATG7 | ENSG00000197548 | 97 |
| 9 | SLC2A1 | ENSG00000117394 | 97 |
| 10 | GZMK | ENSG00000113088 | 97 |
| 11 | S100A9 | ENSG00000163220 | 97 |
| 12 | S100A8 | ENSG00000143546 | 97 |
| 13 | ATM | ENSG00000149311 | 97 |
| 14 | IKBKB | ENSG00000104365 | 97 |
| 15 | BCAP31 | ENSG00000185825 | 97 |
| 16 | TAPBP | ENSG00000231925 | 97 |
| 17 | PPP3CB | ENSG00000107758 | 97 |
| 18 | ANXA1 | ENSG00000135046 | 97 |
| 19 | PERM | ENSG00000005381 | 97 |
| 20 | PLEC | ENSG00000178209 | 97 |
| 21 | ACSL1 | ENSG00000151726 | 97 |
| 22 | RAC1 | ENSG00000136238 | 97 |
| 23 | PSMB2 | ENSG00000126067 | 97 |
| 24 | GM2A | ENSG00000196743 | 97 |

Polypeptide Sequences

| SEQ | | | | UniProt | |
|---|---|---|---|---|---|
| ID | POLY- | | UniProt | Version | |
| NO. | PEPTIDE | ISOFORM | ID | Number | SEQUENCE |
| 25 | CTSG | 1 | P08311 | Entry | MQPLLLLLAF LLPTGAEAGE |
| | | | | version 191 | IIGGRESRPH SRPYMAYLQI |
| | | | | (18 Sep. | QSPAGQSRCG GFLVREDFVL |
| | | | | 2019) | TAAHCWGSNI NVTLGAHNIQ |
| | | | | Sequence | RRENTQQHIT ARRAIRHPQY |
| | | | | version 2 | NQRTIQNDIM LLQLSRRVRR |
| | | | | (01 Jan. | NRNVNPVALP RAQEGLRPGT |
| | | | | 1990) | LCTVAGWGRV SMRRGTDTLR |
| | | | | | EVQLRVQRDR QCLRIFGSYD |
| | | | | | PRRQICVGDR RERKAAFKGD |
| | | | | | SGGPLLCNNV AHGIVSYGKS |
| | | | | | SGVPPEVFTR VSSFLPWIRT |
| | | | | | TMRSFKLLDQ METPL |
| 26 | CAP37 | 1 | P20160 | Entry version | MTRLTVLALL AGLLASSRAG |
| | | | | 191 (18 Sep. | SSPLLDIVGG RKARPRQFPF |
| | | | | 2019) | LASIQNQGRH FCGGALIHAR |
| | | | | Sequence | FVMTAASCFQ SQNPGVSTVV |
| | | | | version 3 (01 | LGAYDLRRRE RQSRQTFSIS |
| | | | | Oct. 1993) | SMSENGYDPQ QNLNDLMLLQ |
| | | | | | LDREANLTSS VTILPLPLQN |
| | | | | | ATVEAGTRCQ VAGWGSQRSG |
| | | | | | GRLSRFPRFV NVTVTPEDQC |
| | | | | | RPNNVCTGVL TRRGGICNGD |
| | | | | | GGTPLVCEGL AHGVASFSLG |
| | | | | | PCGRGPDFFT RVALFRDWID |
| | | | | | GVLNNPGPGP A |
| 27 | ITGB1 | 1 | P05556 | Entry version | MNLQPIFWIG LISSVCCVFA |
| | | | | 242 (18 Sep. | QTDENRCLKA NAKSCGECIQ |
| | | | | 2019) | AGPNCGWCTN STFLQEGMPT |
| | | | | Sequence | SARCDDLEAL KKKGCPPDDI |
| | | | | version 2 (16 | ENPRGSKDIK KNKNVTNRSK |
| | | | | Dec. 2008) | GTAEKLKPED ITQIQPQQLV |
| | | | | | LRLRSGEPQT FTLKFKRAED |
| | | | | | YPIDLYYLMD LSYSMKDDLE |
| | | | | | NVKSLGTDLM NEMRRITSDF |
| | | | | | RIGFGSFVEK TVMPYISTTP |
| | | | | | AKLRNPCTSE QNCTSPFSYK |
| | | | | | NVLSLTNKGE VFNELVGKQR |
| | | | | | ISGNLDSPEG GFDAIMQVAV |
| | | | | | CGSLIGWRNV TRLLVFSTDA |
| | | | | | GFHFAGDGKL GGIVLPNDGQ |
| | | | | | CHLENNMYTM SHYYDYPSIA |
| | | | | | HLVQKLSENN IQTIFAVTEE |
| | | | | | FQPVYKELKN LIPKSAVGTL |
| | | | | | SANSSNVIQL IIDAYNSLSS |
| | | | | | EVILENGKLS EGVTISYKSY |
| | | | | | CKNGVNGTGE NGRKCSNISI |
| | | | | | GDEVQFEISI TSNKCPKKDS |
| | | | | | DSFKIRPLGF TEEVEVILQY |
| | | | | | ICECECQSEG IPESPKCHEG |
| | | | | | NGTFECGACR CNEGRVGRHC |
| | | | | | ECSTDEVNSE DMDAYCRKEN |
| | | | | | SSEICSNNGE CVCGQCVCRK |
| | | | | | RDNTNEIYSG KFCECDNFNC |
| | | | | | DRSNGLICGG NGVCKCRVCE |
| | | | | | CNPNYTGSAC DCSLDTSTCE |
| | | | | | ASNGQICNGR GICECGVCKC |
| | | | | | TDPKFQGQTC EMCQTCLGVC |
| | | | | | AEHKECVQCR AFNKGEKKDT |
| | | | | | CTQECSYFNI TKVESRDKLP |
| | | | | | QPVQPDPVSH CKEKDVDDCW |
| | | | | | FYFTYSVNGN NEVMVHVVEN |
| | | | | | PECPTGPDII PIVAGVVAGI |
| | | | | | VLIGLALLLI WKLLMIIHDR |
| | | | | | REFAKFEKEK MNAKWDTGEN |
| | | | | | PIYKSAVTTV VNPKYEGK |
| 28 | ITGB1 | 2 | P05556- | Entry version | MNLQPIFWIG LISSVCCVFA |
| | | | 2 | 242 (18 Sep. | QTDENRCLKA NAKSCGECIQ |
| | | | | 2019) | AGPNCGWCTN STFLQEGMPT |
| | | | | Sequence | SARCDDLEAL KKKGCPPDDI |
| | | | | version 2 (16 | ENPRGSKDIK KNKNVTNRSK |
| | | | | Dec. 2008) | GTAEKLKPED ITQIQPQQLV |
| | | | | | LRLRSGEPQT FTLKFKRAED |
| | | | | | YPIDLYYLMD LSYSMKDDLE |
| | | | | | NVKSLGTDLM NEMRRITSDF |
| | | | | | RIGFGSFVEK TVMPYISTTP |
| | | | | | AKLRNPCTSE QNCTSPFSYK |
| | | | | | NVLSLTNKGE VFNELVGKQR |
| | | | | | ISGNLDSPEG GFDAIMQVAV |
| | | | | | CGSLIGWRNV TRLLVFSTDA |
| | | | | | GFHFAGDGKL GGIVLPNDGQ |
| | | | | | CHLENNMYTM SHYYDYPSIA |
| | | | | | HLVQKLSENN IQTIFAVTEE |
| | | | | | FQPVYKELKN LIPKSAVGTL |
| | | | | | SANSSNVIQL IIDAYNSLSS |
| | | | | | EVILENGKLS EGVTISYKSY |
| | | | | | CKNGVNGTGE NGRKCSNISI |
| | | | | | GDEVQFEISI TSNKCPKKDS |
| | | | | | DSFKIRPLGF TEEVEVILQY |
| | | | | | ICECECQSEG IPESPKCHEG |
| | | | | | NGTFECGACR CNEGRVGRHC |
| | | | | | ECSTDEVNSE DMDAYCRKEN |
| | | | | | SSEICSNNGE CVCGQCVCRK |
| | | | | | RDNTNEIYSG KFCECDNFNC |
| | | | | | DRSNGLICGG NGVCKCRVCE |
| | | | | | CNPNYTGSAC DCSLDTSTCE |
| | | | | | ASNGQICNGR GICECGVCKC |
| | | | | | TDPKFQGQTC EMCQTCLGVC |
| | | | | | AEHKECVQCR AFNKGEKKDT |
| | | | | | CTQECSYFNI TKVESRDKLP |
| | | | | | QPVQPDPVSH CKEKDVDDCW |
| | | | | | FYFTYSVNGN NEVMVHVVEN |
| | | | | | PECPTGPDII PIVAGVVAGI |
| | | | | | VLIGLALLLI WKLLMIIHDR |
| | | | | | REFAKFEKEK MNAKWDTVSY |
| | | | | | KTSKKQSGL |
| 29 | ITGB1 | 3 | P05556- | Entry version | MNLQPIFWIG LISSVCCVFA |
| | | | 3 | 242 (18 Sep. | QTDENRCLKA NAKSCGECIQ |
| | | | | 2019) | AGPNCGWCTN STFLQEGMPT |
| | | | | Sequence | SARCDDLEAL KKKGCPPDDI |
| | | | | version 2 (16 | ENPRGSKDIK KNKNVTNRSK |
| | | | | Dec. 2008) | GTAEKLKPED ITQIQPQQLV |
| | | | | | LRLRSGEPQT FTLKFKRAED |
| | | | | | YPIDLYYLMD LSYSMKDDLE |
| | | | | | NVKSLGTDLM NEMRRITSDF |
| | | | | | RIGFGSFVEK TVMPYISTTP |
| | | | | | AKLRNPCTSE QNCTSPFSYK |
| | | | | | NVLSLTNKGE VFNELVGKQR |
| | | | | | ISGNLDSPEG GFDAIMQVAV |
| | | | | | CGSLIGWRNV TRLLVFSTDA |
| | | | | | GFHFAGDGKL GGIVLPNDGQ |
| | | | | | CHLENNMYTM SHYYDYPSIA |
| | | | | | HLVQKLSENN IQTIFAVTEE |
| | | | | | FQPVYKELKN LIPKSAVGTL |
| | | | | | SANSSNVIQL IIDAYNSLSS |
| | | | | | EVILENGKLS EGVTISYKSY |
| | | | | | CKNGVNGTGE NGRKCSNISI |
| | | | | | GDEVQFEISI TSNKCPKKDS |
| | | | | | DSFKIRPLGF TEEVEVILQY |
| | | | | | ICECECQSEG IPESPKCHEG |
| | | | | | NGTFECGACR CNEGRVGRHC |
| | | | | | ECSTDEVNSE DMDAYCRKEN |
| | | | | | SSEICSNNGE CVCGQCVCRK |
| | | | | | RDNTNEIYSG KFCECDNFNC |
| | | | | | DRSNGLICGG NGVCKCRVCE |
| | | | | | CNPNYTGSAC DCSLDTSTCE |
| | | | | | ASNGQICNGR GICECGVCKC |
| | | | | | ETDPKFQGQT CMCQTCLGVC |
| | | | | | AEHKECVQCR AFNKGEKKDT |
| | | | | | CTQECSYENI TKVESRDKLP |
| | | | | | QPVQPDPVSH CKEKDVDDCW |
| | | | | | FYFTYSVNGN NEVMVHVVEN |
| | | | | | PECPTGPDII PIVAGVVAGI |
| | | | | | VLIGLALLLI WKLLMIIHDR |
| | | | | | REFAKFEKEK MNAKWDTSLS |
| | | | | | VAQPGVQWCD ISSLQPLTSR |
| | | | | | FQQFSCLSLP STWDYRVKIL |
| | | | | | FIRVP |
| 30 | ITGB1 | 4 | P05556- | Entry version | MNLQPIFWIG LISSVCCVFA |
| | | | 4 | 242 (18 Sep. | QTDENRCLKA NAKSCGECIQ |
| | | | | 2019) | AGPNCGWCTN STFLQEGMPT |
| | | | | Sequence | SARCDDLEAL KKKGCPPDDI |
| | | | | version 2 (16 | ENPRGSKDIK KNKNVTNRSK |
| | | | | Dec. 2008) | GTAEKLKPED ITQIQPQQLV |
| | | | | | LRLRSGEPQT FTLKFKRAED |
| | | | | | YPIDLYYLMD LSYSMKDDLE |
| | | | | | NVKSLGTDLM NEMRRITSDF |
| | | | | | RIGFGSFVEK TVMPYISTTP |
| | | | | | AKLRNPCTSE QNCTSPFSYK |
| | | | | | NVLSLTNKGE VFNELVGKQR |
| | | | | | ISGNLDSPEG GFDAIMQVAV |
| | | | | | CGSLIGWRNV TRLLVFSTDA |
| | | | | | GFHFAGDGKL GGIVLPNDGQ |
| | | | | | CHLENNMYTM SHYYDYPSIA |
| | | | | | HLVQKLSENN IQTIFAVTEE |
| | | | | | FQPVYKELKN LIPKSAVGTL |
| | | | | | SANSSNVIQL IIDAYNSLSS |
| | | | | | EVILENGKLS EGVTISYKSY |
| | | | | | CKNGVNGTGE NGRKCSNISI |
| | | | | | GDEVQFEISI TSNKCPKKDS |
| | | | | | DSFKIRPLGF TEEVEVILQY |
| | | | | | ICECECQSEG IPESPKCHEG |
| | | | | | NGTFECGACR CNEGRVGRHC |
| | | | | | ECSTDEVNSE DMDAYCRKEN |
| | | | | | SSEICSNNGE CVCGQCVCRK |
| | | | | | RDNTNEIYSG KFCECDNFNC |
| | | | | | DRSNGLICGG NGVCKCRVCE |
| | | | | | CNPNYTGSAC DCSLDTSTCE |
| | | | | | ASNGQICNGR GICECGVCKC |
| | | | | | TDPKFQGQTC EMCQTCLGVC |
| | | | | | AEHKECVQCR AFNKGEKKDT |
| | | | | | CTQECSYFNI TKVESRDKLP |
| | | | | | QPVQPDPVSH CKEKDVDDCW |
| | | | | | FYFTYSVNGN NEVMVHVVEN |
| | | | | | PECPTGPDII PIVAGVVAGI |
| | | | | | VLIGLALLLI WKLLMIIHDR |
| | | | | | REFAKFEKEK MNAKWDTPGV |
| | | | | | QWCDISSLQP LTSRFQQFSC |
| | | | | | LSLPSTWDYR VKILFIRVP |
| 31 | ITGB1 | 5 | P05556- | Entry version | MNLQPIFWIG LISSVCCVFA |
| | | | 5 | 242 (18 Sep. | QTDENRCLKA NAKSCGECIQ |
| | | | | 2019) | AGPNCGWCTN STFLQEGMPT |
| | | | | Sequence | SARCDDLEAL KKKGCPPDDI |
| | | | | version 2 (16 | ENPRGSKDIK KNKNVTNRSK |
| | | | | Dec. 2008) | GTAEKLKPED ITQIQPQQLV |
| | | | | | LRLRSGEPQT FTLKFKRAED |
| | | | | | YPIDLYYLMD LSYSMKDDLE |
| | | | | | NVKSLGTDLM NEMRRITSDF |
| | | | | | RIGFGSFVEK TVMPYISTTP |
| | | | | | AKLRNPCTSE QNCTSPFSYK |
| | | | | | NVLSLTNKGE VFNELVGKQR |
| | | | | | ISGNLDSPEG GFDAIMQVAV |
| | | | | | CGSLIGWRNV TRLLVFSTDA |
| | | | | | GFHFAGDGKL GGIVLPNDGQ |
| | | | | | CHLENNMYTM SHYYDYPSIA |
| | | | | | HLVQKLSENN IQTIFAVTEE |
| | | | | | FQPVYKELKN LIPKSAVGTL |
| | | | | | SANSSNVIQL IIDAYNSLSS |
| | | | | | EVILENGKLS EGVTISYKSY |
| | | | | | CKNGVNGTGE NGRKCSNISI |
| | | | | | GDEVQFEISI TSNKCPKKDS |
| | | | | | DSFKIRPLGF TEEVEVILQY |
| | | | | | ICECECQSEG IPESPKCHEG |
| | | | | | NGTFECGACR CNEGRVGRHC |
| | | | | | ECSTDEVNSE DMDAYCRKEN |
| | | | | | SSEICSNNGE CVCGQCVCRK |
| | | | | | RDNTNEIYSG KFCECDNFNC |
| | | | | | DRSNGLICGG NGVCKCRVCE |
| | | | | | CNPNYTGSAC DCSLDTSTCE |
| | | | | | ASNGQICNGR GICECGVCKC |
| | | | | | TDPKFQGQTC EMCQTCLGVC |
| | | | | | AEHKECVQCR AFNKGEKKDT |
| | | | | | CTQECSYFNI TKVESRDKLP |
| | | | | | QPVQPDPVSH CKEKDVDDCW |
| | | | | | FYFTYSVNGN NEVMVHVVEN |
| | | | | | PECPTGPDII PIVAGVVAGI |
| | | | | | VLIGLALLLI WKLLMIIHDR |
| | | | | | REFAKFEKEK MNAKWDTQEN |
| | | | | | PIYKSPINNF KNPNYGRKAG |
| | | | | | L |
| 32 | CYBB | 1 | P04839 | Entry version | MGNWAVNEGL SIFVILVWLG |
| | | | | 213 (18 Sep. | LNVFLFVWYY RVYDIPPKFF |
| | | | | 2019) | YTRKLLGSAL ALARAPAACL |
| | | | | Sequence | NFNCMLILLP VCRNLLSFLR |
| | | | | version 2 (23 | GSSACCSTRV RRQLDRNLTF |
| | | | | Jan. 2007) | HKMVAWMIAL HSAIHTIAHL |
| | | | | | FNVEWCVNAR VNNSDPYSVA |
| | | | | | LSELGDRQNE SYLNFARKRI |
| | | | | | KNPEGGLYLA VTLLAGITGV |
| | | | | | VITLCLILII TSSTKTIRRS |
| | | | | | YFEVFWYTHH LFVIFFIGLA |
| | | | | | IHGAERIVRG QTAESLAVHN |
| | | | | | ITVCEQKISE WGKIKECPIP |
| | | | | | QFAGNPPMTW KWIVGPMFLY |
| | | | | | LCERLVRFWR SQQKVVITKV |
| | | | | | VTHPFKTIEL QMKKKGFKME |
| | | | | | VGQYIFVKCP KVSKLEWHPF |
| | | | | | TLTSAPEEDF FSIHIRIVGD |
| | | | | | WTEGLFNACG CDKQEFQDAW |
| | | | | | KLPKIAVDGP FGTASEDVFS |
| | | | | | YEVVMLVGAG IGVTPFASIL |
| | | | | | KSVWYKYCNN ATNLKLKKIY |
| | | | | | FYWLCRDTHA FEWFADLLQL |
| | | | | | LESQMQERNN AGFLSYNIYL |
| | | | | | TGWDESQANH FAVHHDEEKD |
| | | | | | VITGLKQKTL YGRPNWDNEF |
| | | | | | KTIASQHPNT RIGVFLCGPE |
| | | | | | ALAETLSKQS ISNSESGPRG |
| | | | | | VHFIFNKENF |
| 33 | SYK | 1 | P43405 | Entry version | MASSGMADSA NHLPFFFGNI |
| | | | | 224 (18 Sep. | TREEAEDYLV QGGMSDGLYL |
| | | | | 2019) | LRQSRNYLGG FALSVAHGRK |
| | | | | Sequence | AHHYTIEREL NGTYAIAGGR |
| | | | | version 1 (01 | THASPADLCH YHSQESDGLV |
| | | | | Nov. 1995) | CLLKKPFNRP QGVQPKTGPF |
| | | | | | EDLKENLIRE YVKQTWNLQG |
| | | | | | QALEQAIISQ KPQLEKLIAT |
| | | | | | TAHEKMPWFH GKISREESEQ |
| | | | | | IVLIGSKTNG KFLIRARDNN |
| | | | | | GSYALCLLHE GKVLHYRIDK |
| | | | | | DKTGKLSIPE GKKFDTLWQL |
| | | | | | VEHYSYKADG LLRVLTVPCQ |
| | | | | | KIGTQGNVNF GGRPQLPGSH |
| | | | | | PATWSAGGII SRIKSYSFPK |
| | | | | | PGHRKSSPAQ GNRQESTVSF |
| | | | | | NPYEPELAPW AADKGPQREA |
| | | | | | LPMDTEVYES PYADPEEIRP |
| | | | | | KEVYLDRKLL TLEDKELGSG |
| | | | | | NFGTVKKGYY QMKKVVKTVA |
| | | | | | VKILKNEAND PALKDELLAE |
| | | | | | ANVMQQLDNP YIVRMIGICE |
| | | | | | AESWMLVMEM AELGPLNKYL |
| | | | | | QQNRHVKDKN IIELVHQVSM |
| | | | | | GMKYLEESNF VHRDLAARNV |
| | | | | | LLVTQHYAKI SDFGLSKALR |
| | | | | | ADENYYKAQT HGKWPVKWYA |
| | | | | | PECINYYKES SKSDVWSFGV |
| | | | | | LMWEAFSYGQ KPYRGMKGSE |
| | | | | | VTAMLEKGER MGCPAGCPRE |
| | | | | | MYDLMNLCWT YDVENRPGFA |
| | | | | | AVELRLRNYY YDVVN |
| 34 | SYK | 2 | P43405- | Entry version | MASSGMADSA NHLPFFFGNI |
| | | | 2 | 224 (18 Sep. | TREEAEDYLV QGGMSDGLYL |
| | | | | 2019) | LRQSRNYLGG FALSVAHGRK |
| | | | | Sequence | AHHYTIEREL NGTYAIAGGR |
| | | | | version 1 (01 | THASPADLCH YHSQESDGLV |
| | | | | Nov. 1995) | CLLKKPFNRP QGVQPKTGPF |
| | | | | | EDLKENLIRE YVKQTWNLQG |
| | | | | | QALEQAIISQ KPQLEKLIAT |
| | | | | | TAHEKMPWFH GKISREESEQ |
| | | | | | IVLIGSKTNG KFLIRARDNN |
| | | | | | GSYALCLLHE GKVLHYRIDK |
| | | | | | DKTGKLSIPE GKKFDTLWQL |
| | | | | | VEHYSYKADG LLRVLTVPCQ |
| | | | | | KIGTQGNVNF GGRPQLPGSH |
| | | | | | PASSPAQGNR QESTVSFNPY |
| | | | | | EPELAPWAAD KGPQREALPM |
| | | | | | DTEVYESPYA DPEEIRPKEV |
| | | | | | YLDRKLLTLE DKELGSGNFG |
| | | | | | TVKKGYYQMK KVVKTVAVKI |
| | | | | | LKNEANDPAL KDELLAEANV |
| | | | | | MQQLDNPYIV RMIGICEAES |
| | | | | | WMLVMEMAEL GPLNKYLQQN |
| | | | | | RHVKDKNIIE LVHQVSMGMK |
| | | | | | YLEESNFVHR DLAARNVLLV |
| | | | | | TQHYAKISDF GLSKALRADE |
| | | | | | NYYKAQTHGK WPVKWYAPEC |
| | | | | | INYYKFSSKS DVWSFGVLMW |
| | | | | | EAFSYGQKPY RGMKGSEVTA |
| | | | | | MLEKGERMGC PAGCPREMYD |
| | | | | | LMNLCWTYDV ENRPGFAAVE |
| | | | | | LRLRNYYYDV VN |
| 35 | DQCK8 | 1 | Q8NF50 | Entry version | MATLPSAERR AFALKINRYS |
| | | | | 160 (18 Sep. | SAEIRKQFTL PPNLGQYHRQ |
| | | | | 2019) | SISTSGFPSL QLPQFYDPVE |
| | | | | Sequence | PVDFEGLLMT HLNSLDVQLA |
| | | | | version 3 (21 | QELGDFTDDD LDVVFTPKEC |
| | | | | Aug. 2007) | RTLQPSLPEE GVELDPHVRD |
| | | | | | CVQTYIREWL IVNRKNQGSP |
| | | | | | EICGFKKTGS RKDFHKTLPK |
| | | | | | QTFESETLEC SEPAAQAGPR |
| | | | | | HLNVLCDVSG KGPVTACDFD |
| | | | | | LRSLQPDKRL ENLLQQVSAE |
| | | | | | DFEKQNEEAR RTNRQAELFA |
| | | | | | LYPSVDEEDA VEIRPVPECP |
| | | | | | KEHLGNRILV KLLTLKFEIE |
| | | | | | IEPLFASIAL YDVKERKKIS |
| | | | | | ENFHCDLNSD QFKGFLRAHT |
| | | | | | PSVAASSQAR SAVFSVTYPS |
| | | | | | SDIYLVVKIE KVLQQGEIGD |
| | | | | | CAEPYTVIKE SDGGKSKEKI |
| | | | | | EKLKLQAESF CQRLGKYRMP |
| | | | | | FAWAPISLSS FFNVSTLERE |
| | | | | | VTDVDSVVGR SSVGERRTLA |
| | | | | | QSRRLSERAL SLEENGVGSN |
| | | | | | FKTSTLSVSS FFKQEGDRLS |
| | | | | | DEDLFKFLAD YKRSSSLQRR |
| | | | | | VKSIPGLLRL EISTAPEIIN |
| | | | | | CCLTPEMLPV KPFPENRTRP |
| | | | | | HKEILEFPTR EVYVPHTVYR |
| | | | | | NLLYVYPQRL NFVNKLASAR |
| | | | | | NITIKIQFMC GEDASNAMPV |
| | | | | | IFGKSSGPEF LQEVYTAVTY |
| | | | | | HNKSPDFYEE VKIKLPAKLT |
| | | | | | VNHHLLFTFY HISCQQKQGA |
| | | | | | SVETLLGYSW LPILLNERLQ |
| | | | | | TGSYCLPVAL EKLPPNYSMH |
| | | | | | SAEKVPLQNP PIKWAEGHKG |
| | | | | | VFNIEVQAVS SVHTQDNHLE |
| | | | | | KFFTLCHSLE SQVTFPIRVL |
| | | | | | DQKISEMALE HELKLSIICL |
| | | | | | NSSRLEPLVL FLHLVLDKLF |
| | | | | | QLSVQPMVIA GQTANFSQFA |
| | | | | | FESVVAIANS LHNSKDLSKD |
| | | | | | QHGRNCLLAS YVHYVFRLPE |
| | | | | | VQRDVPKSGA PTALLDPRSY |
| | | | | | HTYGRTSAAA VSSKLLQARV |
| | | | | | MSSSNPDLAG THSAADEEVK |
| | | | | | NIMSSKIADR NCSRMSYYCS |
| | | | | | GSSDAPSSPA APRPASKKHF |
| | | | | | HEELALQMVV STGMVRETVF |
| | | | | | KYAWFFFELL VKSMAQHVHN |
| | | | | | MDKRDSFRRT RFSDRFMDDI |
| | | | | | TTIVNVVTSE IAALLVKPQK |
| | | | | | ENEQAEKMNI SLAFFLYDLL |
| | | | | | SLMDRGFVFN LIRHYCSQLS |
| | | | | | AKLSNLPTLI SMRLEFLRIL |
| | | | | | CSHEHYLNLN LFFMNADTAP |
| | | | | | TSPCPSISSQ NSSSCSSFQD |
| | | | | | QKIASMFDLT SEYRQQHFLT |
| | | | | | GLLFTELAAA LDAEGEGISK |
| | | | | | VQRKAVSAIH SLLSSHDLDP |
| | | | | | RCVKPEVKVK IAALYLPLVG |
| | | | | | IILDALPQLC DFTVADTRRY |
| | | | | | RTSGSDEEQE GAGAINQNVA |
| | | | | | LAIAGNNFNL KTSGIVLSSL |
| | | | | | PYKQYNMLNA DTTRNLMICF |
| | | | | | LWIMKNADQS LIRKWIADLP |
| | | | | | STQLNRILDL LFICVLCFEY |
| | | | | | KGKQSSDKVS TQVLQKSRDV |
| | | | | | KARLEEALLR GEGARGEMMR |
| | | | | | RRAPGNDRFP GLNENLRWKK |
| | | | | | EQTHWRQANE KLDKTKAELD |
| | | | | | QEALISGNLA TEAHLIILDM |
| | | | | | QENIIQASSA LDCKDSLLGG |
| | | | | | VLRVLVNSLN CDQSTTYLTH |
| | | | | | CFATLRALIA KFGDLLFEEE |
| | | | | | VEQCFDLCHQ VLHHCSSSMD |
| | | | | | VTRSQACATL YLLMRFSFGA |
| | | | | | TSNFARVKMQ VTMSLASLVG |
| | | | | | RAPDFNEEHL RRSLRTILAY |
| | | | | | SEEDTAMQMT PFPTQVEELL |
| | | | | | CNLNSILYDT VKMREFQEDP |
| | | | | | EMLMDLMYRI AKSYQASPDL |
| | | | | | RLTWLQNMAE KHTKKKCYTE |
| | | | | | AAMCLVHAAA LVAEYLSMLE |
| | | | | | DHSYLPVGSV SFQNISSNVL |
| | | | | | EESVVSEDTL SPDEDGVCAG |
| | | | | | QYFTESGLVG LLEQAAELFS |
| | | | | | TGGLYETVNE VYKLVIPILE |
| | | | | | AHREFRKLTL THSKLQRAFD |
| | | | | | SIVNKDHKRM FGTYFRVGFF |
| | | | | | GSKFGDLDEQ EFVYKEPAIT |
| | | | | | KLPEISHRLE AFYGQCFGAE |
| | | | | | FVEVIKDSTP VDKTKLDPNK |
| | | | | | AYIQITFVEP YFDEYEMKDR |
| | | | | | VTYFEKNFNL RRFMYTTPFT |
| | | | | | LEGRPRGELH EQYRRNTVLT |
| | | | | | TMHAFPYIKT RISVIQKEEF |
| | | | | | VLTPIEVAIE DMKKKTLQLA |
| | | | | | VAINQEPPDA KMLQMVLQGS |
| | | | | | VGATVNQGPL EVAQVFLAEI |
| | | | | | PADPKLYRHH NKLRLCFKEF |
| | | | | | IMRCGEAVEK NKRLITADQR |
| | | | | | EYQQELKKNY NKLKENLRPM |
| | | | | | IERKIPELYK PIFRVESQKR |
| | | | | | DSFHRSSFRK CETQLSQGS |
| 36 | DQCK8 | 2 | Q8NF50- | Entry version | MATLPSAERR AFALKINRYS |
| | | | 2 | 160 (18 Sep. | SAEIRKQFTL PPNLGQYHRQ |
| | | | | 2019) | SISTSGFPSL QLPQFYDPVE |
| | | | | Sequence | PVDFEGLLMT HLNSLDVQLA |
| | | | | version 3 (21 | QELGDFTDDD LDVVFTPKEC |
| | | | | Aug. 2007) | RTLQPSLPEE GVELDPHVRD |
| | | | | | CVQTYIREWL IVNRKNQGSP |
| | | | | | EICGFKKTGS RKDFHKTLPK |
| | | | | | QTFESETLEC SEPAAQAGPR |
| | | | | | HLNVLCDVSG KGPVTACDFD |
| | | | | | LRSLQPDKRL ENLLQQVSAE |
| | | | | | DFEKQNEEAR RTNRQAELFA |
| | | | | | LYPSVDEEDA VEIRPVPECP |
| | | | | | KEHLGNRILV KLLTLKFEIE |
| | | | | | IEPLFASIAL YDVKERKKIS |
| | | | | | ENFHCDLNSD QFKGFLRAHT |
| | | | | | PSVAASSQAR SAVFSVTYPS |
| | | | | | SDIYLVVKIE KVLQQGEIGD |
| | | | | | CAEPYTVIKE SDGGKSKEKI |
| | | | | | EKLKLQAESF CQRLGKYRMP |
| | | | | | FAWAPISLSS FFNVSTLERE |
| | | | | | VTDVDSVVGR SSVGERRTLA |
| | | | | | QSRRLSERAL SLEENGVGSN |
| | | | | | FKTSTLSVSS FFKQEGDRLS |
| | | | | | DEDLFKFLAD YKRSSSLQRR |
| | | | | | VKSIPGLLRL EISTAPEIIN |
| | | | | | CCLTPEMLPV KPFPENRTRP |
| | | | | | HKEILEFPTR EVYVPHTVYR |
| | | | | | NLLYVYPQRL NFVNKLASAR |
| | | | | | NITIKIQFMC GEDASNAMPV |
| | | | | | IFGKSSGPEF LQEVYTAVTY |
| | | | | | HNKSPDFYEE VKIKLPAKLT |
| | | | | | VNHHLLFTFY HISCQQKQGA |
| | | | | | SVETLLGYSW LPILLNERLQ |
| | | | | | TGSYCLPVAL EKLPPNYSMH |
| | | | | | SAEKVPLQNP PIKWAEGHKG |
| | | | | | VFNIEVQAVS SVHTQDNHLE |
| | | | | | KFFTLCHSLE SQVTFPIRVL |
| | | | | | DQKISEMALE HELKLSIICL |
| | | | | | NSSRLEPLVL FLHLVLDKLF |
| | | | | | QLSVQPMVIA GQTANFSQFA |
| | | | | | FESVVAIANS LHNSKDLSKD |
| | | | | | QHGRNCLLAS YVHYVFRLPE |
| | | | | | VQRDVPKSGA PTALLDPRSY |
| | | | | | HTYGRTSAAA VSSKLLQARV |
| | | | | | MSSSNPDLAG THSAADEEVK |
| | | | | | NIMSSKHFHE ELALQMVVST |
| | | | | | GMVRETVFKY AWFFFELLVK |
| | | | | | SMAQHVHNMD KRDSFRRTRF |
| | | | | | SDRFMDDITT IVNVVTSEIA |
| | | | | | ALLVKPQKEN EQAEKMNISL |
| | | | | | AFFLYDLLSL MDRGFVFNLI |
| | | | | | RHYCSQLSAK LSNLPTLISM |
| | | | | | RLEFLRILCS HEHYLNLNLF |
| | | | | | FMNADTAPTS PCPSISSQNS |
| | | | | | SSCSSFQDQK IASMFDLTSE |
| | | | | | YRQQHFLTGL LFTELAAALD |
| | | | | | AEGEGISKVQ RKAVSAIHSL |
| | | | | | LSSHDLDPRC VKPEVKVKIA |
| | | | | | ALYLPLVGII LDALPQLCDF |
| | | | | | TVADTRRYRT SGSDEEQEGA |
| | | | | | GAINQNVALA IAGNNFNLKT |
| | | | | | SGIVLSSLPY KQYNMLNADT |
| | | | | | TRNLMICFLW IMKNADQSLI |
| | | | | | RKWIADLPST QLNRILDLLF |
| | | | | | ICVLCFEYKG KQSSDKVSTQ |
| | | | | | VLQKSRDVKA RLEEALLRGE |
| | | | | | GARGEMMRRR APGNDRFPGL |
| | | | | | NENLRWKKEQ THWRQANEKL |
| | | | | | DKTKAELDQE ALISGNLATE |
| | | | | | AHLIILDMQE NIIQASSALD |
| | | | | | CKDSLLGGVL RVLVNSLNCD |
| | | | | | QSTTYLTHCF ATLRALIAKF |
| | | | | | GDLLFEEEVE QCFDLCHQVL |
| | | | | | HHCSSSMDVT RSQACATLYL |
| | | | | | LMRFSFGATS NFARVKMQVT |
| | | | | | MSLASLVGRA PDFNEEHLRR |
| | | | | | SLRTILAYSE EDTAMQMTPF |
| | | | | | PTQVEELLCN LNSILYDTVK |
| | | | | | MREFQEDPEM LMDLMYRIAK |
| | | | | | SYQASPDLRL TWLQNMAEKH |
| | | | | | TKKKCYTEAA MCLVHAAALV |
| | | | | | AEYLSMLEDH SYLPVGSVSF |
| | | | | | QNISSNVLEE SVVSEDTLSP |
| | | | | | DEDGVCAGQY FTESGLVGLL |
| | | | | | EQAAELFSTG GLYETVNEVY |
| | | | | | KLVIPILEAH REFRKLTLTH |
| | | | | | SKLQRAFDSI VNKDHKRMFG |
| | | | | | TYFRVGFFGS KFGDLDEQEF |
| | | | | | VYKEPAITKL PEISHRLEAF |
| | | | | | YGQCFGAEFV EVIKDSTPVD |
| | | | | | KTKLDPNKAY IQITFVEPYF |
| | | | | | DEYEMKDRVT YFEKNFNLRR |
| | | | | | FMYTTPFTLE GRPRGELHEQ |
| | | | | | YRRNTVLTTM HAFPYIKTRI |
| | | | | | SVIQKEEFVL TPIEVAIEDM |
| | | | | | KKKTLQLAVA INQEPPDAKM |
| | | | | | LQMVLQGSVG ATVNQGPLEV |
| | | | | | AQVFLAEIPA DPKLYRHHNK |
| | | | | | LRLCFKEFIM RCGEAVEKNK |
| | | | | | RLITADQREY QQELKKNYNK |
| | | | | | LKENLRPMIE RKIPELYKPI |
| | | | | | FRVESQKRDS FHRSSFRKCE |
| | | | | | TQLSQGS |
| 37 | DQCK8 | 3 | Q8NF50- | Entry version | MTHLNSLDVQ LAQELGDFTD |
| | | | 3 | 160 (18 Sep. | DDLDVVFTPK ECRTLQPSLP |
| | | | | 2019) | EEGVELDPHV RDCVQTYIRE |
| | | | | Sequence | WLIVNRKNQG SPEICGFKKT |
| | | | | version 3 (21 | GSRKDFHKTL PKQTFESETL |
| | | | | Aug. 2007) | ECSEPAAQAG PRHLNVLCDV |
| | | | | | SGKGPVTACD FDLRSLQPDK |
| | | | | | RLENLLQQVS AEDFEKQNEE |
| | | | | | ARRTNRQAEL FALYPSVDEE |
| | | | | | DAVEIRPVPE CPKEHLGNRI |
| | | | | | LVKLLTLKFE IEIEPLFASI |
| | | | | | ALYDVKERKK ISENFHCDLN |
| | | | | | SDQFKGFLRA HTPSVAASSQ |
| | | | | | ARSAVFSVTY PSSDIYLVVK |
| | | | | | IEKVLQQGEI GDCAEPYTVI |
| | | | | | KESDGGKSKE KIEKLKLQAE |
| | | | | | SFCQRLGKYR MPFAWAPISL |
| | | | | | SSFFNVSTLE REVTDVDSVV |
| | | | | | GRSSVGERRT LAQSRRLSER |
| | | | | | ALSLEENGVG SNFKTSTLSV |
| | | | | | SSFFKQEGDR LSDEDLFKFL |
| | | | | | ADYKRSSSLQ RRVKSIPGLL |
| | | | | | RLEISTAPEI INCCLTPEML |
| | | | | | PVKPFPENRT RPHKEILEFP |
| | | | | | TREVYVPHTV YRNLLYVYPQ |
| | | | | | RLNFVNKLAS ARNITIKIQF |
| | | | | | MCGEDASNAM PVIFGKSSGP |
| | | | | | EFLQEVYTAV TYHNKSPDFY |
| | | | | | EEVKIKLPAK LTVNHHLLFT |
| | | | | | FYHISCQQKQ GASVETLLGY |
| | | | | | SWLPILLNER LQTGSYCLPV |
| | | | | | ALEKLPPNYS MHSAEKVPLQ |
| | | | | | NPPIKWAEGH KGVFNIEVQA |
| | | | | | VSSVHTQDNH LEKFFTLCHS |
| | | | | | LESQVTFPIR VLDQKISEMA |
| | | | | | LEHELKLSII CLNSSRLEPL |
| | | | | | VLFLHLVLDK LFQLSVQPMV |
| | | | | | IAGQTANFSQ FAFESVVAIA |
| | | | | | NSLHNSKDLS KDQHGRNCLL |
| | | | | | ASYVHYVFRL PEVQRDVPKS |
| | | | | | GAPTALLDPR SYHTYGRTSA |
| | | | | | AAVSSKLLQA RVMSSSNPDL |
| | | | | | AGTHSAADEE VKNIMSSKIA |
| | | | | | DRNCSRMSYY CSGSSDAPSS |
| | | | | | PAAPRPASKK HFHEELALQM |
| | | | | | VVSTGMVRET VFKYAWFFFE |
| | | | | | LLVKSMAQHV HNMDKRDSFR |
| | | | | | RTRFSDREMD DITTIVNVVT |
| | | | | | SEIAALLVKP QKENEQAEKM |
| | | | | | NISLAFFLYD LLSLMDRGFV |
| | | | | | FNLIRHYCSQ LSAKLSNLPT |
| | | | | | LISMRLEFLR ILCSHEHYLN |
| | | | | | LNLFFMNADT APTSPCPSIS |
| | | | | | SQNSSSCSSF QDQKIASMED |
| | | | | | LTSEYRQQHF LTGLLFTELA |
| | | | | | AALDAEGEGI SKVQRKAVSA |
| | | | | | IHSLLSSHDL DPRCVKPEVK |
| | | | | | VKIAALYLPL VGIILDALPQ |
| | | | | | LCDFTVADTR RYRTSGSDEE |
| | | | | | QEGAGAINQN VALAIAGNNF |
| | | | | | NLKTSGIVLS SLPYKQYNML |
| | | | | | NADTTRNLMI CFLWIMKNAD |
| | | | | | QSLIRKWIAD LPSTQLNRIL |
| | | | | | DLLFICVLCF EYKGKQSSDK |
| | | | | | VSTQVLQKSR DVKARLEEAL |
| | | | | | LRGEGARGEM MRRRAPGNDR |
| | | | | | FPGLNENLRW KKEQTHWRQA |
| | | | | | NEKLDKTKAE LDQEALISGN |
| | | | | | LATEAHLIIL DMQENIIQAS |
| | | | | | SALDCKDSLL GGVLRVLVNS |
| | | | | | LNCDQSTTYL THCFATLRAL |
| | | | | | IAKFGDLLFE EEVEQCFDLC |
| | | | | | HQVLHHCSSS MDVTRSQACA |
| | | | | | TLYLLMRFSF GATSNFARVK |
| | | | | | MQVTMSLASL VGRAPDFNEE |
| | | | | | HLRRSLRTIL AYSEEDTAMQ |
| | | | | | MTPFPTQVEE LLCNLNSILY |
| | | | | | DTVKMREFQE DPEMLMDLMY |
| | | | | | RIAKSYQASP DLRLTWLQNM |
| | | | | | AEKHTKKKCY TEAAMCLVHA |
| | | | | | AALVAEYLSM LEDHSYLPVG |
| | | | | | SVSFQNISSN VLEESVVSED |
| | | | | | TLSPDEDGVC AGQYFTESGL |
| | | | | | VGLLEQAAEL FSTGGLYETV |
| | | | | | NEVYKLVIPI LEAHREFRKL |
| | | | | | TLTHSKLQRA FDSIVNKDHK |
| | | | | | RMFGTYFRVG FFGSKFGDLD |
| | | | | | EQEFVYKEPA ITKLPEISHR |
| | | | | | LEAFYGQCFG AEFVEVIKDS |
| | | | | | TPVDKTKLDP NKAYIQITFV |
| | | | | | EPYFDEYEMK DRVTYFEKNF |
| | | | | | NLRRFMYTTP FTLEGRPRGE |
| | | | | | LHEQYRRNTV LTTMHAFPYI |
| | | | | | KTRISVIQKE EFVLTPIEVA |
| | | | | | IEDMKKKTLQ LAVAINQEPP |
| | | | | | DAKMLQMVLQ GSVGATVNQG |
| | | | | | PLEVAQVFLA EIPADPKLYR |
| | | | | | HHNKLRLCFK EFIMRCGEAV |
| | | | | | EKNKRLITAD QREYQQELKK |
| | | | | | NYNKLKENLR PMIERKIPEL |
| | | | | | YKPIFRVESQ KRDSFHRSSF |
| | | | | | RKCETQLSQG S |
| 38 | DQCK8 | 4 | Q8NF50- | Entry version | MTHLNSLDVQ LAQELGDFTD |
| | | | 4 | 160 (18 Sep. | DDLDVVFTPK ECRTLQPSLP |
| | | | | 2019) | EEGVELDPHV RDCVQTYIRE |
| | | | | Sequence | WLIVNRKNQG SPEICGFKKT |
| | | | | version 3 (21 | GSRKDFHKTL PKQTFESETL |
| | | | | Aug. 2007) | ECSEPAAQAG PRHLNVLCDV |
| | | | | | SGKGPVTACD FDLRSLQPDK |
| | | | | | RLENLLQQVS AEDFEKQNEE |
| | | | | | ARRTNRQAEL FALYPSVDEE |
| | | | | | DAVEIRPVPE CPKEHLGNRI |
| | | | | | LVKLLTLKFE IEIEPLFASI |
| | | | | | ALYDVKERKK ISENFHCDLN |
| | | | | | SDQFKGFLRA HTPSVAASSQ |
| | | | | | ARSAVFSVTY PSSDIYLVVK |
| | | | | | IEKVLQQGEI GDCAEPYTVI |
| | | | | | KESDGGKSKE KIEKLKLQAE |
| | | | | | SFCQRLGKYR MPFAWAPISL |
| | | | | | SSFFNVSTLE REVTDVDSVV |
| | | | | | GRSSVGERRT LAQSRRLSER |
| | | | | | ALSLEENGVG SNFKTSTLSV |
| | | | | | SSFFKQEGDR LSDEDLFKFL |
| | | | | | ADYKRSSSLQ RRVKSIPGLL |
| | | | | | RLEISTAPEI INCCLTPEML |
| | | | | | PVKPFPENRT RPHKEILEFP |
| | | | | | TREVYVPHTV YRNLLYVYPQ |
| | | | | | RLNFVNKLAS ARNITIKIQF |
| | | | | | MCGEDASNAM PVIFGKSSGP |
| | | | | | EFLQEVYTAV TYHNKSPDFY |
| | | | | | EEVKIKLPAK LTVNHHLLFT |
| | | | | | FYHISCQQKQ GASVETLLGY |
| | | | | | SWLPILLNER LQTGSYCLPV |
| | | | | | ALEKLPPNYS MHSAEKVPLQ |
| | | | | | NPPIKWAEGH KGVFNIEVQA |
| | | | | | VSSVHTQDNH LEKFFTLCHS |
| | | | | | LESQVTFPIR VLDQKISEMA |
| | | | | | LEHELKLSII CLNSSRLEPL |
| | | | | | VLFLHLVLDK LFQLSVQPMV |
| | | | | | IAGQTANFSQ FAFESVVAIA |
| | | | | | NSLHNSKDLS KDQHGRNCLL |
| | | | | | ASYVHYVFRL PEVQRDVPKS |
| | | | | | GAPTALLDPR SYHTYGRTSA |
| | | | | | AAVSSKLLQA RVMSSSNPDL |
| | | | | | AGTHSAADEE VKNIMSSKIA |
| | | | | | DRNCSRMSYY CSGSSDAPSS |
| | | | | | PAAPRPASKK HFHEELALQM |
| | | | | | VVSTGMVRET VFKYAWFFFE |
| | | | | | LLVKSMAQHV HNMDKRDSFR |
| | | | | | RTRFSDRFMD DITTIVNVVT |
| | | | | | SEIAALLVKP QKENEQAEKM |
| | | | | | NISLAFFLYD LLSLMDRGFV |
| | | | | | FNLIRHYCSQ LSAKLSNLPT |
| | | | | | LISMRLEFLR ILCSHEHYLN |
| | | | | | LNLFFMNADT APTSPCPSIS |
| | | | | | SQNSSSCSSF QDQKIASMED |
| | | | | | LTSEYRQQHF LTGLLFTELA |
| | | | | | AALDAEGEGI SKVQRKAVSA |
| | | | | | IHSLLSSHDL DPRCVKPEVK |
| | | | | | VKIAALYLPL VGIILDALPQ |
| | | | | | LCDFTVADTR RYRTSGSDEE |
| | | | | | QEGAGAINQN VALAIAGNNF |
| | | | | | NLKTSGIVLS SLPYKQYNML |
| | | | | | NADTTRNLMI CFLWIMKNAD |
| | | | | | QSLIRKWIAD LPSTQLNRIL |
| | | | | | DLLFICVLCF EYKGKQSSDK |
| | | | | | VSTQVLQKSR DVKARLEEAL |
| | | | | | LRGEGARGEM MRRRAPGNDR |
| | | | | | FPGLNENLRW KKEQTHWRQA |
| | | | | | NEKLDKTKAE LDQEALISGN |
| | | | | | LATEAHLIIL DMQENIIQAS |
| | | | | | SALDCKDSLL GGVLRVLVNS |
| | | | | | LNCDQSTTYL THCFATLRAL |
| | | | | | IAKFGDLLFE EEVEQCFDLC |
| | | | | | HQVLHHCSSS MDVTRSQACA |
| | | | | | TLYLLMRFSF GATSNFARVK |
| | | | | | MQVTMSLASL VGRAPDFNEE |
| | | | | | HLRRSLRTIL AYSEEDTAMQ |
| | | | | | MTPFPTQVEE LLCNLNSILY |
| | | | | | DTVKMREFQE DPEMLMDLMY |
| | | | | | RIAKSYQASP DLRLTWLQNM |
| | | | | | AEKHTKKKCY TEAAMCLVHA |
| | | | | | AALVAEYLSM LEDHSYLPVG |
| | | | | | SVSFQNISSN VLEESVVSED |
| | | | | | TLSPDEDGVC AGQYFTESGL |
| | | | | | VGLLEQAAEL FSTGGLYETV |
| | | | | | NEVYKLVIPI LEAHREFRKL |
| | | | | | TLTHSKLQRA FDSIVNKDHK |
| | | | | | RMFGTYFRVG FFGSKFGDLD |
| | | | | | EQEFVYKEPA ITKLPEISHR |
| | | | | | LEAFYGQCFG AEFVEVIKDS |
| | | | | | TPVDKTKLDP NKAYIQITFV |
| | | | | | EPYFDEYEMK DRVTYFEKNF |
| | | | | | NLRRFMYTTP FTLEGRPRGE |
| | | | | | LHEQYRRNTV LTTMHAFPYI |
| | | | | | KTRISVIQKE EFVLTPIEVA |
| | | | | | IEDMKKKTLQ LAVAINQEPP |
| | | | | | DAKMLQMVLQ GSVGATVNQG |
| | | | | | PLEVAQVFLA EIPADPKLYR |
| | | | | | HHNKLRLCFK EFIMRCGEAV |
| | | | | | EKNKRLITAD QREYQQELKK |
| | | | | | NYNKLKENLR PMIERKIPEL |
| | | | | | YKPIFRVESQ KRDSFHRSSF |
| | | | | | RKCETQLSQG S |
| 39 | COMP | 1 | P49747 | Entry version | MVPDTACVLL LTLAALGASG |
| | | | | 195 (18 Sep. | QGQSPLGSDL GPQMLRELQE |
| | | | | 2019) | TNAALQDVRE LLRQQVREIT |
| | | | | Sequence | FLKNTVMECD ACGMQQSVRT |
| | | | | version 2 (14 | GLPSVRPLLH CAPGFCFPGV |
| | | | | Oct. 2008) | ACIQTESGAR CGPCPAGFTG |
| | | | | | NGSHCTDVNE CNAHPCFPRV |
| | | | | | RCINTSPGFR CEACPPGYSG |
| | | | | | PTHQGVGLAF AKANKQVCTD |
| | | | | | INECETGQHN CVPNSVCINT |
| | | | | | RGSFQCGPCQ PGFVGDQASG |
| | | | | | CQRRAQRFCP DGSPSECHEH |
| | | | | | ADCVLERDGS RSCVCAVGWA |
| | | | | | GNGILCGRDT DLDGFPDEKL |
| | | | | | RCPERQCRKD NCVTVPNSGQ |
| | | | | | EDVDRDGIGD ACDPDADGDG |
| | | | | | VPNEKDNCPL VRNPDQRNTD |
| | | | | | EDKWGDACDN CRSQKNDDQK |
| | | | | | DTDQDGRGDA CDDDIDGDRI |
| | | | | | RNQADNCPRV PNSDQKDSDG |
| | | | | | DGIGDACDNC PQKSNPDQAD |
| | | | | | VDHDFVGDAC DSDQDQDGDG |
| | | | | | HQDSRDNCPT VPNSAQEDSD |
| | | | | | HDGQGDACDD DDDNDGVPDS |
| | | | | | RDNCRLVPNP GQEDADRDGV |
| | | | | | GDVCQDDFDA DKVVDKIDVC |
| | | | | | PENAEVTLTD FRAFQTVVLD |
| | | | | | PEGDAQIDPN WVVLNQGREI |
| | | | | | VQTMNSDPGL AVGYTAFNGV |
| | | | | | DFEGTFHVNT VTDDDYAGFI |
| | | | | | FGYQDSSSFY VVMWKQMEQT |
| | | | | | YWQANPFRAV AEPGIQLKAV |
| | | | | | KSSTGPGEQL RNALWHTGDT |
| | | | | | ESQVRLLWKD PRNVGWKDKK |
| | | | | | SYRWFLQHRP QVGYIRVRFY |
| | | | | | EGPELVADSN VVLDTTMRGG |
| | | | | | RLGVFCFSQE NIIWANLRYR |
| | | | | | CNDTIPEDYE THQLRQA |
| 40 | COMP | 2 | P49747- | Entry version | MVPDTACVLL LTLAALGASG |
| | | | 2 | 195 (18 Sep. | QGQSPLGSDL GPQMLRELQE |
| | | | | 2019) | TNAALQDVRE LLRQQVREIT |
| | | | | Sequence | FLKNTVMECD ACGMQQSVRT |
| | | | | version 2 (14 | GLPSVRPLLH CAPGFCFPGV |
| | | | | Oct. 2008) | ACIQTESGAR CGPCPAGFTG |
| | | | | | NGSHCTDVNE CETGQHNCVP |
| | | | | | NSVCINTRGS FQCGPCQPGF |
| | | | | | VGDQASGCQR RAQRFCPDGS |
| | | | | | PSECHEHADC VLERDGSRSC |
| | | | | | VCAVGWAGNG ILCGRDTDLD |
| | | | | | GFPDEKLRCP ERQCRKDNCV |
| | | | | | TVPNSGQEDV DRDGIGDACD |
| | | | | | PDADGDGVPN EKDNCPLVRN |
| | | | | | PDQRNTDEDK WGDACDNCRS |
| | | | | | QKNDDQKDTD QDGRGDACDD |
| | | | | | DIDGDRIRNQ ADNCPRVPNS |
| | | | | | DQKDSDGDGI GDACDNCPQK |
| | | | | | SNPDQADVDH DFVGDACDSD |
| | | | | | QDQDGDGHQD SRDNCPTVPN |
| | | | | | SAQEDSDHDG QGDACDDDDD |
| | | | | | NDGVPDSRDN CRLVPNPGQE |
| | | | | | DADRDGVGDV CQDDFDADKV |
| | | | | | VDKIDVCPEN AEVTLTDFRA |
| | | | | | FQTVVLDPEG DAQIDPNWVV |
| | | | | | LNQGREIVQT MNSDPGLAVG |
| | | | | | YTAFNGVDFE GTFHVNTVTD |
| | | | | | DDYAGFIFGY QDSSSFYVVM |
| | | | | | WKQMEQTYWQ ANPFRAVAEP |
| | | | | | GIQLKAVKSS TGPGEQLRNA |
| | | | | | LWHTGDTESQ VRLLWKDPRN |
| | | | | | VGWKDKKSYR WFLQHRPQVG |
| | | | | | YIRVRFYEGP ELVADSNVVL |
| | | | | | DTTMRGGRLG VFCFSQENII |
| | | | | | WANLRYRCND TIPEDYETHQ |
| | | | | | LRQA |
| 41 | ATG7 | 1 | O95352 | Entry version | MAAATGDPGL SKLQFAPFSS |
| | | | | 168 (18 Sep. | ALDVGFWHEL TQKKLNEYRL |
| | | | | 2019) | DEAPKDIKGY YYNGDSAGLP |
| | | | | Sequence | ARLTLEFSAF DMSAPTPARC |
| | | | | version 1 (01 | CPAIGTLYNT NTLESFKTAD |
| | | | | May 1999) | KKLLLEQAAN EIWESIKSGT |
| | | | | | ALENPVLINK FLLLTFADLK |
| | | | | | KYHFYYWFCY PALCLPESLP |
| | | | | | LIQGPVGLDQ RFSLKQIEAL |
| | | | | | ECAYDNLCQT EGVTALPYFL |
| | | | | | IKYDENMVLV SLLKHYSDFF |
| | | | | | QGQRTKITIG VYDPCNLAQY |
| | | | | | PGWPLRNFLV LAAHRWSSSF |
| | | | | | QSVEVVCFRD RTMQGARDVA |
| | | | | | HSIIFEVKLP EMAFSPDCPK |
| | | | | | AVGWEKNQKG GMGPRMVNLS |
| | | | | | ECMDPKRLAE SSVDLNLKLM |
| | | | | | CWRLVPTLDL DKVVSVKCLL |
| | | | | | LGAGTLGCNV ARTLMGWGVR |
| | | | | | HITFVDNAKI SYSNPVRQPL |
| | | | | | YEFEDCLGGG KPKALAAADR |
| | | | | | LQKIFPGVNA RGFNMSIPMP |
| | | | | | GHPVNFSSVT LEQARRDVEQ |
| | | | | | LEQLIESHDV VFLLMDTRES |
| | | | | | RWLPAVIAAS KRKLVINAAL |
| | | | | | GFDTFVVMRH GLKKPKQQGA |
| | | | | | GDLCPNHPVA SADLLGSSLF |
| | | | | | ANIPGYKLGC YFCNDVVAPG |
| | | | | | DSTRDRTLDQ QCTVSRPGLA |
| | | | | | VIAGALAVEL MVSVLQHPEG |
| | | | | | GYAIASSSDD RMNEPPTSLG |
| | | | | | LVPHQIRGFL SRFDNVLPVS |
| | | | | | LAFDKCTACS SKVLDQYERE |
| | | | | | GENFLAKVFN SSHSFLEDLT |
| | | | | | GLTLLHQETQ AAEIWDMSDD |
| | | | | | ETI |
| 42 | ATG7 | 2 | O95352- | Entry version | MAAATGDPGL SKLQFAPFSS |
| | | | 2 | 168 (18 Sep. | ALDVGFWHEL TQKKLNEYRL |
| | | | | 2019) | DEAPKDIKGY YYNGDSAGLP |
| | | | | Sequence | ARLTLEFSAF DMSAPTPARC |
| | | | | version 1 (01 | CPAIGTLYNT NTLESFKTAD |
| | | | | May 1999) | KKLLLEQAAN EIWESIKSGT |
| | | | | | ALENPVLLNK FLLLTFADLK |
| | | | | | KYHFYYWFCY PALCLPESLP |
| | | | | | LIQGPVGLDQ RFSLKQIEAL |
| | | | | | ECAYDNLCQT EGVTALPYFL |
| | | | | | IKYDENMVLV SLLKHYSDFF |
| | | | | | QGQRTKITIG VYDPCNLAQY |
| | | | | | PGWPLRNFLV LAAHRWSSSF |
| | | | | | QSVEVVCFRD RTMQGARDVA |
| | | | | | HSIIFEVKLP EMAFSPDCPK |
| | | | | | AVGWEKNQKG GMGPRMVNLS |
| | | | | | ECMDPKRLAE SSVDLNLKLM |
| | | | | | CWRLVPTLDL DKVVSVKCLL |
| | | | | | LGAGTLGCNV ARTLMGWGVR |
| | | | | | HITFVDNAKI SYSNPVRQPL |
| | | | | | YEFEDCLGGG KPKALAAADR |
| | | | | | LQKIFPGVNA RGFNMSIPMP |
| | | | | | GHPVNFSSVT LEQARRDVEQ |
| | | | | | LEQLIESHDV VFLLMDTRES |
| | | | | | RWLPAVIAAS KRKLVINAAL |
| | | | | | GFDTFVVMRH GLKKPKQQGA |
| | | | | | GDLCPNHPVA SADLLGSSLF |
| | | | | | ANIPGYKLGC YFCNDVVAPG |
| | | | | | DSTRDRTLDQ QCTVSRPGLA |
| | | | | | VIAGALAVEL MVSVLQHPEG |
| | | | | | GYAIASSSDD RMNEPPTSLG |
| | | | | | LVPHQVLDQY EREGFNFLAK |
| | | | | | VFNSSHSFLE DLTGLTLLHQ |
| | | | | | ETQAAEIWDM SDDETI |
| 43 | ATG7 | 3 | O95352- | Entry version | MAAATGDPGL SKLQFAPFSS |
| | | | 3 | 168 (18 Sep. | ALDVGFWHEL TQKKLNEYRL |
| | | | | 2019) | DEAPKDIKGY YYNGDSAGLP |
| | | | | Sequence | ARLTLEFSAF DMSAPTPARC |
| | | | | version 1 (01 | CPAIGTLYNT NTLESFKTAD |
| | | | | May 1999) | KKLLLEQAAN EIWESIKSGT |
| | | | | | ALENPVLLNK FLLLTFAIEA |
| | | | | | LECAYDNLCQ TEGVTALPYF |
| | | | | | LIKYDENMVL VSLLKHYSDF |
| | | | | | FQGQRTKITI GVYDPCNLAQ |
| | | | | | YPGWPLRNFL VLAAHRWSSS |
| | | | | | FQSVEVVCFR DRTMQGARDV |
| | | | | | AHSIIFEVKL PEMAFSPDCP |
| | | | | | KAVGWEKNQK GGMGPRMVNL |
| | | | | | SECMDPKRLA ESSVDLNLKL |
| | | | | | MCWRLVPTLD LDKVVSVKCL |
| | | | | | LLGAGTLGCN VARTLMGWGV |
| | | | | | RHITFVDNAK ISYSNPVRQP |
| | | | | | LYEFEDCLGG GKPKALAAAD |
| | | | | | RLQKIFPGVN ARGFNMSIPM |
| | | | | | PGHPVNFSSV TLEQARRDVE |
| | | | | | QLEQLIESHD VVFLLMDTRE |
| | | | | | SRWLPAVIAA SKRKLVINAA |
| | | | | | LGFDTFVVMR HGLKKPKQQG |
| | | | | | AGDLCPNHPV ASADLLGSSL |
| | | | | | FANIPGYKLG CYFCNDVVAP |
| | | | | | GDSTRDRTLD QQCTVSRPGL |
| | | | | | AVIAGALAVE LMVSVLQHPE |
| | | | | | GGYAIASSSD DRMNEPPTSL |
| | | | | | GLVPHQIRGF LSRFDNVLPV |
| | | | | | SLAFDKCTAC SSKIWDMSDD |
| | | | | | ETI |
| 44 | SLC2A1 | 1 | P11166 | Entry version | MEPSSKKLTG RLMLAVGGAV |
| | | | | 230 (18 Sep. | LGSLQFGYNT GVINAPQKVI |
| | | | | 2019) | EEFYNQTWVH RYGESILPTT |
| | | | | Sequence | LTTLWSLSVA IFSVGGMIGS |
| | | | | version 2 (03 | FSVGLFVNRF GRRNSMLMMN |
| | | | | Oct. 2006) | LLAFVSAVLM GFSKLGKSFE |
| | | | | | MLILGRFIIG VYCGLTTGFV |
| | | | | | PMYVGEVSPT ALRGALGTLH |
| | | | | | QLGIVVGILI AQVFGLDSIM |
| | | | | | GNKDLWPLLL SIIFIPALLQ |
| | | | | | CIVLPFCPES PRFLLINRNE |
| | | | | | ENRAKSVLKK LRGTADVTHD |
| | | | | | LQEMKEESRQ MMREKKVTIL |
| | | | | | ELFRSPAYRQ PILIAVVLQL |
| | | | | | SQQLSGINAV FYYSTSIFEK |
| | | | | | AGVQQPVYAT IGSGIVNTAF |
| | | | | | TVVSLFVVER AGRRTLHLIG |
| | | | | | LAGMAGCAIL MTIALALLEQ |
| | | | | | LPWMSYLSIV AIFGFVAFFE |
| | | | | | VGPGPIPWFI VAELFSQGPR |
| | | | | | PAAIAVAGFS NWTSNFIVGM |
| | | | | | CFQYVEQLCG PYVFIIFTVL |
| | | | | | LVLFFIFTYF KVPETKGRTF |
| | | | | | DEIASGFRQG GASQSDKTPE |
| | | | | | ELFHPLGADS QV |
| 45 | GZMK | 1 | P49863 | Entry version | MTKFSSFSLF FLIVGAYMTH |
| | | | | 152 (18 Sep. | VCFNMEIIGG KEVSPHSRPF |
| | | | | 2019) | MASIQYGGHH VCGGVLIDPQ |
| | | | | Sequence | WVLTAAHCQY RFTKGQSPTV |
| | | | | version 1 (01 | VLGAHSLSKN EASKQTLEIK |
| | | | | Oct. 1996) | KFIPFSRVTS DPQSNDIMLV |
| | | | | | KLQTAAKLNK HVKMLHIRSK |
| | | | | | TSLRSGTKCK VTGWGATDPD |
| | | | | | SLRPSDTLRE VTVTVLSRKL |
| | | | | | CNSQSYYNGD PFITKDMVCA |
| | | | | | GDAKGQKDSC KGDSGGPLIC |
| | | | | | KGVFHAIVSG GHECGVATKP |
| | | | | | GIYTLLTKKY QTWIKSNLVP |
| | | | | | PHTN |
| 46 | S100A9 | 1 | P06702 | Entry version | MTCKMSQLER NIETIINTFH |
| | | | | 218 (18 Sep. | QYSVKLGHPD TLNQGEFKEL |
| | | | | 2019) | VRKDLQNFLK KENKNEKVIE |
| | | | | Sequence | HIMEDLDTNA DKQLSFEEFI |
| | | | | version 1 (01 | MLMARLTWAS HEKMHEGDEG |
| | | | | Jan. 1988) | PGHHHKPGLG EGTP |
| 47 | S100A8 | 1 | P05109 | Entry version | MLTELEKALN SIIDVYHKYS |
| | | | | 209 (18 Sep. | LIKGNFHAVY RDDLKKLLET |
| | | | | 2019) | ECPQYIRKKG ADVWFKELDI |
| | | | | Sequence | NTDGAVNFQE FLILVIKMGV |
| | | | | version 1 (01 | AAHKKSHEES HKE |
| | | | | Jan. 1988) | |
| 48 | ATM | 1 | Q13315 | Entry version | MSLVLNDLLI CCRQLEHDRA |
| | | | | 236 (18 Sep. | TERKKEVEKF KRLIRDPETI |
| | | | | 2019) | KHLDRHSDSK QGKYLNWDAV |
| | | | | Sequence | FRFLQKYIQK ETECLRIAKP |
| | | | | version 4 (22 | NVSASTQASR QKKMQEISSL |
| | | | | Jan. 2014) | VKYFIKCANR RAPRLKCQEL |
| | | | | | LNYIMDTVKD SSNGAIYGAD |
| | | | | | CSNILLKDIL SVRKYWCEIS |
| | | | | | QQQWLELFSV YFRLYLKPSQ |
| | | | | | DVHRVLVARI IHAVTKGCCS |
| | | | | | QTDGLNSKFL DFFSKAIQCA |
| | | | | | RQEKSSSGLN HILAALTIFL |
| | | | | | KTLAVNFRIR VCELGDEILP |
| | | | | | TLLYIWTQHR LNDSLKEVII |
| | | | | | ELFQLQIYIH HPKGAKTQEK |
| | | | | | GAYESTKWRS ILYNLYDLLV |
| | | | | | NEISHIGSRG KYSSGFRNIA |
| | | | | | VKENLIELMA DICHQVFNED |
| | | | | | TRSLEISQSY TTTQRESSDY |
| | | | | | SVPCKRKKIE LGWEVIKDHL |
| | | | | | QKSQNDFDLV PWLQIATQLI |
| | | | | | SKYPASLPNC ELSPLLMILS |
| | | | | | QLLPQQRHGE RTPYVLRCLT |
| | | | | | EVALCQDKRS NLESSQKSDL |
| | | | | | LKLWNKIWCI TFRGISSEQI |
| | | | | | QAENFGLLGA IIQGSLVEVD |
| | | | | | REFWKLFTGS ACRPSCPAVC |
| | | | | | CLTLALTTSI VPGTVKMGIE |
| | | | | | QNMCEVNRSF SLKESIMKWL |
| | | | | | LFYQLEGDLE NSTEVPPILH |
| | | | | | SNFPHLVLEK ILVSLTMKNC |
| | | | | | KAAMNFFQSV PECEHHQKDK |
| | | | | | EELSFSEVEE LFLQTTFDKM |
| | | | | | DFLTIVRECG IEKHQSSIGF |
| | | | | | SVHQNLKESL DRCLLGLSEQ |
| | | | | | LLNNYSSEIT NSETLVRCSR |
| | | | | | LLVGVLGCYC YMGVIAEEEA |
| | | | | | YKSELFQKAK SLMQCAGESI |
| | | | | | TLFKNKTNEE FRIGSLRNMM |
| | | | | | QLCTRCLSNC TKKSPNKIAS |
| | | | | | GFFLRLLTSK LMNDIADICK |
| | | | | | SLASFIKKPF DRGEVESMED |
| | | | | | DTNGNLMEVE DQSSMNLEND |
| | | | | | YPDSSVSDAN EPGESQSTIG |
| | | | | | AINPLAEEYL SKQDLLFLDM |
| | | | | | LKFLCLCVTT AQTNTVSFRA |
| | | | | | ADIRRKLLML IDSSTLEPTK |
| | | | | | SLHLHMYLML LKELPGEEYP |
| | | | | | LPMEDVLELL KPLSNVCSLY |
| | | | | | RRDQDVCKTI LNHVLHVVKN |
| | | | | | LGQSNMDSEN TRDAQGQFLT |
| | | | | | VIGAFWHLTK ERKYIFSVRM |
| | | | | | ALVNCLKTLL EADPYSKWAI |
| | | | | | LNVMGKDFPV NEVFTQFLAD |
| | | | | | NHHQVRMLAA ESINRLFQDT |
| | | | | | KGDSSRLLKA LPLKLQQTAF |
| | | | | | ENAYLKAQEG MREMSHSAEN |
| | | | | | PETLDEIYNR KSVLLTLIAV |
| | | | | | VLSCSPICEK QALFALCKSV |
| | | | | | KENGLEPHLV KKVLEKVSET |
| | | | | | FGYRRLEDFM ASHLDYLVLE |
| | | | | | WLNLQDTEYN LSSFPFILLN |
| | | | | | YTNIEDFYRS CYKVLIPHLV |
| | | | | | IRSHFDEVKS IANQIQEDWK |
| | | | | | SLLTDCFPKI LVNILPYFAY |
| | | | | | EGTRDSGMAQ QRETATKVYD |
| | | | | | MLKSENLLGK QIDHLFISNL |
| | | | | | PEIVVELLMT LHEPANSSAS |
| | | | | | QSTDLCDFSG DLDPAPNPPH |
| | | | | | FPSHVIKATF AYISNCHKTK |
| | | | | | LKSILEILSK SPDSYQKILL |
| | | | | | AICEQAAETN NVYKKHRILK |
| | | | | | IYHLFVSLLL KDIKSGLGGA |
| | | | | | WAFVLRDVIY TLIHYINQRP |
| | | | | | SCIMDVSLRS FSLCCDLLSQ |
| | | | | | VCQTAVTYCK DALENHLHVI |
| | | | | | VGTLIPLVYE QVEVQKQVLD |
| | | | | | LLKYLVIDNK DNENLYITIK |
| | | | | | LLDPFPDHVV FKDLRITQQK |
| | | | | | IKYSRGPFSL LEEINHFLSV |
| | | | | | SVYDALPLTR LEGLKDLRRQ |
| | | | | | LELHKDQMVD IMRASQDNPQ |
| | | | | | DGIMVKLVVN LLQLSKMAIN |
| | | | | | HTGEKEVLEA VGSCLGEVGP |
| | | | | | IDFSTIAIQH SKDASYTKAL |
| | | | | | KLFEDKELQW TFIMLTYLNN |
| | | | | | TLVEDCVKVR SAAVTCLKNI |
| | | | | | LATKTGHSFW EIYKMTTDPM |
| | | | | | LAYLQPFRTS RKKFLEVPRF |
| | | | | | DKENPFEGLD DINLWIPLSE |
| | | | | | NHDIWIKTLT CAFLDSGGTK |
| | | | | | CEILQLLKPM CEVKTDFCQT |
| | | | | | VLPYLIHDIL LQDTNESWRN |
| | | | | | LLSTHVQGFF TSCLRHFSQT |
| | | | | | SRSTTPANLD SESEHFFRCC |
| | | | | | LDKKSQRTML AVVDYMRRQK |
| | | | | | RPSSGTIFND AFWLDLNYLE |
| | | | | | VAKVAQSCAA HFTALLYAEI |
| | | | | | YADKKSMDDQ EKRSLAFEEG |
| | | | | | SQSTTISSLS EKSKEETGIS |
| | | | | | LQDLLLEIYR SIGEPDSLYG |
| | | | | | CGGGKMLQPI TRLRTYEHEA |
| | | | | | MWGKALVTYD LETAIPSSTR |
| | | | | | QAGIIQALQN LGLCHILSVY |
| | | | | | LKGLDYENKD WCPELEELHY |
| | | | | | QAAWRNMQWD HCTSVSKEVE |
| | | | | | GTSYHESLYN ALQSLRDREF |
| | | | | | STFYESLKYA RVKEVEEMCK |
| | | | | | RSLESVYSLY PTLSRLQAIG |
| | | | | | ELESIGELFS RSVTHRQLSE |
| | | | | | VYIKWQKHSQ LLKDSDFSFQ |
| | | | | | EPIMALRTVI LEILMEKEMD |
| | | | | | NSQRECIKDI LTKHLVELSI |
| | | | | | LARTFKNTQL PERAIFQIKQ |
| | | | | | YNSVSCGVSE WQLEEAQVFW |
| | | | | | AKKEQSLALS ILKQMIKKLD |
| | | | | | ASCAANNPSL KLTYTECLRV |
| | | | | | CGNWLAETCL ENPAVIMQTY |
| | | | | | LEKAVEVAGN YDGESSDELR |
| | | | | | NGKMKAFLSL ARFSDTQYQR |
| | | | | | IENYMKSSEF ENKQALLKRA |
| | | | | | KEEVGLLREH KIQTNRYTVK |
| | | | | | VQRELELDEL ALRALKEDRK |
| | | | | | RFLCKAVENY INCLLSGEEH |
| | | | | | DMWVFRLCSL WLENSGVSEV |
| | | | | | NGMMKRDGMK IPTYKFLPLM |
| | | | | | YQLAARMGTK MMGGLGFHEV |
| | | | | | LNNLISRISM DHPHHTLFII |
| | | | | | LALANANRDE FLTKPEVARR |
| | | | | | SRITKNVPKQ SSQLDEDRTE |
| | | | | | AANRIICTIR SRRPQMVRSV |
| | | | | | EALCDAYIIL ANLDATQWKT |
| | | | | | QRKGINIPAD QPITKLKNLE |
| | | | | | DVVVPTMEIK VDHTGEYGNL |
| | | | | | VTIQSFKAEF RLAGGVNLPK |
| | | | | | IIDCVGSDGK ERRQLVKGRD |
| | | | | | DLRQDAVMQQ VFQMCNTLLQ |
| | | | | | RNTETRKRKL TICTYKVVPL |
| | | | | | SQRSGVLEWC TGTVPIGEFL |
| | | | | | VNNEDGAHKR YRPNDFSAFQ |
| | | | | | CQKKMMEVQK KSFEEKYEVF |
| | | | | | MDVCQNFQPV FRYFCMEKFL |
| | | | | | DPAIWFEKRL AYTRSVATSS |
| | | | | | IVGYILGLGD RHVQNILINE |
| | | | | | QSAELVHIDL GVAFEQGKIL |
| | | | | | PTPETVPFRL TRDIVDGMGI |
| | | | | | TGVEGVFRRC CEKTMEVMRN |
| | | | | | SQETLLTIVE VLLYDPLFDW |
| | | | | | TMNPLKALYL QQRPEDETEL |
| | | | | | HPTLNADDQE CKRNLSDIDQ |
| | | | | | SFNKVAERVL MRLQEKLKGV |
| | | | | | EEGTVLSVGG QVNLLIQQAI |
| | | | | | DPKNLSRLFP GWKAWV |
| 49 | IKBKB | 1 | O14920 | Entry version | MSWSPSLTTQ TCGAWEMKER |
| | | | | 217 (18 Sep. | LGTGGFGNVI RWHNQETGEQ |
| | | | | 2019) | IAIKQCRQEL SPRNRERWCL |
| | | | | Sequence | EIQIMRRLTH PNVVAARDVP |
| | | | | version 1 (01 | EGMQNLAPND LPLLAMEYCQ |
| | | | | Jan. 1998) | GGDLRKYLNQ FENCCGLREG |
| | | | | | AILTLLSDIA SALRYLHENR |
| | | | | | IIHRDLKPEN IVLQQGEQRL |
| | | | | | IHKIIDLGYA KELDQGSLCT |
| | | | | | SFVGTLQYLA PELLEQQKYT |
| | | | | | VTVDYWSFGT LAFECITGFR |
| | | | | | PFLPNWQPVQ WHSKVRQKSE |
| | | | | | VDIVVSEDLN GTVKFSSSLP |
| | | | | | YPNNLNSVLA ERLEKWLQLM |
| | | | | | LMWHPRQRGT DPTYGPNGCF |
| | | | | | KALDDILNLK LVHILNMVTG |
| | | | | | TIHTYPVTED ESLQSLKARI |
| | | | | | QQDTGIPEED QELLQEAGLA |
| | | | | | LIPDKPATQC ISDGKLNEGH |
| | | | | | TLDMDLVFLF DNSKITYETQ |
| | | | | | ISPRPQPESV SCILQEPKRN |
| | | | | | LAFFQLRKVW GQVWHSIQTL |
| | | | | | KEDCNRLQQG QRAAMMNLLR |
| | | | | | NNSCLSKMKN SMASMSQQLK |
| | | | | | AKLDFFKTSI QIDLEKYSEQ |
| | | | | | TEFGITSDKL LLAWREMEQA |
| | | | | | VELCGRENEV KLLVERMMAL |
| | | | | | QTDIVDLQRS PMGRKQGGTL |
| | | | | | DDLEEQAREL YRRLREKPRD |
| | | | | | QRTEGDSQEM VRLLLQAIQS |
| | | | | | FEKKVRVIYT QLSKTVVCKQ |
| | | | | | KALELLPKVE EVVSLMNEDE |
| | | | | | KTVVRLQEKR QKELWNLLKI |
| | | | | | ACSKVRGPVS GSPDSMNASR |
| | | | | | LSQPGQLMSQ PSTASNSLPE |
| | | | | | PAKKSEELVA EAHNLCTLLE |
| | | | | | NAIQDTVREQ DQSFTALDWS |
| | | | | | WLQTEEEEHS CLEQAS |
| 50 | IKBKB | 2 | O14920- | Entry version | MFSGGCHSPG FGRPSPAFPA |
| | | | 2 | 217 (18 Sep. | PGSPPPAPRP CRQETGEQIA |
| | | | | 2019) | IKQCRQELSP RNRERWCLEI |
| | | | | Sequence | QIMRRLTHPN VVAARDVPEG |
| | | | | version 1 (01 | MQNLAPNDLP LLAMEYCQGG |
| | | | | Jan. 1998) | DLRKYLNQFE NCCGLREGAI |
| | | | | | LTLLSDIASA LRYLHENRII |
| | | | | | HRDLKPENIV LQQGEQRLIH |
| | | | | | KIIDLGYAKE LDQGSLCTSF |
| | | | | | VGTLQYLAPE LLEQQKYTVT |
| | | | | | VDYWSFGTLA FECITGFRPF |
| | | | | | LPNWQPVQWH SKVRQKSEVD |
| | | | | | IVVSEDLNGT VKFSSSLPYP |
| | | | | | NNLNSVLAER LEKWLQLMLM |
| | | | | | WHPRQRGTDP TYGPNGCFKA |
| | | | | | LDDILNLKLV HILNMVTGTI |
| | | | | | HTYPVTEDES LQSLKARIQQ |
| | | | | | DTGIPEEDQE LLQEAGLALI |
| | | | | | PDKPATQCIS DGKLNEGHTL |
| | | | | | DMDLVFLFDN SKITYETQIS |
| | | | | | PRPQPESVSC ILQEPKRNLA |
| | | | | | FFQLRKVWGQ VWHSIQTLKE |
| | | | | | DCNRLQQGQR AAMMNLLRNN |
| | | | | | SCLSKMKNSM ASMSQQLKAK |
| | | | | | LDFFKTSIQI DLEKYSEQTE |
| | | | | | FGITSDKLLL AWREMEQAVE |
| | | | | | LCGRENEVKL LVERMMALQT |
| | | | | | DIVDLQRSPM GRKQGGTLDD |
| | | | | | LEEQARELYR RLREKPRDQR |
| | | | | | TEGDSQEMVR LLLQAIQSFE |
| | | | | | KKVRVIYTQL SKTVVCKQKA |
| | | | | | LELLPKVEEV VSLMNEDEKT |
| | | | | | VVRLQEKRQK ELWNLLKIAC |
| | | | | | SKVRGPVSGS PDSMNASRLS |
| | | | | | QPGQLMSQPS TASNSLPEPA |
| | | | | | KKSEELVAEA HNLCTLLENA |
| | | | | | IQDTVREQDQ SFTALDWSWL |
| | | | | | QTEEEEHSCL EQAS |
| 51 | IKBKB | 3 | O14920- | Entry version | MSWSPSLTTQ TCGAWEMKER |
| | | | 3 | 217 (18 Sep. | LGTGGFGNVI RWHNQETGEQ |
| | | | | 2019) | IAIKQCRQEL SPRNRERWCL |
| | | | | Sequence | |
| | | | | version 1 (01 | EIQIMRRLTH PNVVAARDVP |
| | | | | Jan. 1998) | EGMQNLAPND LPLLAMEYCQ |
| | | | | | GGDLRKYLNQ FENCCGLREG |
| | | | | | AILTLLSDIA SALRYLHENR |
| | | | | | IIHRDLKPEN IVLQQGEQRL |
| | | | | | IHKIIDLGYA KELDQGSLCT |
| | | | | | SFVGTLQYLA PELLEQQKYT |
| | | | | | VTVDYWSFGT LAFECITGER |
| | | | | | PFLPNWQPVQ CVRMWPGTVA |
| | | | | | HSCNPSTLGG RGRWIS |
| 52 | IKBKB | 4 | O14920- | Entry version | MSSDGTIRLT HPNVVAARDV |
| | | | 4 | 217 (18 Sep. | PEGMQNLAPN DLPLLAMEYC |
| | | | | 2019) | QGGDLRKYLN QFENCCGLRE |
| | | | | Sequence | GAILTLLSDI ASALRYLHEN |
| | | | | version 1 (01 | RIIHRDLKPE NIVLQQGEQR |
| | | | | Jan. 1998) | LIHKIIDLGY AKELDQGSLC |
| | | | | | TSFVGTLQYL APELLEQQKY |
| | | | | | TVTVDYWSFG TLAFECITGF |
| | | | | | RPFLPNWQPV QWHSKVRQKS |
| | | | | | EVDIVVSEDL NGTVKFSSSL |
| | | | | | PYPNNLNSVL AERLEKWLQL |
| | | | | | MLMWHPRQRG TDPTYGPNGC |
| | | | | | FKALDDILNL KLVHILNMVT |
| | | | | | GTIHTYPVTE DESLQSLKAR |
| | | | | | IQQDTGIPEE DQELLQEAGL |
| | | | | | ALIPDKPATQ CISDGKLNEG |
| | | | | | HTLDMDLVFL FDNSKITYET |
| | | | | | QISPRPQPES VSCILQEPKR |
| | | | | | NLAFFQLRKV WGQVWHSIQT |
| | | | | | LKEDCNRLQQ GQRAAMMNLL |
| | | | | | RNNSCLSKMK NSMASMSQQL |
| | | | | | KAKLDFFKTS IQIDLEKYSE |
| | | | | | QTEFGITSDK LLLAWREMEQ |
| | | | | | AVELCGRENE VKLLVERMMA |
| | | | | | LQTDIVDLQR SPMGRKQGGT |
| | | | | | LDDLEEQARE LYRRLREKPR |
| | | | | | DQRTEGDSQE MVRLLLQAIQ |
| | | | | | SFEKKVRVIY TQLSKTVVCK |
| | | | | | QKALELLPKV EEVVSLMNED |
| | | | | | EKTVVRLQEK RQKELWNLLK |
| | | | | | IACSKVRGPV SGSPDSMNAS |
| | | | | | RLSQPGQLMS QPSTASNSLP |
| | | | | | EPAKKSEELV AEAHNLCTLL |
| | | | | | ENAIQDTVRE QDQSFTALDW |
| | | | | | SWLQTEEEEH SCLEQAS |
| 53 | BCAP31 | 1 | P51572 | Entry version | MSLQWTAVAT FLYAEVFVVL |
| | | | | 186 (18 Sep. | LLCIPFISPK RWQKIFKSRL |
| | | | | 2019) | VELLVSYGNT FFVVLIVILV |
| | | | | Sequence | LLVIDAVREI RKYDDVTEKV |
| | | | | version 3 (23 | NLQNNPGAME HFHMKLFRAQ |
| | | | | Jan. 2007) | RNLYIAGESL LLSFLLRRLV |
| | | | | | TLISQQATLL ASNEAFKKQA |
| | | | | | ESASEAAKKY MEENDQLKKG |
| | | | | | AAVDGGKLDV GNAEVKLEEE |
| | | | | | NRSLKADLQK LKDELASTKQ |
| | | | | | KLEKAENQVL AMRKQSEGLT |
| | | | | | KEYDRLLEEH AKLQAAVDGP |
| | | | | | MDKKEE |
| 54 | BCAP31 | 2 | P51572- | Entry version | MGAEASSSWC PGTALPEERL |
| | | | 2 | 186 (18 Sep. | SVKRASEISG FLGQGSSGEA |
| | | | | 2019) | ALDVLTHVLE GAGNKLTSSC |
| | | | | Sequence | GKPSSNRMSL QWTAVATFLY |
| | | | | version 3 (23 | AEVFVVLLLC IPFISPKRWQ |
| | | | | Jan. 2007) | KIFKSRLVEL LVSYGNTFFV |
| | | | | | VLIVILVLLV IDAVREIRKY |
| | | | | | DDVTEKVNLQ NNPGAMEHFH |
| | | | | | MKLFRAQRNL YIAGFSLLLS |
| | | | | | FLLRRLVTLI SQQATLLASN |
| | | | | | EAFKKQAESA SEAAKKYMEE |
| | | | | | NDQLKKGAAV DGGKLDVGNA |
| | | | | | EVKLEEENRS LKADLQKLKD |
| | | | | | ELASTKQKLE KAENQVLAMR |
| | | | | | KQSEGLTKEY DRLLEEHAKL |
| | | | | | QAAVDGPMDK KEE |
| 55 | TAPBP | 1 | O15533 | Entry version | MKSLSLLLAV ALGLATAVSA |
| | | | | 190 (18 Sep. | GPAVIECWFV EDASGKGLAK |
| | | | | 2019) | RPGALLLRQG PGEPPPRPDL |
| | | | | Sequence | DPELYLSVHD PAGALQAAFR |
| | | | | version 1 (01 | RYPRGAPAPH CEMSRFVPLP |
| | | | | Jan. 1998) | ASAKWASGLT PAQNCPRALD |
| | | | | | GAWLMVSISS PVLSLSSLLR |
| | | | | | PQPEPQQEPV LITMATVVLT |
| | | | | | VLTHTPAPRV RLGQDALLDL |
| | | | | | SFAYMPPTSE AASSLAPGPP |
| | | | | | PFGLEWRRQH LGKGHLLLAA |
| | | | | | TPGLNGQMPA AQEGAVAFAA |
| | | | | | WDDDEPWGPW TGNGTFWLPR |
| | | | | | VQPFQEGTYL ATIHLPYLQG |
| | | | | | QVTLELAVYK PPKVSLMPAT |
| | | | | | LARAAPGEAP PELLCLVSHF |
| | | | | | YPSGGLEVEW ELRGGPGGRS |
| | | | | | QKAEGQRWLS ALRHHSDGSV |
| | | | | | SLSGHLQPPP VTTEQHGARY |
| | | | | | ACRIHHPSLP ASGRSAEVTL |
| | | | | | EVAGLSGPSL EDSVGLFLSA |
| | | | | | FLLLGLFKAL GWAAVYLSTC |
| | | | | | KDSKKKAE |
| 56 | TAPBP | 2 | O15533- | Entry version | MKSLSLLLAV ALGLATAVSA |
| | | | 2 | 190 (18 Sep. | GPAVIECWFV EDASGKGLAK |
| | | | | 2019) | RPGALLLRQG PGEPPPRPDL |
| | | | | Sequence | DPELYLSVHD PAGALQAAFR |
| | | | | version 1 (01 | RYPRGAPAPH CEMSRFVPLP |
| | | | | Jan. 1998) | ASAKWASGLT PAQNCPRALD |
| | | | | | GAWLMVSISS PVLSLSSLLR |
| | | | | | PQPEPQQEPV LITMATVVLT |
| | | | | | VLTHTPAPRV RLGQDALLDL |
| | | | | | SFAYMPPTSE AASSLAPGPP |
| | | | | | PFGLEWRRQH LGKGHLLLAA |
| | | | | | TPGLNGQMPA AQEGAVAFAA |
| | | | | | WDDDEPWGPW TGNGTFWLPR |
| | | | | | VQPFQEGTYL ATIHLPYLQG |
| | | | | | QVTLELAVYK PPKVSLMPAT |
| | | | | | LARAAPGEAP PELLCLVSHF |
| | | | | | YPSGGLEVEW ELRGGPGGRS |
| | | | | | QKAEGQRWLS ALRHHSDGSV |
| | | | | | SLSGHLQPPP VTTEQHGARY |
| | | | | | ACRIHHPSLP ASGRSAEVTL |
| | | | | | EVAGKSWELC GI |
| 57 | TAPBP | 3 | O15533- | Entry version | MKSLSLLLAV ALGLATAVSA |
| | | | 3 | 190 (18 Sep. | GPAVIECWFV EDASGKGLAK |
| | | | | 2019) | RPGALLLRQG PGEPPPRPDL |
| | | | | Sequence | DPELYLSVHD PAGALQAAFR |
| | | | | version 1 (01 | RYPRGAPAPH CEMSRFVPLP |
| | | | | Jan. 1998) | ASAKWASGLT PAQNCPRALD |
| | | | | | GAWLMVSISS PVLSLSSLLR |
| | | | | | PQPEPQQEPV LITMATVVLT |
| | | | | | VLTHTPAPRV RLGQDALLDL |
| | | | | | SFAYMPPTSE AASSLAPGPP |
| | | | | | PFGLEWRRQH LGKGHLLLAA |
| | | | | | TPGLNGQMPA AQEGAVAFAA |
| | | | | | WDDDEPWGPW TGNGTFWLPR |
| | | | | | VQPFQEGTYL ATIHLPYLQG |
| | | | | | QVTLELAVYK PPKVSLMPAT |
| | | | | | LARAAPGEAP PELLCLVSHF |
| | | | | | YPSGGLEVEW ELRGGPGGRS |
| | | | | | QKAEGQRWLS ALRHHSDGSV |
| | | | | | SLSGHLQPPP VTTEQHGARY |
| | | | | | ACRIHHPSLP ASGRSAEVTL |
| | | | | | EVAGLSGPSL EDSVGLFLSA |
| | | | | | FLLLGLFKAL GWAAVYLSTC |
| | | | | | KDSKKVQCST SLYLSLVTLS |
| | | | | | PHPISKPMEG GCWCGRQNLG |
| | | | | | LEFTLIWVKT WHYILTVGLF |
| | | | | | EHAT |
| 58 | TAPBP | 4 | O15533- | Entry version | MKSLSLLLAV ALGLATAVSA |
| | | | 4 | 190 (18 Sep. | GPAVIECWFV EDASGKGLAK |
| | | | | 2019) | RPGALLLRQG PGEPPPRPDL |
| | | | | Sequence | DPELYLSVHV VLTVLTHTPA |
| | | | | version 1 (01 | PRVRLGQDAL LDLSFAYMPP |
| | | | | Jan. 1998) | TSEAASSLAP GPPPFGLEWR |
| | | | | | RQHLGKGHLL LAATPGLNGQ |
| | | | | | MPAAQEGAVA FAAWDDDEPW |
| | | | | | GPWTGNGTFW LPRVQPFQEG |
| | | | | | TYLATIHLPY LQGQVTLELA |
| | | | | | VYKPPKVSLM PATLARAAPG |
| | | | | | EAPPELLCLV SHFYPSGGLE |
| | | | | | VEWELRGGPG GRSQKAEGQR |
| | | | | | WLSALRHHSD GSVSLSGHLQ |
| | | | | | PPPVTTEQHG ARYACRIHHP |
| | | | | | SLPASGRSAE VTLEVAGLSG |
| | | | | | PSLEDSVGLF LSAFLLLGLF |
| | | | | | KALGWAAVYL STCKDSKKKA |
| | | | | | E |
| 59 | PPP3CB | 1 | P16298 | Entry version | MAAPEPARAA PPPPPPPPPP |
| | | | | 166 (18 Sep. | PGADRVVKAV PFPPTHRLTS |
| | | | | 2019) | EEVFDLDGIP RVDVLKNHLV |
| | | | | Sequence | KEGRVDEEIA LRIINEGAAI |
| | | | | version 2 (01 | LRREKTMIEV EAPITVCGDI |
| | | | | Feb. 2005) | HGQFFDLMKL FEVGGSPANT |
| | | | | | RYLFLGDYVD RGYFSIECVL |
| | | | | | YLWVLKILYP STLFLLRGNH |
| | | | | | ECRHLTEYFT FKQECKIKYS |
| | | | | | ERVYEACMEA FDSLPLAALL |
| | | | | | NQQFLCVHGG LSPEIHTLDD |
| | | | | | IRRLDRFKEP PAFGPMCDLL |
| | | | | | WSDPSEDFGN EKSQEHFSHN |
| | | | | | TVRGCSYFYN YPAVCEFLQN |
| | | | | | NNLLSIIRAH EAQDAGYRMY |
| | | | | | RKSQTTGFPS LITIFSAPNY |
| | | | | | LDVYNNKAAV LKYENNVMNI |
| | | | | | RQFNCSPHPY WLPNFMDVFT |
| | | | | | WSLPFVGEKV TEMLVNVLSI |
| | | | | | CSDDELMTEG EDQFDGSAAA |
| | | | | | RKEIIRNKIR AIGKMARVFS |
| | | | | | VLREESESVL TLKGLTPTGM |
| | | | | | LPSGVLAGGR QTLQSATVEA |
| | | | | | IEAEKAIRGF SPPHRICSFE |
| | | | | | EAKGLDRINE RMPPRKDAVQ |
| | | | | | QDGFNSLNTA HATENHGTGN |
| | | | | | HTAQ |
| 60 | PPP3CB | 2 | P16298- | Entry version | MAAPEPARAA PPPPPPPPPP |
| | | | 2 | 166 (18 Sep. | PGADRVVKAV PFPPTHRLTS |
| | | | | 2019) | EEVFDLDGIP RVDVLKNHLV |
| | | | | Sequence | KEGRVDEEIA LRIINEGAAI |
| | | | | version 2 (01 | LRREKTMIEV EAPITVCGDI |
| | | | | Feb. 2005) | HGQFFDLMKL FEVGGSPANT |
| | | | | | RYLFLGDYVD RGYFSIEHVL |
| | | | | | GTEDISINPH NNINECVLYL |
| | | | | | WVLKILYPST LFLLRGNHEC |
| | | | | | RHLTEYFTFK QECKIKYSER |
| | | | | | VYEACMEAFD SLPLAALLNQ |
| | | | | | QFLCVHGGLS PEIHTLDDIR |
| | | | | | RLDRFKEPPA FGPMCDLLWS |
| | | | | | DPSEDFGNEK SQEHFSHNTV |
| | | | | | RGCSYFYNYP AVCEFLQNNN |
| | | | | | LLSIIRAHEA QDAGYRMYRK |
| | | | | | SQTTGFPSLI TIFSAPNYLD |
| | | | | | VYNNKAAVLK YENNVMNIRQ |
| | | | | | FNCSPHPYWL PNFMDVFTWS |
| | | | | | LPFVGEKVTE MLVNVLSICS |
| | | | | | DDELMTEGED QFDGSAAARK |
| | | | | | EIIRNKIRAI GKMARVFSVL |
| | | | | | REESESVLTL KGLTPTGMLP |
| | | | | | SGVLAGGRQT LQSGNDVMQL |
| | | | | | AVPQMDWGTP HSFANNSHNA |
| | | | | | CREFLLFFSS CLSS |
| 61 | PPP3CB | 3 | P16298- | Entry version | MAAPEPARAA PPPPPPPPPP |
| | | | 3 | 166 (18 Sep. | PGADRVVKAV PFPPTHRLTS |
| | | | | 2019) | EEVFDLDGIP RVDVLKNHLV |
| | | | | Sequence | KEGRVDEEIA LRIINEGAAI |
| | | | | version 2 (01 | LRREKTMIEV EAPITVCGDI |
| | | | | Feb. 2005) | HGQFFDLMKL FEVGGSPANT |
| | | | | | RYLFLGDYVD RGYFSIECVL |
| | | | | | YLWVLKILYP STLFLLRGNH |
| | | | | | ECRHLTEYFT FKQECKIKYS |
| | | | | | ERVYEACMEA FDSLPLAALL |
| | | | | | NQQFLCVHGG LSPEIHTLDD |
| | | | | | IRRLDRFKEP PAFGPMCDLL |
| | | | | | WSDPSEDFGN EKSQEHFSHN |
| | | | | | TVRGCSYFYN YPAVCEFLQN |
| | | | | | NNLLSIIRAH EAQDAGYRMY |
| | | | | | RKSQTTGFPS LITIFSAPNY |
| | | | | | LDVYNNKAAV LKYENNVMNI |
| | | | | | RQFNCSPHPY WLPNFMDVFT |
| | | | | | WSLPFVGEKV TEMLVNVLSI |
| | | | | | CSDDELMTEG EDQFDVGSAA |
| | | | | | ARKEIIRNKI RAIGKMARVF |
| | | | | | SVLREESESV LTLKGLTPTG |
| | | | | | MLPSGVLAGG RQTLQSAIRG |
| | | | | | FSPPHRICSF EEAKGLDRIN |
| | | | | | ERMPPRKDAV QQDGFNSLNT |
| | | | | | AHATENHGTG NHTAQ |
| 62 | PPP3CB | 4 | P16298- | Entry version | MAAPEPARAA PPPPPPPPPP |
| | | | 4 | 166 (18 Sep. | PGADRVVKAV PFPPTHRLTS |
| | | | | 2019) | EEVFDLDGIP RVDVLKNHLV |
| | | | | Sequence | KEGRVDEEIA LRIINEGAAI |
| | | | | version 2 (01 | LRREKTMIEV EAPITVCGDI |
| | | | | Feb. 2005) | HGQFFDLMKL FEVGGSPANT |
| | | | | | RYLFLGDYVD RGYFSIECVL |
| | | | | | YLWVLKILYP STLFLLRGNH |
| | | | | | ECRHLTEYFT FKQECKIKYS |
| | | | | | ERVYEACMEA FDSLPLAALL |
| | | | | | NQQFLCVHGG LSPEIHTLDD |
| | | | | | IRRLDRFKEP PAFGPMCDLL |
| | | | | | WSDPSEDFGN EKSQEHFSHN |
| | | | | | TVRGCSYFYN YPAVCEFLQN |
| | | | | | NNLLSIIRAH EAQDAGYRMY |
| | | | | | RKSQTTGFPS LITIFSAPNY |
| | | | | | LDVYNNKAAV LKYENNVMNI |
| | | | | | RQFNCSPHPY WLPNFMDVFT |
| | | | | | WSLPFVGEKV TEMLVNVLSI |
| | | | | | CSDDELMTEG EDQFDVGSAA |
| | | | | | ARKEIIRNKI RAIGKMARVF |
| | | | | | SVLREESESV LTLKGLTPTG |
| | | | | | MLPSGVLAGG RQTLQSATVE |
| | | | | | AIEAEKAIRG FSPPHRICSF |
| | | | | | EEAKGLDRIN ERMPPRKDAV |
| | | | | | QQDGFNSLNT AHATENHGTG |
| | | | | | NHTAQ |
| 63 | ANXA1 | 1 | P04083 | Entry version | MAMVSEFLKQ AWFIENEEQE |
| | | | | 244 (18 Sep. | YVQTVKSSKG GPGSAVSPYP |
| | | | | 2019) | TFNPSSDVAA LHKAIMVKGV |
| | | | | Sequence | DEATIIDILT KRNNAQRQQI |
| | | | | version 2 (23 | KAAYLQETGK PLDETLKKAL |
| | | | | Jan. 2007) | TGHLEEVVLA LLKTPAQFDA |
| | | | | | DELRAAMKGL GTDEDTLIEI |
| | | | | | LASRTNKEIR DINRVYREEL |
| | | | | | KRDLAKDITS DTSGDFRNAL |
| | | | | | LSLAKGDRSE DFGVNEDLAD |
| | | | | | SDARALYEAG ERRKGTDVNV |
| | | | | | FNTILTTRSY PQLRRVFQKY |
| | | | | | TKYSKHDMNK VLDLELKGDI |
| | | | | | EKCLTAIVKC ATSKPAFFAE |
| | | | | | KLHQAMKGVG TRHKALIRIM |
| | | | | | VSRSEIDMND IKAFYQKMYG |
| | | | | | ISLCQAILDE TKGDYEKILV |
| | | | | | ALCGGN |
| 64 | PERM | 1 | P05164 | Entry version | MGVPFFSSLR CMVDLGPCWA |
| | | | | 221 (18 Sep. | GGLTAEMKLL LALAGLLAIL |
| | | | | 2019) | ATPQPSEGAA PAVLGEVDTS |
| | | | | Sequence | LVLSSMEEAK QLVDKAYKER |
| | | | | version 1 (13 | RESIKQRLRS GSASPMELLS |
| | | | | Aug. 1987) | YFKQPVAATR TAVRAADYLH |
| | | | | | VALDLLERKL RSLWRRPFNV |
| | | | | | TDVLTPAQLN VLSKSSGCAY |
| | | | | | QDVGVTCPEQ DKYRTITGMC |
| | | | | | NNRRSPTLGA SNRAFVRWLP |
| | | | | | AEYEDGFSLP YGWTPGVKRN |
| | | | | | GFPVALARAV SNEIVRFPTD |
| | | | | | QLTPDQERSL MFMQWGQLLD |
| | | | | | HDLDFTPEPA ARASFVTGVN |
| | | | | | CETSCVQQPP CFPLKIPPND |
| | | | | | PRIKNQADCI PFFRSCPACP |
| | | | | | GSNITIRNQI NALTSFVDAS |
| | | | | | MVYGSEEPLA RNLRNMSNQL |
| | | | | | GLLAVNQRFQ DNGRALLPFD |
| | | | | | NLHDDPCLLT NRSARIPCFL |
| | | | | | AGDTRSSEMP ELTSMHTLLL |
| | | | | | REHNRLATEL KSLNPRWDGE |
| | | | | | RLYQEARKIV GAMVQIITYR |
| | | | | | DYLPLVLGPT AMRKYLPTYR |
| | | | | | SYNDSVDPRI ANVFTNAFRY |
| | | | | | GHTLIQPFMF RLDNRYQPME |
| | | | | | PNPRVPLSRV FFASWRVVLE |
| | | | | | GGIDPILRGL MATPAKLNRQ |
| | | | | | NQIAVDEIRE RLFEQVMRIG |
| | | | | | LDLPALNMQR SRDHGLPGYN |
| | | | | | AWRRFCGLPQ PETVGQLGTV |
| | | | | | LRNLKLARKL MEQYGTPNNI |
| | | | | | DIWMGGVSEP LKRKGRVGPL |
| | | | | | LACIIGTQFR KLRDGDRFWW |
| | | | | | ENEGVESMQQ RQALAQISLP |
| | | | | | RIICDNTGIT TVSKNNIFMS |
| | | | | | NSYPRDFVNC STLPALNLAS |
| | | | | | WREAS |
| 65 | PERM | 2 | P05164- | Entry version | MELLSYFKQP VAATRTAVRA |
| | | | 2 | 221 (18 Sep. | ADYLHVALDL LERKLRSLWR |
| | | | | 2019) | RPFNVTDVLT PAQLNVLSKS |
| | | | | Sequence | SGCAYQDVGV TCPEQDKYRT |
| | | | | version 1 (13 | ITGMCNNRRS PTLGASNRAF |
| | | | | Aug. 1987) | VRWLPAEYED GFSLPYGWTP |
| | | | | | GVKRNGFPVA LARAVSNEIV |
| | | | | | RFPTDQLTPD QERSLMFMQW |
| | | | | | GQLLDHDLDF TPEPAARASF |
| | | | | | VTGVNCETSC VQQPPCFPLK |
| | | | | | IPPNDPRIKN QADCIPFFRS |
| | | | | | CPACPGSNIT IRNQINALTS |
| | | | | | FVDASMVYGS EEPLARNLRN |
| | | | | | MSNQLGLLAV NQRFQDNGRA |
| | | | | | LLPFDNLHDD PCLLTNRSAR |
| | | | | | IPCFLAGDTR SSEMPELTSM |
| | | | | | HTLLLREHNR LATELKSLNP |
| | | | | | RWDGERLYQE ARKIVGAMVQ |
| | | | | | IITYRDYLPL VLGPTAMRKY |
| | | | | | LPTYRSYNDS VDPRIANVFT |
| | | | | | NAFRYGHTLI QPFMFRLDNR |
| | | | | | YQPMEPNPRV PLSRVFFASW |
| | | | | | RVVLEGGIDP ILRGLMATPA |
| | | | | | KLNRQNQIAV DEIRERLFEQ |
| | | | | | VMRIGLDLPA LNMQRSRDHG |
| | | | | | LPGYNAWRRF CGLPQPETVG |
| | | | | | QLGTVLRNLK LARKLMEQYG |
| | | | | | TPNNIDIWMG GVSEPLKRKG |
| | | | | | RVGPLLACII GTQFRKLRDG |
| | | | | | DRFWWENEGV FSMQQRQALA |
| | | | | | QISLPRIICD NTGITTVSKN |
| | | | | | NIFMSNSYPR DFVNCSTLPA |
| | | | | | LNLASWREAS |
| 66 | PERM | 3 | P05164- | Entry version | MGVPFFSSLR CMVDLGPCWA |
| | | | 3 | 221 (18 Sep. | GGLTAEMKLL LALAGLLAIL |
| | | | | 2019) | ATPQPSEGAA PAVLGEVDTS |
| | | | | Sequence | LVLSSMEEAK QLVDKAYKER |
| | | | | version 1 (13 | RESIKQRLRS GSASPMELLS |
| | | | | Aug. 1987) | YFKQPVAATR TAVRAADYLH |
| | | | | | VALDLLERKL RSLWRRPFNV |
| | | | | | TDVLTPAQLN VLSKSSGCAY |
| | | | | | QDVGVTCPEQ DKYRTITGMC |
| | | | | | NNRCGWLGVA AGTGLREASR |
| | | | | | TPQASRCQRP VLPCRRSPTL |
| | | | | | GASNRAFVRW LPAEYEDGFS |
| | | | | | LPYGWTPGVK RNGFPVALAR |
| | | | | | AVSNEIVREP TDQLTPDQER |
| | | | | | SLMFMQWGQL LDHDLDFTPE |
| | | | | | PAARASFVTG VNCETSCVQQ |
| | | | | | PPCFPLKIPP NDPRIKNQAD |
| | | | | | CIPFFRSCPA CPGSNITIRN |
| | | | | | QINALTSFVD ASMVYGSEEP |
| | | | | | LARNLRNMSN QLGLLAVNQR |
| | | | | | FQDNGRALLP FDNLHDDPCL |
| | | | | | LTNRSARIPC FLAGDTRSSE |
| | | | | | MPELTSMHTL LLREHNRLAT |
| | | | | | ELKSLNPRWD GERLYQEARK |
| | | | | | IVGAMVQIIT YRDYLPLVLG |
| | | | | | PTAMRKYLPT YRSYNDSVDP |
| | | | | | RIANVFTNAF RYGHTLIQPF |
| | | | | | MFRLDNRYQP MEPNPRVPLS |
| | | | | | RVFFASWRVV LEGGIDPILR |
| | | | | | GLMATPAKLN RQNQIAVDEI |
| | | | | | RERLFEQVMR IGLDLPALNM |
| | | | | | QRSRDHGLPG YNAWRRFCGL |
| | | | | | PQPETVGQLG TVLRNLKLAR |
| | | | | | KLMEQYGTPN NIDIWMGGVS |
| | | | | | EPLKRKGRVG PLLACIIGTQ |
| | | | | | FRKLRDGDRF WWENEGVFSM |
| | | | | | QQRQALAQIS LPRIICDNTG |
| | | | | | ITTVSKNNIF MSNSYPRDFV |
| | | | | | NCSTLPALNL ASWREAS |
| 67 | PLEC | 1 | Q15149 | Entry version | MVAGMLMPRD QLRAIYEVLF |
| | | | | 224 (18 Sep. | REGVMVAKKD RRPRSLHPHV |
| | | | | 2019) | PGVTNLQVMR AMASLRARGL |
| | | | | Sequence | VRETFAWCHF YWYLTNEGIA |
| | | | | version 3 (14 | HLRQYLHLPP EIVPASLQRV |
| | | | | Oct. 2008) | RRPVAMVMPA RRTPHVQAVQ |
| | | | | | GPLGSPPKRG PLPTEEQRVY |
| | | | | | RRKELEEVSP ETPVVPATTQ |
| | | | | | RTLARPGPEP APATDERDRV |
| | | | | | QKKTFTKWVN KHLIKAQRHI |
| | | | | | SDLYEDLRDG HNLISLLEVL |
| | | | | | SGDSLPREKG RMRFHKLQNV |
| | | | | | QIALDYLRHR QVKLVNIRND |
| | | | | | DIADGNPKLT LGLIWTIILH |
| | | | | | FQISDIQVSG QSEDMTAKEK |
| | | | | | LLLWSQRMVE GYQGLRCDNF |
| | | | | | TSSWRDGRLF NAIIHRHKPL |
| | | | | | LIDMNKVYRQ TNLENLDQAF |
| | | | | | SVAERDLGVT RLLDPEDVDV |
| | | | | | PQPDEKSIIT YVSSLYDAMP |
| | | | | | RVPDVQDGVR ANELQLRWQE |
| | | | | | YRELVLLLLQ WMRHHTAAFE |
| | | | | | ERRFPSSFEE IEILWSQFLK |
| | | | | | FKEMELPAKE ADKNRSKGIY |
| | | | | | QSLEGAVQAG QLKVPPGYHP |
| | | | | | LDVEKEWGKL HVAILEREKQ |
| | | | | | LRSEFERLEC LQRIVTKLQM |
| | | | | | EAGLCEEQLN QADALLQSDV |
| | | | | | RLLAAGKVPQ RAGEVERDLD |
| | | | | | KADSMIRLLF NDVQTLKDGR |
| | | | | | HPQGEQMYRR VYRLHERLVA |
| | | | | | IRTEYNLRLK AGVAAPATQV |
| | | | | | AQVTLQSVQR RPELEDSTLR |
| | | | | | YLQDLLAWVE ENQHRVDGAE |
| | | | | | WGVDLPSVEA QLGSHRGLHQ |
| | | | | | SIEEFRAKIE RARSDEGQLS |
| | | | | | PATRGAYRDC LGRLDLQYAK |
| | | | | | LLNSSKARLR SLESLHSFVA |
| | | | | | AATKELMWLN EKEEEEVGFD |
| | | | | | WSDRNTNMTA KKESYSALMR |
| | | | | | ELELKEKKIK ELQNAGDRLL |
| | | | | | REDHPARPTV ESFQAALQTQ |
| | | | | | WSWMLQLCCC IEAHLKENAA |
| | | | | | YFQFFSDVRE AEGQLQKLQE |
| | | | | | ALRRKYSCDR SATVTRLEDL |
| | | | | | LQDAQDEKEQ LNEYKGHLSG |
| | | | | | LAKRAKAVVQ LKPRHPAHPM |
| | | | | | RGRLPLLAVC DYKQVEVTVH |
| | | | | | KGDECQLVGP AQPSHWKVLS |
| | | | | | SSGSEAAVPS VCFLVPPPNQ |
| | | | | | EAQEAVTRLE AQHQALVTLW |
| | | | | | HQLHVDMKSL LAWQSLRRDV |
| | | | | | QLIRSWSLAT FRTLKPEEQR |
| | | | | | QALHSLELHY QAFLRDSQDA |
| | | | | | GGFGPEDRLM AEREYGSCSH |
| | | | | | HYQQLLQSLE QGAQEESRCQ |
| | | | | | RCISELKDIR LQLEACETRT |
| | | | | | VHRLRLPLDK EPARECAQRI |
| | | | | | AEQQKAQAEV EGLGKGVARL |
| | | | | | SAEAEKVLAL PEPSPAAPTL |
| | | | | | RSELELTLGK LEQVRSLSAI |
| | | | | | YLEKLKTISL VIRGTQGAEE |
| | | | | | VLRAHEEQLK EAQAVPATLP |
| | | | | | ELEATKASLK KLRAQAEAQQ |
| | | | | | PTFDALRDEL RGAQEVGERL |
| | | | | | QQRHGERDVE VERWRERVAQ |
| | | | | | LLERWQAVLA QTDVRQRELE |
| | | | | | QLGRQLRYYR ESADPLGAWL |
| | | | | | QDARRRQEQI QAMPLADSQA |
| | | | | | VREQLRQEQA LLEEIERHGE |
| | | | | | KVEECQRFAK QYINAIKDYE |
| | | | | | LQLVTYKAQL EPVASPAKKP |
| | | | | | KVQSGSESVI QEYVDLRTHY |
| | | | | | SELTTLTSQY IKFISETLRR |
| | | | | | MEEEERLAEQ QRAEERERLA |
| | | | | | EVEAALEKQR QLAEAHAQAK |
| | | | | | AQAEREAKEL QQRMQEEVVR |
| | | | | | REEAAVDAQQ QKRSIQEELQ |
| | | | | | QLRQSSEAEI QAKARQAEAA |
| | | | | | ERSRLRIEEE IRVVRLQLEA |
| | | | | | TERQRGGAEG ELQALRARAE |
| | | | | | EAEAQKRQAQ EEAERLRRQV |
| | | | | | QDESQRKRQA EVELASRVKA |
| | | | | | EAEAAREKQR ALQALEELRL |
| | | | | | QAEEAERRLR QAEVERARQV |
| | | | | | QVALETAQRS AEAELQSKRA |
| | | | | | SFAEKTAQLE RSLQEEHVAV |
| | | | | | AQLREEAERR AQQQAEAERA |
| | | | | | REEAERELER WQLKANEALR |
| | | | | | LRLQAEEVAQ QKSLAQAEAE |
| | | | | | KQKEEAEREA RRRGKAEEQA |
| | | | | | VRQRELAEQE LEKQRQLAEG |
| | | | | | TAQQRLAAEQ ELIRLRAETE |
| | | | | | QGEQQRQLLE EELARLQREA |
| | | | | | AAATQKRQEL EAELAKVRAE |
| | | | | | MEVLLASKAR AEEESRSTSE |
| | | | | | KSKQRLEAEA GRFRELAEEA |
| | | | | | ARLRALAEEA KRQRQLAEED |
| | | | | | AARQRAEAER VLAEKLAAIG |
| | | | | | EATRLKTEAE IALKEKEAEN |
| | | | | | ERLRRLAEDE AFQRRRLEEQ |
| | | | | | AAQHKADIEE RLAQLRKASD |
| | | | | | SELERQKGLV EDTLRQRRQV |
| | | | | | EEEILALKAS FEKAAAGKAE |
| | | | | | LELELGRIRS NAEDTLRSKE |
| | | | | | QAELEAARQR QLAAEEERRR |
| | | | | | REAEERVQKS LAAEEEAARQ |
| | | | | | RKAALEEVER LKAKVEEARR |
| | | | | | LRERAEQESA RQLQLAQEAA |
| | | | | | QKRLQAEEKA HAFAVQQKEQ |
| | | | | | ELQQTLQQEQ SVLDQLRGEA |
| | | | | | EAARRAAEEA EEARVQAERE |
| | | | | | AAQSRRQVEE AERLKQSAEE |
| | | | | | QAQARAQAQA AAEKLRKEAE |
| | | | | | QEAARRAQAE QAALRQKQAA |
| | | | | | DAEMEKHKKF AEQTLRQKAQ |
| | | | | | VEQELTTLRL QLEETDHQKN |
| | | | | | LLDEELQRLK AEATEAARQR |
| | | | | | SQVEEELFSV RVQMEELSKL |
| | | | | | KARIEAENRA LILRDKDNTQ |
| | | | | | RFLQEEAEKM KQVAEEAARL |
| | | | | | SVAAQEAARL RQLAEEDLAQ |
| | | | | | QRALAEKMLK EKMQAVQEAT |
| | | | | | RLKAEAELLQ QQKELAQEQA |
| | | | | | RRLQEDKEQM AQQLAEETQG |
| | | | | | FQRTLEAERQ RQLEMSAEAE |
| | | | | | RLKLRVAEMS RAQARAEEDA |
| | | | | | QRFRKQAEEI GEKLHRTELA |
| | | | | | TQEKVTLVQT LEIQRQQSDH |
| | | | | | DAERLREAIA ELEREKEKLQ |
| | | | | | QEAKLLQLKS EEMQTVQQEQ |
| | | | | | LLQETQALQQ SFLSEKDSLL |
| | | | | | QRERFIEQEK AKLEQLFQDE |
| | | | | | VAKAQQLREE QQRQQQQMEQ |
| | | | | | ERQRLVASME EARRRQHEAE |
| | | | | | EGVRRKQEEL QQLEQQRRQQ |
| | | | | | EELLAEENQR LREQLQLLEE |
| | | | | | QHRAALAHSE EVTASQVAAT |
| | | | | | KTLPNGRDAL DGPAAEAEPE |
| | | | | | HSFDGLRRKV SAQRLQEAGI |
| | | | | | LSAEELQRLA QGHTTVDELA |
| | | | | | RREDVRHYLQ GRSSIAGLLL |
| | | | | | KATNEKLSVY AALQRQLLSP |
| | | | | | GTALILLEAQ AASGFLLDPV |
| | | | | | RNRRLTVNEA VKEGVVGPEL |
| | | | | | HHKLLSAERA VTGYKDPYTG |
| | | | | | QQISLFQAMQ KGLIVREHGI |
| | | | | | RLLEAQIATG GVIDPVHSHR |
| | | | | | VPVDVAYRRG YFDEEMNRVL |
| | | | | | ADPSDDTKGF FDPNTHENLT |
| | | | | | YLQLLERCVE DPETGLCLLP |
| | | | | | LTDKAAKGGE LVYTDSEARD |
| | | | | | VFEKATVSAP FGKFQGKTVT |
| | | | | | IWEIINSEYF TAEQRRDLLR |
| | | | | | QFRTGRITVE KIIKIIITVV |
| | | | | | EEQEQKGRLC FEGLRSLVPA |
| | | | | | AELLESRVID RELYQQLQRG |
| | | | | | ERSVRDVAEV DTVRRALRGA |
| | | | | | NVIAGVWLEE AGQKLSIYNA |
| | | | | | LKKDLLPSDM AVALLEAQAG |
| | | | | | TGHIIDPATS ARLTVDEAVR |
| | | | | | AGLVGPEFHE KLLSAEKAVT |
| | | | | | GYRDPYTGQS VSLFQALKKG |
| | | | | | LIPREQGLRL LDAQLSTGGI |
| | | | | | VDPSKSHRVP LDVACARGCL |
| | | | | | DEETSRALSA PRADAKAYSD |
| | | | | | PSTGEPATYG ELQQRCRPDQ |
| | | | | | LTGLSLLPLS EKAARARQEE |
| | | | | | LYSELQARET FEKTPVEVPV |
| | | | | | GGFKGRTVTV WELISSEYFT |
| | | | | | AEQRQELLRQ FRTGKVTVEK |
| | | | | | VIKILITIVE EVETLRQERL |
| | | | | | SFSGLRAPVP ASELLASGVL |
| | | | | | SRAQFEQLKD GKTTVKDLSE |
| | | | | | LGSVRTLLQG SGCLAGIYLE |
| | | | | | DTKEKVSIYE AMRRGLLRAT |
| | | | | | TAALLLEAQA ATGFLVDPVR |
| | | | | | NQRLYVHEAV KAGVVGPELH |
| | | | | | EQLLSAEKAV TGYRDPYSGS |
| | | | | | TISLFQAMQK GLVLRQHGIR |
| | | | | | LLEAQIATGG IIDPVHSHRV |
| | | | | | PVDVAYQRGY FSEEMNRVLA |
| | | | | | DPSDDTKGFF DPNTHENLTY |
| | | | | | RQLLERCVED PETGLRLLPL |
| | | | | | KGAEKAEVVE TTQVYTEEET |
| | | | | | RRAFEETQID IPGGGSHGGS |
| | | | | | TMSLWEVMQS DLIPEEQRAQ |
| | | | | | LMADFQAGRV TKERMIIIII |
| | | | | | EIIEKTEIIR QQGLASYDYV |
| | | | | | RRRLTAEDLF EARIISLETY |
| | | | | | NLLREGTRSL REALEAESAW |
| | | | | | CYLYGTGSVA GVYLPGSRQT |
| | | | | | LSIYQALKKG LLSAEVARLL |
| | | | | | LEAQAATGFL LDPVKGERLT |
| | | | | | VDEAVRKGLV GPELHDRLLS |
| | | | | | AERAVTGYRD PYTEQTISLF |
| | | | | | QAMKKELIPT EEALRLLDAQ |
| | | | | | LATGGIVDPR LGFHLPLEVA |
| | | | | | YQRGYLNKDT HDQLSEPSEV |
| | | | | | RSYVDPSTDE RLSYTQLLRR |
| | | | | | CRRDDGTGQL LLPLSDARKL |
| | | | | | TFRGLRKQIT MEELVRSQVM |
| | | | | | DEATALQLRE GLTSIEEVTK |
| | | | | | NLQKFLEGTS CIAGVFVDAT |
| | | | | | KERLSVYQAM KKGIIRPGTA |
| | | | | | FELLEAQAAT GYVIDPIKGL |
| | | | | | KLTVEEAVRM GIVGPEFKDK |
| | | | | | LLSAERAVTG YKDPYSGKLI |
| | | | | | SLFQAMKKGL ILKDHGIRLL |
| | | | | | EAQIATGGII DPEESHRLPV |
| | | | | | EVAYKRGLFD EEMNEILTDP |
| | | | | | SDDTKGFFDP NTEENLTYLQ |
| | | | | | LMERCITDPQ TGLCLLPLKE |
| | | | | | KKRERKTSSK SSVRKRRVVI |
| | | | | | VDPETGKEMS VYEAYRKGLI |
| | | | | | DHQTYLELSE QECEWEEITI |
| | | | | | SSSDGVVKSM IIDRRSGRQY |
| | | | | | DIDDAIAKNL IDRSALDQYR |
| | | | | | AGTLSITEFA DMLSGNAGGF |
| | | | | | RSRSSSVGSS SSYPISPAVS |
| | | | | | RTQLASWSDP TEETGPVAGI |
| | | | | | LDTETLEKVS ITEAMHRNLV |
| | | | | | DNITGQRLLE AQACTGGIID |
| | | | | | PSTGERFPVT DAVNKGLVDK |
| | | | | | IMVDRINLAQ KAFCGFEDPR |
| | | | | | TKTKMSAAQA LKKGWLYYEA |
| | | | | | GQRFLEVQYL TGGLIEPDTP |
| | | | | | GRVPLDEALQ RGTVDARTAQ |
| | | | | | KLRDVGAYSK YLTCPKTKLK |
| | | | | | ISYKDALDRS MVEEGTGLRL |
| | | | | | LEAAAQSTKG YYSPYSVSGS |
| | | | | | GSTAGSRTGS RTGSRAGSRR |
| | | | | | GSFDATGSGF SMTFSSSSYS |
| | | | | | SSGYGRRYAS GSSASLGGPE |
| | | | | | SAVA |
| 68 | PLEC | 2 | Q15149- | Entry version | MSGEDAEVRA VSEDVSNGSS |
| | | | 2 | 224 (18 Sep. | GSPSPGDTLP WNLGKTQRSR |
| | | | | 2019) | RSGGGAGSNG SVLDPAERAV |
| | | | | Sequence | IRIADERDRV QKKTFTKWVN |
| | | | | version 3 (14 | KHLIKAQRHI SDLYEDLRDG |
| | | | | Oct. 2008) | HNLISLLEVL SGDSLPREKG |
| | | | | | RMRFHKLQNV QIALDYLRHR |
| | | | | | QVKLVNIRND DIADGNPKLT |
| | | | | | LGLIWTIILH FQISDIQVSG |
| | | | | | QSEDMTAKEK LLLWSQRMVE |
| | | | | | GYQGLRCDNF TSSWRDGRLF |
| | | | | | NAIIHRHKPL LIDMNKVYRQ |
| | | | | | TNLENLDQAF SVAERDLGVT |
| | | | | | RLLDPEDVDV PQPDEKSIIT |
| | | | | | YVSSLYDAMP RVPDVQDGVR |
| | | | | | ANELQLRWQE YRELVLLLLQ |
| | | | | | WMRHHTAAFE ERRFPSSFEE |
| | | | | | IEILWSQFLK FKEMELPAKE |
| | | | | | ADKNRSKGIY QSLEGAVQAG |
| | | | | | QLKVPPGYHP LDVEKEWGKL |
| | | | | | HVAILEREKQ LRSEFERLEC |
| | | | | | LQRIVTKLQM EAGLCEEQLN |
| | | | | | QADALLQSDV RLLAAGKVPQ |
| | | | | | RAGEVERDLD KADSMIRLLF |
| | | | | | NDVQTLKDGR HPQGEQMYRR |
| | | | | | VYRLHERLVA IRTEYNLRLK |
| | | | | | AGVAAPATQV AQVTLQSVQR |
| | | | | | RPELEDSTLR YLQDLLAWVE |
| | | | | | ENQHRVDGAE WGVDLPSVEA |
| | | | | | QLGSHRGLHQ SIEEFRAKIE |
| | | | | | RARSDEGQLS PATRGAYRDC |
| | | | | | LGRLDLQYAK LLNSSKARLR |
| | | | | | SLESLHSFVA AATKELMWLN |
| | | | | | EKEEEEVGFD WSDRNTNMTA |
| | | | | | KKESYSALMR ELELKEKKIK |
| | | | | | ELQNAGDRLL REDHPARPTV |
| | | | | | ESFQAALQTQ WSWMLQLCCC |
| | | | | | IEAHLKENAA YFQFFSDVRE |
| | | | | | AEGQLQKLQE ALRRKYSCDR |
| | | | | | SATVTRLEDL LQDAQDEKEQ |
| | | | | | LNEYKGHLSG LAKRAKAVVQ |
| | | | | | LKPRHPAHPM RGRLPLLAVC |
| | | | | | DYKQVEVTVH KGDECQLVGP |
| | | | | | AQPSHWKVLS SSGSEAAVPS |
| | | | | | VCFLVPPPNQ EAQEAVTRLE |
| | | | | | AQHQALVTLW HQLHVDMKSL |
| | | | | | LAWQSLRRDV QLIRSWSLAT |
| | | | | | FRTLKPEEQR QALHSLELHY |
| | | | | | QAFLRDSQDA GGFGPEDRLM |
| | | | | | AEREYGSCSH HYQQLLQSLE |
| | | | | | QGAQEESRCQ RCISELKDIR |
| | | | | | LQLEACETRT VHRLRLPLDK |
| | | | | | EPARECAQRI AEQQKAQAEV |
| | | | | | EGLGKGVARL SAEAEKVLAL |
| | | | | | PEPSPAAPTL RSELELTLGK |
| | | | | | LEQVRSLSAI YLEKLKTISL |
| | | | | | VIRGTQGAEE VLRAHEEQLK |
| | | | | | EAQAVPATLP ELEATKASLK |
| | | | | | KLRAQAEAQQ PTFDALRDEL |
| | | | | | RGAQEVGERL QQRHGERDVE |
| | | | | | VERWRERVAQ LLERWQAVLA |
| | | | | | QTDVRQRELE QLGRQLRYYR |
| | | | | | ESADPLGAWL QDARRRQEQI |
| | | | | | QAMPLADSQA VREQLRQEQA |
| | | | | | LLEEIERHGE KVEECQRFAK |
| | | | | | QYINAIKDYE LQLVTYKAQL |
| | | | | | EPVASPAKKP KVQSGSESVI |
| | | | | | QEYVDLRTHY SELTTLTSQY |
| | | | | | IKFISETLRR MEEEERLAEQ |
| | | | | | QRAEERERLA EVEAALEKQR |
| | | | | | QLAEAHASAK AQAEREAKEL |
| | | | | | QQRMQEEVVR REEAAVDAQQ |
| | | | | | QKRSIQEELQ QLRQSSEAEI |
| | | | | | QAKARQAEAA ERSRLRIEEE |
| | | | | | IRVVRLQLEA TERQRGGAEG |
| | | | | | ELQALRARAE EAEAQKRQAQ |
| | | | | | EEAERLRRQV QDESQRKRQA |
| | | | | | EVELASRVKA EAEAAREKQR |
| | | | | | ALQALEELRL QAEEAERRLR |
| | | | | | QAEVERARQV QVALETAQRS |
| | | | | | AEAELQSKRA SFAEKTAQLE |
| | | | | | RSLQEEHVAV AQLREEAERR |
| | | | | | AQQQAEAERA REEAERELER |
| | | | | | WQLKANEALR LRLQAEEVAQ |
| | | | | | QKSLAQAEAE KQKEEAEREA |
| | | | | | RRRGKAEEQA VRQRELAEQE |
| | | | | | LEKQRQLAEG TAQQRLAAEQ |
| | | | | | ELIRLRAETE QGEQQRQLLE |
| | | | | | EELARLQREA AAATQKRQEL |
| | | | | | EAELAKVRAE MEVLLASKAR |
| | | | | | AEEESRSTSE KSKQRLEAEA |
| | | | | | GRFRELAEEA ARLRALAEEA |
| | | | | | KRQRQLAEED AARQRAEAER |
| | | | | | VLAEKLAAIG EATRLKTEAE |
| | | | | | IALKEKEAEN ERLRRLAEDE |
| | | | | | AFQRRRLEEQ AAQHKADIEE |
| | | | | | RLAQLRKASD SELERQKGLV |
| | | | | | EDTLRQRRQV EEEILALKAS |
| | | | | | FEKAAAGKAE LELELGRIRS |
| | | | | | NAEDTLRSKE QAELEAARQR |
| | | | | | QLAAEEERRR REAEERVQKS |
| | | | | | LAAEEEAARQ RKAALEEVER |
| | | | | | LKAKVEEARR LRERAEQESA |
| | | | | | RQLQLAQEAA QKRLQAEEKA |
| | | | | | HAFAVQQKEQ ELQQTLQQEQ |
| | | | | | SVLDQLRGEA EAARRAAEEA |
| | | | | | EEARVQAERE AAQSRRQVEE |
| | | | | | AERLKQSAEE QAQARAQAQA |
| | | | | | AAEKLRKEAE QEAARRAQAE |
| | | | | | QAALRQKQAA DAEMEKHKKF |
| | | | | | AEQTLRQKAQ VEQELTTLRL |
| | | | | | QLEETDHQKN LLDEELQRLK |
| | | | | | AEATEAARQR SQVEEELFSV |
| | | | | | RVQMEELSKL KARIEAENRA |
| | | | | | LILRDKDNTQ RFLQEEAEKM |
| | | | | | KQVAEEAARL SVAAQEAARL |
| | | | | | RQLAEEDLAQ QRALAEKMLK |
| | | | | | EKMQAVQEAT RLKAEAELLQ |
| | | | | | QQKELAQEQA RRLQEDKEQM |
| | | | | | AQQLAEETQG FQRTLEAERQ |
| | | | | | RQLEMSAEAE RLKLRVAEMS |
| | | | | | RAQARAEEDA QRFRKQAEEI |
| | | | | | GEKLHRTELA TQEKVTLV?T |
| | | | | | LEIQRQQSDH DAERLREAIA |
| | | | | | ELEREKEKLQ QEAKLLQLKS |
| | | | | | EEMQTVQQEQ LLQETQALQQ |
| | | | | | SFLSEKDSLL QRERFIEQEK |
| | | | | | AKLEQLFQDE VAKAQQLREE |
| | | | | | QQRQQQQMEQ ERQRLVASME |
| | | | | | EARRRQHEAE EGVRRKQEEL |
| | | | | | QQLEQQRRQQ EELLAEENQR |
| | | | | | LREQLQLLEE QHRAALAHSE |
| | | | | | EVTASQVAAT KTLPNGRDAL |
| | | | | | DGPAAEAEPE HSFDGLRRKV |
| | | | | | SAQRLQEAGI LSAEELQRLA |
| | | | | | QGHTTVDELA RREDVRHYLQ |
| | | | | | GRSSIAGLLL KATNEKLSVY |
| | | | | | AALQRQLLSP GTALILLEAQ |
| | | | | | AASGFLLDPV RNRRLTVNEA |
| | | | | | VKEGVVGPEL HHKLLSAERA |
| | | | | | VTGYKDPYTG QQISLFQAMQ |
| | | | | | KGLIVREHGI RLLEAQIATG |
| | | | | | GVIDPVHSHR VPVDVAYRRG |
| | | | | | YFDEEMNRVL ADPSDDTKGF |
| | | | | | FDPNTHENLT YLQLLERCVE |
| | | | | | DPETGLCLLP LTDKAAKGGE |
| | | | | | LVYTDSEARD VFEKATVSAP |
| | | | | | FGKFQGKTVT IWEIINSEYF |
| | | | | | TAEQRRDLLR QFRTGRITVE |
| | | | | | KIIKIIITVV EEQEQKGRLC |
| | | | | | FEGLRSLVPA AELLESRVID |
| | | | | | RELYQQLQRG ERSVRDVAEV |
| | | | | | DTVRRALRGA NVIAGVWLEE |
| | | | | | AGQKLSIYNA LKKDLLPSDM |
| | | | | | AVALLEAQAG TGHIIDPATS |
| | | | | | ARLTVDEAVR AGLVGPEFHE |
| | | | | | KLLSAEKAVT GYRDPYTGQS |
| | | | | | VSLFQALKKG LIPREQGLRL |
| | | | | | LDAQLSTGGI VDPSKSHRVP |
| | | | | | LDVACARGCL DEETSRALSA |
| | | | | | PRADAKAYSD PSTGEPATYG |
| | | | | | ELQQRCRPDQ LTGLSLLPLS |
| | | | | | EKAARARQEE LYSELQARET |
| | | | | | FEKTPVEVPV GGFKGRTVTV |
| | | | | | WELISSEYFT AEQRQELLRQ |
| | | | | | FRTGKVTVEK VIKILITIVE |
| | | | | | EVETLRQERL SFSGLRAPVP |
| | | | | | ASELLASGVL SRAQFEQLKD |
| | | | | | GKTTVKDLSE LGSVRTLLQG |
| | | | | | SGCLAGIYLE DTKEKVSIYE |
| | | | | | AMRRGLLRAT TAALLLEAQA |
| | | | | | ATGFLVDPVR NQRLYVHEAV |
| | | | | | KAGVVGPELH EQLLSAEKAV |
| | | | | | TGYRDPYSGS TISLFQAMQK |
| | | | | | GLVLRQHGIR LLEAQIATGG |
| | | | | | IIDPVHSHRV PVDVAYQRGY |
| | | | | | FSEEMNRVLA DPSDDTKGFF |
| | | | | | DPNTHENLTY RQLLERCVED |
| | | | | | PETGLRLLPL KGAEKAEVVE |
| | | | | | TTQVYTEEET RRAFEETQID |
| | | | | | IPGGGSHGGS TMSLWEVMQS |
| | | | | | DLIPEEQRAQ LMADFQAGRV |
| | | | | | TKERMIIIII EIIEKTEIIR |
| | | | | | QQGLASYDYV RRRLTAEDLF |
| | | | | | EARIISLETY NLLREGTRSL |
| | | | | | REALEAESAW CYLYGTGSVA |
| | | | | | GVYLPGSRQT LSIYQALKKG |
| | | | | | LLSAEVARLL LEAQAATGEL |
| | | | | | LDPVKGERLT VDEAVRKGLV |
| | | | | | GPELHDRLLS AERAVTGYRD |
| | | | | | PYTEQTISLF QAMKKELIPT |
| | | | | | EEALRLLDAQ LATGGIVDPR |
| | | | | | LGFHLPLEVA YQRGYLNKDT |
| | | | | | HDQLSEPSEV RSYVDPSTDE |
| | | | | | RLSYTQLLRR CRRDDGTGQL |
| | | | | | LLPLSDARKL TFRGLRKQIT |
| | | | | | MEELVRSQVM DEATALQLRE |
| | | | | | GLTSIEEVTK NLQKFLEGTS |
| | | | | | CIAGVFVDAT KERLSVYQAM |
| | | | | | KKGIIRPGTA FELLEAQAAT |
| | | | | | GYVIDPIKGL KLTVEEAVRM |
| | | | | | GIVGPEFKDK LLSAERAVTG |
| | | | | | YKDPYSGKLI SLFQAMKKGL |
| | | | | | ILKDHGIRLL EAQIATGGII |
| | | | | | DPEESHRLPV EVAYKRGLFD |
| | | | | | EEMNEILTDP SDDTKGFFDP |
| | | | | | NTEENLTYLQ LMERCITDPQ |
| | | | | | TGLCLLPLKE KKRERKTSSK |
| | | | | | SSVRKRRVVI VDPETGKEMS |
| | | | | | VYEAYRKGLI DHQTYLELSE |
| | | | | | QECEWEEITI SSSDGVVKSM |
| | | | | | IIDRRSGRQY DIDDAIAKNL |
| | | | | | IDRSALDQYR AGTLSITEFA |
| | | | | | DMLSGNAGGF RSRSSSVGSS |
| | | | | | SSYPISPAVS RTQLASWSDP |
| | | | | | TEETGPVAGI LDTETLEKVS |
| | | | | | ITEAMHRNLV DNITGQRLLE |
| | | | | | AQACTGGIID PSTGERFPVT |
| | | | | | DAVNKGLVDK IMVDRINLAQ |
| | | | | | KAFCGFEDPR TKTKMSAAQA |
| | | | | | LKKGWLYYEA GQRFLEVQYL |
| | | | | | TGGLIEPDTP GRVPLDEALQ |
| | | | | | RGTVDARTAQ KLRDVGAYSK |
| | | | | | YLTCPKTKLK ISYKDALDRS |
| | | | | | MVEEGTGLRL LEAAAQSTKG |
| | | | | | YYSPYSVSGS GSTAGSRTGS |
| | | | | | RTGSRAGSRR GSFDATGSGF |
| | | | | | SMTFSSSSYS SSGYGRRYAS |
| | | | | | GSSASLGGPE SAVA |
| 69 | PLEC | 3 | Q15149- | Entry version | MSGEDAEVRA VSEDVSNGSS |
| | | | 3 | 224 (18 Sep. | GSPSPGDTLP WNLGKTQRSR |
| | | | | 2019) | RSGGGAGSNG SVLDPAERAV |
| | | | | Sequence | IRIADERDRV QKKTFTKWVN |
| | | | | version 3 (14 | KHLIKAQRHI SDLYEDLRDG |
| | | | | Oct. 2008) | HNLISLLEVL SGDSLPREKG |
| | | | | | RMRFHKLQNV QIALDYLRHR |
| | | | | | QVKLVNIRND DIADGNPKLT |
| | | | | | LGLIWTIILH FQISDIQVSG |
| | | | | | QSEDMTAKEK LLLWSQRMVE |
| | | | | | GYQGLRCDNF TSSWRDGRLF |
| | | | | | NAIIHRHKPL LIDMNKVYRQ |
| | | | | | TNLENLDQAF SVAERDLGVT |
| | | | | | RLLDPEDVDV PQPDEKSIIT |
| | | | | | YVSSLYDAMP RVPDVQDGEL |
| | | | | | QLRWQEYREL VLLLLQWMRH |
| | | | | | HTAAFEERRF PSSFEEIEIL |
| | | | | | WSQFLKFKEM ELPAKEADKN |
| | | | | | RSKGIYQSLE GAVQAGQLKV |
| | | | | | PPGYHPLDVE KEWGKLHVAI |
| | | | | | LEREKQLRSE FERLECLQRI |
| | | | | | VTKLQMEAGL CEEQLNQADA |
| | | | | | LLQSDVRLLA AGKVPQRAGE |
| | | | | | VERDLDKADS MIRLLFNDVQ |
| | | | | | TLKDGRHPQG EQMYRRVYRL |
| | | | | | HERLVAIRTE YNLRLKAGVA |
| | | | | | APATQVAQVT LQSVQRRPEL |
| | | | | | EDSTLRYLQD LLAWVEENQH |
| | | | | | RVDGAEWGVD LPSVEAQLGS |
| | | | | | HRGLHQSIEE FRAKIERARS |
| | | | | | DEGQLSPATR GAYRDCLGRL |
| | | | | | DLQYAKLLNS SKARLRSLES |
| | | | | | LHSFVAAATK ELMWLNEKEE |
| | | | | | EEVGFDWSDR NTNMTAKKES |
| | | | | | YSALMRELEL KEKKIKELQN |
| | | | | | AGDRLLREDH PARPTVESFQ |
| | | | | | AALQTQWSWM LQLCCCIEAH |
| | | | | | LKENAAYFQF FSDVREAEGQ |
| | | | | | LQKLQEALRR KYSCDRSATV |
| | | | | | TRLEDLLQDA QDEKEQLNEY |
| | | | | | KGHLSGLAKR AKAVVQLKPR |
| | | | | | HPAHPMRGRL PLLAVCDYKQ |
| | | | | | VEVTVHKGDE CQLVGPAQPS |
| | | | | | HWKVLSSSGS EAAVPSVCFL |
| | | | | | VPPPNQEAQE AVTRLEAQHQ |
| | | | | | ALVTLWHQLH VDMKSLLAWQ |
| | | | | | SLRRDVQLIR SWSLATFRTL |
| | | | | | KPEEQRQALH SLELHYQAFL |
| | | | | | RDSQDAGGFG PEDRLMAERE |
| | | | | | YGSCSHHYQQ LLQSLEQGAQ |
| | | | | | EESRCQRCIS ELKDIRLQLE |
| | | | | | ACETRTVHRL RLPLDKEPAR |
| | | | | | ECAQRIAEQQ KAQAEVEGLG |
| | | | | | KGVARLSAEA EKVLALPEPS |
| | | | | | PAAPTLRSEL ELTLGKLEQV |
| | | | | | RSLSAIYLEK LKTISLVIRG |
| | | | | | TQGAEEVLRA HEEQLKEAQA |
| | | | | | VPATLPELEA TKASLKKLRA |
| | | | | | QAEAQQPTED ALRDELRGAQ |
| | | | | | EVGERLQQRH GERDVEVERW |
| | | | | | RERVAQLLER WQAVLAQTDV |
| | | | | | RQRELEQLGR QLRYYRESAD |
| | | | | | PLGAWLQDAR RRQEQIQAMP |
| | | | | | LADSQAVREQ LRQEQALLEE |
| | | | | | IERHGEKVEE CQRFAKQYIN |
| | | | | | AIKDYELQLV TYKAQLEPVA |
| | | | | | SPAKKPKVQS GSESVIQEYV |
| | | | | | DLRTHYSELT TLTSQYIKFI |
| | | | | | SETLRRMEEE ERLAEQQRAE |
| | | | | | ERERLAEVEA ALEKQRQLAE |
| | | | | | AHAQAKAQAE REAKELQQRM |
| | | | | | QEEVVRREEA AVDAQQQKRS |
| | | | | | IQEELQQLRQ SSEAEIQAKA |
| | | | | | RQAEAAERSR LRIEEEIRVV |
| | | | | | RLQLEATERQ RGGAEGELQA |
| | | | | | LRARAEEAEA QKRQAQEEAE |
| | | | | | RLRRQVQDES QRKRQAEVEL |
| | | | | | ASRVKAEAEA AREKQRALQA |
| | | | | | LEELRLQAEE AERRLRQAEV |
| | | | | | ERARQVQVAL ETAQRSAEAE |
| | | | | | LQSKRASFAE KTAQLERSLQ |
| | | | | | EEHVAVAQLR EEAERRAQQQ |
| | | | | | AEAERAREEA ERELERWQLK |
| | | | | | ANEALRLRLQ AEEVAQQKSL |
| | | | | | AQAEAEKQKE EAEREARRRG |
| | | | | | KAEEQAVRQR ELAEQELEKQ |
| | | | | | RQLAEGTAQQ RLAAEQELIR |
| | | | | | LRAETEQGEQ QRQLLEEELA |
| | | | | | RLQREAAAAT QKRQELEAEL |
| | | | | | AKVRAEMEVL LASKARAEEE |
| | | | | | SRSTSEKSKQ RLEAEAGRER |
| | | | | | ELAEEAARLR ALAEEAKRQR |
| | | | | | QLAEEDAARQ RAEAERVLAE |
| | | | | | KLAAIGEATR LKTEAEIALK |
| | | | | | EKEAENERLR RLAEDEAFQR |
| | | | | | RRLEEQAAQH KADIEERLAQ |
| | | | | | LRKASDSELE RQKGLVEDTL |
| | | | | | RQRRQVEEEI LALKASFEKA |
| | | | | | AAGKAELELE LGRIRSNAED |
| | | | | | TLRSKEQAEL EAARQRQLAA |
| | | | | | EEERRRREAE ERVQKSLAAE |
| | | | | | EEAARQRKAA LEEVERLKAK |
| | | | | | VEEARRLRER AEQESARQLQ |
| | | | | | LAQEAAQKRL QAEEKAHAFA |
| | | | | | VQQKEQELQQ TLQQEQSVLD |
| | | | | | QLRGEAEAAR RAAEEAEEAR |
| | | | | | VQAEREAAQS RRQVEEAERL |
| | | | | | KQSAEEQAQA RAQAQAAAEK |
| | | | | | LRKEAEQEAA RRAQAEQAAL |
| | | | | | RQKQAADAEM EKHKKFAEQT |
| | | | | | LRQKAQVEQE LTTLRLQLEE |
| | | | | | TDHQKNLLDE ELQRLKAEAT |
| | | | | | EAARQRSQVE EELFSVRVQM |
| | | | | | EELSKLKARI EAENRALILR |
| | | | | | DKDNTQRFLQ EEAEKMKQVA |
| | | | | | EEAARLSVAA QEAARLRQLA |
| | | | | | EEDLAQQRAL AEKMLKEKMQ |
| | | | | | AVQEATRLKA EAELLQQQKE |
| | | | | | LAQEQARRLQ EDKEQMAQQL |
| | | | | | AEETQGFQRT LEAERQRQLE |
| | | | | | MSAEAERLKL RVAEMSRAQA |
| | | | | | RAEEDAQRFR KQAEEIGEKL |
| | | | | | HRTELATQEK VTLVQTLEIQ |
| | | | | | RQQSDHDAER LREAIAELER |
| | | | | | EKEKLQQEAK LLQLKSEEMQ |
| | | | | | TVQQEQLLQE TQALQQSFLS |
| | | | | | EKDSLLQRER FIEQEKAKLE |
| | | | | | QLFQDEVAKA QQLREEQQRQ |
| | | | | | QQQMEQERQR LVASMEEARR |
| | | | | | RQHEAEEGVR RKQEELQQLE |
| | | | | | QQRRQQEELL AEENQRLREQ |
| | | | | | LQLLEEQHRA ALAHSEEVTA |
| | | | | | SQVAATKTLP NGRDALDGPA |
| | | | | | AEAEPEHSFD GLRRKVSAQR |
| | | | | | LQEAGILSAE ELQRLAQGHT |
| | | | | | TVDELARRED VRHYLQGRSS |
| | | | | | IAGLLLKATN EKLSVYAALQ |
| | | | | | RQLLSPGTAL ILLEAQAASG |
| | | | | | FLLDPVRNRR LTVNEAVKEG |
| | | | | | VVGPELHHKL LSAERAVTGY |
| | | | | | KDPYTGQQIS LFQAMQKGLI |
| | | | | | VREHGIRLLE AQIATGGVID |
| | | | | | PVHSHRVPVD VAYRRGYFDE |
| | | | | | EMNRVLADPS DDTKGFFDPN |
| | | | | | THENLTYLQL LERCVEDPET |
| | | | | | GLCLLPLTDK AAKGGELVYT |
| | | | | | DSEARDVFEK ATVSAPFGKF |
| | | | | | QGKTVTIWEI INSEYFTAEQ |
| | | | | | RRDLLRQFRT GRITVEKIIK |
| | | | | | IIITVVEEQE QKGRLCFEGL |
| | | | | | RSLVPAAELL ESRVIDRELY |
| | | | | | QQLQRGERSV RDVAEVDTVR |
| | | | | | RALRGANVIA GVWLEEAGQK |
| | | | | | LSIYNALKKD LLPSDMAVAL |
| | | | | | LEAQAGTGHI IDPATSARLT |
| | | | | | VDEAVRAGLV GPEFHEKLLS |
| | | | | | AEKAVTGYRD PYTGQSVSLF |
| | | | | | QALKKGLIPR EQGLRLLDAQ |
| | | | | | LSTGGIVDPS KSHRVPLDVA |
| | | | | | CARGCLDEET SRALSAPRAD |
| | | | | | AKAYSDPSTG EPATYGELQQ |
| | | | | | RCRPDQLTGL SLLPLSEKAA |
| | | | | | RARQEELYSE LQARETFEKT |
| | | | | | PVEVPVGGFK GRTVTVWELI |
| | | | | | SSEYFTAEQR QELLRQFRTG |
| | | | | | KVTVEKVIKI LITIVEEVET |
| | | | | | LRQERLSFSG LRAPVPASEL |
| | | | | | LASGVLSRAQ FEQLKDGKTT |
| | | | | | VKDLSELGSV RTLLQGSGCL |
| | | | | | AGIYLEDTKE KVSIYEAMRR |
| | | | | | GLLRATTAAL LLEAQAATGF |
| | | | | | LVDPVRNQRL YVHEAVKAGV |
| | | | | | VGPELHEQLL SAEKAVTGYR |
| | | | | | DPYSGSTISL FQAMQKGLVL |
| | | | | | RQHGIRLLEA QIATGGIIDP |
| | | | | | VHSHRVPVDV AYQRGYFSEE |
| | | | | | MNRVLADPSD DTKGFFDPNT |
| | | | | | HENLTYRQLL ERCVEDPETG |
| | | | | | LRLLPLKGAE KAEVVETTQV |
| | | | | | YTEEETRRAF EETQIDIPGG |
| | | | | | GSHGGSTMSL WEVMQSDLIP |
| | | | | | EEQRAQLMAD FQAGRVTKER |
| | | | | | MIIIIIEIIE KTEIIRQQGL |
| | | | | | ASYDYVRRRL TAEDLFEARI |
| | | | | | ISLETYNLLR EGTRSLREAL |
| | | | | | EAESAWCYLY GTGSVAGVYL |
| | | | | | PGSRQTLSIY QALKKGLLSA |
| | | | | | EVARLLLEAQ AATGFLLDPV |
| | | | | | KGERLTVDEA VRKGLVGPEL |
| | | | | | HDRLLSAERA VTGYRDPYTE |
| | | | | | QTISLFQAMK KELIPTEEAL |
| | | | | | RLLDAQLATG GIVDPRLGFH |
| | | | | | LPLEVAYQRG YLNKDTHDQL |
| | | | | | SEPSEVRSYV DPSTDERLSY |
| | | | | | TQLLRRCRRD DGTGQLLLPL |
| | | | | | SDARKLTFRG LRKQITMEEL |
| | | | | | VRSQVMDEAT ALQLREGLTS |
| | | | | | IEEVTKNLQK FLEGTSCIAG |
| | | | | | VFVDATKERL SVYQAMKKGI |
| | | | | | IRPGTAFELL EAQAATGYVI |
| | | | | | DPIKGLKLTV EEAVRMGIVG |
| | | | | | PEFKDKLLSA ERAVTGYKDP |
| | | | | | YSGKLISLFQ AMKKGLILKD |
| | | | | | HGIRLLEAQI ATGGIIDPEE |
| | | | | | SHRLPVEVAY KRGLFDEEMN |
| | | | | | EILTDPSDDT KGFFDPNTEE |
| | | | | | NLTYLQLMER CITDPQTGLC |
| | | | | | LLPLKEKKRE RKTSSKSSVR |
| | | | | | KRRVVIVDPE TGKEMSVYEA |
| | | | | | YRKGLIDHQT YLELSEQECE |
| | | | | | WEEITISSSD GVVKSMIIDR |
| | | | | | RSGRQYDIDD AIAKNLIDRS |
| | | | | | ALDQYRAGTL SITEFADMLS |
| | | | | | GNAGGFRSRS SSVGSSSSYP |
| | | | | | ISPAVSRTQL ASWSDPTEET |
| | | | | | GPVAGILDTE TLEKVSITEA |
| | | | | | MHRNLVDNIT GQRLLEAQAC |
| | | | | | TGGIIDPSTG ERFPVTDAVN |
| | | | | | KGLVDKIMVD RINLAQKAFC |
| | | | | | GFEDPRTKTK MSAAQALKKG |
| | | | | | WLYYEAGQRF LEVQYLTGGL |
| | | | | | IEPDTPGRVP LDEALQRGTV |
| | | | | | DARTAQKLRD VGAYSKYLTC |
| | | | | | PKTKLKISYK DALDRSMVEE |
| | | | | | GTGLRLLEAA AQSTKGYYSP |
| | | | | | YSVSGSGSTA GSRTGSRTGS |
| | | | | | RAGSRRGSED ATGSGFSMTF |
| | | | | | SSSSYSSSGY GRRYASGSSA |
| | | | | | SLGGPESAVA |
| 70 | PLEC | 4 | Q15149- | Entry version | MSQHQLRVPQ PEGLGRKRTS |
| | | | 4 | 224 (18 Sep. | SEDNLYLAVL RASEGKKDER |
| | | | | 2019) | DRVQKKTFTK WVNKHLIKAQ |
| | | | | Sequence | RHISDLYEDL RDGHNLISLL |
| | | | | version 3 (14 | EVLSGDSLPR EKGRMRFHKL |
| | | | | Oct. 2008) | QNVQIALDYL RHRQVKLVNI |
| | | | | | RNDDIADGNP KLTLGLIWTI |
| | | | | | ILHFQISDIQ VSGQSEDMTA |
| | | | | | KEKLLLWSQR MVEGYQGLRC |
| | | | | | DNFTSSWRDG RLFNAIIHRH |
| | | | | | KPLLIDMNKV YRQTNLENLD |
| | | | | | QAFSVAERDL GVTRLLDPED |
| | | | | | VDVPQPDEKS IITYVSSLYD |
| | | | | | AMPRVPDVQD GVRANELQLR |
| | | | | | WQEYRELVLL LLQWMRHHTA |
| | | | | | AFEERRFPSS FEEIEILWSQ |
| | | | | | FLKFKEMELP AKEADKNRSK |
| | | | | | GIYQSLEGAV QAGQLKVPPG |
| | | | | | YHPLDVEKEW GKLHVAILER |
| | | | | | EKQLRSEFER LECLQRIVTK |
| | | | | | LQMEAGLCEE QLNQADALLQ |
| | | | | | SDVRLLAAGK VPQRAGEVER |
| | | | | | DLDKADSMIR LLFNDVQTLK |
| | | | | | DGRHPQGEQM YRRVYRLHER |
| | | | | | LVAIRTEYNL RLKAGVAAPA |
| | | | | | TQVAQVTLQS VQRRPELEDS |
| | | | | | TLRYLQDLLA WVEENQHRVD |
| | | | | | GAEWGVDLPS VEAQLGSHRG |
| | | | | | LHQSIEEFRA KIERARSDEG |
| | | | | | QLSPATRGAY RDCLGRLDLQ |
| | | | | | YAKLLNSSKA RLRSLESLHS |
| | | | | | FVAAATKELM WLNEKEEEEV |
| | | | | | GFDWSDRNTN MTAKKESYSA |
| | | | | | LMRELELKEK KIKELQNAGD |
| | | | | | RLLREDHPAR PTVESFQAAL |
| | | | | | QTQWSWMLQL CCCIEAHLKE |
| | | | | | NAAYFQFFSD VREAEGQLQK |
| | | | | | LQEALRRKYS CDRSATVTRL |
| | | | | | EDLLQDAQDE KEQLNEYKGH |
| | | | | | LSGLAKRAKA VVQLKPRHPA |
| | | | | | HPMRGRLPLL AVCDYKQVEV |
| | | | | | TVHKGDECQL VGPAQPSHWK |
| | | | | | VLSSSGSEAA VPSVCFLVPP |
| | | | | | PNQEAQEAVT RLEAQHQALV |
| | | | | | TLWHQLHVDM KSLLAWQSLR |
| | | | | | RDVQLIRSWS LATFRTLKPE |
| | | | | | EQRQALHSLE LHYQAFLRDS |
| | | | | | QDAGGFGPED RLMAEREYGS |
| | | | | | CSHHYQQLLQ SLEQGAQEES |
| | | | | | RCQRCISELK DIRLQLEACE |
| | | | | | TRTVHRLRLP LDKEPARECA |
| | | | | | QRIAEQQKAQ AEVEGLGKGV |
| | | | | | ARLSAEAEKV LALPEPSPAA |
| | | | | | PTLRSELELT LGKLEQVRSL |
| | | | | | SAIYLEKLKT ISLVIRGTQG |
| | | | | | AEEVLRAHEE QLKEAQAVPA |
| | | | | | TLPELEATKA SLKKLRAQAE |
| | | | | | AQQPTFDALR DELRGAQEVG |
| | | | | | ERLQQRHGER DVEVERWRER |
| | | | | | VAQLLERWQA VLAQTDVRQR |
| | | | | | ELEQLGRQLR YYRESADPLG |
| | | | | | AWLQDARRRQ EQIQAMPLAD |
| | | | | | SQAVREQLRQ EQALLEEIER |
| | | | | | HGEKVEECQR FAKQYINAIK |
| | | | | | DYELQLVTYK AQLEPVASPA |
| | | | | | KKPKVQSGSE SVIQEYVDLR |
| | | | | | THYSELTTLT SQYIKFISET |
| | | | | | LRRMEEEERL AEQQRAEERE |
| | | | | | RLAEVEAALE KQRQLAEAHA |
| | | | | | QAKAQAEREA KELQQRMQEE |
| | | | | | VVRREEAAVD AQQQKRSIQE |
| | | | | | ELQQLRQSSE AEIQAKARQA |
| | | | | | EAAERSRLRI EEEIRVVRLQ |
| | | | | | LEATERQRGG AEGELQALRA |
| | | | | | RAEEAEAQKR QAQEEAERLR |
| | | | | | RQVQDESQRK RQAEVELASR |
| | | | | | VKAEAEAARE KQRALQALEE |
| | | | | | LRLQAEEAER RLRQAEVERA |
| | | | | | RQVQVALETA QRSAEAELQS |
| | | | | | KRASFAEKTA QLERSLQEEH |
| | | | | | VAVAQLREEA ERRAQQQAEA |
| | | | | | ERAREEAERE LERWQLKANE |
| | | | | | ALRLRLQAEE VAQQKSLAQA |
| | | | | | EAEKQKEEAE REARRRGKAE |
| | | | | | EQAVRQRELA EQELEKQRQL |
| | | | | | AEGTAQQRLA AEQELIRLRA |
| | | | | | ETEQGEQQRQ LLEEELARLQ |
| | | | | | REAAAATQKR QELEAELAKV |
| | | | | | RAEMEVLLAS KARAEEESRS |
| | | | | | TSEKSKQRLE AEAGRFRELA |
| | | | | | EEAARLRALA EEAKRQRQLA |
| | | | | | EEDAARQRAE AERVLAEKLA |
| | | | | | AIGEATRLKT EAEIALKEKE |
| | | | | | AENERLRRLA EDEAFQRRRL |
| | | | | | EEQAAQHKAD IEERLAQLRK |
| | | | | | ASDSELERQK GLVEDTLRQR |
| | | | | | RQVEEEILAL KASFEKAAAG |
| | | | | | KAELELELGR IRSNAEDTLR |
| | | | | | SKEQAELEAA RQRQLAAEEE |
| | | | | | RRRREAEERV QKSLAAEEEA |
| | | | | | ARQRKAALEE VERLKAKVEE |
| | | | | | ARRLRERAEQ ESARQLQLAQ |
| | | | | | EAAQKRLQAE EKAHAFAVQQ |
| | | | | | KEQELQQTLQ QEQSVLDQLR |
| | | | | | GEAEAARRAA EEAEEARVQA |
| | | | | | EREAAQSRRQ VEEAERLKQS |
| | | | | | AEEQAQARAQ AQAAAEKLRK |
| | | | | | EAEQEAARRA QAEQAALRQK |
| | | | | | QAADAEMEKH KKFAEQTLRQ |
| | | | | | KAQVEQELTT LRLQLEETDH |
| | | | | | QKNLLDEELQ RLKAEATEAA |
| | | | | | RQRSQVEEEL FSVRVQMEEL |
| | | | | | SKLKARIEAE NRALILRDKD |
| | | | | | NTQRFLQEEA EKMKQVAEEA |
| | | | | | ARLSVAAQEA ARLRQLAEED |
| | | | | | LAQQRALAEK MLKEKMQAVQ |
| | | | | | EATRLKAEAE LLQQQKELAQ |
| | | | | | EQARRLQEDK EQMAQQLAEE |
| | | | | | TQGFQRTLEA ERQRQLEMSA |
| | | | | | EAERLKLRVA EMSRAQARAE |
| | | | | | EDAQRFRKQA EEIGEKLHRT |
| | | | | | ELATQEKVTL VQTLEIQRQQ |
| | | | | | SDHDAERLRE AIAELEREKE |
| | | | | | KLQQEAKLLQ LKSEEMQTVQ |
| | | | | | QEQLLQETQA LQQSFLSEKD |
| | | | | | SLLQRERFIE QEKAKLEQLF |
| | | | | | QDEVAKAQQL REEQQRQQQQ |
| | | | | | MEQERQRLVA SMEEARRRQH |
| | | | | | EAEEGVRRKQ EELQQLEQQR |
| | | | | | RQQEELLAEE NQRLREQLQL |
| | | | | | LEEQHRAALA HSEEVTASQV |
| | | | | | AATKTLPNGR DALDGPAAEA |
| | | | | | EPEHSFDGLR RKVSAQRLQE |
| | | | | | AGILSAEELQ RLAQGHTTVD |
| | | | | | ELARREDVRH YLQGRSSIAG |
| | | | | | LLLKATNEKL SVYAALQRQL |
| | | | | | LSPGTALILL EAQAASGELL |
| | | | | | DPVRNRRLTV NEAVKEGVVG |
| | | | | | PELHHKLLSA ERAVTGYKDP |
| | | | | | YTGQQISLFQ AMQKGLIVRE |
| | | | | | HGIRLLEAQI ATGGVIDPVH |
| | | | | | SHRVPVDVAY RRGYFDEEMN |
| | | | | | RVLADPSDDT KGFFDPNTHE |
| | | | | | NLTYLQLLER CVEDPETGLC |
| | | | | | LLPLTDKAAK GGELVYTDSE |
| | | | | | ARDVFEKATV SAPFGKFQGK |
| | | | | | TVTIWEIINS EYFTAEQRRD |
| | | | | | LLRQFRTGRI TVEKIIKIII |
| | | | | | TVVEEQEQKG RLCFEGLRSL |
| | | | | | VPAAELLESR VIDRELYQQL |
| | | | | | QRGERSVRDV AEVDTVRRAL |
| | | | | | RGANVIAGVW LEEAGQKLSI |
| | | | | | YNALKKDLLP SDMAVALLEA |
| | | | | | QAGTGHIIDP ATSARLTVDE |
| | | | | | AVRAGLVGPE FHEKLLSAEK |
| | | | | | AVTGYRDPYT GQSVSLFQAL |
| | | | | | KKGLIPREQG LRLLDAQLST |
| | | | | | GGIVDPSKSH RVPLDVACAR |
| | | | | | GCLDEETSRA LSAPRADAKA |
| | | | | | YSDPSTGEPA TYGELQQRCR |
| | | | | | PDQLTGLSLL PLSEKAARAR |
| | | | | | QEELYSELQA RETFEKTPVE |
| | | | | | VPVGGFKGRT VTVWELISSE |
| | | | | | YFTAEQRQEL LRQFRTGKVT |
| | | | | | VEKVIKILIT IVEEVETLRQ |
| | | | | | ERLSFSGLRA PVPASELLAS |
| | | | | | GVLSRAQFEQ LKDGKTTVKD |
| | | | | | LSELGSVRTL LQGSGCLAGI |
| | | | | | YLEDTKEKVS IYEAMRRGLL |
| | | | | | RATTAALLLE AQAATGFLVD |
| | | | | | PVRNQRLYVH EAVKAGVVGP |
| | | | | | ELHEQLLSAE KAVTGYRDPY |
| | | | | | SGSTISLFQA MQKGLVLRQH |
| | | | | | GIRLLEAQIA TGGIIDPVHS |
| | | | | | HRVPVDVAYQ RGYFSEEMNR |
| | | | | | VLADPSDDTK GFFDPNTHEN |
| | | | | | LTYRQLLERC VEDPETGLRL |
| | | | | | LPLKGAEKAE VVETTQVYTE |
| | | | | | EETRRAFEET QIDIPGGGSH |
| | | | | | GGSTMSLWEV MQSDLIPEEQ |
| | | | | | RAQLMADFQA GRVTKERMII |
| | | | | | IIIEIIEKTE IIRQQGLASY |
| | | | | | DYVRRRLTAE DLFEARIISL |
| | | | | | ETYNLLREGT RSLREALEAE |
| | | | | | SAWCYLYGTG SVAGVYLPGS |
| | | | | | RQTLSIYQAL KKGLLSAEVA |
| | | | | | RLLLEAQAAT GFLLDPVKGE |
| | | | | | RLTVDEAVRK GLVGPELHDR |
| | | | | | LLSAERAVTG YRDPYTEQTI |
| | | | | | SLFQAMKKEL IPTEEALRLL |
| | | | | | DAQLATGGIV DPRLGFHLPL |
| | | | | | EVAYQRGYLN KDTHDQLSEP |
| | | | | | SEVRSYVDPS TDERLSYTQL |
| | | | | | LRRCRRDDGT GQLLLPLSDA |
| | | | | | RKLTFRGLRK QITMEELVRS |
| | | | | | QVMDEATALQ LREGLTSIEE |
| | | | | | VTKNLQKFLE GTSCIAGVFV |
| | | | | | DATKERLSVY QAMKKGIIRP |
| | | | | | GTAFELLEAQ AATGYVIDPI |
| | | | | | KGLKLTVEEA VRMGIVGPEF |
| | | | | | KDKLLSAERA VTGYKDPYSG |
| | | | | | KLISLFQAMK KGLILKDHGI |
| | | | | | RLLEAQIATG GIIDPEESHR |
| | | | | | LPVEVAYKRG LFDEEMNEIL |
| | | | | | TDPSDDTKGF FDPNTEENLT |
| | | | | | YLQLMERCIT DPQTGLCLLP |
| | | | | | LKEKKRERKT SSKSSVRKRR |
| | | | | | VVIVDPETGK EMSVYEAYRK |
| | | | | | GLIDHQTYLE LSEQECEWEE |
| | | | | | ITISSSDGVV KSMIIDRRSG |
| | | | | | RQYDIDDAIA KNLIDRSALD |
| | | | | | QYRAGTLSIT EFADMLSGNA |
| | | | | | GGFRSRSSSV GSSSSYPISP |
| | | | | | AVSRTQLASW SDPTEETGPV |
| | | | | | AGILDTETLE KVSITEAMHR |
| | | | | | NLVDNITGQR LLEAQACTGG |
| | | | | | IIDPSTGERF PVTDAVNKGL |
| | | | | | VDKIMVDRIN LAQKAFCGFE |
| | | | | | DPRTKTKMSA AQALKKGWLY |
| | | | | | YEAGQRFLEV QYLTGGLIEP |
| | | | | | DTPGRVPLDE ALQRGTVDAR |
| | | | | | TAQKLRDVGA YSKYLTCPKT |
| | | | | | KLKISYKDAL DRSMVEEGTG |
| | | | | | LRLLEAAAQS TKGYYSPYSV |
| | | | | | SGSGSTAGSR TGSRTGSRAG |
| | | | | | SRRGSFDATG SGFSMTFSSS |
| | | | | | SYSSSGYGRR YASGSSASLG |
| | | | | | GPESAVA |
| 71 | PLEC | 5 | Q15149- | Entry version | MEPSGSLFPS LVVVGHVVTL |
| | | | 5 | 224 (18 Sep. | AAVWHWRRGR RWAQDEQDER |
| | | | | 2019) | DRVQKKTFTK WVNKHLIKAQ |
| | | | | Sequence | RHISDLYEDL RDGHNLISLL |
| | | | | version 3 (14 | EVLSGDSLPR EKGRMRFHKL |
| | | | | Oct. 2008) | QNVQIALDYL RHRQVKLVNI |
| | | | | | RNDDIADGNP KLTLGLIWTI |
| | | | | | ILHFQISDIQ VSGQSEDMTA |
| | | | | | KEKLLLWSQR MVEGYQGLRC |
| | | | | | DNFTSSWRDG RLFNAIIHRH |
| | | | | | KPLLIDMNKV YRQTNLENLD |
| | | | | | QAFSVAERDL GVTRLLDPED |
| | | | | | VDVPQPDEKS IITYVSSLYD |
| | | | | | AMPRVPDVQD GVRANELQLR |
| | | | | | WQEYRELVLL LLQWMRHHTA |
| | | | | | AFEERRFPSS FEEIEILWSQ |
| | | | | | FLKFKEMELP AKEADKNRSK |
| | | | | | GIYQSLEGAV QAGQLKVPPG |
| | | | | | YHPLDVEKEW GKLHVAILER |
| | | | | | EKQLRSEFER LECLQRIVTK |
| | | | | | LQMEAGLCEE QLNQADALLQ |
| | | | | | SDVRLLAAGK VPQRAGEVER |
| | | | | | DLDKADSMIR LLFNDVQTLK |
| | | | | | DGRHPQGEQM YRRVYRLHER |
| | | | | | LVAIRTEYNL RLKAGVAAPA |
| | | | | | TQVAQVTLQS VQRRPELEDS |
| | | | | | TLRYLQDLLA WVEENQHRVD |
| | | | | | GAEWGVDLPS VEAQLGSHRG |
| | | | | | LHQSIEEFRA KIERARSDEG |
| | | | | | QLSPATRGAY RDCLGRLDLQ |
| | | | | | YAKLLNSSKA RLRSLESLHS |
| | | | | | FVAAATKELM WLNEKEEEEV |
| | | | | | GFDWSDRNTN MTAKKESYSA |
| | | | | | LMRELELKEK KIKELQNAGD |
| | | | | | RLLREDHPAR PTVESFQAAL |
| | | | | | QTQWSWMLQL CCCIEAHLKE |
| | | | | | NAAYFQFFSD VREAEGQLQK |
| | | | | | LQEALRRKYS CDRSATVTRL |
| | | | | | EDLLQDAQDE KEQLNEYKGH |
| | | | | | LSGLAKRAKA VVQLKPRHPA |
| | | | | | HPMRGRLPLL AVCDYKQVEV |
| | | | | | TVHKGDECQL VGPAQPSHWK |
| | | | | | VLSSSGSEAA VPSVCFLVPP |
| | | | | | PNQEAQEAVT RLEAQHQALV |
| | | | | | TLWHQLHVDM KSLLAWQSLR |
| | | | | | RDVQLIRSWS LATERTLKPE |
| | | | | | EQRQALHSLE LHYQAFLRDS |
| | | | | | QDAGGFGPED RLMAEREYGS |
| | | | | | CSHHYQQLLQ SLEQGAQEES |
| | | | | | RCQRCISELK DIRLQLEACE |
| | | | | | TRTVHRLRLP LDKEPARECA |
| | | | | | QRIAEQQKAQ AEVEGLGKGV |
| | | | | | ARLSAEAEKV LALPEPSPAA |
| | | | | | PTLRSELELT LGKLEQVRSL |
| | | | | | SAIYLEKLKT ISLVIRGTQG |
| | | | | | AEEVLRAHEE QLKEAQAVPA |
| | | | | | TLPELEATKA SLKKLRAQAE |
| | | | | | AQQPTFDALR DELRGAQEVG |
| | | | | | ERLQQRHGER DVEVERWRER |
| | | | | | VAQLLERWQA VLAQTDVRQR |
| | | | | | ELEQLGRQLR YYRESADPLG |
| | | | | | AWLQDARRRQ EQIQAMPLAD |
| | | | | | SQAVREQLRQ EQALLEEIER |
| | | | | | HGEKVEECQR FAKQYINAIK |
| | | | | | DYELQLVTYK AQLEPVASPA |
| | | | | | KKPKVQSGSE SVIQEYVDLR |
| | | | | | THYSELTTLT SQYIKFISET |
| | | | | | LRRMEEEERL AEQQRAEERE |
| | | | | | RLAEVEAALE KQRQLAEAHA |
| | | | | | QAKAQAEREA KELQQRMQEE |
| | | | | | VVRREEAAVD AQQQKRSIQE |
| | | | | | ELQQLRQSSE AEIQAKARQA |
| | | | | | EAAERSRLRI EEEIRVVRLQ |
| | | | | | LEATERQRGG AEGELQALRA |
| | | | | | RAEEAEAQKR QAQEEAERLR |
| | | | | | RQVQDESQRK RQAEVELASR |
| | | | | | VKAEAEAARE KQRALQALEE |
| | | | | | LRLQAEEAER RLRQAEVERA |
| | | | | | RQVQVALETA QRSAEAELQS |
| | | | | | KRASFAEKTA QLERSLQEEH |
| | | | | | VAVAQLREEA ERRAQQQAEA |
| | | | | | ERAREEAERE LERWQLKANE |
| | | | | | ALRLRLQAEE VAQQKSLAQA |
| | | | | | EAEKQKEEAE REARRRGKAE |
| | | | | | EQAVRQRELA EQELEKQRQL |
| | | | | | AEGTAQQRLA AEQELIRLRA |
| | | | | | ETEQGEQQRQ LLEEELARLQ |
| | | | | | REAAAATQKR QELEAELAKV |
| | | | | | RAEMEVLLAS KARAEEESRS |
| | | | | | TSEKSKQRLE AEAGRFRELA |
| | | | | | EEAARLRALA EEAKRQRQLA |
| | | | | | EEDAARQRAE AERVLAEKLA |
| | | | | | AIGEATRLKT EAEIALKEKE |
| | | | | | AENERLRRLA EDEAFQRRRL |
| | | | | | EEQAAQHKAD IEERLAQLRK |
| | | | | | ASDSELERQK GLVEDTLRQR |
| | | | | | RQVEEEILAL KASFEKAAAG |
| | | | | | KAELELELGR IRSNAEDTLR |
| | | | | | SKEQAELEAA RQRQLAAEEE |
| | | | | | RRRREAEERV QKSLAAEEEA |
| | | | | | ARQRKAALEE VERLKAKVEE |
| | | | | | ARRLRERAEQ ESARQLQLAQ |
| | | | | | EAAQKRLQAE EKAHAFAVQQ |
| | | | | | KEQELQQTLQ QEQSVLDQLR |
| | | | | | GEAEAARRAA EEAEEARVQA |
| | | | | | EREAAQSRRQ VEEAERLKQS |
| | | | | | AEEQAQARAQ AQAAAEKLRK |
| | | | | | EAEQEAARRA QAEQAALRQK |
| | | | | | QAADAEMEKH KKFAEQTLRQ |
| | | | | | KAQVEQELTT LRLQLEETDH |
| | | | | | QKNLLDEELQ RLKAEATEAA |
| | | | | | RQRSQVEEEL FSVRVQMEEL |
| | | | | | SKLKARIEAE NRALILRDKD |
| | | | | | NTQRFLQEEA EKMKQVAEEA |
| | | | | | ARLSVAAQEA ARLRQLAEED |
| | | | | | LAQQRALAEK MLKEKMQAVQ |
| | | | | | EATRLKAEAE LLQQQKELAQ |
| | | | | | EQARRLQEDK EQMAQQLAEE |
| | | | | | TQGFQRTLEA ERQRQLEMSA |
| | | | | | EAERLKLRVA EMSRAQARAE |
| | | | | | EDAQRFRKQA EEIGEKLHRT |
| | | | | | ELATQEKVTL VQTLEIQRQQ |
| | | | | | SDHDAERLRE AIAELEREKE |
| | | | | | KLQQEAKLLQ LKSEEMQTVQ |
| | | | | | QEQLLQETQA LQQSFLSEKD |
| | | | | | SLLQRERFIE QEKAKLEQLF |
| | | | | | QDEVAKAQQL REEQQRQQQQ |
| | | | | | MEQERQRLVA SMEEARRRQH |
| | | | | | EAEEGVRRKQ EELQQLEQQR |
| | | | | | RQQEELLAEE NQRLREQLQL |
| | | | | | LEEQHRAALA HSEEVTASQV |
| | | | | | AATKTLPNGR DALDGPAAEA |
| | | | | | EPEHSFDGLR RKVSAQRLQE |
| | | | | | AGILSAEELQ RLAQGHTTVD |
| | | | | | ELARREDVRH YLQGRSSIAG |
| | | | | | LLLKATNEKL SVYAALQRQL |
| | | | | | LSPGTALILL EAQAASGELL |
| | | | | | DPVRNRRLTV NEAVKEGVVG |
| | | | | | PELHHKLLSA ERAVTGYKDP |
| | | | | | YTGQQISLFQ AMQKGLIVRE |
| | | | | | HGIRLLEAQI ATGGVIDPVH |
| | | | | | SHRVPVDVAY RRGYFDEEMN |
| | | | | | RVLADPSDDT KGFFDPNTHE |
| | | | | | NLTYLQLLER CVEDPETGLC |
| | | | | | LLPLTDKAAK GGELVYTDSE |
| | | | | | ARDVFEKATV SAPFGKFQGK |
| | | | | | TVTIWEIINS EYFTAEQRRD |
| | | | | | LLRQFRTGRI TVEKIIKIII |
| | | | | | TVVEEQEQKG RLCFEGLRSL |
| | | | | | VPAAELLESR VIDRELYQQL |
| | | | | | QRGERSVRDV AEVDTVRRAL |
| | | | | | RGANVIAGVW LEEAGQKLSI |
| | | | | | YNALKKDLLP SDMAVALLEA |
| | | | | | QAGTGHIIDP ATSARLTVDE |
| | | | | | AVRAGLVGPE FHEKLLSAEK |
| | | | | | AVTGYRDPYT GQSVSLFQAL |
| | | | | | KKGLIPREQG LRLLDAQLST |
| | | | | | GGIVDPSKSH RVPLDVACAR |
| | | | | | GCLDEETSRA LSAPRADAKA |
| | | | | | YSDPSTGEPA TYGELQQRCR |
| | | | | | PDQLTGLSLL PLSEKAARAR |
| | | | | | QEELYSELQA RETFEKTPVE |
| | | | | | VPVGGFKGRT VTVWELISSE |
| | | | | | YFTAEQRQEL LRQFRTGKVT |
| | | | | | VEKVIKILIT IVEEVETLRQ |
| | | | | | ERLSFSGLRA PVPASELLAS |
| | | | | | GVLSRAQFEQ LKDGKTTVKD |
| | | | | | LSELGSVRTL LQGSGCLAGI |
| | | | | | YLEDTKEKVS IYEAMRRGLL |
| | | | | | RATTAALLLE AQAATGFLVD |
| | | | | | PVRNQRLYVH EAVKAGVVGP |
| | | | | | ELHEQLLSAE KAVTGYRDPY |
| | | | | | SGSTISLFQA MQKGLVLRQH |
| | | | | | GIRLLEAQIA TGGIIDPVHS |
| | | | | | HRVPVDVAYQ RGYFSEEMNR |
| | | | | | VLADPSDDTK GFFDPNTHEN |
| | | | | | LTYRQLLERC VEDPETGLRL |
| | | | | | LPLKGAEKAE VVETTQVYTE |
| | | | | | EETRRAFEET QIDIPGGGSH |
| | | | | | GGSTMSLWEV MQSDLIPEEQ |
| | | | | | RAQLMADFQA GRVTKERMII |
| | | | | | IIIEIIEKTE IIRQQGLASY |
| | | | | | DYVRRRLTAE DLFEARIISL |
| | | | | | ETYNLLREGT RSLREALEAE |
| | | | | | SAWCYLYGTG SVAGVYLPGS |
| | | | | | RQTLSIYQAL KKGLLSAEVA |
| | | | | | RLLLEAQAAT GFLLDPVKGE |
| | | | | | RLTVDEAVRK GLVGPELHDR |
| | | | | | LLSAERAVTG YRDPYTEQTI |
| | | | | | SLFQAMKKEL IPTEEALRLL |
| | | | | | DAQLATGGIV DPRLGFHLPL |
| | | | | | EVAYQRGYLN KDTHDQLSEP |
| | | | | | SEVRSYVDPS TDERLSYTQL |
| | | | | | LRRCRRDDGT GQLLLPLSDA |
| | | | | | RKLTFRGLRK QITMEELVRS |
| | | | | | QVMDEATALQ LREGLTSIEE |
| | | | | | VTKNLQKFLE GTSCIAGVFV |
| | | | | | DATKERLSVY QAMKKGIIRP |
| | | | | | GTAFELLEAQ AATGYVIDPI |
| | | | | | KGLKLTVEEA VRMGIVGPEF |
| | | | | | KDKLLSAERA VTGYKDPYSG |
| | | | | | KLISLFQAMK KGLILKDHGI |
| | | | | | RLLEAQIATG GIIDPEESHR |
| | | | | | LPVEVAYKRG LFDEEMNEIL |
| | | | | | TDPSDDTKGF FDPNTEENLT |
| | | | | | YLQLMERCIT DPQTGLCLLP |
| | | | | | LKEKKRERKT SSKSSVRKRR |
| | | | | | VVIVDPETGK EMSVYEAYRK |
| | | | | | GLIDHQTYLE LSEQECEWEE |
| | | | | | ITISSSDGVV KSMIIDRRSG |
| | | | | | RQYDIDDAIA KNLIDRSALD |
| | | | | | QYRAGTLSIT EFADMLSGNA |
| | | | | | GGFRSRSSSV GSSSSYPISP |
| | | | | | AVSRTQLASW SDPTEETGPV |
| | | | | | AGILDTETLE KVSITEAMHR |
| | | | | | NLVDNITGQR LLEAQACTGG |
| | | | | | IIDPSTGERF PVTDAVNKGL |
| | | | | | VDKIMVDRIN LAQKAFCGFE |
| | | | | | DPRTKTKMSA AQALKKGWLY |
| | | | | | YEAGQRFLEV QYLTGGLIEP |
| | | | | | DTPGRVPLDE ALQRGTVDAR |
| | | | | | TAQKLRDVGA YSKYLTCPKT |
| | | | | | KLKISYKDAL DRSMVEEGTG |
| | | | | | LRLLEAAAQS TKGYYSPYSV |
| | | | | | SGSGSTAGSR TGSRTGSRAG |
| | | | | | SRRGSFDATG SGFSMTFSSS |
| | | | | | SYSSSGYGRR YASGSSASLG |
| | | | | | GPESAVA |
| 72 | PLEC | 6 | Q15149- | Entry version | MSGAGGAFAS PREVLLERPC |
| | | | 6 | 224 (18 Sep. | WLDGGCEPAR RGYLYQQLCC |
| | | | | 2019) | VDERDRVQKK TFTKWVNKHL |
| | | | | Sequence | IKAQRHISDL YEDLRDGHNL |
| | | | | version 3 (14 | ISLLEVLSGD SLPREKGRMR |
| | | | | Oct. 2008) | FHKLQNVQIA LDYLRHRQVK |
| | | | | | LVNIRNDDIA DGNPKLTLGL |
| | | | | | IWTIILHFQI SDIQVSGQSE |
| | | | | | DMTAKEKLLL WSQRMVEGYQ |
| | | | | | GLRCDNFTSS WRDGRLFNAI |
| | | | | | IHRHKPLLID MNKVYRQTNL |
| | | | | | ENLDQAFSVA ERDLGVTRLL |
| | | | | | DPEDVDVPQP DEKSIITYVS |
| | | | | | SLYDAMPRVP DVQDGVRANE |
| | | | | | LQLRWQEYRE LVLLLLQWMR |
| | | | | | HHTAAFEERR FPSSFEEIEI |
| | | | | | LWSQFLKFKE MELPAKEADK |
| | | | | | NRSKGIYQSL EGAVQAGQLK |
| | | | | | VPPGYHPLDV EKEWGKLHVA |
| | | | | | ILEREKQLRS EFERLECLQR |
| | | | | | IVTKLQMEAG LCEEQLNQAD |
| | | | | | ALLQSDVRLL AAGKVPQRAG |
| | | | | | EVERDLDKAD SMIRLLFNDV |
| | | | | | QTLKDGRHPQ GEQMYRRVYR |
| | | | | | LHERLVAIRT EYNLRLKAGV |
| | | | | | AAPATQVAQV TLQSVQRRPE |
| | | | | | LEDSTLRYLQ DLLAWVEENQ |
| | | | | | HRVDGAEWGV DLPSVEAQLG |
| | | | | | SHRGLHQSIE EFRAKIERAR |
| | | | | | SDEGQLSPAT RGAYRDCLGR |
| | | | | | LDLQYAKLLN SSKARLRSLE |
| | | | | | SLHSFVAAAT KELMWLNEKE |
| | | | | | EEEVGFDWSD RNTNMTAKKE |
| | | | | | SYSALMRELE LKEKKIKELQ |
| | | | | | NAGDRLLRED HPARPTVESF |
| | | | | | QAALQTQWSW MLQLCCCIEA |
| | | | | | HLKENAAYFQ FFSDVREAEG |
| | | | | | QLQKLQEALR RKYSCDRSAT |
| | | | | | VTRLEDLLQD AQDEKEQLNE |
| | | | | | YKGHLSGLAK RAKAVVQLKP |
| | | | | | RHPAHPMRGR LPLLAVCDYK |
| | | | | | QVEVTVHKGD ECQLVGPAQP |
| | | | | | SHWKVLSSSG SEAAVPSVCF |
| | | | | | LVPPPNQEAQ EAVTRLEAQH |
| | | | | | QALVTLWHQL HVDMKSLLAW |
| | | | | | QSLRRDVQLI RSWSLATFRT |
| | | | | | LKPEEQRQAL HSLELHYQAF |
| | | | | | LRDSQDAGGF GPEDRLMAER |
| | | | | | EYGSCSHHYQ QLLQSLEQGA |
| | | | | | QEESRCQRCI SELKDIRLQL |
| | | | | | EACETRTVHR LRLPLDKEPA |
| | | | | | RECAQRIAEQ QKAQAEVEGI |
| | | | | | GKGVARLSAE AEKVLALPEP |
| | | | | | SPAAPTLRSE LELTLGKLEQ |
| | | | | | VRSLSAIYLE KLKTISLVIR |
| | | | | | GTQGAEEVLR AHEEQLKEAQ |
| | | | | | AVPATLPELE ATKASLKKLR |
| | | | | | AQAEAQQPTF DALRDELRGA |
| | | | | | QEVGERLQQR HGERDVEVER |
| | | | | | WRERVAQLLE RWQAVLAQTD |
| | | | | | VRQRELEQLG RQLRYYRESA |
| | | | | | DPLGAWLQDA RRRQEQIQAM |
| | | | | | PLADSQAVRE QLRQEQALLE |
| | | | | | EIERHGEKVE ECQRFAKQYI |
| | | | | | NAIKDYELQL VTYKAQLEPV |
| | | | | | ASPAKKPKVQ SGSESVIQEY |
| | | | | | VDLRTHYSEL TTLTSQYIKF |
| | | | | | ISETLRRMEE EERLAEQQRA |
| | | | | | EERERLAEVE AALEKQRQLA |
| | | | | | EAHAQAKAQA EREAKELQQR |
| | | | | | MQEEVVRREE AAVDAQQQKR |
| | | | | | SIQEELQQLR QSSEAEIQAK |
| | | | | | ARQAEAAERS RLRIEEEIRV |
| | | | | | VRLQLEATER QRGGAEGELQ |
| | | | | | ALRARAEEAE AQKRQAQEEA |
| | | | | | ERLRRQVQDE SQRKRQAEVE |
| | | | | | LASRVKAEAE AAREKQRALQ |
| | | | | | ALEELRLQAE EAERRLRQAE |
| | | | | | VERARQVQVA LETAQRSAEA |
| | | | | | ELQSKRASFA EKTAQLERSL |
| | | | | | QEEHVAVAQL REEAERRAQQ |
| | | | | | QAEAERAREE AERELERWQL |
| | | | | | KANEALRLRL QAEEVAQQKS |
| | | | | | LAQAEAEKQK EEAEREARRR |
| | | | | | GKAEEQAVRQ RELAEQELEK |
| | | | | | QRQLAEGTAQ QRLAAEQELI |
| | | | | | RLRAETEQGE QQRQLLEEEL |
| | | | | | ARLQREAAAA TQKRQELEAE |
| | | | | | LAKVRAEMEV LLASKARAEE |
| | | | | | ESRSTSEKSK QRLEAEAGRF |
| | | | | | RELAEEAARL RALAEEAKRQ |
| | | | | | RQLAEEDAAR QRAEAERVLA |
| | | | | | EKLAAIGEAT RLKTEAEIAL |
| | | | | | KEKEAENERL RRLAEDEAFQ |
| | | | | | RRRLEEQAAQ HKADIEERLA |
| | | | | | QLRKASDSEL ERQKGLVEDT |
| | | | | | LRQRRQVEEE ILALKASFEK |
| | | | | | AAAGKAELEL ELGRIRSNAE |
| | | | | | DTLRSKEQAE LEAARQRQLA |
| | | | | | AEEERRRREA EERVQKSLAA |
| | | | | | EEEAARQRKA ALEEVERLKA |
| | | | | | KVEEARRLRE RAEQESARQL |
| | | | | | QLAQEAAQKR LQAEEKAHAF |
| | | | | | AVQQKEQELQ QTLQQEQSVL |
| | | | | | DQLRGEAEAA RRAAEEAEEA |
| | | | | | RVQAEREAAQ SRRQVEEAER |
| | | | | | LKQSAEEQAQ ARAQAQAAAE |
| | | | | | KLRKEAEQEA ARRAQAEQAA |
| | | | | | LRQKQAADAE MEKHKKFAEQ |
| | | | | | TLRQKAQVEQ ELTTLRLQLE |
| | | | | | ETDHQKNLLD EELQRLKAEA |
| | | | | | TEAARQRSQV EEELFSVRVQ |
| | | | | | MEELSKLKAR IEAENRALIL |
| | | | | | RDKDNTQRFL QEEAEKMKQV |
| | | | | | AEEAARLSVA AQEAARLRQL |
| | | | | | AEEDLAQQRA LAEKMLKEKM |
| | | | | | QAVQEATRLK AEAELLQQQK |
| | | | | | ELAQEQARRL QEDKEQMAQQ |
| | | | | | LAEETQGFQR TLEAERQRQL |
| | | | | | EMSAEAERLK LRVAEMSRAQ |
| | | | | | ARAEEDAQRF RKQAEEIGEK |
| | | | | | LHRTELATQE KVTLVQTLEI |
| | | | | | QRQQSDHDAE RLREAIAELE |
| | | | | | REKEKLQQEA KLLQLKSEEM |
| | | | | | QTVQQEQLLQ ETQALQQSFL |
| | | | | | SEKDSLLQRE RFIEQEKAKL |
| | | | | | EQLFQDEVAK AQQLREEQQR |
| | | | | | QQQQMEQERQ RLVASMEEAR |
| | | | | | RRQHEAEEGV RRKQEELQQL |
| | | | | | EQQRRQQEEL LAEENQRLRE |
| | | | | | QLQLLEEQHR AALAHSEEVT |
| | | | | | ASQVAATKTL PNGRDALDGP |
| | | | | | AAEAEPEHSF DGLRRKVSAQ |
| | | | | | RLQEAGILSA EELQRLAQGH |
| | | | | | TTVDELARRE DVRHYLQGRS |
| | | | | | SIAGLLLKAT NEKLSVYAAL |
| | | | | | QRQLLSPGTA LILLEAQAAS |
| | | | | | GFLLDPVRNR RLTVNEAVKE |
| | | | | | GVVGPELHHK LLSAERAVTG |
| | | | | | YKDPYTGQQI SLFQAMQKGL |
| | | | | | IVREHGIRLL EAQIATGGVI |
| | | | | | DPVHSHRVPV DVAYRRGYFD |
| | | | | | EEMNRVLADP SDDTKGFFDP |
| | | | | | NTHENLTYLQ LLERCVEDPE |
| | | | | | TGLCLLPLTD KAAKGGELVY |
| | | | | | TDSEARDVFE KATVSAPFGK |
| | | | | | FQGKTVTIWE IINSEYFTAE |
| | | | | | QRRDLLRQFR TGRITVEKII |
| | | | | | KIIITVVEEQ EQKGRLCFEG |
| | | | | | LRSLVPAAEL LESRVIDREL |
| | | | | | YQQLQRGERS VRDVAEVDTV |
| | | | | | RRALRGANVI AGVWLEEAGQ |
| | | | | | KLSIYNALKK DLLPSDMAVA |
| | | | | | LLEAQAGTGH IIDPATSARL |
| | | | | | TVDEAVRAGL VGPEFHEKLL |
| | | | | | SAEKAVTGYR DPYTGQSVSL |
| | | | | | FQALKKGLIP REQGLRLLDA |
| | | | | | QLSTGGIVDP SKSHRVPLDV |
| | | | | | ACARGCLDEE TSRALSAPRA |
| | | | | | DAKAYSDPST GEPATYGELQ |
| | | | | | QRCRPDQLTG LSLLPLSEKA |
| | | | | | ARARQEELYS ELQARETFEK |
| | | | | | TPVEVPVGGF KGRTVTVWEL |
| | | | | | ISSEYFTAEQ RQELLRQFRT |
| | | | | | GKVTVEKVIK ILITIVEEVE |
| | | | | | TLRQERLSFS GLRAPVPASE |
| | | | | | LLASGVLSRA QFEQLKDGKT |
| | | | | | TVKDLSELGS VRTLLQGSGC |
| | | | | | LAGIYLEDTK EKVSIYEAMR |
| | | | | | RGLLRATTAA LLLEAQAATG |
| | | | | | FLVDPVRNQR LYVHEAVKAG |
| | | | | | VVGPELHEQL LSAEKAVTGY |
| | | | | | RDPYSGSTIS LFQAMQKGLV |
| | | | | | LRQHGIRLLE AQIATGGIID |
| | | | | | PVHSHRVPVD VAYQRGYFSE |
| | | | | | EMNRVLADPS DDTKGFFDPN |
| | | | | | THENLTYRQL LERCVEDPET |
| | | | | | GLRLLPLKGA EKAEVVETTQ |
| | | | | | VYTEEETRRA FEETQIDIPG |
| | | | | | GGSHGGSTMS LWEVMQSDLI |
| | | | | | PEEQRAQLMA DFQAGRVTKE |
| | | | | | RMIIIIIEII EKTEIIRQQG |
| | | | | | LASYDYVRRR LTAEDLFEAR |
| | | | | | IISLETYNLL REGTRSLREA |
| | | | | | LEAESAWCYL YGTGSVAGVY |
| | | | | | LPGSRQTLSI YQALKKGLLS |
| | | | | | AEVARLLLEA QAATGFLLDP |
| | | | | | VKGERLTVDE AVRKGLVGPE |
| | | | | | LHDRLLSAER AVTGYRDPYT |
| | | | | | EQTISLFQAM KKELIPTEEA |
| | | | | | LRLLDAQLAT GGIVDPRLGF |
| | | | | | HLPLEVAYQR GYLNKDTHDQ |
| | | | | | LSEPSEVRSY VDPSTDERLS |
| | | | | | YTQLLRRCRR DDGTGQLLLP |
| | | | | | LSDARKLTFR GLRKQITMEE |
| | | | | | LVRSQVMDEA TALQLREGLT |
| | | | | | SIEEVTKNLQ KFLEGTSCIA |
| | | | | | GVFVDATKER LSVYQAMKKG |
| | | | | | IIRPGTAFEL LEAQAATGYV |
| | | | | | IDPIKGLKLT VEEAVRMGIV |
| | | | | | GPEFKDKLLS AERAVTGYKD |
| | | | | | PYSGKLISLF QAMKKGLILK |
| | | | | | DHGIRLLEAQ IATGGIIDPE |
| | | | | | ESHRLPVEVA YKRGLEDEEM |
| | | | | | NEILTDPSDD TKGFFDPNTE |
| | | | | | ENLTYLQLME RCITDPQTGL |
| | | | | | CLLPLKEKKR ERKTSSKSSV |
| | | | | | RKRRVVIVDP ETGKEMSVYE |
| | | | | | AYRKGLIDHQ TYLELSEQEC |
| | | | | | EWEEITISSS DGVVKSMIID |
| | | | | | RRSGRQYDID DAIAKNLIDR |
| | | | | | SALDQYRAGT LSITEFADML |
| | | | | | SGNAGGFRSR SSSVGSSSSY |
| | | | | | PISPAVSRTQ LASWSDPTEE |
| | | | | | TGPVAGILDT ETLEKVSITE |
| | | | | | AMHRNLVDNI TGQRLLEAQA |
| | | | | | CTGGIIDPST GERFPVTDAV |
| | | | | | NKGLVDKIMV DRINLAQKAF |
| | | | | | CGFEDPRTKT KMSAAQALKK |
| | | | | | GWLYYEAGQR FLEVQYLTGG |
| | | | | | LIEPDTPGRV PLDEALQRGT |
| | | | | | VDARTAQKLR DVGAYSKYLT |
| | | | | | CPKTKLKISY KDALDRSMVE |
| | | | | | EGTGLRLLEA AAQSTKGYYS |
| | | | | | PYSVSGSGST AGSRTGSRTG |
| | | | | | SRAGSRRGSF DATGSGFSMT |
| | | | | | FSSSSYSSSG YGRRYASGSS |
| | | | | | ASLGGPESAV A |
| 73 | PLEC | 7 | Q15149- | Entry version | MKIVPDERDR VQKKTFTKWV |
| | | | 7 | 224 (18 Sep. | NKHLIKAQRH ISDLYEDLRD |
| | | | | 2019) | GHNLISLLEV LSGDSLPREK |
| | | | | Sequence | GRMRFHKLQN VQIALDYLRH |
| | | | | version 3 (14 | RQVKLVNIRN DDIADGNPKL |
| | | | | Oct. 2008) | TLGLIWTIIL HFQISDIQVS |
| | | | | | GQSEDMTAKE KLLLWSQRMV |
| | | | | | EGYQGLRCDN FTSSWRDGRL |
| | | | | | FNAIIHRHKP LLIDMNKVYR |
| | | | | | QTNLENLDQA FSVAERDLGV |
| | | | | | TRLLDPEDVD VPQPDEKSII |
| | | | | | TYVSSLYDAM PRVPDVQDGV |
| | | | | | RANELQLRWQ EYRELVLLLL |
| | | | | | QWMRHHTAAF EERRFPSSFE |
| | | | | | EIEILWSQFL KFKEMELPAK |
| | | | | | EADKNRSKGI YQSLEGAVQA |
| | | | | | GQLKVPPGYH PLDVEKEWGK |
| | | | | | LHVAILEREK QLRSEFERLE |
| | | | | | CLQRIVTKLQ MEAGLCEEQL |
| | | | | | NQADALLQSD VRLLAAGKVP |
| | | | | | QRAGEVERDL DKADSMIRLL |
| | | | | | FNDVQTLKDG RHPQGEQMYR |
| | | | | | RVYRLHERLV AIRTEYNLRL |
| | | | | | KAGVAAPATQ VAQVTLQSVQ |
| | | | | | RRPELEDSTL RYLQDLLAWV |
| | | | | | EENQHRVDGA EWGVDLPSVE |
| | | | | | AQLGSHRGLH QSIEEFRAKI |
| | | | | | ERARSDEGQL SPATRGAYRD |
| | | | | | CLGRLDLQYA KLLNSSKARL |
| | | | | | RSLESLHSFV AAATKELMWL |
| | | | | | NEKEEEEVGF DWSDRNTNMT |
| | | | | | AKKESYSALM RELELKEKKI |
| | | | | | KELQNAGDRL LREDHPARPT |
| | | | | | VESFQAALQT QWSWMLQLCC |
| | | | | | CIEAHLKENA AYFQFFSDVR |
| | | | | | EAEGQLQKLQ EALRRKYSCD |
| | | | | | RSATVTRLED LLQDAQDEKE |
| | | | | | QLNEYKGHLS GLAKRAKAVV |
| | | | | | QLKPRHPAHP MRGRLPLLAV |
| | | | | | CDYKQVEVTV HKGDECQLVG |
| | | | | | PAQPSHWKVL SSSGSEAAVP |
| | | | | | SVCFLVPPPN QEAQEAVTRL |
| | | | | | EAQHQALVTL WHQLHVDMKS |
| | | | | | LLAWQSLRRD VQLIRSWSLA |
| | | | | | TFRTLKPEEQ RQALHSLELH |
| | | | | | YQAFLRDSQD AGGFGPEDRL |
| | | | | | MAEREYGSCS HHYQQLLQSL |
| | | | | | EQGAQEESRC QRCISELKDI |
| | | | | | RLQLEACETR TVHRLRLPLD |
| | | | | | KEPARECAQR IAEQQKAQAE |
| | | | | | VEGLGKGVAR LSAEAEKVLA |
| | | | | | LPEPSPAAPT LRSELELTLG |
| | | | | | KLEQVRSLSA IYLEKLKTIS |
| | | | | | LVIRGTQGAE EVLRAHEEQL |
| | | | | | KEAQAVPATL PELEATKASL |
| | | | | | KKLRAQAEAQ QPTFDALRDE |
| | | | | | LRGAQEVGER LQQRHGERDV |
| | | | | | EVERWRERVA QLLERWQAVL |
| | | | | | AQTDVRQREL EQLGRQLRYY |
| | | | | | RESADPLGAW LQDARRRQEQ |
| | | | | | IQAMPLADSQ AVREQLRQEQ |
| | | | | | ALLEEIERHG EKVEECQRFA |
| | | | | | KQYINAIKDY ELQLVTYKAQ |
| | | | | | LEPVASPAKK PKVQSGSESV |
| | | | | | IQEYVDLRTH YSELTTLTSQ |
| | | | | | YIKFISETLR RMEEEERLAE |
| | | | | | QQRAEERERL AEVEAALEKQ |
| | | | | | RQLAEAHAQA KAQAEREAKE |
| | | | | | LQQRMQEEVV RREEAAVDAQ |
| | | | | | QQKRSIQEEL QQLRQSSEAE |
| | | | | | IQAKARQAEA AERSRLRIEE |
| | | | | | EIRVVRLQLE ATERQRGGAE |
| | | | | | GELQALRARA EEAEAQKRQA |
| | | | | | QEEAERLRRQ VQDESQRKRQ |
| | | | | | AEVELASRVK AEAEAAREKQ |
| | | | | | RALQALEELR LQAEEAERRL |
| | | | | | RQAEVERARQ VQVALETAQR |
| | | | | | SAEAELQSKR ASFAEKTAQL |
| | | | | | ERSLQEEHVA VAQLREEAER |
| | | | | | RAQQQAEAER AREEAERELE |
| | | | | | RWQLKANEAL RLRLQAEEVA |
| | | | | | QQKSLAQAEA EKQKEEAERE |
| | | | | | ARRRGKAEEQ AVRQRELAEQ |
| | | | | | ELEKQRQLAE GTAQQRLAAE |
| | | | | | QELIRLRAET EQGEQQRQLL |
| | | | | | EEELARLQRE AAAATQKRQE |
| | | | | | LEAELAKVRA EMEVLLASKA |
| | | | | | RAEEESRSTS EKSKQRLEAE |
| | | | | | AGRFRELAEE AARLRALAEE |
| | | | | | AKRQRQLAEE DAARQRAEAE |
| | | | | | RVLAEKLAAI GEATRLKTEA |
| | | | | | EIALKEKEAE NERLRRLAED |
| | | | | | EAFQRRRLEE QAAQHKADIE |
| | | | | | ERLAQLRKAS DSELERQKGL |
| | | | | | VEDTLRQRRQ VEEEILALKA |
| | | | | | SFEKAAAGKA ELELELGRIR |
| | | | | | SNAEDTLRSK EQAELEAARQ |
| | | | | | RQLAAEEERR RREAEERVQK |
| | | | | | SLAAEEEAAR QRKAALEEVE |
| | | | | | RLKAKVEEAR RLRERAEQES |
| | | | | | ARQLQLAQEA AQKRLQAEEK |
| | | | | | AHAFAVQQKE QELQQTLQQE |
| | | | | | QSVLDQLRGE AEAARRAAEE |
| | | | | | AEEARVQAER EAAQSRRQVE |
| | | | | | EAERLKQSAE EQAQARAQAQ |
| | | | | | AAAEKLRKEA EQEAARRAQA |
| | | | | | EQAALRQKQA ADAEMEKHKK |
| | | | | | FAEQTLRQKA QVEQELTTLR |
| | | | | | LQLEETDHQK NLLDEELQRL |
| | | | | | KAEATEAARQ RSQVEEELFS |
| | | | | | VRVQMEELSK LKARIEAENR |
| | | | | | ALILRDKDNT QRFLQEEAEK |
| | | | | | MKQVAEEAAR LSVAAQEAAR |
| | | | | | LRQLAEEDLA QQRALAEKML |
| | | | | | KEKMQAVQEA TRLKAEAELL |
| | | | | | QQQKELAQEQ ARRLQEDKEQ |
| | | | | | MAQQLAEETQ GFQRTLEAER |
| | | | | | QRQLEMSAEA ERLKLRVAEM |
| | | | | | SRAQARAEED AQRFRKQAEE |
| | | | | | IGEKLHRTEL ATQEKVTLVQ |
| | | | | | TLEIQRQQSD HDAERLREAI |
| | | | | | AELEREKEKL QQEAKLLQLK |
| | | | | | SEEMQTVQQE QLLQETQALQ |
| | | | | | QSFLSEKDSL LQRERFIEQE |
| | | | | | KAKLEQLFQD EVAKAQQLRE |
| | | | | | EQQRQQQQME QERQRLVASM |
| | | | | | EEARRRQHEA EEGVRRKQEE |
| | | | | | LQQLEQQRRQ QEELLAEENQ |
| | | | | | RLREQLQLLE EQHRAALAHS |
| | | | | | EEVTASQVAA TKTLPNGRDA |
| | | | | | LDGPAAEAEP EHSFDGLRRK |
| | | | | | VSAQRLQEAG ILSAEELQRL |
| | | | | | AQGHTTVDEL ARREDVRHYL |
| | | | | | QGRSSIAGLL LKATNEKLSV |
| | | | | | YAALQRQLLS PGTALILLEA |
| | | | | | QAASGFLLDP VRNRRLTVNE |
| | | | | | AVKEGVVGPE LHHKLLSAER |
| | | | | | AVTGYKDPYT GQQISLFQAM |
| | | | | | QKGLIVREHG IRLLEAQIAT |
| | | | | | GGVIDPVHSH RVPVDVAYRR |
| | | | | | GYFDEEMNRV LADPSDDTKG |
| | | | | | FFDPNTHENL TYLQLLERCV |
| | | | | | EDPETGLCLL PLTDKAAKGG |
| | | | | | ELVYTDSEAR DVFEKATVSA |
| | | | | | PFGKFQGKTV TIWEIINSEY |
| | | | | | FTAEQRRDLL RQFRTGRITV |
| | | | | | EKIIKIIITV VEEQEQKGRL |
| | | | | | CFEGLRSLVP AAELLESRVI |
| | | | | | DRELYQQLQR GERSVRDVAE |
| | | | | | VDTVRRALRG ANVIAGVWLE |
| | | | | | EAGQKLSIYN ALKKDLLPSD |
| | | | | | MAVALLEAQA GTGHIIDPAT |
| | | | | | SARLTVDEAV RAGLVGPEFH |
| | | | | | EKLLSAEKAV TGYRDPYTGQ |
| | | | | | SVSLFQALKK GLIPREQGLR |
| | | | | | LLDAQLSTGG IVDPSKSHRV |
| | | | | | PLDVACARGC LDEETSRALS |
| | | | | | APRADAKAYS DPSTGEPATY |
| | | | | | GELQQRCRPD QLTGLSLLPL |
| | | | | | SEKAARARQE ELYSELQARE |
| | | | | | TFEKTPVEVP VGGFKGRTVT |
| | | | | | VWELISSEYF TAEQRQELLR |
| | | | | | QFRTGKVTVE KVIKILITIV |
| | | | | | EEVETLRQER LSFSGLRAPV |
| | | | | | PASELLASGV LSRAQFEQLK |
| | | | | | DGKTTVKDLS ELGSVRTLLQ |
| | | | | | GSGCLAGIYL EDTKEKVSIY |
| | | | | | EAMRRGLLRA TTAALLLEAQ |
| | | | | | AATGFLVDPV RNQRLYVHEA |
| | | | | | VKAGVVGPEL HEQLLSAEKA |
| | | | | | VTGYRDPYSG STISLFQAMQ |
| | | | | | KGLVLRQHGI RLLEAQIATG |
| | | | | | GIIDPVHSHR VPVDVAYQRG |
| | | | | | YFSEEMNRVL ADPSDDTKGF |
| | | | | | FDPNTHENLT YRQLLERCVE |
| | | | | | DPETGLRLLP LKGAEKAEVV |
| | | | | | ETTQVYTEEE TRRAFEETQI |
| | | | | | DIPGGGSHGG STMSLWEVMQ |
| | | | | | SDLIPEEQRA QLMADFQAGR |
| | | | | | VTKERMIIII IEIIEKTEII |
| | | | | | RQQGLASYDY VRRRLTAEDL |
| | | | | | FEARIISLET YNLLREGTRS |
| | | | | | LREALEAESA WCYLYGTGSV |
| | | | | | AGVYLPGSRQ TLSIYQALKK |
| | | | | | GLLSAEVARL LLEAQAATGF |
| | | | | | LLDPVKGERL TVDEAVRKGL |
| | | | | | VGPELHDRLL SAERAVTGYR |
| | | | | | DPYTEQTISL FQAMKKELIP |
| | | | | | TEEALRLLDA QLATGGIVDP |
| | | | | | RLGFHLPLEV AYQRGYLNKD |
| | | | | | THDQLSEPSE VRSYVDPSTD |
| | | | | | ERLSYTQLLR RCRRDDGTGQ |
| | | | | | LLLPLSDARK LTFRGLRKQI |
| | | | | | TMEELVRSQV MDEATALQLR |
| | | | | | EGLTSIEEVT KNLQKFLEGT |
| | | | | | SCIAGVFVDA TKERLSVYQA |
| | | | | | MKKGIIRPGT AFELLEAQAA |
| | | | | | TGYVIDPIKG LKLTVEEAVR |
| | | | | | MGIVGPEFKD KLLSAERAVT |
| | | | | | GYKDPYSGKL ISLFQAMKKG |
| | | | | | LILKDHGIRL LEAQIATGGI |
| | | | | | IDPEESHRLP VEVAYKRGLF |
| | | | | | DEEMNEILTD PSDDTKGFFD |
| | | | | | PNTEENLTYL QLMERCITDP |
| | | | | | QTGLCLLPLK EKKRERKTSS |
| | | | | | KSSVRKRRVV IVDPETGKEM |
| | | | | | SVYEAYRKGL IDHQTYLELS |
| | | | | | EQECEWEEIT ISSSDGVVKS |
| | | | | | MIIDRRSGRQ YDIDDAIAKN |
| | | | | | LIDRSALDQY RAGTLSITEF |
| | | | | | ADMLSGNAGG FRSRSSSVGS |
| | | | | | SSSYPISPAV SRTQLASWSD |
| | | | | | PTEETGPVAG ILDTETLEKV |
| | | | | | SITEAMHRNL VDNITGQRLL |
| | | | | | EAQACTGGII DPSTGERFPV |
| | | | | | TDAVNKGLVD KIMVDRINLA |
| | | | | | QKAFCGFEDP RTKTKMSAAQ |
| | | | | | ALKKGWLYYE AGQRFLEVQY |
| | | | | | LTGGLIEPDT PGRVPLDEAL |
| | | | | | QRGTVDARTA QKLRDVGAYS |
| | | | | | KYLTCPKTKL KISYKDALDR |
| | | | | | SMVEEGTGLR LLEAAAQSTK |
| | | | | | GYYSPYSVSG SGSTAGSRTG |
| | | | | | SRTGSRAGSR RGSFDATGSG |
| | | | | | FSMTFSSSSY SSSGYGRRYA |
| | | | | | SGSSASLGGP ESAVA |
| 74 | PLEC | 8 | Q15149- | Entry version | MDPSRAIQNE ISSLKDERDR |
| | | | 8 | 224 (18 Sep. | VQKKTFTKWV NKHLIKAQRH |
| | | | | 2019) | ISDLYEDLRD GHNLISLLEV |
| | | | | Sequence | LSGDSLPREK GRMRFHKLQN |
| | | | | version 3 (14 | VQIALDYLRH RQVKLVNIRN |
| | | | | Oct. 2008) | DDIADGNPKL TLGLIWTIIL |
| | | | | | HFQISDIQVS GQSEDMTAKE |
| | | | | | KLLLWSQRMV EGYQGLRCDN |
| | | | | | FTSSWRDGRL FNAIIHRHKP |
| | | | | | LLIDMNKVYR QTNLENLDQA |
| | | | | | FSVAERDLGV TRLLDPEDVD |
| | | | | | VPQPDEKSII TYVSSLYDAM |
| | | | | | PRVPDVQDGV RANELQLRWQ |
| | | | | | EYRELVLLLL QWMRHHTAAF |
| | | | | | EERRFPSSFE EIEILWSQFL |
| | | | | | KFKEMELPAK EADKNRSKGI |
| | | | | | YQSLEGAVQA GQLKVPPGYH |
| | | | | | PLDVEKEWGK LHVAILEREK |
| | | | | | QLRSEFERLE CLQRIVTKLQ |
| | | | | | MEAGLCEEQL NQADALLQSD |
| | | | | | VRLLAAGKVP QRAGEVERDL |
| | | | | | DKADSMIRLL FNDVQTLKDG |
| | | | | | RHPQGEQMYR RVYRLHERLV |
| | | | | | AIRTEYNLRL KAGVAAPATQ |
| | | | | | VAQVTLQSVQ RRPELEDSTL |
| | | | | | RYLQDLLAWV EENQHRVDGA |
| | | | | | EWGVDLPSVE AQLGSHRGLH |
| | | | | | QSIEEFRAKI ERARSDEGQL |
| | | | | | SPATRGAYRD CLGRLDLQYA |
| | | | | | KLLNSSKARL RSLESLHSFV |
| | | | | | AAATKELMWL NEKEEEEVGF |
| | | | | | DWSDRNTNMT AKKESYSALM |
| | | | | | RELELKEKKI KELQNAGDRL |
| | | | | | LREDHPARPT VESFQAALQT |
| | | | | | QWSWMLQLCC CIEAHLKENA |
| | | | | | AYFQFFSDVR EAEGQLQKLQ |
| | | | | | EALRRKYSCD RSATVTRLED |
| | | | | | LLQDAQDEKE QLNEYKGHLS |
| | | | | | GLAKRAKAVV QLKPRHPAHP |
| | | | | | MRGRLPLLAV CDYKQVEVTV |
| | | | | | HKGDECQLVG PAQPSHWKVL |
| | | | | | SSSGSEAAVP SVCFLVPPPN |
| | | | | | QEAQEAVTRL EAQHQALVTL |
| | | | | | WHQLHVDMKS LLAWQSLRRD |
| | | | | | VQLIRSWSLA TFRTLKPEEQ |
| | | | | | RQALHSLELH YQAFLRDSQD |
| | | | | | AGGFGPEDRL MAEREYGSCS |
| | | | | | HHYQQLLQSL EQGAQEESRC |
| | | | | | QRCISELKDI RLQLEACETR |
| | | | | | TVHRLRLPLD KEPARECAQR |
| | | | | | IAEQQKAQAE VEGLGKGVAR |
| | | | | | LSAEAEKVLA LPEPSPAAPT |
| | | | | | LRSELELTLG KLEQVRSLSA |
| | | | | | IYLEKLKTIS LVIRGTQGAE |
| | | | | | EVLRAHEEQL KEAQAVPATL |
| | | | | | PELEATKASL KKLRAQAEAQ |
| | | | | | QPTFDALRDE LRGAQEVGER |
| | | | | | LQQRHGERDV EVERWRERVA |
| | | | | | QLLERWQAVL AQTDVRQREL |
| | | | | | EQLGRQLRYY RESADPLGAW |
| | | | | | LQDARRRQEQ IQAMPLADSQ |
| | | | | | AVREQLRQEQ ALLEEIERHG |
| | | | | | EKVEECQRFA KQYINAIKDY |
| | | | | | ELQLVTYKAQ LEPVASPAKK |
| | | | | | PKVQSGSESV IQEYVDLRTH |
| | | | | | YSELTTLTSQ YIKFISETLR |
| | | | | | RMEEEERLAE QQRAEERERL |
| | | | | | AEVEAALEKQ RQLAEAHAQA |
| | | | | | KAQAEREAKE LQQRMQEEVV |
| | | | | | RREEAAVDAQ QQKRSIQEEL |
| | | | | | QQLRQSSEAE IQAKARQAEA |
| | | | | | AERSRLRIEE EIRVVRLQLE |
| | | | | | ATERQRGGAE GELQALRARA |
| | | | | | EEAEAQKRQA QEEAERLRRQ |
| | | | | | VQDESQRKRQ AEVELASRVK |
| | | | | | AEAEAAREKQ RALQALEELR |
| | | | | | LQAEEAERRL RQAEVERARQ |
| | | | | | VQVALETAQR SAEAELQSKR |
| | | | | | ASFAEKTAQL ERSLQEEHVA |
| | | | | | VAQLREEAER RAQQQAEAER |
| | | | | | AREEAERELE RWQLKANEAL |
| | | | | | RLRLQAEEVA QQKSLAQAEA |
| | | | | | EKQKEEAERE ARRRGKAEEQ |
| | | | | | AVRQRELAEQ ELEKQRQLAE |
| | | | | | GTAQQRLAAE QELIRLRAET |
| | | | | | EQGEQQRQLL EEELARLQRE |
| | | | | | AAAATQKRQE LEAELAKVRA |
| | | | | | EMEVLLASKA RAEEESRSTS |
| | | | | | EKSKQRLEAE AGRFRELAEE |
| | | | | | AARLRALAEE AKRQRQLAEE |
| | | | | | DAARQRAEAE RVLAEKLAAI |
| | | | | | GEATRLKTEA EIALKEKEAE |
| | | | | | NERLRRLAED EAFQRRRLEE |
| | | | | | QAAQHKADIE ERLAQLRKAS |
| | | | | | DSELERQKGL VEDTLRQRRQ |
| | | | | | VEEEILALKA SFEKAAAGKA |
| | | | | | ELELELGRIR SNAEDTLRSK |
| | | | | | EQAELEAARQ RQLAAEEERR |
| | | | | | RREAEERVQK SLAAEEEAAR |
| | | | | | QRKAALEEVE RLKAKVEEAR |
| | | | | | RLRERAEQES ARQLQLAQEA |
| | | | | | AQKRLQAEEK AHAFAVQQKE |
| | | | | | QELQQTLQQE QSVLDQLRGE |
| | | | | | AEAARRAAEE AEEARVQAER |
| | | | | | EAAQSRRQVE EAERLKQSAE |
| | | | | | EQAQARAQAQ AAAEKLRKEA |
| | | | | | EQEAARRAQA EQAALRQKQA |
| | | | | | ADAEMEKHKK FAEQTLRQKA |
| | | | | | QVEQELTTLR LQLEETDHQK |
| | | | | | NLLDEELQRL KAEATEAARQ |
| | | | | | RSQVEEELFS VRVQMEELSK |
| | | | | | LKARIEAENR ALILRDKDNT |
| | | | | | QRFLQEEAEK MKQVAEEAAR |
| | | | | | LSVAAQEAAR LRQLAEEDLA |
| | | | | | QQRALAEKML KEKMQAVQEA |
| | | | | | TRLKAEAELL QQQKELAQEQ |
| | | | | | ARRLQEDKEQ MAQQLAEETQ |
| | | | | | GFQRTLEAER QRQLEMSAEA |
| | | | | | ERLKLRVAEM SRAQARAEED |
| | | | | | AQRFRKQAEE IGEKLHRTEL |
| | | | | | ATQEKVTLVQ TLEIQRQQSD |
| | | | | | HDAERLREAI AELEREKEKL |
| | | | | | QQEAKLLQLK SEEMQTVQQE |
| | | | | | QLLQETQALQ QSFLSEKDSL |
| | | | | | LQRERFIEQE KAKLEQLFQD |
| | | | | | EVAKAQQLRE EQQRQQQQME |
| | | | | | QERQRLVASM EEARRRQHEA |
| | | | | | EEGVRRKQEE LQQLEQQRRQ |
| | | | | | QEELLAEENQ RLREQLQLLE |
| | | | | | EQHRAALAHS EEVTASQVAA |
| | | | | | TKTLPNGRDA LDGPAAEAEP |
| | | | | | EHSFDGLRRK VSAQRLQEAG |
| | | | | | ILSAEELQRL AQGHTTVDEL |
| | | | | | ARREDVRHYL QGRSSIAGLL |
| | | | | | LKATNEKLSV YAALQRQLLS |
| | | | | | PGTALILLEA QAASGFLLDP |
| | | | | | VRNRRLTVNE AVKEGVVGPE |
| | | | | | LHHKLLSAER AVTGYKDPYT |
| | | | | | GQQISLFQAM QKGLIVREHG |
| | | | | | IRLLEAQIAT GGVIDPVHSH |
| | | | | | RVPVDVAYRR GYFDEEMNRV |
| | | | | | LADPSDDTKG FFDPNTHENL |
| | | | | | TYLQLLERCV EDPETGLCLL |
| | | | | | PLTDKAAKGG ELVYTDSEAR |
| | | | | | DVFEKATVSA PFGKFQGKTV |
| | | | | | TIWEIINSEY FTAEQRRDLL |
| | | | | | RQFRTGRITV EKIIKIIITV |
| | | | | | VEEQEQKGRL CFEGLRSLVP |
| | | | | | AAELLESRVI DRELYQQLQR |
| | | | | | GERSVRDVAE VDTVRRALRG |
| | | | | | ANVIAGVWLE EAGQKLSIYN |
| | | | | | ALKKDLLPSD MAVALLEAQA |
| | | | | | GTGHIIDPAT SARLTVDEAV |
| | | | | | RAGLVGPEFH EKLLSAEKAV |
| | | | | | TGYRDPYTGQ SVSLFQALKK |
| | | | | | GLIPREQGLR LLDAQLSTGG |
| | | | | | IVDPSKSHRV PLDVACARGC |
| | | | | | LDEETSRALS APRADAKAYS |
| | | | | | DPSTGEPATY GELQQRCRPD |
| | | | | | QLTGLSLLPL SEKAARARQE |
| | | | | | ELYSELQARE TFEKTPVEVP |
| | | | | | VGGFKGRTVT VWELISSEYF |
| | | | | | TAEQRQELLR QFRTGKVTVE |
| | | | | | KVIKILITIV EEVETLRQER |
| | | | | | LSFSGLRAPV PASELLASGV |
| | | | | | LSRAQFEQLK DGKTTVKDLS |
| | | | | | ELGSVRTLLQ GSGCLAGIYL |
| | | | | | EDTKEKVSIY EAMRRGLLRA |
| | | | | | TTAALLLEAQ AATGFLVDPV |
| | | | | | RNQRLYVHEA VKAGVVGPEL |
| | | | | | HEQLLSAEKA VTGYRDPYSG |
| | | | | | STISLFQAMQ KGLVLRQHGI |
| | | | | | RLLEAQIATG GIIDPVHSHR |
| | | | | | VPVDVAYQRG YFSEEMNRVL |
| | | | | | ADPSDDTKGF FDPNTHENLT |
| | | | | | YRQLLERCVE DPETGLRLLP |
| | | | | | LKGAEKAEVV ETTQVYTEEE |
| | | | | | TRRAFEETQI DIPGGGSHGG |
| | | | | | STMSLWEVMQ SDLIPEEQRA |
| | | | | | QLMADFQAGR VTKERMIIII |
| | | | | | IEIIEKTEII RQQGLASYDY |
| | | | | | VRRRLTAEDL FEARIISLET |
| | | | | | YNLLREGTRS LREALEAESA |
| | | | | | WCYLYGTGSV AGVYLPGSRQ |
| | | | | | TLSIYQALKK GLLSAEVARL |
| | | | | | LLEAQAATGF LLDPVKGERL |
| | | | | | TVDEAVRKGL VGPELHDRLL |
| | | | | | SAERAVTGYR DPYTEQTISL |
| | | | | | FQAMKKELIP TEEALRLLDA |
| | | | | | QLATGGIVDP RLGFHLPLEV |
| | | | | | AYQRGYLNKD THDQLSEPSE |
| | | | | | VRSYVDPSTD ERLSYTQLLR |
| | | | | | RCRRDDGTGQ LLLPLSDARK |
| | | | | | LTFRGLRKQI TMEELVRSQV |
| | | | | | MDEATALQLR EGLTSIEEVT |
| | | | | | KNLQKFLEGT SCIAGVFVDA |
| | | | | | TKERLSVYQA MKKGIIRPGT |
| | | | | | AFELLEAQAA TGYVIDPIKG |
| | | | | | LKLTVEEAVR MGIVGPEFKD |
| | | | | | KLLSAERAVT GYKDPYSGKL |
| | | | | | ISLFQAMKKG LILKDHGIRL |
| | | | | | LEAQIATGGI IDPEESHRLP |
| | | | | | VEVAYKRGLF DEEMNEILTD |
| | | | | | PSDDTKGFFD PNTEENLTYL |
| | | | | | QLMERCITDP QTGLCLLPLK |
| | | | | | EKKRERKTSS KSSVRKRRVV |
| | | | | | IVDPETGKEM SVYEAYRKGL |
| | | | | | IDHQTYLELS EQECEWEEIT |
| | | | | | ISSSDGVVKS MIIDRRSGRQ |
| | | | | | YDIDDAIAKN LIDRSALDQY |
| | | | | | RAGTLSITEF ADMLSGNAGG |
| | | | | | FRSRSSSVGS SSSYPISPAV |
| | | | | | SRTQLASWSD PTEETGPVAG |
| | | | | | ILDTETLEKV SITEAMHRNL |
| | | | | | VDNITGQRLL EAQACTGGII |
| | | | | | DPSTGERFPV TDAVNKGLVD |
| | | | | | KIMVDRINLA QKAFCGFEDP |
| | | | | | RTKTKMSAAQ ALKKGWLYYE |
| | | | | | AGQRFLEVQY LTGGLIEPDT |
| | | | | | PGRVPLDEAL QRGTVDARTA |
| | | | | | QKLRDVGAYS KYLTCPKTKL |
| | | | | | KISYKDALDR SMVEEGTGLR |
| | | | | | LLEAAAQSTK GYYSPYSVSG |
| | | | | | SGSTAGSRTG SRTGSRAGSR |
| | | | | | RGSFDATGSG FSMTFSSSSY |
| | | | | | SSSGYGRRYA SGSSASLGGP |
| | | | | | ESAVA |
| 75 | PLEC | 9 | Q15149- | Entry version | MAGPLPDEQD FIQAYEEVRE |
| | | | 9 | 224 (18 Sep. | KYKDERDRVQ KKTFTKWVNK |
| | | | | 2019) | HLIKAQRHIS DLYEDLRDGH |
| | | | | Sequence | NLISLLEVLS GDSLPREKGR |
| | | | | version 3 (14 | MRFHKLQNVQ IALDYLRHRQ |
| | | | | Oct. 2008) | VKLVNIRNDD IADGNPKLTL |
| | | | | | GLIWTIILHF QISDIQVSGQ |
| | | | | | SEDMTAKEKL LLWSQRMVEG |
| | | | | | YQGLRCDNFT SSWRDGRLFN |
| | | | | | AIIHRHKPLL IDMNKVYRQT |
| | | | | | NLENLDQAFS VAERDLGVTR |
| | | | | | LLDPEDVDVP QPDEKSIITY |
| | | | | | VSSLYDAMPR VPDVQDGVRA |
| | | | | | NELQLRWQEY RELVLLLLQW |
| | | | | | MRHHTAAFEE RRFPSSFEEI |
| | | | | | EILWSQFLKF KEMELPAKEA |
| | | | | | DKNRSKGIYQ SLEGAVQAGQ |
| | | | | | LKVPPGYHPL DVEKEWGKLH |
| | | | | | VAILEREKQL RSEFERLECL |
| | | | | | QRIVTKLQME AGLCEEQLNQ |
| | | | | | ADALLQSDVR LLAAGKVPQR |
| | | | | | AGEVERDLDK ADSMIRLLFN |
| | | | | | DVQTLKDGRH PQGEQMYRRV |
| | | | | | YRLHERLVAI RTEYNLRLKA |
| | | | | | GVAAPATQVA QVTLQSVQRR |
| | | | | | PELEDSTLRY LQDLLAWVEE |
| | | | | | NQHRVDGAEW GVDLPSVEAQ |
| | | | | | LGSHRGLHQS IEEFRAKIER |
| | | | | | ARSDEGQLSP ATRGAYRDCL |
| | | | | | GRLDLQYAKL LNSSKARLRS |
| | | | | | LESLHSFVAA ATKELMWLNE |
| | | | | | KEEEEVGFDW SDRNTNMTAK |
| | | | | | KESYSALMRE LELKEKKIKE |
| | | | | | LQNAGDRLLR EDHPARPTVE |
| | | | | | SFQAALQTQW SWMLQLCCCI |
| | | | | | EAHLKENAAY FQFFSDVREA |
| | | | | | EGQLQKLQEA LRRKYSCDRS |
| | | | | | ATVTRLEDLL QDAQDEKEQL |
| | | | | | NEYKGHLSGL AKRAKAVVQL |
| | | | | | KPRHPAHPMR GRLPLLAVCD |
| | | | | | YKQVEVTVHK GDECQLVGPA |
| | | | | | QPSHWKVLSS SGSEAAVPSV |
| | | | | | CFLVPPPNQE AQEAVTRLEA |
| | | | | | QHQALVTLWH QLHVDMKSLL |
| | | | | | AWQSLRRDVQ LIRSWSLATF |
| | | | | | RTLKPEEQRQ ALHSLELHYQ |
| | | | | | AFLRDSQDAG GFGPEDRLMA |
| | | | | | EREYGSCSHH YQQLLQSLEQ |
| | | | | | GAQEESRCQR CISELKDIRL |
| | | | | | QLEACETRTV HRLRLPLDKE |
| | | | | | PARECAQRIA EQQKAQAEVE |
| | | | | | GLGKGVARLS AEAEKVLALP |
| | | | | | EPSPAAPTLR SELELTLGKL |
| | | | | | EQVRSLSAIY LEKLKTISLV |
| | | | | | IRGTQGAEEV LRAHEEQLKE |
| | | | | | AQAVPATLPE LEATKASLKK |
| | | | | | LRAQAEAQQP TFDALRDELR |
| | | | | | GAQEVGERLQ QRHGERDVEV |
| | | | | | ERWRERVAQL LERWQAVLAQ |
| | | | | | TDVRQRELEQ LGRQLRYYRE |
| | | | | | SADPLGAWLQ DARRRQEQIQ |
| | | | | | AMPLADSQAV REQLRQEQAL |
| | | | | | LEEIERHGEK VEECQRFAKQ |
| | | | | | YINAIKDYEL QLVTYKAQLE |
| | | | | | PVASPAKKPK VQSGSESVIQ |
| | | | | | EYVDLRTHYS ELTTLTSQYI |
| | | | | | KFISETLRRM EEEERLAEQQ |
| | | | | | RAEERERLAE VEAALEKQRQ |
| | | | | | LAEAHAQAKA QAEREAKELQ |
| | | | | | QRMQEEVVRR EEAAVDAQQQ |
| | | | | | KRSIQEELQQ LRQSSEAEIQ |
| | | | | | AKARQAEAAE RSRLRIEEEI |
| | | | | | RVVRLQLEAT ERQRGGAEGE |
| | | | | | LQALRARAEE AEAQKRQAQE |
| | | | | | EAERLRRQVQ DESQRKRQAE |
| | | | | | VELASRVKAE AEAAREKQRA |
| | | | | | LQALEELRLQ AEEAERRLRQ |
| | | | | | AEVERARQVQ VALETAQRSA |
| | | | | | EAELQSKRAS FAEKTAQLER |
| | | | | | SLQEEHVAVA QLREEAERRA |
| | | | | | QQQAEAERAR EEAERELERW |
| | | | | | QLKANEALRL RLQAEEVAQQ |
| | | | | | KSLAQAEAEK QKEEAEREAR |
| | | | | | RRGKAEEQAV RQRELAEQEL |
| | | | | | EKQRQLAEGT AQQRLAAEQE |
| | | | | | LIRLRAETEQ GEQQRQLLEE |
| | | | | | ELARLQREAA AATQKRQELE |
| | | | | | AELAKVRAEM EVLLASKARA |
| | | | | | EEESRSTSEK SKQRLEAEAG |
| | | | | | RFRELAEEAA RLRALAEEAK |
| | | | | | RQRQLAEEDA ARQRAEAERV |
| | | | | | LAEKLAAIGE ATRLKTEAEI |
| | | | | | ALKEKEAENE RLRRLAEDEA |
| | | | | | FQRRRLEEQA AQHKADIEER |
| | | | | | LAQLRKASDS ELERQKGLVE |
| | | | | | DTLRQRRQVE EEILALKASF |
| | | | | | EKAAAGKAEL ELELGRIRSN |
| | | | | | AEDTLRSKEQ AELEAARQRQ |
| | | | | | LAAEEERRRR EAEERVQKSL |
| | | | | | AAEEEAARQR KAALEEVERL |
| | | | | | KAKVEEARRL RERAEQESAR |
| | | | | | QLQLAQEAAQ KRLQAEEKAH |
| | | | | | AFAVQQKEQE LQQTLQQEQS |
| | | | | | VLDQLRGEAE AARRAAEEAE |
| | | | | | EARVQAEREA AQSRRQVEEA |
| | | | | | ERLKQSAEEQ AQARAQAQAA |
| | | | | | AEKLRKEAEQ EAARRAQAEQ |
| | | | | | AALRQKQAAD AEMEKHKKFA |
| | | | | | EQTLRQKAQV EQELTTLRLQ |
| | | | | | LEETDHQKNL LDEELQRLKA |
| | | | | | EATEAARQRS QVEEELFSVR |
| | | | | | VQMEELSKLK ARIEAENRAL |
| | | | | | ILRDKDNTQR FLQEEAEKMK |
| | | | | | QVAEEAARLS VAAQEAARLR |
| | | | | | QLAEEDLAQQ RALAEKMLKE |
| | | | | | KMQAVQEATR LKAEAELLQQ |
| | | | | | QKELAQEQAR RLQEDKEQMA |
| | | | | | QQLAEETQGF QRTLEAERQR |
| | | | | | QLEMSAEAER LKLRVAEMSR |
| | | | | | AQARAEEDAQ RFRKQAEEIG |
| | | | | | EKLHRTELAT QEKVTLVQTL |
| | | | | | EIQRQQSDHD AERLREAIAE |
| | | | | | LEREKEKLQQ EAKLLQLKSE |
| | | | | | EMQTVQQEQL LQETQALQQS |
| | | | | | FLSEKDSLLQ RERFIEQEKA |
| | | | | | KLEQLFQDEV AKAQQLREEQ |
| | | | | | QRQQQQMEQE RQRLVASMEE |
| | | | | | ARRRQHEAEE GVRRKQEELQ |
| | | | | | QLEQQRRQQE ELLAEENQRL |
| | | | | | REQLQLLEEQ HRAALAHSEE |
| | | | | | VTASQVAATK TLPNGRDALD |
| | | | | | GPAAEAEPEH SFDGLRRKVS |
| | | | | | AQRLQEAGIL SAEELQRLAQ |
| | | | | | GHTTVDELAR REDVRHYLQG |
| | | | | | RSSIAGLLLK ATNEKLSVYA |
| | | | | | ALQRQLLSPG TALILLEAQA |
| | | | | | ASGFLLDPVR NRRLTVNEAV |
| | | | | | KEGVVGPELH HKLLSAERAV |
| | | | | | TGYKDPYTGQ QISLFQAMQK |
| | | | | | GLIVREHGIR LLEAQIATGG |
| | | | | | VIDPVHSHRV PVDVAYRRGY |
| | | | | | FDEEMNRVLA DPSDDTKGFF |
| | | | | | DPNTHENLTY LQLLERCVED |
| | | | | | PETGLCLLPL TDKAAKGGEL |
| | | | | | VYTDSEARDV FEKATVSAPF |
| | | | | | GKFQGKTVTI WEIINSEYFT |
| | | | | | AEQRRDLLRQ FRTGRITVEK |
| | | | | | IIKIIITVVE EQEQKGRLCF |
| | | | | | EGLRSLVPAA ELLESRVIDR |
| | | | | | ELYQQLQRGE RSVRDVAEVD |
| | | | | | TVRRALRGAN VIAGVWLEEA |
| | | | | | GQKLSIYNAL KKDLLPSDMA |
| | | | | | VALLEAQAGT GHIIDPATSA |
| | | | | | RLTVDEAVRA GLVGPEFHEK |
| | | | | | LLSAEKAVTG YRDPYTGQSV |
| | | | | | SLFQALKKGL IPREQGLRLL |
| | | | | | DAQLSTGGIV DPSKSHRVPL |
| | | | | | DVACARGCLD EETSRALSAP |
| | | | | | RADAKAYSDP STGEPATYGE |
| | | | | | LQQRCRPDQL TGLSLLPLSE |
| | | | | | KAARARQEEL YSELQARETF |
| | | | | | EKTPVEVPVG GFKGRTVTVW |
| | | | | | ELISSEYFTA EQRQELLRQF |
| | | | | | RTGKVTVEKV IKILITIVEE |
| | | | | | VETLRQERLS FSGLRAPVPA |
| | | | | | SELLASGVLS RAQFEQLKDG |
| | | | | | KTTVKDLSEL GSVRTLLQGS |
| | | | | | GCLAGIYLED TKEKVSIYEA |
| | | | | | MRRGLLRATT AALLLEAQAA |
| | | | | | TGFLVDPVRN QRLYVHEAVK |
| | | | | | AGVVGPELHE QLLSAEKAVT |
| | | | | | GYRDPYSGST ISLFQAMQKG |
| | | | | | LVLRQHGIRL LEAQIATGGI |
| | | | | | IDPVHSHRVP VDVAYQRGYF |
| | | | | | SEEMNRVLAD PSDDTKGFFD |
| | | | | | PNTHENLTYR QLLERCVEDP |
| | | | | | ETGLRLLPLK GAEKAEVVET |
| | | | | | TQVYTEEETR RAFEETQIDI |
| | | | | | PGGGSHGGST MSLWEVMQSD |
| | | | | | LIPEEQRAQL MADFQAGRVT |
| | | | | | KERMIIIIIE IIEKTEIIRQ |
| | | | | | QGLASYDYVR RRLTAEDLFE |
| | | | | | ARIISLETYN LLREGTRSLR |
| | | | | | EALEAESAWC YLYGTGSVAG |
| | | | | | VYLPGSRQTL SIYQALKKGL |
| | | | | | LSAEVARLLL EAQAATGELL |
| | | | | | DPVKGERLTV DEAVRKGLVG |
| | | | | | PELHDRLLSA ERAVTGYRDP |
| | | | | | YTEQTISLFQ AMKKELIPTE |
| | | | | | EALRLLDAQL ATGGIVDPRL |
| | | | | | GFHLPLEVAY QRGYLNKDTH |
| | | | | | DQLSEPSEVR SYVDPSTDER |
| | | | | | LSYTQLLRRC RRDDGTGQLL |
| | | | | | LPLSDARKLT FRGLRKQITM |
| | | | | | EELVRSQVMD EATALQLREG |
| | | | | | LTSIEEVTKN LQKFLEGTSC |
| | | | | | IAGVFVDATK ERLSVYQAMK |
| | | | | | KGIIRPGTAF ELLEAQAATG |
| | | | | | YVIDPIKGLK LTVEEAVRMG |
| | | | | | IVGPEFKDKL LSAERAVTGY |
| | | | | | KDPYSGKLIS LFQAMKKGLI |
| | | | | | LKDHGIRLLE AQIATGGIID |
| | | | | | PEESHRLPVE VAYKRGLEDE |
| | | | | | EMNEILTDPS DDTKGFFDPN |
| | | | | | TEENLTYLQL MERCITDPQT |
| | | | | | GLCLLPLKEK KRERKTSSKS |
| | | | | | SVRKRRVVIV DPETGKEMSV |
| | | | | | YEAYRKGLID HQTYLELSEQ |
| | | | | | ECEWEEITIS SSDGVVKSMI |
| | | | | | IDRRSGRQYD IDDAIAKNLI |
| | | | | | DRSALDQYRA GTLSITEFAD |
| | | | | | MLSGNAGGER SRSSSVGSSS |
| | | | | | SYPISPAVSR TQLASWSDPT |
| | | | | | EETGPVAGIL DTETLEKVSI |
| | | | | | TEAMHRNLVD NITGQRLLEA |
| | | | | | QACTGGIIDP STGERFPVTD |
| | | | | | AVNKGLVDKI MVDRINLAQK |
| | | | | | AFCGFEDPRT KTKMSAAQAL |
| | | | | | KKGWLYYEAG QRFLEVQYLT |
| | | | | | GGLIEPDTPG RVPLDEALQR |
| | | | | | GTVDARTAQK LRDVGAYSKY |
| | | | | | LTCPKTKLKI SYKDALDRSM |
| | | | | | VEEGTGLRLL EAAAQSTKGY |
| | | | | | YSPYSVSGSG STAGSRTGSR |
| | | | | | TGSRAGSRRG SFDATGSGFS |
| | | | | | MTFSSSSYSS SGYGRRYASG |
| | | | | | SSASLGGPES AVA |
| 76 | ACSLI | 1 | P33121 | Entry version | MQAHELFRYF RMPELVDERQ |
| | | | | 186 (18 Sep. | YVRTLPTNTL MGFGAFAALT |
| | | | | 2019) | TFWYATRPKP LKPPCDLSMQ |
| | | | | Sequence | SVEVAGSGGA RRSALLDSDE |
| | | | | version 1 (01 | PLVYFYDDVT TLYEGFQRGI |
| | | | | Oct. 1993) | QVSNNGPCLG SRKPDQPYEW |
| | | | | | LSYKQVAELS ECIGSALIQK |
| | | | | | GFKTAPDQFI GIFAQNRPEW |
| | | | | | VIIEQGCFAY SMVIVPLYDT |
| | | | | | LGNEAITYIV NKAELSLVFV |
| | | | | | DKPEKAKLLL EGVENKLIPG |
| | | | | | LKIIVVMDAY GSELVERGQR |
| | | | | | CGVEVTSMKA MEDLGRANRR |
| | | | | | KPKPPAPEDL AVICFTSGTT |
| | | | | | GNPKGAMVTH RNIVSDCSAF |
| | | | | | VKATENTVNP CPDDTLISFL |
| | | | | | PLAHMFERVV ECVMLCHGAK |
| | | | | | IGFFQGDIRL LMDDLKVLQP |
| | | | | | TVFPVVPRLL NRMFDRIFGQ |
| | | | | | ANTTLKRWLL DFASKRKEAE |
| | | | | | LRSGIIRNNS LWDRLIFHKV |
| | | | | | QSSLGGRVRL MVTGAAPVSA |
| | | | | | TVLTFLRAAL GCQFYEGYGQ |
| | | | | | TECTAGCCLT MPGDWTAGHV |
| | | | | | GAPMPCNLIK LVDVEEMNYM |
| | | | | | AAEGEGEVCV KGPNVFQGYL |
| | | | | | KDPAKTAEAL DKDGWLHTGD |
| | | | | | IGKWLPNGTL KIIDRKKHIF |
| | | | | | KLAQGEYIAP EKIENIYMRS |
| | | | | | EPVAQVFVHG ESLQAFLIAI |
| | | | | | VVPDVETLCS WAQKRGFEGS |
| | | | | | FEELCRNKDV KKAILEDMVR |
| | | | | | LGKDSGLKPF EQVKGITLHP |
| | | | | | ELFSIDNGLL TPTMKAKRPE |
| | | | | | LRNYFRSQID DLYSTIK |
| 77 | ACSLI | 2 | P33121- | Entry version | MQAHELFRYF RMPELVDFRQ |
| | | | 2 | 186 (18 Sep. | YVRTLPTNTL MGFGAFAALT |
| | | | | 2019) | TFWYATRPKP LKPPCDLSMQ |
| | | | | Sequence | SVEVAGSGGA RRSALLDSDE |
| | | | | version 1 (01 | PLVYFYDDVT TLYEGFQRGI |
| | | | | Oct. 1993) | QVSNNGPCLG SRKPDQPYEW |
| | | | | | LSYKQVAELS ECIGSALIQK |
| | | | | | GFKTAPDQFI GIFAQNRPEW |
| | | | | | VIIEQGCFAY SMVIVPLYDT |
| | | | | | LGNEAITYIV NKAELSLVFV |
| | | | | | DKPEKAKLLL EGVENKLIPG |
| | | | | | LKIIVVMDAY GSELVERGQR |
| | | | | | CGVEVTSMKA MEDLGRANRR |
| | | | | | KPKPPAPEDL AVICFTSGTT |
| | | | | | GNPKGAMVTH RNIVSDCSAF |
| | | | | | VKATENTVNP CPDDTLISFL |
| | | | | | PLAHMFERVV ECVMLCHGAK |
| | | | | | IGFFQGDIRL LMDDLKVLQP |
| | | | | | TVFPVVPRLL NRMFDRIFGQ |
| | | | | | ANTTLKRWLL DFASKRKEAE |
| | | | | | LRSGIIRNNS LWDRLIFHKV |
| | | | | | QSSLGGRVRL MVTGAAPVSA |
| | | | | | TVLTFLRAAL GCQFYEGYGQ |
| | | | | | TECTAGCCLT MPGDWTAGHV |
| | | | | | GAPMPCNLIK LVDVEEMNYM |
| | | | | | AAEGEGEGYL KDPAKTAEAL |
| | | | | | DKDGWLHTGD IGKWLPNGTL |
| | | | | | KIIDRKKHIF KLAQGEYIAP |
| | | | | | EKIENIYMRS EPVAQVFVHG |
| | | | | | ESLQAFLIAI VVPDVETLCS |
| | | | | | WAQKRGFEGS FEELCRNKDV |
| | | | | | KKAILEDMVR LGKDSGLKPF |
| | | | | | EQVKGITLHP ELFSIDNGLL |
| | | | | | TPTMKAKRPE LRNYFRSQID |
| | | | | | DLYSTIKV |
| 78 | ACSLI | 3 | P33121- | Entry version | MQAHELFRYF RMPELVDERQ |
| | | | 3 | 186 (18 Sep. | YVRTLPTNTL MGFGAFAALT |
| | | | | 2019) | TFWYATRPKP LKPPCDLSMQ |
| | | | | Sequence | SVEVAGSGGA RRSALLDSDE |
| | | | | version 1 (01 | PLVYFYDDVT TLYEGFQRGI |
| | | | | Oct. 1993) | QVSNNGPCLG SRKPDQPYEW |
| | | | | | LSYKQVAELS ECIGSALIQK |
| | | | | | GFKTAPDQFI GIFAQNRPEW |
| | | | | | VIIEQGCFAY SMVIVPLYDT |
| | | | | | LGNEAITYIV NKAELSLVFV |
| | | | | | DKPEKAKLLL EGVENKLIPG |
| | | | | | LKIIVVMDAY GSELVERGQR |
| | | | | | CGVEVTSMKA MEDLGRANRR |
| | | | | | KPKPPAPEDL AVICFTSGTT |
| | | | | | GNPKGAMVTH RNIVSDCSAF |
| | | | | | VKATEKALPL SASDTHISYL |
| | | | | | PLAHIYEQLL KCVMLCHGAK |
| | | | | | IGFFQGDIRL LMDDLKVLQP |
| | | | | | TVFPVVPRLL NRMEDRIFGQ |
| | | | | | ANTTLKRWLL DFASKRKEAE |
| | | | | | LRSGIIRNNS LWDRLIFHKV |
| | | | | | QSSLGGRVRL MVTGAAPVSA |
| | | | | | TVLTFLRAAL GCQFYEGYGQ |
| | | | | | TECTAGCCLT MPGDWTAGHV |
| | | | | | GAPMPCNLIK LVDVEEMNYM |
| | | | | | AAEGEGEVCV KGPNVFQGYL |
| | | | | | KDPAKTAEAL DKDGWLHTGD |
| | | | | | IGKWLPNGTL KIIDRKKHIF |
| | | | | | KLAQGEYIAP EKIENIYMRS |
| | | | | | EPVAQVFVHG ESLQAFLIAI |
| | | | | | VVPDVETLCS WAQKRGFEGS |
| | | | | | FEELCRNKDV KKAILEDMVR |
| | | | | | LGKDSGLKPF EQVKGITLHP |
| | | | | | ELFSIDNGLL TPTMKAKRPE |
| | | | | | LRNYFRSQID DLYSTIKV |
| 79 | RAC1 | 1 | P63000 | Entry version | MQAIKCVVVG DGAVGKTCLL |
| | | | | 192 (18 Sep. | ISYTTNAFPG EYIPTVFDNY |
| | | | | 2019) | SANVMVDGKP VNLGLWDTAG |
| | | | | Sequence | QEDYDRLRPL SYPQTDVELI |
| | | | | version 1 (31 | CFSLVSPASF ENVRAKWYPE |
| | | | | Aug. 2004) | VRHHCPNTPI ILVGTKLDLR |
| | | | | | DDKDTIEKLK EKKLTPITYP |
| | | | | | QGLAMAKEIG AVKYLECSAL |
| | | | | | TQRGLKTVED EAIRAVLCPP |
| | | | | | PVKKRKRKCL LL |
| 80 | RAC1 | 2 | P63000- | Entry version | MQAIKCVVVG DGAVGKTCLL |
| | | | 2 | 192 (18 Sep. | ISYTTNAFPG EYIPTVFDNY |
| | | | | 2019) | SANVMVDGKP VNLGLWDTAG |
| | | | | Sequence | QEDYDRLRPL SYPQTVGETY |
| | | | | version 1 (31 | GKDITSRGKD KPIADVFLIC |
| | | | | Aug. 2004) | FSLVSPASFE NVRAKWYPEV |
| | | | | | RHHCPNTPII LVGTKLDLRD |
| | | | | | DKDTIEKLKE KKLTPITYPQ |
| | | | | | GLAMAKEIGA VKYLECSALT |
| | | | | | QRGLKTVEDE AIRAVLCPPP |
| | | | | | VKKRKRKCLL L |
| 81 | PSMB2 | 1 | P49721 | Entry version | MEYLIGIQGP DYVLVASDRV |
| | | | | 201 (18 Sep. | AASNIVQMKD DHDKMFKMSE |
| | | | | 2019) | KILLLCVGEA GDTVQFAEYI |
| | | | | Sequence | QKNVQLYKMR NGYELSPTAA |
| | | | | version 1 (01 | ANFTRRNLAD CLRSRTPYHV |
| | | | | Oct. 1996) | NLLLAGYDEH EGPALYYMDY |
| | | | | | LAALAKAPFA AHGYGAFLTL |
| | | | | | SILDRYYTPT ISRERAVELL |
| | | | | | RKCLEELQKR FILNLPTFSV |
| | | | | | RIIDKNGIHD LDNISFPKQG |
| | | | | | S |
| 82 | GM2A | 1 | P17900 | Entry version | MQSLMQAPLL IALGLLLAAP |
| | | | | 190 (18 Sep. | AQAHLKKPSQ LSSFSWDNCD |
| | | | | 2019) | EGKDPAVIRS LTLEPDPIIV |
| | | | | Sequence | PGNVTLSVMG STSVPLSSPL |
| | | | | version 4 (13 | KVDLVLEKEV AGLWIKIPCT |
| | | | | Nov. 2007) | DYIGSCTFEH FCDVLDMLIP |
| | | | | | TGEPCPEPLR TYGLPCHCPF |
| | | | | | KEGTYSLPKS EFVVPDLELP |
| | | | | | SWLTTGNYRI ESVLSSSGKR |
| | | | | | LGCIKIAASL KGI |

Variant Gene Sequences

| SEQ ID NO. | GENE | Accession No. | Ensembl Release No. |
|---|---|---|---|
| 83 | CAP37 | ENSG00000278624 | 98 |
| 84 | TAPBP | ENSG00000112493 | 98 |
| 85 | TAPBP | ENSG00000206281 | 98 |
| 86 | TAPBP | ENSG00000112493 | 98 |
| 87 | TAPBP | ENSG00000236490 | 98 |

### EXAMPLES

### Materials and Methods

Collection of Granulocytes from Donors

Neutrophils and stem cells collected from twenty healthy human volunteers were selected with equal weighting between the following 4 groups:
Group 1: Male Over 40
Group 2: Male Under 40
Group 3: Female Over 40
Group 4: Female Under 40

All donors were healthy and had confirmed that no anti-inflammatory drugs had been taken up to 10 days prior to blood donation. Cells were isolated using standard techniques.

### Bacterial Culture

24 hours prior to use, overnight cultures (ONCs) of all strains (P. aeruginosa: multidrug resistant Cystic Fibrosis isolate RP73 (Di Lorenzo et al (2015), Mol. Immunol., 63, 166-175); MRSA: Community acquired strain USA300 (Diep et al (2006), Lancet, 367, 731-739)) were prepared by inoculating 20 ml tryptic soy broth (3% w/v TSB in deionised water) with 2 stock cryobeads for 24 hours at 37° C., under shaking at 120 rpm (Sciquip mini incu shake). After incubation, ONCs were centrifuged at 2,800 g for 20 minutes at 4° C. The pellet was then resuspended in 10 ml Roswell Park Memorial Institute (RPMI) 1640 medium (commercially available from Sigma-Aldrich, UK). Optical density readings were then taken for all strains and diluted in RPMI 1640 to an OD of 0.015, equivalent of 1×10⁷ cfu/ml.

### Neutrophil Bacterial Co-Culture Assay

Bacterial cultures were prepared as described above. 100 μl of 1×10⁷ cfu/ml bacterial strains were added to either 100 μl RPMI 1640, 100 μl 1×10⁷/ml neutrophils or increasing concentrations of Tobramycin (for P. aeruginosa) or Vancomycin (for MRSA) (1, 10, 100 μg/ml). These cultures were then incubated at 37° C., under shaking at 120 rpm for up to 24 hours. At 2, 6 and 24 hours, 20 μl aliquots of each sample were diluted in sterile RPMI at 1/10, 1/100, 1/1000, 1/10000, 1/100000 and 1/1000000 and plated on Tryptic Soy Agar (TSA) and incubated at 37° C. for 24 hours. Post incubation, bacterial colonies were manually counted and the total cfu content quantified.

### Example 1

### Validation of Neutrophil-Mediated Bacterial Killing

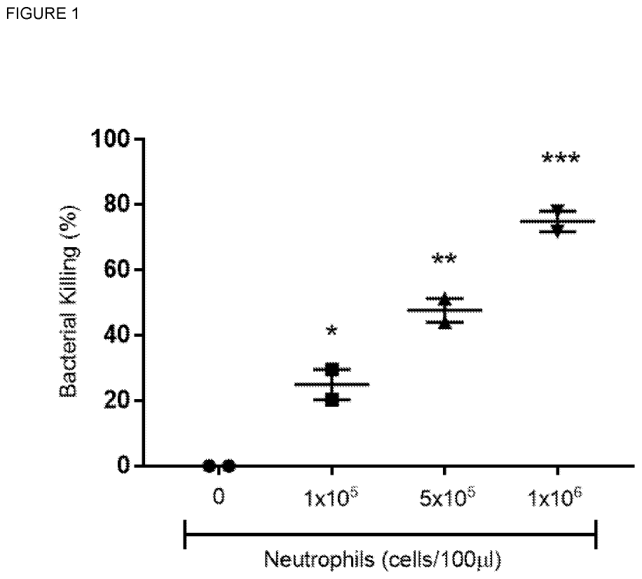

To validate an in vitro model of neutrophil bacterial killing activity (BKA) assay, increasing concentrations of neutrophils (1×10⁵, 5×10⁵ and 1×10⁶ neutrophils per sample) were incubated with 1×10⁶ cfu/ml of the P. aeruginosa strain RP73 under suspension. These neutrophil/bacterial cultures were then incubated for 2 hours at 37° C. under 120 rpm of shaking. After incubation 20 μl aliquots of each sample were diluted in sterile RPMI at 1/10, 1/100, 1/1000, 1/10000, 1/100000 and 1/1000000-fold dilutions and plated on Tryptic Soy Agar (TSA) and incubated at 37° C. for 24 hours. Post incubation, bacterial colonies were manually counted and the total cfu content quantified. A concentration-dependent increase in bacterial killing was observed compared to negative controls (0: 0±0%; 1×10⁵: 25.00±3.81, P<0.05; 5×10⁵: 47.67±2.54%, P<0.01; 1×10⁶: 74.93±1.98, P<0.001, FIG. 1). From these data, for all future experiments a concentration of 1×10⁶ neutrophils present in suspension was used to assess neutrophil bacterial killing.

### Example 2

### Age and Gender Differences in Neutrophil Mediated Bacterial Killing

Neutrophils from the various donors indicated in the Materials & Methods section were assessed (using the assay described in Example 1) for bacterial killing activity (BKA) against the multi-drug resistant clinical isolate of the Gram-negative bacterium P. aeruginosa RP73 and the community acquired Methicillin Resistant Staphylococcus aureus (MRSA) strain USA300 over 2 hours.

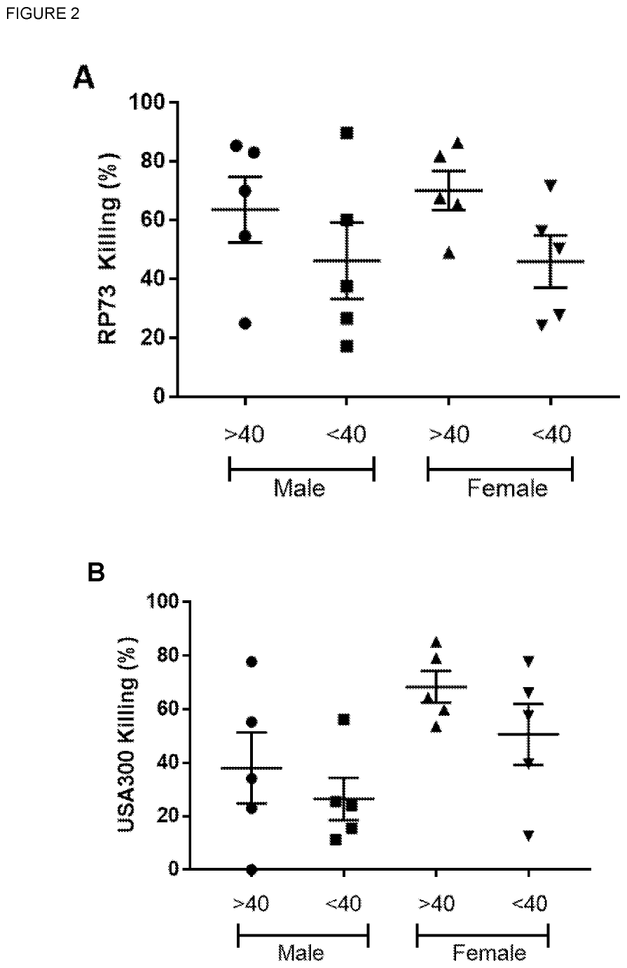

FIG. 2A shows that neutrophils from all donors induced at least some killing of RP73, and surprisingly a trend was observed demonstrating the highest level of killing in males and females over 40 years old, particularly in females (Male>40: 63.70±11.12%; Male<40: 46.32±13.00%; Female>40: 70.14±6.63: Female<40: 46.06±8.91%, FIG. 2A).

FIG. 2B shows that neutrophils from all donors demonstrated at least some ability to kill the MRSA strain USA300. Similarly to the P. aeruginosa strain RP73, a surprising trend was observed suggesting that for both males and females (but particularly females), the over 40 cohort demonstrated enhanced killing over the gender controlled under 40 cohort (Male>40: 38.03±13.35%; Male<40: 26.46±7.89%; Female>40: 68.41±5.93%; Female<40: 50.69±11.39%, FIG. 2B)

### Example 3

Neutrophil-Mediated Killing of Bacteria is Greater than Antibiotic Treatment at 2 Hours

It was observed that neutrophils cultured from some human donors possessed a superior BKA compared to neutrophils cultured from other donors with 25% of tested donors demonstrating greater than 80% killing of RP73 in 2 hours. These donors were chosen to compare the BKA against the activity of the most common antibiotics used for the bacteria of interest (Tobramycin for P. aeruginosa and Vancomycin for MRSA).

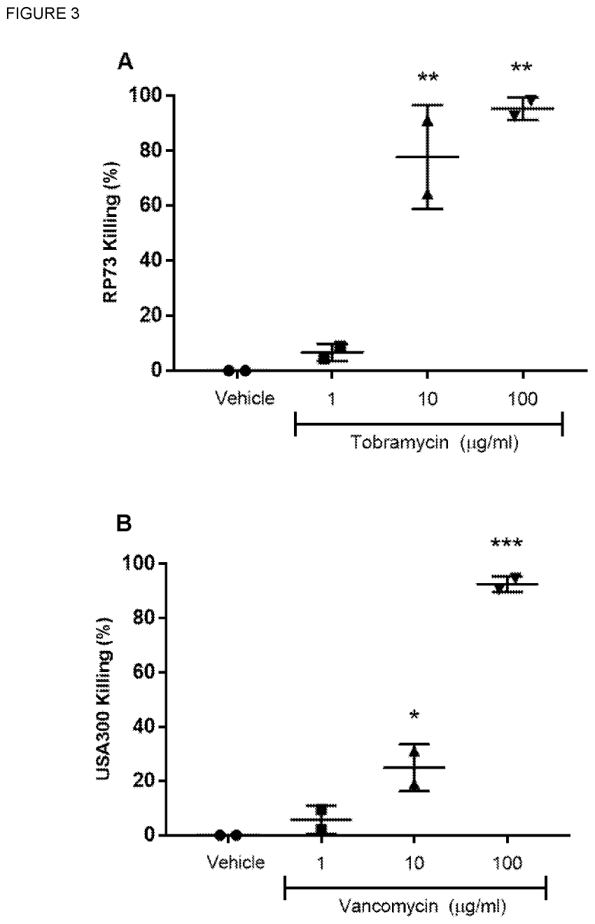

Initial experiments were performed to produce a dose response for said most common antibiotics to be used against the bacterial strains RP73 (Tobramycin) and USA300 (Vancomycin) at 1, 10 and 100 μg/ml over 2 hours. The multidrug resistant P. aeruginosa strain RP73 was only significantly killed at Tobramycin concentrations of 10 and 100 μg/ml (1 μg/ml: 6.60±2.22%; 10 μg/ml: 77.78±13.40%, P<0.01; 100 μg/ml: 95.37±2.87%, P<0.01, FIG. 3A).

The community acquired strain of MRSA USA300 was only significantly killed at Vancomycin concentrations of 10 and 100 μg/ml (1 μg/ml: 5.73±3.70%; 10 μg/ml: 250.5±6.13%, P<0.05; 100 μg/ml: 92.58±2.01%, P<0.01, FIG. 3B).

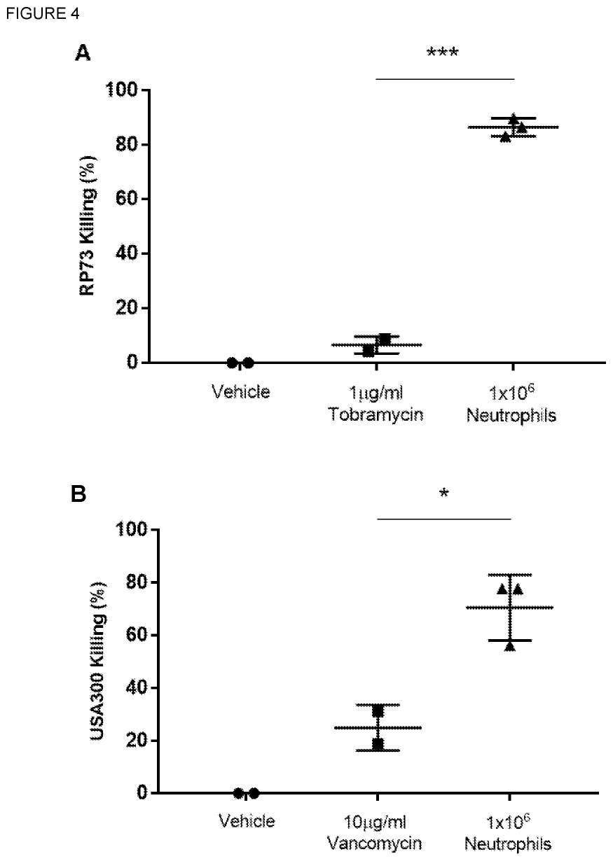

Both of these antibiotics are known to have cytotoxic side effects when given at high doses as demonstrated by the recommended serum trough concentrations of 2 μg/ml for Tobramycin and 10-20 μg/ml for Vancomycin by the British National Formulary. Therefore, in experiments comparing the BKA of neutrophils against the antibiotics, 1 μg/ml was chosen for Tobramycin and 10 μg/ml for Vancomycin. Neutrophils cultured from donors 12, 16 and 19 were selected for the comparison against antibiotic treatment as they had previously demonstrated superior BKA activity compared to the neutrophils cultured from other donors. (FIG. 4).

When compared to the standard of care (SOC) serum trough dose of Tobramycin (1 μg/ml) against the tobramycin resistant strain of P. aeruginosa RP73, neutrophils demonstrated significantly enhanced bacterial killing at 2 hours (1 μg/ml Tobramycin: 6.60±2.22% vs. 1×10⁶ Neutrophils: 86.51±1.89%, P<0.001, FIG. 4A).

Similarly, neutrophils with superior BKA demonstrated significantly increased levels of killing of the Gram-positive bacterial strain of MRSA USA300 when compared to the SOC serum trough dose of Vancomycin at 2 hours (10 μg/ml Tobramycin: 25.05±6.13% vs. 1×10⁶ Neutrophils: 70.55±7.18%, P<0.05, FIG. 4B).

Advantageously, this shows that granulocytes (and preferably neutrophils) cultured from donors shown to produce granulocytes with higher BKA are particularly effective in the treatment of bacterial infections. This advantageous property is in contrast to the standard (chemical) antibiotics which show lower bacterial killing and are known to be associated with side effects, such as cytotoxic side effects (even at the doses typically used in the clinic).

### Example 4

### Demonstrating Variable BKA in Donor Derived Neutrophils

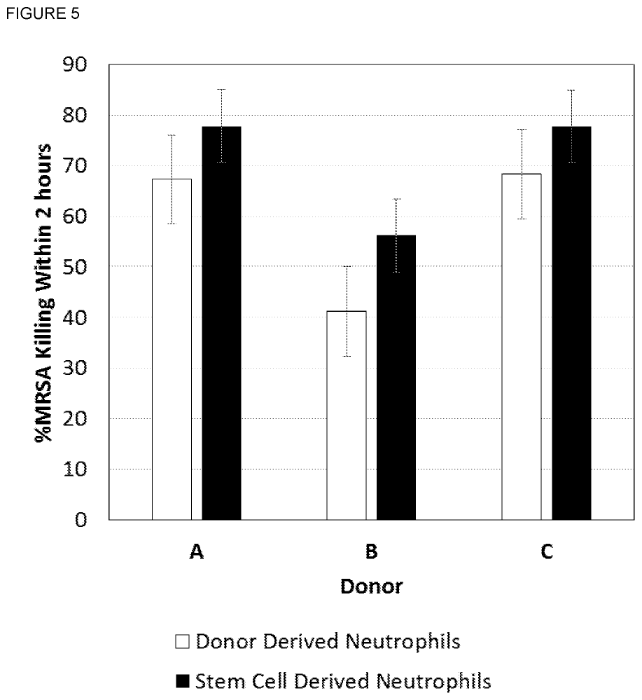

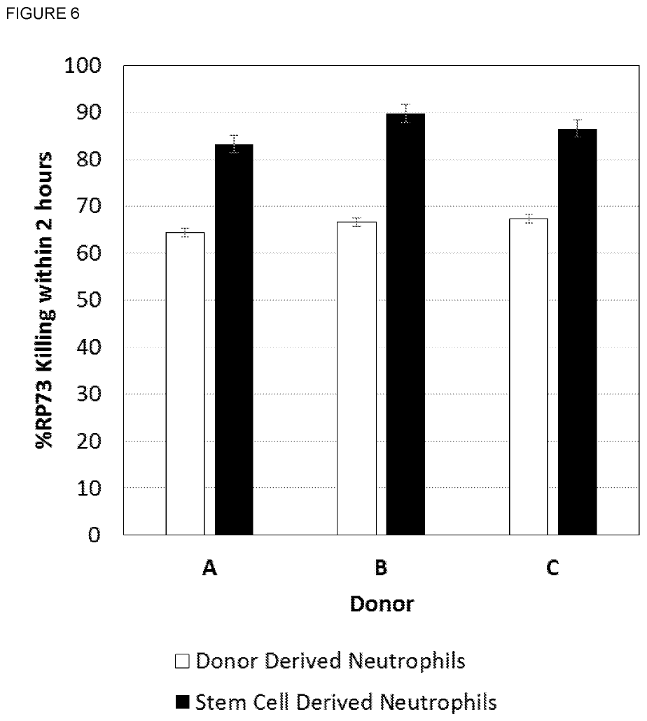

FIGS. 5 and 6 show the MRSA and P. aeruginosa RP73 (respectively) cytotoxicity (recorded by the BKA assay described in Example 1) for different donors. This assay is able to demonstrate differences in BKA between neutrophils from different donors. The assay can also demonstrate the most suitable donor based on the bacteria type to be targeted—donors D12 and D19 provided neutrophils with the highest BKA against MRSA, whereas donor D16 provided donors with the highest BKA against P. aeruginosa RP73.

### Example 5

### Demonstrating BKA of Stem Cell Derived Neutrophils

Demonstrating that BKA of Neutrophils is Genetically Encoded

Neutrophils isolated from three different donors (DDNs) mentioned above, as well as stem cell derived neutrophils (SCDNs) derived from CD34+ stem cells of the same donors according to standard techniques were tested for BKA as described in Example 1.

FIGS. 5 and 6 show the percentage cytotoxicity recorded by the BKA assay (after 2 hours) against MRSA and P. aeruginosa RP73 for DDNs and SCDNs from donors A, B and C. Surprisingly, the SCDNs demonstrated a BKA which had a highly similar profile to that of the DDNs from the same donor, demonstrating that BKA is encoded at the genetic level. Donors A and C provided neutrophils (and SCDNs) with the highest BKA against MRSA, while donor B provided neutrophils (and SCDNs) having lower BKA against MRSA.

Interestingly, the converse was demonstrated in a BKA assay with P. aeruginosa RP73, in which donor B provided neutrophils with the highest BKA, demonstrating the suitability for this method in optimising selection of donors based on pathogen type.

Results for P. aeruginosa RP73 are summarised in Table 1.

TABLE 1

| | Bacterial Killing (%) | | | | | | | |
|---|---|---|---|---|---|---|---|---|
| | Tobramycin | | Donor | | | Stem Cell | | |
| | (μg/ml) | | Derived Neutrophils | | | Derived Neutrophils | | |
| RP73 | 1 | 10 | A | B | C | A | B | C |
| RP73 | 6.60 | 77.78 | 64.37 | 66.58 | 67.26 | 83.23 | 89.76 | 86.55 |

Results for MRSA are summarised in Table 2.

TABLE 2

| | Bacterial Killing (%) | | | | | | | |
|---|---|---|---|---|---|---|---|---|
| | Vancomycin | | Donor | | | Stem Cell | | |
| | (μg/ml) | | Derived Neutrophils | | | Derived Neutrophils | | |
| MRSA | 1 | 10 | A | B | C | A | B | C |
| MRSA | 5.73 | 25.05 | 67.23 | 41.23 | 68.25 | 77.78 | 56.19 | 77.69 |

This demonstrates that donors found to have neutrophils (e.g. DDNs) with a high BKA may also be used as a source of CD34+ stem cells which can be differentiated into neutrophils (e.g. SCDNs) with similarly high BKA.

Additionally, the results demonstrate that stem cells from different donors can a) be differentiated in vitro to produce neutrophils that demonstrate bacteria killing abilities, and b) that this bacteria killing activity varies by the source donor. Interestingly, the bacterial killing activity varies not only by the source donor, but also by the bacteria type (donor B was the best donor for RP73, but not for MRSA).

The results support the fact that the bacteria killing activity (BKA) by the innate immune system varies by individual and that the same innate variance in BKA seen in neutrophils taken directly from donors is also shown in a donor's stem cells. By selecting donors with proven high bacteria killing activity of their innate immune system, and using their stem cells (i.e. haematopoietic stem cells) for ex vivo expansion and differentiation, a cell bank can be created with said stem cells/neutrophils with high bacteria killing activity to be used in the treatment of an infection.

### Example 6

### Isolation of High-Density Neutrophils

10 ml of heparinized (20 U/ml) human blood is mixed with an equal volume of 3% Dextran T500 in saline and incubated for 30 minutes at room temperature to sediment erythrocytes. A 50 ml conical polypropylene tube is prepared with 10 ml sucrose 1.077 g/ml and slowly layered with a leukocyte-rich supernatant on top of the 1.077 g/ml sucrose layer prior to centrifuging at 400×g for 30 minutes at room temperature without brake. The high-density neutrophils (HDN) appear in the pellet. Low-density neutrophils (LDN) co-purify with monocytes and lymphocytes at the interface between the 1.077 g/ml sucrose layer and plasma.

The HDNs may be tested in a BKA assay described herein. Haematopoietic cells are suitably obtained from a donor having HDNs.

### Example 7

Differentiation of Induced Pluripotent Stem Cells (iPSCs) into Neutrophils with High BKA

A donor comprising neutrophils with high BKA is identified. A somatic cell (e.g. fibroblast) is isolated from the donor and used to establish a culture of iPSCs. The iPSCs are differentiated into mature neutrophils, e.g. using the protocol as described by Sweeney C L, Merling R K, Choi U, Priel D B, Kuhns D B, Wang H and Malech H L, Generation of functionally mature neutrophils from induced pluripotent stem cells. Neutrophil Methods and Protocols, Methods in Molecular Biology. 2014; 1124:189-206, and Sweeney et al (2016), Stem Cells, 34(6), 1513-1526 (the teaching of which is incorporated herein by reference).

The resulting mature neutrophils are shown to have similar BKA levels to those of the DDNs and SCDNs from HSCs from the same donor.

The mature neutrophils are subsequently injected into the donor from which the iPSCs have been originally derived, and do not provoke any immune response.

### Example 8

Treatment of a Patient with MRSA

A patient diagnosed with an MRSA infection is tested for suitability for treatment with the granulocytes of the invention. A blood sample is obtained from the patient and analysed in a BKA assay (according to the method of Example 1).

The patient's granulocytes are unsuitable for treatment of infections and therefore the patient is found to be suitable for treatment with the granulocytes of the invention. The patient's details are processed through a cell database for a cell bank and suitable granulocytes identified (suitable granulocytes are from a donor with the same blood group as the patient and that demonstrated>41.23% MRSA BKA).

The patient is treated once a week with the granulocytes of the invention. An infusion of 2×10⁹ granulocytes is administered to the patient in the first week and the dose increased incrementally for 3 subsequent weeks to a final dose of 2×10¹¹ granulocytes in week 4. A change in symptoms (such as: redness and swelling of the skin; pus; pain; aches; confusion; fever; chills; and dizziness) is monitored. After 4 weeks of treatment the symptoms are vastly reduced/eliminated.

### Example 9

Treatment of a Patient with Vancomycin-Resistant Enterococcus (VRE)

A patient diagnosed with a vancomycin-resistant Enterococcus infection is tested for suitability for treatment with the granulocytes of the invention. A blood sample is obtained from the patient and analysed in a BKA assay (according to the method of Example 1).

The patient is found to be suitable for treatment with the granulocytes of the invention. The patient's details are processed through a cell database for a cell bank and suitable granulocytes identified (suitable granulocytes are from a donor with the same blood group as the patient and that demonstrated>41.23% MRSA BKA).

The patient is treated once a week with the granulocytes of the invention. An infusion of 2×10⁹ granulocytes is administered to the patient in the first week and the dose increased incrementally for 3 subsequent weeks to a final dose of 2×10¹¹ granulocytes in week 4. A change in symptoms (such as: redness and swelling of the skin; elevated heart rate; malaise; nausea; fever; chills) is monitored. After 4 weeks of treatment the symptoms are vastly reduced/eliminated.

### Example 10

### Infrared-Light Stimulates Neutrophil BKA

A patient is diagnosed with a diabetic foot ulcer that is not healing. The patient is administered with the granulocytes of the invention and to increase the function and proliferation of the granulocytes is also subjected to short bursts of high-power near-infrared light (1000 W) for 30 minutes 3 times a day for 4 weeks. The infrared light is directed at the wound site. After the treatment course, the ulcer shows signs of significant healing, which is surprisingly improved when compared to a patient administered the granulocytes without the infrared light treatment.

### Example 11

### Isolation of Stem Cell-Derived Neutrophils

Stem cell-derived neutrophils (SCDN) were synthesised according to standard techniques and cultured ex vivo for 25 days following Ficoll-separation to obtain PBMCs and CD34+ isolates from ten one-off donor buffy leukocyte cones. Aliquots of the SCDN (50×10⁶/ml) were frozen at −80° C. in cryopreservative (10% FBS in DMSO).

Evaluation of Healthy Cell Killing Using the xCelligence Assay

SCDN were thawed and decanted into complete Dulbecco's modified Eagle's medium (DMEM) before incubation for 72 hours with ‘healthy’ breast epithelial cells (MCF-12F) (commercially available from the American Type Culture Collection—United Kingdom (U.K.), Guernsey, Ireland, Jersey and Liechtenstein, LGC Standards, Queens Road, Teddington, Middlesex TW11 0LY, UK). Cell killing activity was recorded regularly throughout the 72 hour culture period by xCelligence Assay.

The ACEA Biosciences xCELLigence RTCA DP Analyzer system® was used and the manufacturer's instructions were followed. The xCELLigence System is a real-time cell analyser, allowing for label-free and dynamic monitoring of cellular phenotypic changes continuously by measuring electrical impedance. The system measures impedance using interdigitated gold microelectrodes integrated into the bottom of each well of the tissue culture E-Plates. Impedance measurements are displayed as Cell Index (CI) values, providing quantitative information about the biological status of the cells, including viability. Impedance-based monitoring of cell viability correlates with cell number and MTT-based readout. The kinetic aspect of impedance-based cell viability measurements provides the necessary temporal information when neutrophils are used to induce cytotoxic effects. In particular, the xCELLigence System can also pinpoint the optimal time points when the neutrophils achieve their maximal effect (where such data is desired), as indicated by the lowest Cl values, in cytotoxicity and cell death assays. 6,000 healthy cells (MCF-12F) are placed in the bottom of a 16 well plate (the system can read up to 3 plates simultaneously). For the first few hours after cells have been added to a well there is a rapid increase in impedance. This is caused by cells falling out of suspension, depositing onto the electrodes, and forming focal adhesions. If the initial number of cells added is low and there is empty space on the well bottom, cells will proliferate, causing a gradual yet steady increase in Cl. When cells reach confluence the Cl value plateaus, reflecting the fact that the electrode surface area that is accessible to bulk media is no longer changing. At this point, which is called the ‘normalization point’, the neutrophils (60,000 cells) are added (giving a 10:1 effector:target ratio) and incubated at 37° C. The percentage of cytolysis is readily calculated using a simple formula: Percentage of cytolysis=((Cell Index_{no effector}−Cell Index_{effector})/Cell Index_{no effector})×100.

SCDNs that demonstrated>41.23% BKA against MRSA by 2 hours in the assay carried out as per Example 1, and <10% non-bacterial target killing (i.e. killed <10% of ‘healthy’ breast epithelial cells (MCF-12F)) were designated high BKA neutrophils and cells that demonstrated less than or equal to 41.23% BKA against MRSA were designated low BKA control neutrophils.

Table 3 shows BKA by 2 hours.

| BKA type | Donor ID | BKA % MRSA | BKA % RP73 |
|---|---|---|---|
| High BKA Neutrophil | A | 77.78 | 83.23 |
| High BKA Neutrophil | B | 56.19 | 89.76 |
| High BKA Neutrophil | C | 77.69 | 86.55 |
| High BKA Neutrophil | D | 68.25 | 67.26 |
| Low BKA Control | E | 41.23 | 28.67 |
| Low BKA Control | F | 10.21 | 17.52 |

### Proteomic Analysis

Neutrophils were lysed and underwent sonication and were analysed using the Pierce bicinchoninic acid (BCA) protein assay according to manufacturer's instructions (commercially available from ThermoFisher, Waltham, MA, catalgoue number: 23225) to determine protein concentration. Typically samples contained around 20 micrograms of protein in <500 μl. Samples were digested, desalted and lyophilised prior to liquid chromatography and mass spectrometry (LC-MS/MS) using a Thermo Q-Exactive (Orbitrap) Plus Mass Spectrometer (Thermo Scientific™). First, chromatography separates the peptides in solution, the smaller hydrophilic peptides come off the column in the first fraction, and bigger hydrophobic peptides come off last over a 2 hour period. Secondly, a strongly acidic pH2 solution ensures all peptides have protons and are thus given a positive charge, the Mass Spectrometer only allows through positively charged ions of a given fraction to hit the detector. The Orbitrap device fluctuates between isolate and fragment, at around 20 Hz so the least ‘sticky’ peptides of a given mass/charge ratio are quantified first. The fluctuations are proportional to the intensity of the peptides detected, thus providing protein quantities for each cell type.

Bioinformatics was performed using the online DAVID system (Huang D W, Sherman B T, Lempicki R A. Bioinformatics enrichment tools: paths toward the comprehensive functional analysis of large gene lists. Nucleic Acids Res. 2009; 37:1-13; and Huang D W, Sherman B T, Lempicki R A. Systematic and integrative analysis of large gene lists using DAVID bioinformatics resources. Nat Protoc. 2009; 4:44-57).

Advantageously, the high BKA neutrophils showed significant upregulation of a number of polypeptides when compared to low BKA controls.

The following polypeptides (and thus genes) were upregulated compared to low BKA controls:
S100A9, S100A8, ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2.

The following polypeptides (and thus genes) were downregulated compared to low BKA controls:
ANXA1 and PPP3CB.

Table 4 presents a number of polypeptides with changed expression in high BKA cells compared to the typical low BKA cells.

| Polypeptide | log2 (High BKA/Typical) | p-value (t-test) |
|---|---|---|
| GM2A | 1.943649 | 7.66E−06 |
| PLEC | 0.855651 | 0.000199 |
| CYBB | 1.609576 | 0.00024 |
| DOCK8 | 1.454172 | 0.000455 |
| ATG7 | 1.163505 | 0.000737 |
| SLC2A1 | 1.4505 | 0.001259 |
| S100A9 | 1.435349 | 0.001681 |
| ACSL1 | 1.065324 | 0.001746 |
| CTSG | 2.155855 | 0.002295 |
| PSMB2 | 1.058384 | 0.002481 |
| ATM | 2.011409 | 0.002754 |
| BCAP31 | 2.707073 | 0.003322 |
| S100A8 | 0.699763 | 0.003776 |
| ITGB1 | 1.143777 | 0.005138 |
| TAPBP | 1.277103 | 0.005596 |
| COMP | 0.99287 | 0.005695 |
| SYK | 1.722188 | 0.006851 |
| GZMK | 1.8696069 | 0.040591 |
| IKBKB | 2.2958852 | 0.017181 |
| PPP3CB | −1.36037 | 0.000127 |
| ANXA1 | −1.17725 | 0.000792 |
| PERM | 2.00962408 | 0.0379030 |
| RAC1 | 1.9456240 | 0.0100812 |
| CAP37 | 2.70390702 | 0.01459156 |

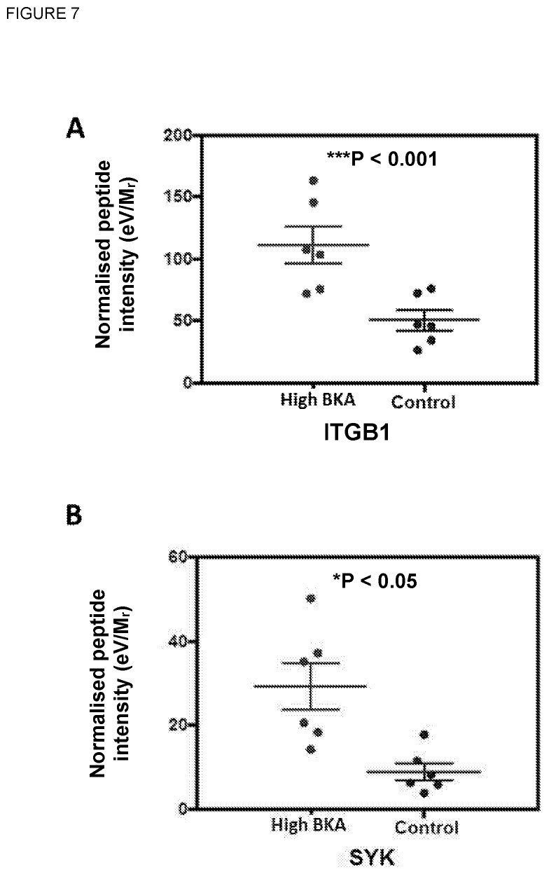

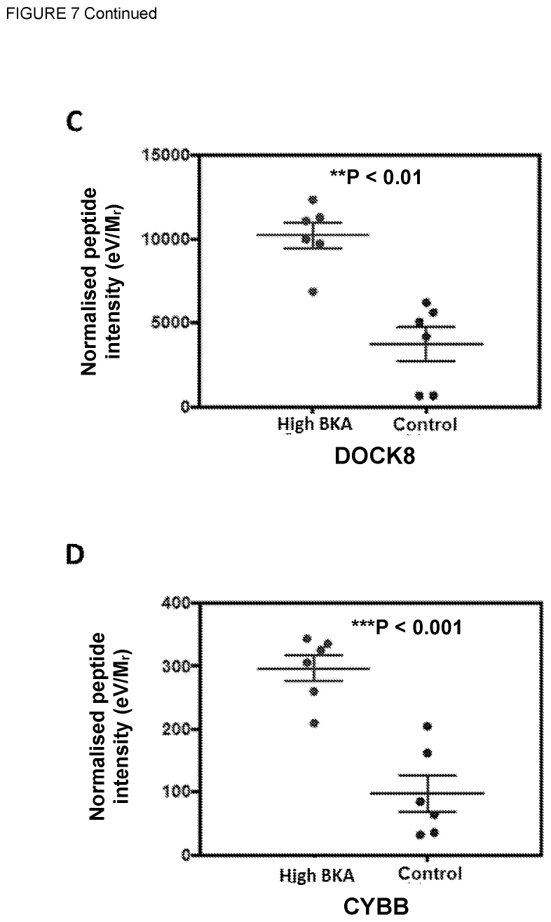

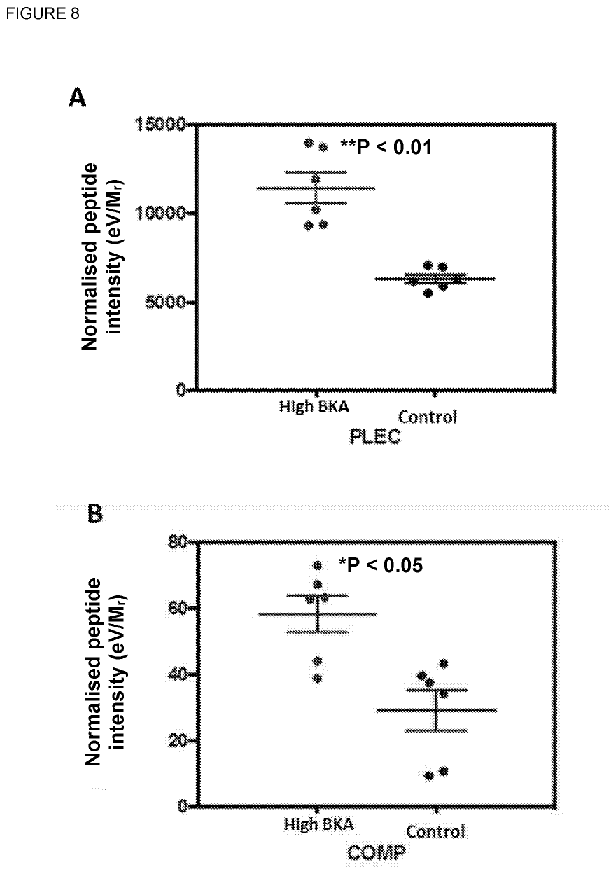

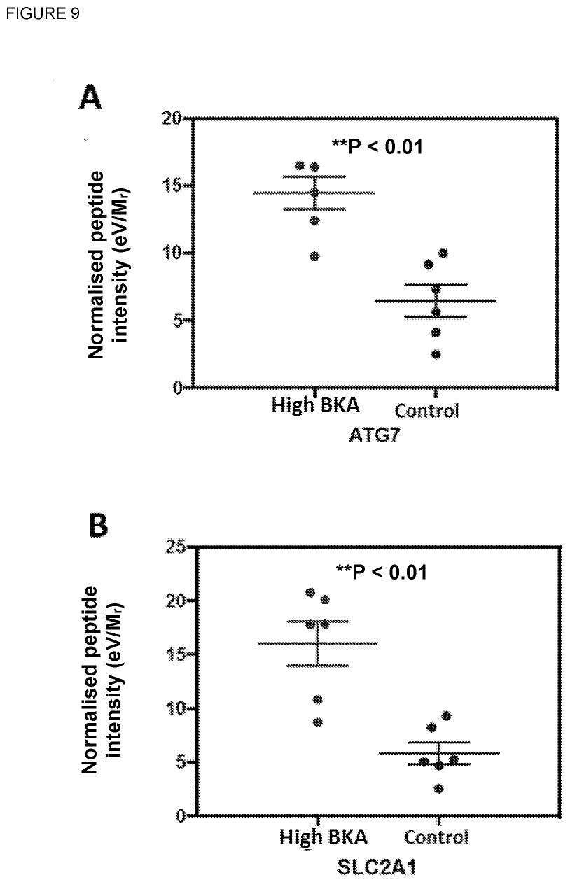

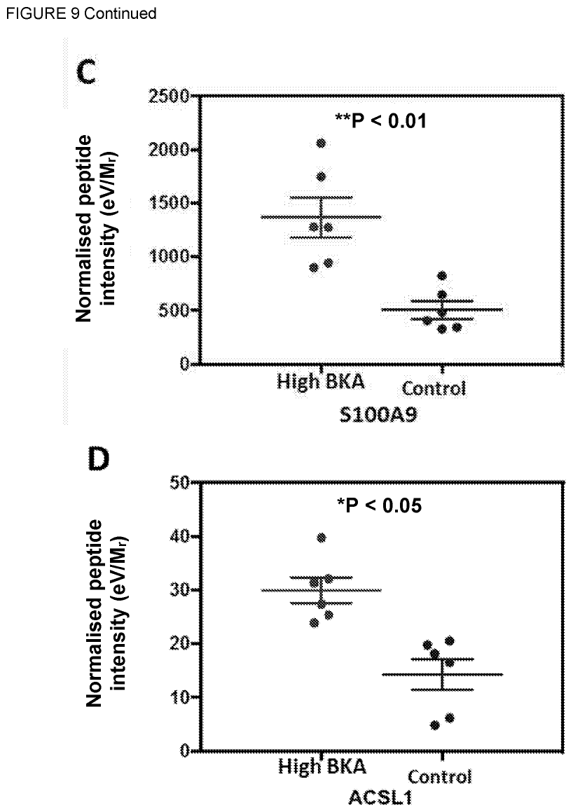

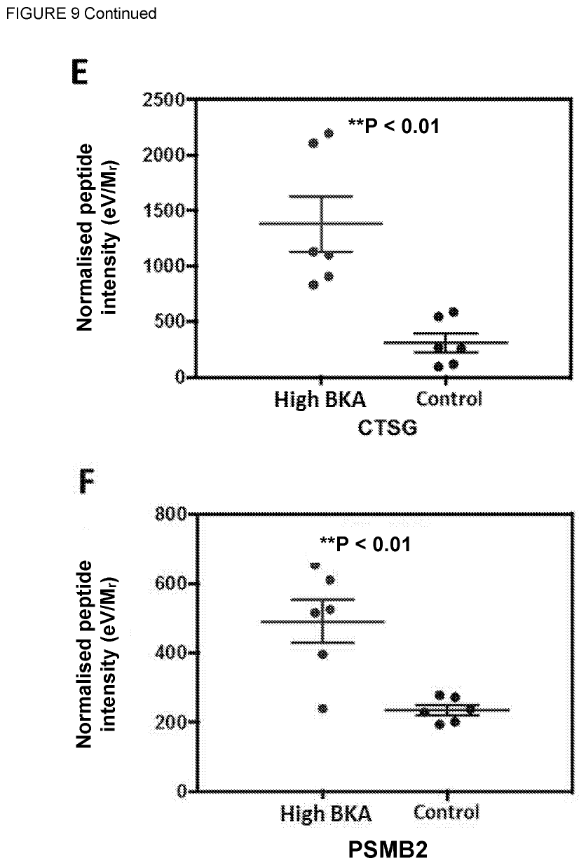

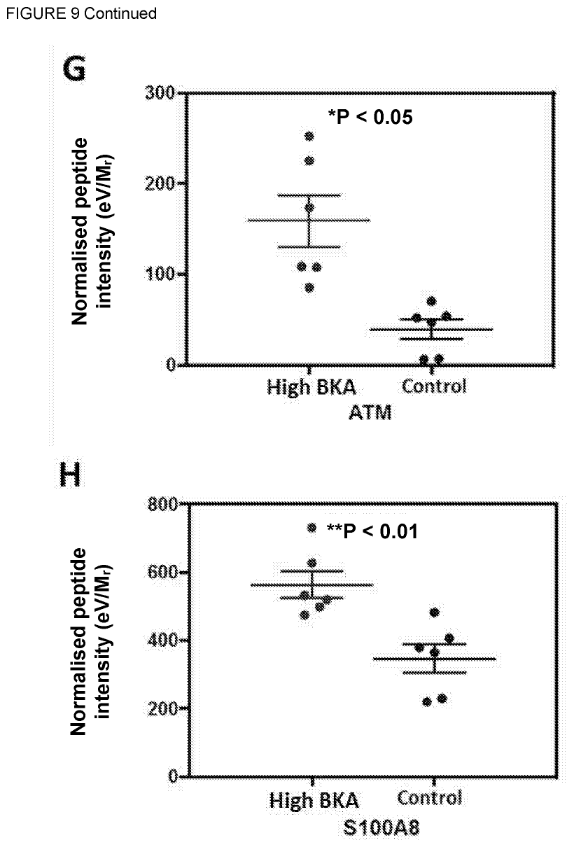

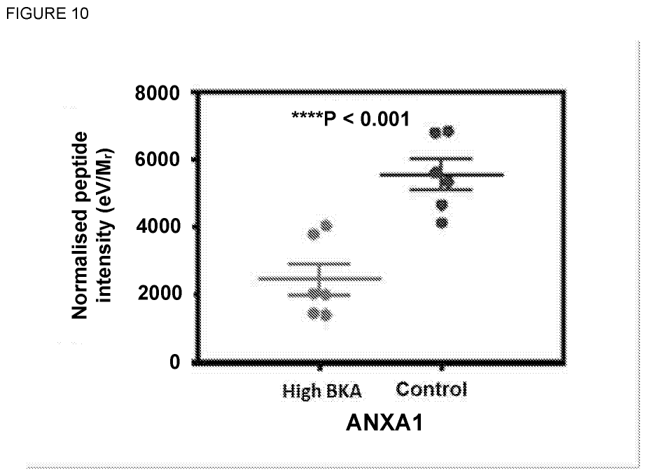

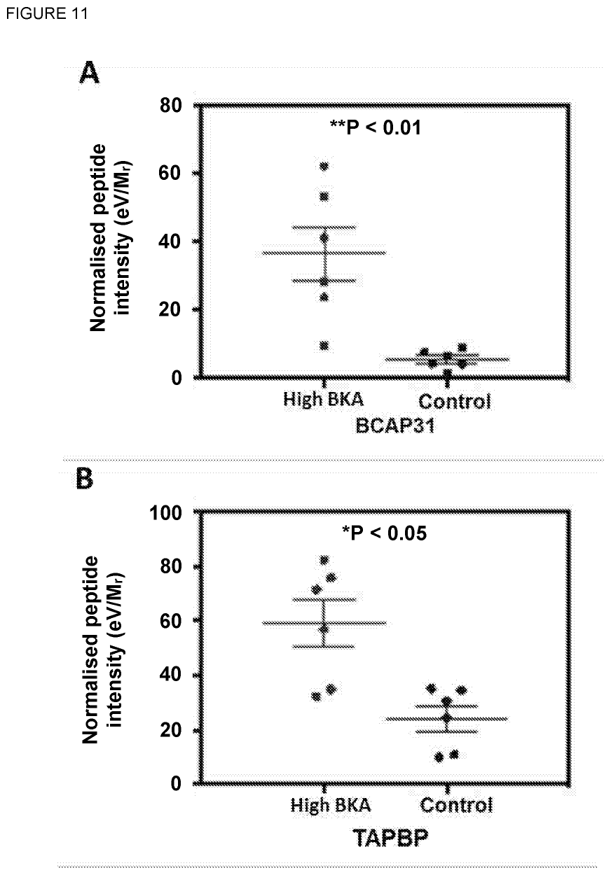

The results are presented graphically in FIGS. 7-11 for a number of the proteins, including two-way ANOVA statistical analyses. Advantageously, said protein levels (and thus gene expression levels) provided a robust means for identifying and differentiating high BKA cells from low BKA cells.

Advantageously, the expression of many of the genes (i.e. at the protein level) was highly statistically-significantly different (e.g. GM2A) between high BKA cells and low BKA cells, indicating that high BKA granulocytes could be identified using just one of the indicated genes.

### Example 12

Extracting Haematopoietic Stem Cells from Peripheral Blood

Upon giving consent the donors are given a granulocyte-colony stimulating factor (G-CSF) and/or a granulocyte-macrophage colony-stimulating factor (GM-CSF), e.g. Neupogen® (commercially available from Amgen Inc. USA) to help harvest peripheral haematopoietic stem cells with minimal possible discomfort to donors. Cell surface polypeptide markers are used for identifying long-lasting multipotent stem-cells. Suitably markers may include CD 34⁺, CD59⁺, Thy1⁺, CD38^{low/-}, C-kit^{-/low}, and lin⁻.

### Example 13

### Expansion and Differentiation of Haematopoietic Cells

The haematopoietic cells (e.g. haematopoietic stem cells) are stimulated using a supernatant growth factor suspension, to either develop more stem cells or differentiate into precursor cells (e.g. myeloid or granulocyte progenitor cells) or granulocytes. Suitable neutrophil synthesis methods are disclosed in Lieber et al, Blood, 2004 Feb. 1; 103(3):852-9, and Choi et al, Nat. Protoc., 2011 March; 6(3):296-313.

The protocol is composed of four major stages:
culturing and proliferation of haematopoietic cells;
short-term expansion of multipotent myeloid progenitors with a high dose of granulocyte-macrophage colony-stimulating factor (GM-CSF), a granulocyte colony-stimulating factor (G-CSF), a human growth hormone (HGH); serotonin, vitamin C, vitamin D, glutamine (Gln), arachidonic acid, AGE-albumin, interleukin-3 (IL-3), interleukin 8 (IL-8), Interleukin-4 (IL-4), Interleukin-6 (IL-6), interleukin-18 (IL-18), TNF-alpha, Flt-3 ligand, thrombopoietin, foetal bovine serum (FBS), or combinations thereof; and
directed differentiation of myeloid progenitors into neutrophils, eosinophils, dendritic cells (DCs), Langerhans cells (LCs), macrophages and osteoclasts.

### Example 14

Preparation of Cell Banks Haematopoietic stem cells, granulocyte precursor cells and granulocytes obtainable therefrom, are cryogenically frozen and stored in appropriate cell banks.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1.-68. (canceled)

69. A method of treating an infection in a subject comprising:
a. administering neutrophils or stem cells to the subject, wherein the neutrophils or stem cells comprise:
i. increased expression of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from neutrophils or stem cells unsuitable for treating an infection; and/or
ii. decreased expression of ANXA1 and/or PPP3CB when compared to a reference standard, wherein the reference standard is from neutrophils or stem cells unsuitable for treating an infection; or
b. administering stem cells which are capable of differentiating into said neutrophils to the subject,
thereby treating the infection in the subject.

70. The method of claim 69, wherein the neutrophils are differentiated from stem cells that have been derived from a sample from a donor.

71. The method of claim 69, wherein the infection comprises a bacterial infection, a fungal infection, a viral infection, a macroparasitic infection.

72. The method of claim 71, wherein the bacterial infection comprises an antibiotic resistant bacterial infection.

73. The method of claim 72, wherein the antibiotic resistant bacterial infection is selected from methicillin-resistant Staphylococcus aureus (MRSA), multidrug resistant Gram-negative bacteria (MDRGN bacteria), vancomycin-resistant Enterococcus (VRE), multi-drug-resistant Mycobacterium tuberculosis (MDR-TB), carbapenem-resistant Enterobacteriaceae (CRE) gut bacteria, or a combination thereof.

74. The method of claim 71, wherein the viral infection is selected from one or more viral families selected from Adenoviridae, Picornaviridae, Herpesviridae, Coronaviridae, Hepadnaviridae, Flaviviridae, Retroviridae, Orthomyxoviridae, Paramyxoviridae, Papovaviridae, Polyomavirus, Rhabdoviridae, Togaviridae and Bunyaviridae.

75. The method of claim 71, wherein the viral infection is selected from one or more of HIV-1 (Human immunodeficiency virus), HIV-2, Junin virus, BK virus, Machupo virus, Sabiá virus, Varicella zoster virus (VZV), Alphavirus, Colorado tick fever virus (CTFV), Rhinoviruses, Crimean-Congo hemorrhagic fever virus, Cytomegalovirus, Dengue virus, Ebolavirus (EBOV), Parvovirus B19, Human herpesvirus 6 (HHV-6), Human herpesvirus 7 (HHV-7), Enteroviruses (e.g. EV71), Coxsackie A virus, Sin Nombre virus, Heartland virus, Hanta virus, Hendra virus, Hepatitis A virus, Hepatitis B virus, Hepatitis C virus, Hepatitis D Virus, Hepatitis E virus, Herpes simplex virus 1 and 2 (HSV-1 and HSV-2), Human bocavirus (HBoV), Human metapneumovirus (hMPV), Human papillomaviruses, Human parainfluenza viruses (HPIV), Epstein-Barr virus (EBV), Lassa virus, Lymphocytic choriomeningitis virus (LCMV), Marburg virus, Measles virus, Middle East respiratory syndrome coronavirus, Molluscum contagiosum virus (MCV), Monkeypox virus, Mumps virus, Nipah virus, Norovirus, Poliovirus, JC virus, Respiratory syncytial virus (RSV), Rhinovirus, Rift Valley fever virus, Rotavirus, Rubella virus, SARS coronavirus, Variola major, Variola minor, Venezuelan equine encephalitis virus, Guanarito virus, West Nile virus, Yellow fever virus, and Zika virus.

76. The method of claim 69, wherein the stem cells comprise an induced pluripotent stem cell, a haematopoietic stem cell, or a precursor cell.

77. A method for obtaining a stem cell or neutrophil population for treating an infection, said method comprising:
a. admixing neutrophils obtainable from a donor with an infective agent or a cell infected by an infective agent;
b. incubating said admixture;
c. measuring the % of infective agent or cells infected by an infective agent killed in said admixture; and
d. obtaining stem cells or neutrophils from a sample from said donor when the % of infective agent or cells infected by an infective agent killed in the admixture is greater than the % of infective agent or cells infected by an infective agent killed in a control sample, wherein the control sample comprises an infective agent or a cell infected by an infective agent of the same type and neutrophils obtainable from a different donor.

78. The method of claim 77, wherein the neutrophil kills greater than 41.23% of the infective agent or cells infected by an infective agent in the admixture.

79. The method of claim 77, wherein the infective agent comprises a bacterium, fungi, virus, or macroparasite.

80. The method of claim 77, wherein the infective agent comprises a bacterium or virus.

81. The method of claim 77, wherein the stem cells or neutrophils have
i. increased expression of one or more genes selected from: GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from a neutrophil unsuitable for treating an infection; and/or
ii. decreased expression of ANXA1 and/or PPP3CB when compared to a reference standard, wherein the reference standard is from a neutrophil unsuitable for treating an infection.

82. The method of claim 81, wherein the expression level is measured by proteomic techniques.

83. The method of claim 81, wherein the expression level is measured by transcriptomic techniques.

84. The method of claim 77, wherein the stem cells comprise an induced pluripotent stem cell, a haematopoietic stem cell, or a precursor cell.

85. A method of formulating an infection killing formulation comprising:
selecting stem cells or neutrophils obtainable by the method of claim 77; and
formulating the selected neutrophils or stem cells within a carrier;
thereby formulating the infection killing formulation.

86. The method of claim 85, wherein the stem cells comprise an induced pluripotent stem cell, a haematopoietic stem cell, or a precursor cell.

87. An infection killing formulation produced according to the method of claim 85.

88. The infection killing formulation of claim 87, wherein the stem cells comprise an induced pluripotent stem cell, a haematopoietic stem cell, or a precursor cell.
